SEQUENCE LISTING (244 sequences)

SEQ ID NO: 1 - DNA, 28 bp, Artificial Sequence
  primer 204
attgacgtcaatgggagtttgttttggc

SEQ ID NO: 2 - DNA, 27 bp, Artificial Sequence
  primer 469
gcatttaggtgacactatagaataggg

SEQ ID NO: 3 - DNA, 55 bp, Artificial Sequence
  primer 894
gatcagggtgaggaacagcacactttaccaatgaaagtcgtgaccaggccacgtt

SEQ ID NO: 4 - DNA, 55 bp, Artificial Sequence
  primer 895
agctaacgtggcctggtcacgactttcattggtaaagtgtgctgttcctcaccct

SEQ ID NO: 5 - DNA, 61 bp, Artificial Sequence
  primer 944
cctttttaacccatcacatatacctgccgttctcaggtcactaatactatctaagtagttg

SEQ ID NO: 6 - DNA, 64 bp, Artificial Sequence
  primer 945
cgtttggattgcaactggtctattttcctctcgacaaatgattttattttgactaataatgacc

SEQ ID NO: 7 - DNA, 31 bp, Artificial Sequence
  primer 1021
gcagcagtgcagaggcgccagcagcagcgag

SEQ ID NO: 8 - DNA, 31 bp, Artificial Sequence
  primer 1022
ctcgctgctgctggcgcctctgcactgctgc

SEQ ID NO: 9 - DNA, 32 bp, Artificial Sequence
  primer 1023
ctgccaggctgtacggcaaccagatcggcgac

SEQ ID NO: 10 - DNA, 32 bp, Artificial Sequence
  primer 1024
gtcgccgatctggttgccgtacagcctggcag

SEQ ID NO: 11 - DNA, 32 bp, Artificial Sequence
  primer 1025
ctgggccacaagagcgtgagcatggccgccag

SEQ ID NO: 12 - DNA, 32 bp, Artificial Sequence
  primer 1026
ctggcggccatgctcacgctcttgtggcccag

SEQ ID NO: 13 - protein, 357 aa, Bacteriophage HK022
  MISC_FEATURE: wt HK022 integrase
MGRRRSHERRDLPPNLYIRNNGYYCYRDPRTGKEFGLGRDRRIAITEAIQANIELLSGNRRESLIDRIKGADAITLHAWLDRYETILSERGIRPKTLLDYASKIRAIRRKLPDKPLADISTKEVAAMLNTYVAEGKSASAKLIRSTLVDVFREAIAEGHVATNPVTATRTAKSEVRRSRLTANEYVAIYHAAEPLPIWLRLAMDLAVVTGQRVGDLCRMKWSDINDNHLHIEQSKTGAKLAIPLTLTIDALNISLADTLQQCREASSSETIIASKHHDPLSPKTVSKYFTKARNASGLSFDGNPPTFHELRSLSARLYRNQIGDKFAQRLLGHKSDSMAARYRDSRGREWDKIEIDK

SEQ ID NO: 14 - protein, 357 aa, Artificial Sequence
  E174K mutant of the HK022 integrase
MGRRRSHERRDLPPNLYIRNNGYYCYRDPRTGKEFGLGRDRRIAITEAIQANIELLSGNRRESLIDRIKGADAITLHAWLDRYETILSERGIRPKTLLDYASKIRAIRRKLPDKPLADISTKEVAAMLNTYVAEGKSASAKLIRSTLVDVFREAIAEGHVATNPVTATRTAKSKVRRSRLTANEYVAIYHAAEPLPIWLRLAMDLAVVTGQRVGDLCRMKWSDINDNHLHIEQSKTGAKLAIPLTLTIDALNISLADTLQQCREASSSETIIASKHHDPLSPKTVSKYFTKARNASGLSFDGNPPTFHELRSLSARLYRNQIGDKFAQRLLGHKSDSMAARYRDSRGREWDKIEIDK

SEQ ID NO: 15 - DNA, 1071 bp, Artificial Sequence
  E174K mutant of the HK022 integrase
atgggcaggcggcggagccacgagcggagagacctgccccccaacctgtacatccggaacaacggctactactgctaccgggacccccggaccggcaaagagttcggcctgggccgggacaggcggatcgccatcaccgaggccatccaggccaacatcgagctgctgtccggcaaccggcgggagagcctgatcgaccggatcaagggcgccgacgccatcaccctgcacgcctggctggacagatacgagaccatcctgagcgagcggggcatccggcccaagaccctgctggactacgcctctaagatccgggccatcagacggaagctgcccgacaagcccctggccgacatcagcaccaaagaagtggccgccatgctgaacacctacgtggccgagggcaagagcgccagcgccaagctgatccggtccaccctggtggacgtgttccgggaggccatcgccgagggccacgtcgccaccaaccccgtgaccgccacccggaccgccaagagcaaagtgcggcggagcaggctgaccgccaacgagtacgtggccatctaccatgccgctgagcccctgcccatctggctgcggctggccatggacctggccgtggtgaccggccagagagtgggcgacctgtgccggatgaagtggagcgacatcaacgacaaccacctgcacatcgagcagagcaagaccggcgccaaactggccatccccctgaccctgaccatcgacgccctgaacatcagcctggccgataccctgcagcagtgcagagaggccagcagcagcgagaccatcatcgccagcaagcaccacgaccccctgagccccaagaccgtgagcaagtacttcaccaaggcccggaacgccagcggcctgagcttcgacggcaacccccccaccttccacgagctgcggagcctgtctgccaggctgtaccggaaccagatcggcgacaagttcgctcagcggctcctgggccacaagagcgacagcatggccgccagataccgggacagccggggacgggagtgggacaagatcgagatcgacaag

SEQ ID NO: 16 - DNA, 10 bp, Artificial Sequence
  Consensus sequence of B
  misc_feature (4)..(4): w is a or t
  misc_feature (5)..(10): n is null
cttwnnnnnn

SEQ ID NO: 17 - DNA, 10 bp, Artificial Sequence
  Consensus sequence of B′
  misc_feature (5)..(10): n is null
aaagnnnnnn

SEQ ID NO: 18 - DNA, 10 bp, Artificial Sequence
  Tay-Sachs Hexa3 O
  misc_feature (8)..(10): n is null
accaatgnnn

SEQ ID NO: 19 - DNA, 10 bp, Artificial Sequence
  Tay-Sachs Hexa7 O
  misc_feature (8)..(10): n is null
taaaaatnnn

SEQ ID NO: 20 - DNA, 10 bp, Artificial Sequence
  Ataxia ATM4 O
  misc_feature (8)..(10): n is null
gactcagnnn

SEQ ID NO: 21 - DNA, 10 bp, Artificial Sequence
  Ataxia ATM8 O
  misc_feature (8)..(10): n i s null
gtgaggtnnn

SEQ ID NO: 22 - DNA, 10 bp, Artificial Sequence
  Sickle cell anemia haem1 O
  misc_feature (8)..(10): n is null
tctgaacnnn

SEQ ID NO: 23 - DNA, 10 bp, Artificial Sequence
  Sickle cell anemia haem13 O
  misc_feature (8)..(10): n is null
gactaggnnn

SEQ ID NO: 24 - DNA, 10 bp, Artificial Sequence
  Lesch-Nyhan syndrome hgprt1 O
  misc_feature (8)..(10): n is null
tatccctnnn

SEQ ID NO: 25 - DNA, 10 bp, Artificial Sequence
  Lesch-Nyhan syndrome hgprt13 O
  misc_feature (8)..(10): n is null
cttttagnnn

SEQ ID NO: 26 - DNA, 21 bp, Artificial Sequence
  Tay-Sachs Hexa3
acactttaccaatgaaagtcg

SEQ ID NO: 27 - DNA, 21 bp, Artificial Sequence
  Tay-Sachs Hexa7
gaacttttaaaaataaagggc

SEQ ID NO: 28 - DNA, 21 bp, Artificial Sequence
  Ataxia ATM4
tttctttgactcagaaaggga

SEQ ID NO: 29 - DNA, 21 bp, Artificial Sequence
  Ataxia ATM8
tgacttagtgaggtaaagtaa

SEQ ID NO: 30 - DNA, 21 bp, Artificial Sequence
  Sickle cell anemia haem1 or hbb1
gtacttatctgaacaaaggag

SEQ ID NO: 31 - DNA, 21 bp, Artificial Sequence
  Sickle cell anemia haem13 or hbb13
tttctttgactaggaaaggga

SEQ ID NO: 32 - DNA, 21 bp, Artificial Sequence
  Lesch-Nyhan syndrome hgprt1
agtcttttatccctaaaggag

SEQ ID NO: 33 - DNA, 21 bp, Artificial Sequence
  Lesch-Nyhan syndrome hgprt13
aaactttcttttagaaaggtg

SEQ ID NO: 34 - DNA, 32 bp, Artificial Sequence
  Primer 1030
ggaccgccaagagcaaagtgcggcggagcagg

SEQ ID NO: 35 - DNA, 32 bp, Artificial Sequence
  Primer 1031
cctgctccgccgcactttgctcttggcggtcc

SEQ ID NO: 36 - DNA, 40 bp, Artificial Sequence
  Primer 1032
ctgggccgggacaggcggttcgccatcaccgaggccatcc

SEQ ID NO: 37 - DNA, 40 bp, Artificial Sequence
  Primer 1033
ggatggcctcggtgatggcgaaccgcctgtcccggcccag

SEQ ID NO: 38 - DNA, 51 bp, Artificial Sequence
  Primer 1051
atgtatttagaaaaataaacaaataggggtcgtgaggctccggtgcccgtc

SEQ ID NO: 39 - DNA, 52 bp, Artificial Sequence
  Primer 1052
atctcccgatccgtcgacgtcaggtggcacacctagccagcttgggtctccc

SEQ ID NO: 40 - DNA, 52 bp, Artificial Sequence
  Primer 1064
tcgagtctagagggcccgtttaaacccgctatggtgagcaagggcgaggagg

SEQ ID NO: 41 - DNA, 56 bp, Artificial Sequence
  Primer 1065
gtcaaggaaggcacgggggaggggcaaacaggacaaaccacaactagaatgcagtg

SEQ ID NO: 42 - protein, 357 aa, Artificial Sequence
  I43F mutant of the HK022 integrase
MGRRRSHERRDLPPNLYIRNNGYYCYRDPRTGKEFGLGRDRRFAITEAIQANIELLSGNRRESLIDRIKGADAITLHAWLDRYETILSERGIRPKTLLDYASKIRAIRRKLPDKPLADISTKEVAAMLNTYVAEGKSASAKLIRSTLVDVFREAIAEGHVATNPVTATRTAKSEVRRSRLTANEYVAIYHAAEPLPIWLRLAMDLAVVTGQRVGDLCRMKWSDINDNHLHIEQSKTGAKLAIPLTLTIDALNISLADTLQQCREASSSETIIASKHHDPLSPKTVSKYFTKARNASGLSFDGNPPTFHELRSLSARLYRNQIGDKFAQRLLGHKSDSMAARYRDSRGREWDKIEIDK

SEQ ID NO: 43 - DNA, 1071 bp, Artificial Sequence
  I43F mutant of the HK022 integrase
atgggcaggcggcggagccacgagcggagagacctgccccccaacctgtacatccggaacaacggctactactgctaccgggacccccggaccggcaaagagttcggcctgggccgggacaggcggttcgccatcaccgaggccatccaggccaacatcgagctgctgtccggcaaccggcgggagagcctgatcgaccggatcaagggcgccgacgccatcaccctgcacgcctggctggacagatacgagaccatcctgagcgagcggggcatccggcccaagaccctgctggactacgcctctaagatccgggccatcagacggaagctgcccgacaagcccctggccgacatcagcaccaaagaagtggccgccatgctgaacacctacgtggccgagggcaagagcgccagcgccaagctgatccggtccaccctggtggacgtgttccgggaggccatcgccgagggccacgtcgccaccaaccccgtgaccgccacccggaccgccaagagcgaagtgcggcggagcaggctgaccgccaacgagtacgtggccatctaccatgccgctgagcccctgcccatctggctgcggctggccatggacctggccgtggtgaccggccagagagtgggcgacctgtgccggatgaagtggagcgacatcaacgacaaccacctgcacatcgagcagagcaagaccggcgccaaactggccatccccctgaccctgaccatcgacgccctgaacatcagcctggccgataccctgcagcagtgcagagaggccagcagcagcgagaccatcatcgccagcaagcaccacgaccccctgagccccaagaccgtgagcaagtacttcaccaaggcccggaacgccagcggcctgagcttcgacggcaacccccccaccttccacgagctgcggagcctgtctgccaggctgtaccggaaccagatcggcgacaagttcgctcagcggctcctgggccacaagagcgacagcatggccgccagataccgggacagccggggacgggagtgggacaagatcgagatcgacaag

SEQ ID NO: 44 - protein, 357 aa, Artificial Sequence
  E264G mutant of the HK022 integrase
MGRRRSHERRDLPPNLYIRNNGYYCYRDPRTGKEFGLGRDRRIAITEAIQANIELLSGNRRESLIDRIKGADAITLHAWLDRYETILSERGIRPKTLLDYASKIRAIRRKLPDKPLADISTKEVAAMLNTYVAEGKSASAKLIRSTLVDVFREAIAEGHVATNPVTATRTAKSEVRRSRLTANEYVAIYHAAEPLPIWLRLAMDLAVVTGQRVGDLCRMKWSDINDNHLHIEQSKTGAKLAIPLTLTIDALNISLADTLQQCRGASSSETIIASKHHDPLSPKTVSKYFTKARNASGLSFDGNPPTFHELRSLSARLYRNQIGDKFAQRLLGHKSDSMAARYRDSRGREWDKIEIDK

SEQ ID NO: 45 - DNA, 1071 bp, Artificial Sequence
  E264G mutant of the HK022 integrase
atgggcaggcggcggagccacgagcggagagacctgccccccaacctgtacatccggaacaacggctactactgctaccgggacccccggaccggcaaagagttcggcctgggccgggacaggcggatcgccatcaccgaggccatccaggccaacatcgagctgctgtccggcaaccggcgggagagcctgatcgaccggatcaagggcgccgacgccatcaccctgcacgcctggctggacagatacgagaccatcctgagcgagcggggcatccggcccaagaccctgctggactacgcctctaagatccgggccatcagacggaagctgcccgacaagcccctggccgacatcagcaccaaagaagtggccgccatgctgaacacctacgtggccgagggcaagagcgccagcgccaagctgatccggtccaccctggtggacgtgttccgggaggccatcgccgagggccacgtcgccaccaaccccgtgaccgccacccggaccgccaagagcgaagtgcggcggagcaggctgaccgccaacgagtacgtggccatctaccatgccgctgagcccctgcccatctggctgcggctggccatggacctggccgtggtgaccggccagagagtgggcgacctgtgccggatgaagtggagcgacatcaacgacaaccacctgcacatcgagcagagcaagaccggcgccaaactggccatccccctgaccctgaccatcgacgccctgaacatcagcctggccgataccctgcagcagtgcagaggcgccagcagcagcgagaccatcatcgccagcaagcaccacgaccccctgagccccaagaccgtgagcaagtacttcaccaaggcccggaacgccagcggcctgagcttcgacggcaacccccccaccttccacgagctgcggagcctgtctgccaggctgtaccggaaccagatcggcgacaagttcgctcagcggctcctgggccacaagagcgacagcatggccgccagataccgggacagccggggacgggagtgggacaagatcgagatcgacaag

SEQ ID NO: 46 - protein, 357 aa, Artificial Sequence
  R319G mutant of the HK022 integrase
MGRRRSHERRDLPPNLYIRNNGYYCYRDPRTGKEFGLGRDRRIAITEAIQANIELLSGNRRESLIDRIKGADAITLHAWLDRYETILSERGIRPKTLLDYASKIRAIRRKLPDKPLADISTKEVAAMLNTYVAEGKSASAKLIRSTLVDVFREAIAEGHVATNPVTATRTAKSEVRRSRLTANEYVAIYHAAEPLPIWLRLAMDLAVVTGQRVGDLCRMKWSDINDNHLHIEQSKTGAKLAIPLTLTIDALNISLADTLQQCREASSSETIIASKHHDPLSPKTVSKYFTKARNASGLSFDGNPPTFHELRSLSARLYGNQIGDKFAQRLLGHKSDSMAARYRDSRGREWDKIEIDK

SEQ ID NO: 47 - DNA, 1071 bp, Artificial Sequence
  R319G mutant of the HK022 integrase
atgggcaggcggcggagccacgagcggagagacctgccccccaacctgtacatccggaacaacggctactactgctaccgggacccccggaccggcaaagagttcggcctgggccgggacaggcggatcgccatcaccgaggccatccaggccaacatcgagctgctgtccggcaaccggcgggagagcctgatcgaccggatcaagggcgccgacgccatcaccctgcacgcctggctggacagatacgagaccatcctgagcgagcggggcatccggcccaagaccctgctggactacgcctctaagatccgggccatcagacggaagctgcccgacaagcccctggccgacatcagcaccaaagaagtggccgccatgctgaacacctacgtggccgagggcaagagcgccagcgccaagctgatccggtccaccctggtggacgtgttccgggaggccatcgccgagggccacgtcgccaccaaccccgtgaccgccacccggaccgccaagagcgaagtgcggcggagcaggctgaccgccaacgagtacgtggccatctaccatgccgctgagcccctgcccatctggctgcggctggccatggacctggccgtggtgaccggccagagagtgggcgacctgtgccggatgaagtggagcgacatcaacgacaaccacctgcacatcgagcagagcaagaccggcgccaaactggccatccccctgaccctgaccatcgacgccctgaacatcagcctggccgataccctgcagcagtgcagagaggccagcagcagcgagaccatcatcgccagcaagcaccacgaccccctgagccccaagaccgtgagcaagtacttcaccaaggcccggaacgccagcggcctgagcttcgacggcaacccccccaccttccacgagctgcggagcctgtctgccaggctgtacggcaaccagatcggcgacaagttcgctcagcggctcctgggccacaagagcgacagcatggccgccagataccgggacagccggggacgggagtgggacaagatcgagatcgacaag

SEQ ID NO: 48 - protein, 357 aa, Artificial Sequence
  D336V mutant of the HK022 integrase
MGRRRSHERRDLPPNLYIRNNGYYCYRDPRTGKEFGLGRDRRIAITEAIQANIELLSGNRRESLIDRIKGADAITLHAWLDRYETILSERGIRPKTLLDYASKIRAIRRKLPDKPLADISTKEVAAMLNTYVAEGKSASAKLIRSTLVDVFREAIAEGHVATNPVTATRTAKSEVRRSRLTANEYVAIYHAAEPLPIWLRLAMDLAVVTGQRVGDLCRMKWSDINDNHLHIEQSKTGAKLAIPLTLTIDALNISLADTLQQCREASSSETIIASKHHDPLSPKTVSKYFTKARNASGLSFDGNPPTFHELRSLSARLYRNQIGDKFAQRLLGHKSVSMAARYRDSRGREWDKIEIDK

SEQ ID NO: 49 - DNA, 1071 bp, Artificial Sequence
  D336V mutant of the HK022 integrase
atgggcaggcggcggagccacgagcggagagacctgccccccaacctgtacatccggaacaacggctactactgctaccgggacccccggaccggcaaagagttcggcctgggccgggacaggcggatcgccatcaccgaggccatccaggccaacatcgagctgctgtccggcaaccggcgggagagcctgatcgaccggatcaagggcgccgacgccatcaccctgcacgcctggctggacagatacgagaccatcctgagcgagcggggcatccggcccaagaccctgctggactacgcctctaagatccgggccatcagacggaagctgcccgacaagcccctggccgacatcagcaccaaagaagtggccgccatgctgaacacctacgtggccgagggcaagagcgccagcgccaagctgatccggtccaccctggtggacgtgttccgggaggccatcgccgagggccacgtcgccaccaaccccgtgaccgccacccggaccgccaagagcgaagtgcggcggagcaggctgaccgccaacgagtacgtggccatctaccatgccgctgagcccctgcccatctggctgcggctggccatggacctggccgtggtgaccggccagagagtgggcgacctgtgccggatgaagtggagcgacatcaacgacaaccacctgcacatcgagcagagcaagaccggcgccaaactggccatccccctgaccctgaccatcgacgccctgaacatcagcctggccgataccctgcagcagtgcagagaggccagcagcagcgagaccatcatcgccagcaagcaccacgaccccctgagccccaagaccgtgagcaagtacttcaccaaggcccggaacgccagcggcctgagcttcgacggcaacccccccaccttccacgagctgcggagcctgtctgccaggctgtaccggaaccagatcggcgacaagttcgctcagcggctcctgggccacaagagcgtgagcatggccgccagataccgggacagccggggacgggagtgggacaagatcgagatcgacaag

SEQ ID NO: 50 - DNA, 21 bp, Artificial Sequence
  Ataxia ATM2
gaacttataccacgaaaggta

SEQ ID NO: 51 - DNA, 10 bp, Artificial Sequence
  Ataxia ATM2 O
  misc_feature (8)..(10): n is null
taccacgnnn

SEQ ID NO: 52 - DNA, 21 bp, Artificial Sequence
  ALS SOD-1
taacttacatgctgaaaggaa

SEQ ID NO: 53 - DNA, 21 bp, Artificial Sequence
  ALS SOD-2
aatctttactgataaaaggta

SEQ ID NO: 54 - DNA, 10 bp, Artificial Sequence
  ALS SOD-1 O
  misc_feature (8)..(10): n is null
catgctgnnn

SEQ ID NO: 55 - DNA, 10 bp, Artificial Sequence
  ALS SOD-2 O
  misc_feature (8)..(10): n is null
actgatannn

SEQ ID NO: 56 - DNA, 21 bp, Artificial Sequence
  ALS TARDBP4
caccttagcctcccaaagtgc

SEQ ID NO: 57 - DNA, 21 bp, Artificial Sequence
  ALS TARDBP5
gtccttagtaggaaaaagtag

SEQ ID NO: 58 - DNA, 10 bp, Artificial Sequence
  ALS TARDBP4 O
  misc_feature (8)..(10): n is null
gcctcccnnn

SEQ ID NO: 59 - DNA, 10 bp, Artificial Sequence
  ALS TARDBP5 O
  misc_feature (8)..(10): n is null
gtaggaannn

SEQ ID NO: 60 - DNA, 21 bp, Artificial Sequence
  ALS VAPB5
tgcctttctcttccaaagcaa

SEQ ID NO: 61 - DNA, 21 bp, Artificial Sequence
  ALS VAPB6
ttactttgtgggagaaagcta

SEQ ID NO: 62 - DNA, 10 bp, Artificial Sequence
  ALS VAPB5 O
  misc_feature (8)..(10): n is null
ctcttccnnn

SEQ ID NO: 63 - DNA, 10 bp, Artificial Sequence
  ALS VAPB6 O
  misc_feature (8)..(10): n is null
gtgggagnnn

SEQ ID NO: 64 - DNA, 21 bp, Artificial Sequence
  ALS c9ORF 71-1
ctacttagagagtgaaagctg

SEQ ID NO: 65 - DNA, 21 bp, Artificial Sequence
  ALS c9ORF 71-2
acactttcatctgcaaagcta

SEQ ID NO: 66 - DNA, 10 bp, Artificial Sequence
  ALS c9ORF 71-1, O
  misc_feature (8)..(10): n is null
gagagtgnnn

SEQ ID NO: 67 - DNA, 10 bp, Artificial Sequence
  ALS c9ORF 71-2, O
  misc_feature (8)..(10): n is null
catctgcnnn

SEQ ID NO: 68 - DNA, 21 bp, Artificial Sequence
  Cystinosis CTNS2
gagcttactaagcaaaaggag

SEQ ID NO: 69 - DNA, 21 bp, Artificial Sequence
  Cystinosis CTNS3
gaacttttactacaaaagcac

SEQ ID NO: 70 - DNA, 10 bp, Artificial Sequence
  Cystinosis CTNS2 O
  misc_feature (8)..(10): n is null
ctaagcannn

SEQ ID NO: 71 - DNA, 10 bp, Artificial Sequence
  Cystinosis CTNS3 O
  misc_feature (8)..(10): n is null
tactacannn

SEQ ID NO: 72 - DNA, 21 bp, Artificial Sequence
  Cystinosis CTNS4
atacttatgagtgaaaagtat

SEQ ID NO: 73 - DNA, 10 bp, Artificial Sequence
  Cystinosis CTNS4 O
  misc_feature (8)..(10): n is null
tgagtgannn

SEQ ID NO: 74 - DNA, 24 bp, Artificial Sequence
  Primer 1069
gaaagcaggtagcttgcagtgggc

SEQ ID NO: 75 - DNA, 29 bp, Artificial Sequence
  Primer 1070
ggcgacacggaaatgttgaatactcatac

SEQ ID NO: 76 - DNA, 78 bp, Artificial Sequence
  Primer 1143
tcaggttactcatatatactttagattgatgaattccaggatatccgacaaatgattttattttgactaataatgacc

SEQ ID NO: 77 - DNA, 75 bp, Artificial Sequence
  Primer 1144
acggggtctgacgctcagtggaacgaaaacccgcggcagcccgggctcaggtcactaatactatctaagtagttg

SEQ ID NO: 78 - DNA, 58 bp, Artificial Sequence
  Primer 1167
caggttactcatatatactttagattgatgaattccgcgatgtacgggccagatatac

SEQ ID NO: 79 - DNA, 58 bp, Artificial Sequence
  Primer 1169
cattattagtcaaaataaaatcatttgtcggatatcgcagtgggttctctagttagcc

SEQ ID NO: 80 - DNA, 8867 bp, Artificial Sequence
  docking plasmid EF1alfa-attBHEXA3-PuroR- attBATM4-mCherry
gacggatcgggagatcagggtgaggaacagcacactttaccaatgaaagtcgtgaccaggcctcgttagcttggtaccgagctcggatccgaattcgtcgacctcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcatccatctagatctcgagcagctgaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgatgatatcagcttacttaccatgtcagatccagacatgataagatacattgatgagtttggacaaaccacaactagaatgcagtgaaaaaaatgctttatttgtgaaatttgtgtgctattgctttatttgtaaccattataagctgcaataaacaagttaacaacaacaattcattcattttatgtttcaggttcagggggaggtgtgggaggttttttaaagcaagtaaacctctacaaatgtggtatggctgattatgatctctagtcaaggcactatacatcaaatatccttattaacccctttacaaattaaaaagctaaaggtacacaatttttgagcatagttttaatagcagacactctatgcctgtgtggagtaagaaaaaacagtatgttatgattatactgttatgcctacttataaaggttacagaatatttttccataattttcttgtatagcaggcagctttttcctttgtggtgtaaatagcaaagcaagcaagagttctattactaaacacgcatgactcaaaaaacttagcaattctgaaggaaagtccttggggtcttctacctttctttcttttttggaggagtagaatgttgagagtcagcagtagcctcatcatcactagatggatttcttctgagcaaaacaggttttcctcattaaaggcattccaccactgctcccattctcagttccataggttggaatctaaaatacacaaacaattagaatcagtagtttaacacatatacacttaaaaattttatatttaccttagagctttaaatctctgtaggtagtttgtcaattatgtcacaccacagaagtaaggttccttcacaaagatccctcgagaaaaaaaataaaaagagatggaggaacgggaaaaagttagttgtggtgataggtggcaagtggtattcctaagaacaacaagaaaagcatttcatattatggctgaactgagcgaacaagtgcaaaattaagcatcaacgacaacaacgagaatggttatgttcctcctcacttaagaggaaaaccagaagtgccagaaataacatgagcaactacaataacaacaacggcggctacaacggtggcgtggcggtggcagcttctttagcaacaaccgtcgtggtggttacggcaacggtggtttctcggtggaaacaacggtggcagcagatctaacggccgttctggtggtagatggatcgatggcaaacatgtcccagctccaagaaacgaaaaggccgagatcgccatatttggtgtccccgaggatcctctagagtcgacggtatcgataaaggggtcagggagttccctttctgagtcaaagaaaggggggacggacggcgcggccgcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaataagcttggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtcctgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggtccctttagggttccgatttagtgctttacggcacctcgaccccaaaaaacttgattagggtgatggttcacgtacctagaagttcctattccgaagttcctattctctagaaagtataggaacttccttggccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtactacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtgtgccacctgacgtc

SEQ ID NO: 81 - DNA, 6392 bp, Artificial Sequence
  incoming plasmid attPHEXA5-GFP(ORF)-NeoR-CMV promoter-attPATM4
tagttattagatctcgagctcaagcttaagcttacttaccatgtcagatccagacatgataagatacattgatgagtttggacaaaccacaactagaatgcagtgaaaaaaatgctttatttgtgaaatttgtgtgctattgctttatttgtaaccattataagctgcaataaacaagttaacaacaacaattcattcattttatgtttcaggttcagggggaggtgtgggaggttttttaaagcaagtaaacctctacaaatgtggtatggctgattatgatctctagtcaaggcactatacatcaaatatccttattaacccctttacaaattaaaaagctaaaggtacacaatttttgagcatagttttaatagcagacactctatgcctgtgtggagtaagaaaaaacagtatgttatgattatactgttatgcctacttataaaggttacagaatatttttccataattttcttgtatagcaggcagctttttcctttgtggtgtaaatagcaaagcaagcaagagttctattactaaacacgcatgactcaaaaaacttagcaattctgaaggaaagtccttggggtcttctacctttctttcttttttggaggagtagaatgttgagagtcagcagtagcctcatcatcactagatggatttcttctgagcaaaacaggttttcctcattaaaggcattccaccactgctcccattctcagttccataggttggaatctaaaatacacaaacaattagaatcagtagtttaacacatatacacttaaaaattttatatttaccttagagctttaaatctctgtaggtagtttgtcaattatgtcacaccacagaagtaaggttccttcacaaagatccctcgagaaaaaaaataaaaagagatggaggaacgggaaaaagttagttgtggtgataggtggcaagtggtattcctaagaacaacaagaaaagcatttcatattatggctgaactgagcgaacaagtgcaaaattaagcatcaacgacaacaacgagaatggttatgttcctcctcacttaagaggaaaaccagaagtgccagaaataacatgagcaactacaataacaacaacggcggctacaacggtggcgtggcggtggcagcttctttagcaacaaccgtcgtggtggttacggcaacggtggtttctcggtggaaacaacggtggcagcagatctaacggatcctctagagtcgacggtatcgataagcttaagcttgcatgcctgcagaggtcactaatactatctaagtagttgattcatagtgactggatatgttgcgttttgtcgcattatgtagtctatcatttaaccacagattagtgtaatgcgatgatttttaagtgattaatgttattttgtcatcctttaccaatgtaagttgtatatttaaaatctctttaattatcagtaaattaatgtaagtaggtcattattagtcaaaataaaatcatttgaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatgaattccgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgatatccgacaaatgattttattttgactaataatgacctacttacattaatttactgataattaaagagattttaaatatacaacttactgagtcaaaggatgacaaaataacattaatcacttaaaaatcatcgcattacactaatctgtggttaaatgatagactacataatgcgacaaaacgcaacatatccagtcactatgaatcaactacttagatagtattagtgacctgagcccgggctgccgcgggttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 82 - DNA, 1071 bp, Artificial Sequence
  Double mutant E174K/I43F
atgggcaggcggcggagccacgagcggagagacctgccccccaacctgtacatccggaacaacggctactactgctaccgggacccccggaccggcaaagagttcggcctgggccgggacaggcggttcgccatcaccgaggccatccaggccaacatcgagctgctgtccggcaaccggcgggagagcctgatcgaccggatcaagggcgccgacgccatcaccctgcacgcctggctggacagatacgagaccatcctgagcgagcggggcatccggcccaagaccctgctggactacgcctctaagatccgggccatcagacggaagctgcccgacaagcccctggccgacatcagcaccaaagaagtggccgccatgctgaacacctacgtggccgagggcaagagcgccagcgccaagctgatccggtccaccctggtggacgtgttccgggaggccatcgccgagggccacgtcgccaccaaccccgtgaccgccacccggaccgccaagagcaaagtgcggcggagcaggctgaccgccaacgagtacgtggccatctaccatgccgctgagcccctgcccatctggctgcggctggccatggacctggccgtggtgaccggccagagagtgggcgacctgtgccggatgaagtggagcgacatcaacgacaaccacctgcacatcgagcagagcaagaccggcgccaaactggccatccccctgaccctgaccatcgacgccctgaacatcagcctggccgataccctgcagcagtgcagagaggccagcagcagcgagaccatcatcgccagcaagcaccacgaccccctgagccccaagaccgtgagcaagtacttcaccaaggcccggaacgccagcggcctgagcttcgacggcaacccccccaccttccacgagctgcggagcctgtctgccaggctgtaccggaaccagatcggcgacaagttcgctcagcggctcctgggccacaagagcgacagcatggccgccagataccgggacagccggggacgggagtgggacaagatcgagatcgacaag

SEQ ID NO: 83 - protein, 357 aa, Artificial Sequence
  Double mutant E174K/ I43F
MGRRRSHERRDLPPNLYIRNNGYYCYRDPRTGKEFGLGRDRRFAITEAIQANIELLSGNRRESLIDRIKGADAITLHAWLDRYETILSERGIRPKTLLDYASKIRAIRRKLPDKPLADISTKEVAAMLNTYVAEGKSASAKLIRSTLVDVFREAIAEGHVATNPVTATRTAKSKVRRSRLTANEYVAIYHAAEPLPIWLRLAMDLAVVTGQRVGDLCRMKWSDINDNHLHIEQSKTGAKLAIPLTLTIDALNISLADTLQQCREASSSETIIASKHHDPLSPKTVSKYFTKARNASGLSFDGNPPTFHELRSLSARLYRNQIGDKFAQRLLGHKSDSMAARYRDSRGREWDKIEIDK

SEQ ID NO: 84 - DNA, 1071 bp, Artificial Sequence
  Double mutant E174K/R319G
atgggcaggcggcggagccacgagcggagagacctgccccccaacctgtacatccggaacaacggctactactgctaccgggacccccggaccggcaaagagttcggcctgggccgggacaggcggatcgccatcaccgaggccatccaggccaacatcgagctgctgtccggcaaccggcgggagagcctgatcgaccggatcaagggcgccgacgccatcaccctgcacgcctggctggacagatacgagaccatcctgagcgagcggggcatccggcccaagaccctgctggactacgcctctaagatccgggccatcagacggaagctgcccgacaagcccctggccgacatcagcaccaaagaagtggccgccatgctgaacacctacgtggccgagggcaagagcgccagcgccaagctgatccggtccaccctggtggacgtgttccgggaggccatcgccgagggccacgtcgccaccaaccccgtgaccgccacccggaccgccaagagcaaagtgcggcggagcaggctgaccgccaacgagtacgtggccatctaccatgccgctgagcccctgcccatctggctgcggctggccatggacctggccgtggtgaccggccagagagtgggcgacctgtgccggatgaagtggagcgacatcaacgacaaccacctgcacatcgagcagagcaagaccggcgccaaactggccatccccctgaccctgaccatcgacgccctgaacatcagcctggccgataccctgcagcagtgcagagaggccagcagcagcgagaccatcatcgccagcaagcaccacgaccccctgagccccaagaccgtgagcaagtacttcaccaaggcccggaacgccagcggcctgagcttcgacggcaacccccccaccttccacgagctgcggagcctgtctgccaggctgtacggcaaccagatcggcgacaagttcgctcagcggctcctgggccacaagagcgacagcatggccgccagataccgggacagccggggacgggagtgggacaagatcgagatcgacaag

SEQ ID NO: 85 - protein, 357 aa, Artificial Sequence
  Double mutant E174K/ R319G
MGRRRSHERRDLPPNLYIRNNGYYCYRDPRTGKEFGLGRDRRIAITEAIQANIELLSGNRRESLIDRIKGADAITLHAWLDRYETILSERGIRPKTLLDYASKIRAIRRKLPDKPLADISTKEVAAMLNTYVAEGKSASAKLIRSTLVDVFREAIAEGHVATNPVTATRTAKSKVRRSRLTANEYVAIYHAAEPLPIWLRLAMDLAVVTGQRVGDLCRMKWSDINDNHLHIEQSKTGAKLAIPLTLTIDALNISLADTLQQCREASSSETIIASKHHDPLSPKTVSKYFTKARNASGLSFDGNPPTFHELRSLSARLYGNQIGDKFAQRLLGHKSDSMAARYRDSRGREWDKIEIDK

SEQ ID NO: 86 - DNA, 1071 bp, Artificial Sequence
  Double mutant E174K/E264G
atgggcaggcggcggagccacgagcggagagacctgccccccaacctgtacatccggaacaacggctactactgctaccgggacccccggaccggcaaagagttcggcctgggccgggacaggcggatcgccatcaccgaggccatccaggccaacatcgagctgctgtccggcaaccggcgggagagcctgatcgaccggatcaagggcgccgacgccatcaccctgcacgcctggctggacagatacgagaccatcctgagcgagcggggcatccggcccaagaccctgctggactacgcctctaagatccgggccatcagacggaagctgcccgacaagcccctggccgacatcagcaccaaagaagtggccgccatgctgaacacctacgtggccgagggcaagagcgccagcgccaagctgatccggtccaccctggtggacgtgttccgggaggccatcgccgagggccacgtcgccaccaaccccgtgaccgccacccggaccgccaagagcaaagtgcggcggagcaggctgaccgccaacgagtacgtggccatctaccatgccgctgagcccctgcccatctggctgcggctggccatggacctggccgtggtgaccggccagagagtgggcgacctgtgccggatgaagtggagcgacatcaacgacaaccacctgcacatcgagcagagcaagaccggcgccaaactggccatccccctgaccctgaccatcgacgccctgaacatcagcctggccgataccctgcagcagtgcagaggcgccagcagcagcgagaccatcatcgccagcaagcaccacgaccccctgagccccaagaccgtgagcaagtacttcaccaaggcccggaacgccagcggcctgagcttcgacggcaacccccccaccttccacgagctgcggagcctgtctgccaggctgtaccggaaccagatcggcgacaagttcgctcagcggctcctgggccacaagagcgacagcatggccgccagataccgggacagccggggacgggagtgggacaagatcgagatcgacaag

SEQ ID NO: 87 - protein, 357 aa, Artificial Sequence
  Double mutant E174K/E264G
MGRRRSHERRDLPPNLYIRNNGYYCYRDPRTGKEFGLGRDRRIAITEAIQANIELLSGNRRESLIDRIKGADAITLHAWLDRYETILSERGIRPKTLLDYASKIRAIRRKLPDKPLADISTKEVAAMLNTYVAEGKSASAKLIRSTLVDVFREAIAEGHVATNPVTATRTAKSKVRRSRLTANEYVAIYHAAEPLPIWLRLAMDLAVVTGQRVGDLCRMKWSDINDNHLHIEQSKTGAKLAIPLTLTIDALNISLADTLQQCRGASSSETIIASKHHDPLSPKTVSKYFTKARNASGLSFDGNPPTFHELRSLSARLYRNQIGDKFAQRLLGHKSDSMAARYRDSRGREWDKIEIDK

SEQ ID NO: 88 - DNA, 1071 bp, Artificial Sequence
  Double mutant E174K/D336V
atgggcaggcggcggagccacgagcggagagacctgccccccaacctgtacatccggaacaacggctactactgctaccgggacccccggaccggcaaagagttcggcctgggccgggacaggcggatcgccatcaccgaggccatccaggccaacatcgagctgctgtccggcaaccggcgggagagcctgatcgaccggatcaagggcgccgacgccatcaccctgcacgcctggctggacagatacgagaccatcctgagcgagcggggcatccggcccaagaccctgctggactacgcctctaagatccgggccatcagacggaagctgcccgacaagcccctggccgacatcagcaccaaagaagtggccgccatgctgaacacctacgtggccgagggcaagagcgccagcgccaagctgatccggtccaccctggtggacgtgttccgggaggccatcgccgagggccacgtcgccaccaaccccgtgaccgccacccggaccgccaagagcaaagtgcggcggagcaggctgaccgccaacgagtacgtggccatctaccatgccgctgagcccctgcccatctggctgcggctggccatggacctggccgtggtgaccggccagagagtgggcgacctgtgccggatgaagtggagcgacatcaacgacaaccacctgcacatcgagcagagcaagaccggcgccaaactggccatccccctgaccctgaccatcgacgccctgaacatcagcctggccgataccctgcagcagtgcagagaggccagcagcagcgagaccatcatcgccagcaagcaccacgaccccctgagccccaagaccgtgagcaagtacttcaccaaggcccggaacgccagcggcctgagcttcgacggcaacccccccaccttccacgagctgcggagcctgtctgccaggctgtaccggaaccagatcggcgacaagttcgctcagcggctcctgggccacaagagcgtgagcatggccgccagataccgggacagccggggacgggagtgggacaagatcgagatcgacaag

SEQ ID NO: 89 - protein, 357 aa, Artificial Sequence
  Double mutant E174K/D336V
MGRRRSHERRDLPPNLYIRNNGYYCYRDPRTGKEFGLGRDRRIAITEAIQANIELLSGNRRESLIDRIKGADAITLHAWLDRYETILSERGIRPKTLLDYASKIRAIRRKLPDKPLADISTKEVAAMLNTYVAEGKSASAKLIRSTLVDVFREAIAEGHVATNPVTATRTAKSKVRRSRLTANEYVAIYHAAEPLPIWLRLAMDLAVVTGQRVGDLCRMKWSDINDNHLHIEQSKTGAKLAIPLTLTIDALNISLADTLQQCREASSSETIIASKHHDPLSPKTVSKYFTKARNASGLSFDGNPPTFHELRSLSARLYRNQIGDKFAQRLLGHKSVSMAARYRDSRGREWDKIEIDK

SEQ ID NO: 90 - DNA, 23 bp, Artificial Sequence
  Primer 958
ggccagctgtcccaaacgtccag

SEQ ID NO: 91 - DNA, 26 bp, Artificial Sequence
  Primer 1080
cctggcgcagttgcaaacgctgcccc

SEQ ID NO: 92 - DNA, 21 bp, Artificial Sequence
  DMD2 attE1
ttgcttaatggagaaaaggta

SEQ ID NO: 93 - DNA, 21 bp, Artificial Sequence
  DMD 3 attE2
gtgctttaaaaagaaaagggg

SEQ ID NO: 94 - DNA, 10 bp, Artificial Sequence
  DMD2 O1
  misc_feature (8)..(10): n is null
atggagannn

SEQ ID NO: 95 - DNA, 10 bp, Artificial Sequence
  DMD3 O2
  misc_feature (8)..(10): n is null
aaaaagannn

SEQ ID NO: 96 - DNA, 21 bp, Artificial Sequence
  CFTR10 attE1
ctacttttaaaaacaaagtct

SEQ ID NO: 97 - DNA, 21 bp, Artificial Sequence
  CFTR12 attE2
acgctttccccttcaaaggtg

SEQ ID NO: 98 - DNA, 10 bp, Artificial Sequence
  CFTR10 O1
  misc_feature (8)..(10): n is null
taaaaacnnn

SEQ ID NO: 99 - DNA, 10 bp, Artificial Sequence
  CFTR12 O2
  misc_feature (8)..(10): n is null
ccccttcnnn

SEQ ID NO: 100 - DNA, 142 bp, Artificial Sequence
  Int-HK022 attP1 P
tcaggtcactaatactatctaagtagttgattcatagtgactggatatgttgcgttttgtcgcattatgtagtctatcatttaaccacagattagtgtaatgcgatgatttttaagtgattaatgttattttgtcatccttt

SEQ ID NO: 101 - DNA, 81 bp, Artificial Sequence
  Int-HK022 attP2 P′
taagttgtatatttaaaatctctttaattatcagtaaattaatgtaagtaggtcattattagtcaaaataaaatcatttgt

SEQ ID NO: 102 - DNA, 10 bp, Artificial Sequence
  NPC1 O1
  misc_feature (8)..(10): n is null
agatgccnnn

SEQ ID NO: 103 - DNA, 10 bp, Artificial Sequence
  NPC1 O2
  misc_feature (8)..(10): n is null
acactggnnn

SEQ ID NO: 104 - DNA, 10 bp, Artificial Sequence
  SCN1A4 O1
  misc_feature (8)..(10): n is null
gcactgtnnn

SEQ ID NO: 105 - DNA, 10 bp, Artificial Sequence
  SCN1A3 O2
  misc_feature (8)..(10): n is null
acagtgcnnn

SEQ ID NO: 106 - DNA, 10 bp, Artificial Sequence
  COL3A1 O1
  misc_feature (8)..(10): n is null
aaaacagnnn

SEQ ID NO: 107 - DNA, 10 bp, Artificial Sequence
  COL3A1 O2
  misc_feature (8)..(10): n is null
tttaaaannn

SEQ ID NO: 108 - DNA, 21 bp, Artificial Sequence
  DMD4
atactttttgcctaaaagcag

SEQ ID NO: 109 - DNA, 10 bp, Artificial Sequence
  DMD4 O
  misc_feature (8)..(10): n is null
ttgcctannn

SEQ ID NO: 110 - DNA, 21 bp, Artificial Sequence
  DMD5
tttcttttgtaaacaaaggta

SEQ ID NO: 111 - DNA, 10 bp, Artificial Sequence
  DMD5 O
  misc_feature (8)..(10): n is null
tgtaaacnnn

SEQ ID NO: 112 - DNA, 21 bp, Artificial Sequence
  DMD6
cttctttatgttttaaagtat

SEQ ID NO: 113 - DNA, 10 bp, Artificial Sequence
  DMD6 O
  misc_feature (8)..(10): n is null
atgttttnnn

SEQ ID NO: 114 - DNA, 21 bp, Artificial Sequence
  DMD7
actctttcctgacaaaagtag

SEQ ID NO: 115 - DNA, 10 bp, Artificial Sequence
  DMD7 O
  misc_feature (8)..(10): n is null
cctgacannn

SEQ ID NO: 116 - DNA, 21 bp, Artificial Sequence
  CTNS1
tcactttggtacagaaaggta

SEQ ID NO: 117 - DNA, 10 bp, Artificial Sequence
  CTNS1 O
  misc_feature (8)..(10): n is null
ggtacagnnn

SEQ ID NO: 118 - DNA, 21 bp, Artificial Sequence
  NPC1 attE1
tggcttaagatgccaaaggtg

SEQ ID NO: 119 - DNA, 21 bp, Artificial Sequence
  NPC1 attE2
tcacttaacactggaaaggca

SEQ ID NO: 120 - DNA, 21 bp, Artificial Sequence
  SCN1A4 attE1
atactttgcactgtaaagtgt

SEQ ID NO: 121 - DNA, 21 bp, Artificial Sequence
  SCN1A3 attE2
atactttacagtgcaaagtat

SEQ ID NO: 122 - DNA, 21 bp, Artificial Sequence
  COL3A1 attE1
aaacttaaaaacagaaagtgt

SEQ ID NO: 123 - DNA, 21 bp, Artificial Sequence
  COL3A1 attE2
tgacttatttaaaaaaaggta

SEQ ID NO: 124 - DNA, 30 bp, Artificial Sequence
  Primer 788
gggaagcttattccgctttgcgactcaacc

SEQ ID NO: 125 - DNA, 21 bp, Artificial Sequence
  CFTR13
cagcttttcttaataaagcaa

SEQ ID NO: 126 - DNA, 21 bp, Artificial Sequence
  CFTR14
gtactttgttagcaaaagctg

SEQ ID NO: 127 - DNA, 10 bp, Artificial Sequence
  CFTR13 O
  misc_feature (8)..(10): n is null
tcttaatnnn

SEQ ID NO: 128 - DNA, 10 bp, Artificial Sequence
  CFTR14 O
  misc_feature (8)..(10): n is null
gttagcannn

SEQ ID NO: 129 - DNA, 21 bp, Artificial Sequence
  CTNS a
atactttagccccgaaaggca

SEQ ID NO: 130 - DNA, 21 bp, Artificial Sequence
  CTNS d
gcccttaaggcaaaaaagtcc

SEQ ID NO: 131 - DNA, 10 bp, Artificial Sequence
  CTNS a o
  misc_feature (8)..(10): n is null
agccccgnnn

SEQ ID NO: 132 - DNA, 10 bp, Artificial Sequence
  CTNS d o
  misc_feature (8)..(10): n is null
aggcaaannn

SEQ ID NO: 133 - DNA, 28 bp, Artificial Sequence
  oEY400
cgggatccgatgtacgggccagatatac

SEQ ID NO: 134 - DNA, 29 bp, Artificial Sequence
  oEY416
gcggatccgggtctccctatagtgagtcg

SEQ ID NO: 135 - DNA, 29 bp, Artificial Sequence
  oEY606
gggagatctacttaccatgtcagatccag

SEQ ID NO: 136 - DNA, 38 bp, Artificial Sequence
  oEY674
ggaccggtcaaatgattttattttgactaataatgacc

SEQ ID NO: 137 - DNA, 37 bp, Artificial Sequence
  oEY675
ggggctgcagaggtcactaatactatctaagtagttg

SEQ ID NO: 138 - DNA, 42 bp, Artificial Sequence
  oEY736
aggtcactaatactatctaagtagttgattcatagtgactgg

SEQ ID NO: 139 - DNA, 45 bp, Artificial Sequence
  oEY931
cgtgccagctgcattaatgaatcggccaacgaattccagaagctt

SEQ ID NO: 140 - DNA, 30 bp, Artificial Sequence
  oEY1192
gtagcggtcacgctgcgcgtaaccaccaca

SEQ ID NO: 141 - DNA, 39 bp, Artificial Sequence
  oEY1201
cccggatccttagggttccgatttagtgctttacggcac

SEQ ID NO: 142 - DNA, 39 bp, Artificial Sequence
  oEY1202
gggtctagacaaatgattttattttgactaataatgacc

SEQ ID NO: 143 - DNA, 51 bp, Artificial Sequence
  oEY1203
cccggatccaggtcactaatactatctaagtagttgattcatagtgactgg

SEQ ID NO: 144 - DNA, 39 bp, Artificial Sequence
  oEY1215
gggccgcggctcaggtcactaatactatctaagtagttg

SEQ ID NO: 145 - DNA, 38 bp, Artificial Sequence
  oEY1216
gggccgcggctcaaaggcggtaatacggttatccacag

SEQ ID NO: 146 - DNA, 35 bp, Artificial Sequence
  oEY1217
cccgaattcgttggccgattcattaatgcagctgg

SEQ ID NO: 147 - DNA, 42 bp, Artificial Sequence
  oEY1237
cccggatcccaaatgattttattttgactaataatgacctac

SEQ ID NO: 148 - DNA, 51 bp, Artificial Sequence
  oEY1238
ccctctagaaggtcactaatactatctaagtagttgattcatagtgactgg

SEQ ID NO: 149 - DNA, 57 bp, Artificial Sequence
  oEY1240
ctacttagatagtattagtgacctggatccctctgcaaatgcaggaaactatcagag

SEQ ID NO: 150 - DNA, 32 bp, Artificial Sequence
  oEY1241
ttcgcgcgctcaacagatctgtcaaatcgcct

SEQ ID NO: 151 - DNA, 21 bp, Artificial Sequence
  oEY1242
tgttgagcgcgcgaaacgcgg

SEQ ID NO: 152 - DNA, 28 bp, Artificial Sequence
  oEY1243
gctcaccataggtccagggttctcctcc

SEQ ID NO: 153 - DNA, 26 bp, Artificial Sequence
  oEY1244
ctggacctatggtgagcaagggcgag

SEQ ID NO: 154 - DNA, 53 bp, Artificial Sequence
  oEY1245
aaatcatttgtcgaagcttctggaattcggacaaaccacaactagaatgcagt

SEQ ID NO: 155 - DNA, 33 bp, Artificial Sequence
  oEY1246
gggtctagagctgccaccgttgtttccaccgag

SEQ ID NO: 156 - DNA, 46 bp, Artificial Sequence
  oEY1254
tattagtcaaaataaaatcatttgggatccatggtgagcaagggcg

SEQ ID NO: 157 - DNA, 29 bp, Artificial Sequence
  oEY1255
ttcgcgcgcttgtacagctcgtccatgcc

SEQ ID NO: 158 - DNA, 21 bp, Artificial Sequence
  oEY1256
gtacaagcgcgcgaaacgcgg

SEQ ID NO: 159 - DNA, 56 bp, Artificial Sequence
  oEY1257
atttgtcgaagcttctggaattcaacttaccacatttaggtccagggttctcctcc

SEQ ID NO: 160 - DNA, 22 bp, Artificial Sequence
  PRIMER 206
cgtcgccgtccagctcgaccag

SEQ ID NO: 161 - DNA, 21 bp, Artificial Sequence
  attB of coliphage HK022
gcactttaggtgaaaaaggtt

SEQ ID NO: 162 - DNA, 10 bp, Artificial Sequence
  O of attB of coliphage HK022
  misc_feature (8)..(10): n is null
aggtgaannn

SEQ ID NO: 163 - DNA, 17 bp, Artificial Sequence
  consensus sequence of an active attB
  misc_feature (6)..(12): n is a, c, g, or t
actttnnnnnnnaaagg

SEQ ID NO: 164 - DNA, 28 bp, Artificial Sequence
  primer 1265
cagcaagcaccacaaacccctgagcccc

SEQ ID NO: 165 - DNA, 29 bp, Artificial Sequence
  primer 1266
ggggctcaggggtttgtggtgcttgctgg

SEQ ID NO: 166 - DNA, 57 bp, Artificial Sequence
  primer 1280
agctttgatagtttatgcctctacttttaaaaacaaagtctaacagatttttctcag

SEQ ID NO: 167 - DNA, 57 bp, Artificial Sequence
  primer 1281
aattctgagaaaaatctgttagactttgtttttaaaagtagaggcataaactatcaa

SEQ ID NO: 168 - DNA, 57 bp, Artificial Sequence
  primer 1282
agctttgagatgatggaaacacgctttccccttcaaaggtgctgctagttccaaagg

SEQ ID NO: 169 - DNA, 57 bp, Artificial Sequence
  primer 1283
aattcctttggaactagcagcacctttgaaggggaaagcgtgtttccatcatctcaa

SEQ ID NO: 170 - DNA, 23 bp, Artificial Sequence
  primer 143
gcaaaatcaaaagtaaggcgttc

SEQ ID NO: 171 - DNA, 23 bp, Artificial Sequence
  primer 144
gaacgccttacttttgattttgc

SEQ ID NO: 172 - DNA, 19 bp, Artificial Sequence
  primer 203
gctagttattgctcagcgg

SEQ ID NO: 173 - DNA, 18 bp, Artificial Sequence
  primer 513
aagaggatcacatatggg

SEQ ID NO: 174 - DNA, 39 bp, Artificial Sequence
  primer 1351
tttgacagatctgttgaggagagccaagagaggctctgg

SEQ ID NO: 175 - DNA, 41 bp, Artificial Sequence
  primer 1352
gagcctctcttggctctcctcaacagatctgtcaaatcgcc

SEQ ID NO: 176 - DNA, 41 bp, Artificial Sequence
  primer 1353
cttaagcttggactcacctgacgaggtccagggttctcctc

SEQ ID NO: 177 - protein, 63 aa, Bacteriophage HK022
  MISC_FEATURE: ND domain of HK022 Integrase
MGRRRSHERRDLPPNLYIRNNGYYCYRDPRTGKEFGLGRDRRIAITEAIQANIELLSGNRRES

SEQ ID NO: 178 - protein, 101 aa, Bacteriophage HK022
  MISC_FEATURE: CB domain of HK022 Integrase
TLHAWLDRYETILSERGIRPKTLLDYASKIRAIRRKLPDKPLADISTKEVAAMLNTYVAEGKSASAKLIRSTLVDVFREAIAEGHVATNPVTATRTAKSEV

SEQ ID NO: 179 - protein, 181 aa, Bacteriophage HK022
  MISC_FEATURE: CD domain of HK022 Integrase
RRSRLTANEYVAIYHAAEPLPIWLRLAMDLAVVTGQRVGDLCRMKWSDINDNHLHIEQSKTGAKLAIPLTLTIDALNISLADTLQQCREASSSETIIASKHHDPLSPKTVSKYFTKARNASGLSFDGNPPTFHELRSLSARLYRNQIGDKFAQRLLGHKSDSMAARYRDSRGREWDKIEID

SEQ ID NO: 180 - protein, 357 aa, Artificial Sequence
  E134K mutant of the HK022 integrase
MGRRRSHERRDLPPNLYIRNNGYYCYRDPRTGKEFGLGRDRRIAITEAIQANIELLSGNRRESLIDRIKGADAITLHAWLDRYETILSERGIRPKTLLDYASKIRAIRRKLPDKPLADISTKEVAAMLNTYVAKGKSASAKLIRSTLVDVFREAIAEGHVATNPVTATRTAKSEVRRSRLTANEYVAIYHAAEPLPIWLRLAMDLAVVTGQRVGDLCRMKWSDINDNHLHIEQSKTGAKLAIPLTLTIDALNISLADTLQQCREASSSETIIASKHHDPLSPKTVSKYFTKARNASGLSFDGNPPTFHELRSLSARLYRNQIGDKFAQRLLGHKSDSMAARYRDSRGREWDKIEIDK

SEQ ID NO: 181 - DNA, 1071 bp, Artificial Sequence
  E134K mutant of the HK022 integrase
atgggcaggcggcggagccacgagcggagagacctgccccccaacctgtacatccggaacaacggctactactgctaccgggacccccggaccggcaaagagttcggcctgggccgggacaggcggatcgccatcaccgaggccatccaggccaacatcgagctgctgtccggcaaccggcgggagagcctgatcgaccggatcaagggcgccgacgccatcaccctgcacgcctggctggacagatacgagaccatcctgagcgagcggggcatccggcccaagaccctgctggactacgcctctaagatccgggccatcagacggaagctgcccgacaagcccctggccgacatcagcaccaaagaagtggccgccatgctgaacacctacgtggccaaaggcaagagcgccagcgccaagctgatccggtccaccctggtggacgtgttccgggaggccatcgccgagggccacgtcgccaccaaccccgtgaccgccacccggaccgccaagagcgaagtgcggcggagcaggctgaccgccaacgagtacgtggccatctaccatgccgctgagcccctgcccatctggctgcggctggccatggacctggccgtggtgaccggccagagagtgggcgacctgtgccggatgaagtggagcgacatcaacgacaaccacctgcacatcgagcagagcaagaccggcgccaaactggccatccccctgaccctgaccatcgacgccctgaacatcagcctggccgataccctgcagcagtgcagagaggccagcagcagcgagaccatcatcgccagcaagcaccacgaccccctgagccccaagaccgtgagcaagtacttcaccaaggcccggaacgccagcggcctgagcttcgacggcaacccccccaccttccacgagctgcggagcctgtctgccaggctgtaccggaaccagatcggcgacaagttcgctcagcggctcctgggccacaagagcgacagcatggccgccagataccgggacagccggggacgggagtgggacaagatcgagatcgacaag

SEQ ID NO: 182 - protein, 357 aa, Artificial Sequence
  D278K mutant of the HK022 integrase
MGRRRSHERRDLPPNLYIRNNGYYCYRDPRTGKEFGLGRDRRIAITEAIQANIELLSGNRRESLIDRIKGADAITLHAWLDRYETILSERGIRPKTLLDYASKIRAIRRKLPDKPLADISTKEVAAMLNTYVAEGKSASAKLIRSTLVDVFREAIAEGHVATNPVTATRTAKSEVRRSRLTANEYVAIYHAAEPLPIWLRLAMDLAVVTGQRVGDLCRMKWSDINDNHLHIEQSKTGAKLAIPLTLTIDALNISLADTLQQCREASSSETIIASKHHKPLSPKTVSKYFTKARNASGLSFDGNPPTFHELRSLSARLYRNQIGDKFAQRLLGHKSDSMAARYRDSRGREWDKIEIDK

SEQ ID NO: 183 - DNA, 1071 bp, Artificial Sequence
  D278K mutant of the HK022 integrase
atgggcaggcggcggagccacgagcggagagacctgccccccaacctgtacatccggaacaacggctactactgctaccgggacccccggaccggcaaagagttcggcctgggccgggacaggcggatcgccatcaccgaggccatccaggccaacatcgagctgctgtccggcaaccggcgggagagcctgatcgaccggatcaagggcgccgacgccatcaccctgcacgcctggctggacagatacgagaccatcctgagcgagcggggcatccggcccaagaccctgctggactacgcctctaagatccgggccatcagacggaagctgcccgacaagcccctggccgacatcagcaccaaagaagtggccgccatgctgaacacctacgtggccgagggcaagagcgccagcgccaagctgatccggtccaccctggtggacgtgttccgggaggccatcgccgagggccacgtcgccaccaaccccgtgaccgccacccggaccgccaagagcgaagtgcggcggagcaggctgaccgccaacgagtacgtggccatctaccatgccgctgagcccctgcccatctggctgcggctggccatggacctggccgtggtgaccggccagagagtgggcgacctgtgccggatgaagtggagcgacatcaacgacaaccacctgcacatcgagcagagcaagaccggcgccaaactggccatccccctgaccctgaccatcgacgccctgaacatcagcctggccgataccctgcagcagtgcagagaggccagcagcagcgagaccatcatcgccagcaagcaccacaaacccctgagccccaagaccgtgagcaagtacttcaccaaggcccggaacgccagcggcctgagcttcgacggcaacccccccaccttccacgagctgcggagcctgtctgccaggctgtaccggaaccagatcggcgacaagttcgctcagcggctcctgggccacaagagcgacagcatggccgccagataccgggacagccggggacgggagtgggacaagatcgagatcgacaag

SEQ ID NO: 184 - protein, 357 aa, Artificial Sequence
  E174K/D278K double mutant of the HK022 integrase
MGRRRSHERRDLPPNLYIRNNGYYCYRDPRTGKEFGLGRDRRIAITEAIQANIELLSGNRRESLIDRIKGADAITLHAWLDRYETILSERGIRPKTLLDYASKIRAIRRKLPDKPLADISTKEVAAMLNTYVAEGKSASAKLIRSTLVDVFREAIAEGHVATNPVTATRTAKSKVRRSRLTANEYVAIYHAAEPLPIWLRLAMDLAVVTGQRVGDLCRMKWSDINDNHLHIEQSKTGAKLAIPLTLTIDALNISLADTLQQCREASSSETIIASKHHKPLSPKTVSKYFTKARNASGLSFDGNPPTFHELRSLSARLYRNQIGDKFAQRLLGHKSDSMAARYRDSRGREWDKIEIDK

SEQ ID NO: 185 - protein, 357 aa, Artificial Sequence
  E174K/I43F/R319G triple mutant of the HK022 integrase
MGRRRSHERRDLPPNLYIRNNGYYCYRDPRTGKEFGLGRDRRFAITEAIQANIELLSGNRRESLIDRIKGADAITLHAWLDRYETILSERGIRPKTLLDYASKIRAIRRKLPDKPLADISTKEVAAMLNTYVAEGKSASAKLIRSTLVDVFREAIAEGHVATNPVTATRTAKSKVRRSRLTANEYVAIYHAAEPLPIWLRLAMDLAVVTGQRVGDLCRMKWSDINDNHLHIEQSKTGAKLAIPLTLTIDALNISLADTLQQCREASSSETIIASKHHDPLSPKTVSKYFTKARNASGLSFDGNPPTFHELRSLSARLYGNQIGDKFAQRLLGHKSDSMAARYRDSRGREWDKIEIDK

SEQ ID NO: 186 - DNA, 1071 bp, Artificial Sequence
  E174K/D278K double mutant of the HK022 integrase
atgggcaggcggcggagccacgagcggagagacctgccccccaacctgtacatccggaacaacggctactactgctaccgggacccccggaccggcaaagagttcggcctgggccgggacaggcggatcgccatcaccgaggccatccaggccaacatcgagctgctgtccggcaaccggcgggagagcctgatcgaccggatcaagggcgccgacgccatcaccctgcacgcctggctggacagatacgagaccatcctgagcgagcggggcatccggcccaagaccctgctggactacgcctctaagatccgggccatcagacggaagctgcccgacaagcccctggccgacatcagcaccaaagaagtggccgccatgctgaacacctacgtggccgagggcaagagcgccagcgccaagctgatccggtccaccctggtggacgtgttccgggaggccatcgccgagggccacgtcgccaccaaccccgtgaccgccacccggaccgccaagagcaaagtgcggcggagcaggctgaccgccaacgagtacgtggccatctaccatgccgctgagcccctgcccatctggctgcggctggccatggacctggccgtggtgaccggccagagagtgggcgacctgtgccggatgaagtggagcgacatcaacgacaaccacctgcacatcgagcagagcaagaccggcgccaaactggccatccccctgaccctgaccatcgacgccctgaacatcagcctggccgataccctgcagcagtgcagagaggccagcagcagcgagaccatcatcgccagcaagcaccacaaacccctgagccccaagaccgtgagcaagtacttcaccaaggcccggaacgccagcggcctgagcttcgacggcaacccccccaccttccacgagctgcggagcctgtctgccaggctgtaccggaaccagatcggcgacaagttcgctcagcggctcctgggccacaagagcgacagcatggccgccagataccgggacagccggggacgggagtgggacaagatcgagatcgacaag

SEQ ID NO: 187 - DNA, 1071 bp, Artificial Sequence
  E174K/I43F/R319G triple mutant of the HK022 integrase
atgggcaggcggcggagccacgagcggagagacctgccccccaacctgtacatccggaacaacggctactactgctaccgggacccccggaccggcaaagagttcggcctgggccgggacaggcggttcgccatcaccgaggccatccaggccaacatcgagctgctgtccggcaaccggcgggagagcctgatcgaccggatcaagggcgccgacgccatcaccctgcacgcctggctggacagatacgagaccatcctgagcgagcggggcatccggcccaagaccctgctggactacgcctctaagatccgggccatcagacggaagctgcccgacaagcccctggccgacatcagcaccaaagaagtggccgccatgctgaacacctacgtggccgagggcaagagcgccagcgccaagctgatccggtccaccctggtggacgtgttccgggaggccatcgccgagggccacgtcgccaccaaccccgtgaccgccacccggaccgccaagagcaaagtgcggcggagcaggctgaccgccaacgagtacgtggccatctaccatgccgctgagcccctgcccatctggctgcggctggccatggacctggccgtggtgaccggccagagagtgggcgacctgtgccggatgaagtggagcgacatcaacgacaaccacctgcacatcgagcagagcaagaccggcgccaaactggccatccccctgaccctgaccatcgacgccctgaacatcagcctggccgataccctgcagcagtgcagagaggccagcagcagcgagaccatcatcgccagcaagcaccacgaccccctgagccccaagaccgtgagcaagtacttcaccaaggcccggaacgccagcggcctgagcttcgacggcaacccccccaccttccacgagctgcggagcctgtctgccaggctgtacggcaaccagatcggcgacaagttcgctcagcggctcctgggccacaagagcgacagcatggccgccagataccgggacagccggggacgggagtgggacaagatcgagatcgacaag

SEQ ID NO: 188 - protein, 357 aa, Artificial Sequence
  D149K mutant of the HK022 integrase
MGRRRSHERRDLPPNLYIRNNGYYCYRDPRTGKEFGLGRDRRIAITEAIQANIELLSGNRRESLIDRIKGADAITLHAWLDRYETILSERGIRPKTLLDYASKIRAIRRKLPDKPLADISTKEVAAMLNTYVAEGKSASAKLIRSTLVKVFREAIAEGHVATNPVTATRTAKSEVRRSRLTANEYVAIYHAAEPLPIWLRLAMDLAVVTGQRVGDLCRMKWSDINDNHLHIEQSKTGAKLAIPLTLTIDALNISLADTLQQCREASSSETIIASKHHDPLSPKTVSKYFTKARNASGLSFDGNPPTFHELRSLSARLYRNQIGDKFAQRLLGHKSDSMAARYRDSRGREWDKIEIDK

SEQ ID NO: 189 - DNA, 1071 bp, Artificial Sequence
  D149K mutant of the HK022 integrase
atgggcaggcggcggagccacgagcggagagacctgccccccaacctgtacatccggaacaacggctactactgctaccgggacccccggaccggcaaagagttcggcctgggccgggacaggcggatcgccatcaccgaggccatccaggccaacatcgagctgctgtccggcaaccggcgggagagcctgatcgaccggatcaagggcgccgacgccatcaccctgcacgcctggctggacagatacgagaccatcctgagcgagcggggcatccggcccaagaccctgctggactacgcctctaagatccgggccatcagacggaagctgcccgacaagcccctggccgacatcagcaccaaagaagtggccgccatgctgaacacctacgtggccgagggcaagagcgccagcgccaagctgatccggtccaccctggtgaaagtgttccgggaggccatcgccgagggccacgtcgccaccaaccccgtgaccgccacccggaccgccaagagcgaagtgcggcggagcaggctgaccgccaacgagtacgtggccatctaccatgccgctgagcccctgcccatctggctgcggctggccatggacctggccgtggtgaccggccagagagtgggcgacctgtgccggatgaagtggagcgacatcaacgacaaccacctgcacatcgagcagagcaagaccggcgccaaactggccatccccctgaccctgaccatcgacgccctgaacatcagcctggccgataccctgcagcagtgcagagaggccagcagcagcgagaccatcatcgccagcaagcaccacgaccccctgagccccaagaccgtgagcaagtacttcaccaaggcccggaacgccagcggcctgagcttcgacggcaacccccccaccttccacgagctgcggagcctgtctgccaggctgtaccggaaccagatcggcgacaagttcgctcagcggctcctgggccacaagagcgacagcatggccgccagataccgggacagccggggacgggagtgggacaagatcgagatcgacaag

SEQ ID NO: 190 - protein, 357 aa, Artificial Sequence
  D215K mutant of the HK022 integrase
MGRRRSHERRDLPPNLYIRNNGYYCYRDPRTGKEFGLGRDRRIAITEAIQANIELLSGNRRESLIDRIKGADAITLHAWLDRYETILSERGIRPKTLLDYASKIRAIRRKLPDKPLADISTKEVAAMLNTYVAEGKSASAKLIRSTLVDVFREAIAEGHVATNPVTATRTAKSEVRRSRLTANEYVAIYHAAEPLPIWLRLAMDLAVVTGQRVGKLCRMKWSDINDNHLHIEQSKTGAKLAIPLTLTIDALNISLADTLQQCREASSSETIIASKHHDPLSPKTVSKYFTKARNASGLSFDGNPPTFHELRSLSARLYRNQIGDKFAQRLLGHKSDSMAARYRDSRGREWDKIEIDK

SEQ ID NO: 191 - DNA, 1071 bp, Artificial Sequence
  D215K mutant of the HK022 integrase
atgggcaggcggcggagccacgagcggagagacctgccccccaacctgtacatccggaacaacggctactactgctaccgggacccccggaccggcaaagagttcggcctgggccgggacaggcggatcgccatcaccgaggccatccaggccaacatcgagctgctgtccggcaaccggcgggagagcctgatcgaccggatcaagggcgccgacgccatcaccctgcacgcctggctggacagatacgagaccatcctgagcgagcggggcatccggcccaagaccctgctggactacgcctctaagatccgggccatcagacggaagctgcccgacaagcccctggccgacatcagcaccaaagaagtggccgccatgctgaacacctacgtggccgagggcaagagcgccagcgccaagctgatccggtccaccctggtggacgtgttccgggaggccatcgccgagggccacgtcgccaccaaccccgtgaccgccacccggaccgccaagagcgaagtgcggcggagcaggctgaccgccaacgagtacgtggccatctaccatgccgctgagcccctgcccatctggctgcggctggccatggacctggccgtggtgaccggccagagagtgggcaaactgtgccggatgaagtggagcgacatcaacgacaaccacctgcacatcgagcagagcaagaccggcgccaaactggccatccccctgaccctgaccatcgacgccctgaacatcagcctggccgataccctgcagcagtgcagagaggccagcagcagcgagaccatcatcgccagcaagcaccacgaccccctgagccccaagaccgtgagcaagtacttcaccaaggcccggaacgccagcggcctgagcttcgacggcaacccccccaccttccacgagctgcggagcctgtctgccaggctgtaccggaaccagatcggcgacaagttcgctcagcggctcctgggccacaagagcgacagcatggccgccagataccgggacagccggggacgggagtgggacaagatcgagatcgacaag

SEQ ID NO: 192 - protein, 357 aa, Artificial Sequence
  E309K mutant of the HK022 integrase
MGRRRSHERRDLPPNLYIRNNGYYCYRDPRTGKEFGLGRDRRIAITEAIQANIELLSGNRRESLIDRIKGADAITLHAWLDRYETILSERGIRPKTLLDYASKIRAIRRKLPDKPLADISTKEVAAMLNTYVAEGKSASAKLIRSTLVDVFREAIAEGHVATNPVTATRTAKSEVRRSRLTANEYVAIYHAAEPLPIWLRLAMDLAVVTGQRVGDLCRMKWSDINDNHLHIEQSKTGAKLAIPLTLTIDALNISLADTLQQCREASSSETIIASKHHDPLSPKTVSKYFTKARNASGLSFDGNPPTFHKLRSLSARLYRNQIGDKFAQRLLGHKSDSMAARYRDSRGREWDKIEIDK

SEQ ID NO: 193 - DNA, 1071 bp, Artificial Sequence
  E309K mutant of the HK022 integrase
atgggcaggcggcggagccacgagcggagagacctgccccccaacctgtacatccggaacaacggctactactgctaccgggacccccggaccggcaaagagttcggcctgggccgggacaggcggatcgccatcaccgaggccatccaggccaacatcgagctgctgtccggcaaccggcgggagagcctgatcgaccggatcaagggcgccgacgccatcaccctgcacgcctggctggacagatacgagaccatcctgagcgagcggggcatccggcccaagaccctgctggactacgcctctaagatccgggccatcagacggaagctgcccgacaagcccctggccgacatcagcaccaaagaagtggccgccatgctgaacacctacgtggccgagggcaagagcgccagcgccaagctgatccggtccaccctggtggacgtgttccgggaggccatcgccgagggccacgtcgccaccaaccccgtgaccgccacccggaccgccaagagcgaagtgcggcggagcaggctgaccgccaacgagtacgtggccatctaccatgccgctgagcccctgcccatctggctgcggctggccatggacctggccgtggtgaccggccagagagtgggcgacctgtgccggatgaagtggagcgacatcaacgacaaccacctgcacatcgagcagagcaagaccggcgccaaactggccatccccctgaccctgaccatcgacgccctgaacatcagcctggccgataccctgcagcagtgcagagaggccagcagcagcgagaccatcatcgccagcaagcaccacgaccccctgagccccaagaccgtgagcaagtacttcaccaaggcccggaacgccagcggcctgagcttcgacggcaacccccccaccttccacaaactgcggagcctgtctgccaggctgtaccggaaccagatcggcgacaagttcgctcagcggctcctgggccacaagagcgacagcatggccgccagataccgggacagccggggacgggagtgggacaagatcgagatcgacaag

SEQ ID NO: 194 - DNA, 21 bp, Artificial Sequence
  mCF1 attE
actctttgaaaattaaagtcc

SEQ ID NO: 195 - DNA, 10 bp, Artificial Sequence
  mCF1 attE O
  misc_feature (8)..(10): n is null
gaaaattnnn

SEQ ID NO: 196 - DNA, 21 bp, Artificial Sequence
  mCF2 attE
tcacttaaccatgaaaagctt

SEQ ID NO: 197 - DNA, 10 bp, Artificial Sequence
  mCF2 attE O
  misc_feature (8)..(10): n is null
accatgannn

SEQ ID NO: 198 - DNA, 21 bp, Artificial Sequence
  mCF3 attE
tttcttttgccagtaaagtca

SEQ ID NO: 199 - DNA, 10 bp, Artificial Sequence
  mCF3 attE O
  misc_feature (8)..(10): n is null
tgccagtnnn

SEQ ID NO: 200 - DNA, 20 bp, Artificial Sequence
  Primer 635
ggcgccgtccaggcacctcg

SEQ ID NO: 201 - DNA, 26 bp, Artificial Sequence
  Primer 1185
gaactgaggggacaggatgtcccagg

SEQ ID NO: 202 - DNA, 22 bp, Artificial Sequence
  Primer 421
gaggccgcctctgcctctgagc

SEQ ID NO: 203 - DNA, 28 bp, Artificial Sequence
  Primer 1016
cgtgacaccctgtgcacggcgggagatg

SEQ ID NO: 204 - DNA, 35 bp, Artificial Sequence
  Primer 834
ccaccccattgacgtcaatgggagtttgttttggc

SEQ ID NO: 205 - DNA, 29 bp, Artificial Sequence
  Primer 1191
gccgtccgtcccccctttctttgactcag

SEQ ID NO: 206 - DNA, 25 bp, Artificial Sequence
  Primer 432
tcgttgggcggtcagccaggcgggc

SEQ ID NO: 207 - DNA, 24 bp, Artificial Sequence
  Primer 1298
tacatccacgtcgaatcctcgcgc

SEQ ID NO: 208 - DNA, 22 bp, Artificial Sequence
  Primer 1015
ccgccgccgggatcactctcgg

SEQ ID NO: 209 - DNA, 26 bp, Artificial Sequence
  Primer 1300
atcttcctgccttggcctcccaaagc

SEQ ID NO: 210 - DNA, 27 bp, Artificial Sequence
  Primer 1279
gccgttctccagctttacgacaggagg

SEQ ID NO: 211 - DNA, 29 bp, Artificial Sequence
  Primer 1232
gaggcttttctgttacagcgtcctcctcc

SEQ ID NO: 212 - DNA, 35 bp, Artificial Sequence
  Primer 1236
ggtctcttaggaagaccttgtcctgtagtcagtgg

SEQ ID NO: 213 - DNA, 140 bp, Artificial Sequence
  P for attP donor cassette
aggtcactaatactatctaagtagttgattcatagtgactggatatgttgcgttttgtcgcattatgtagtctatcatttaaccacagattagtgtaatgcgatgatttttaagtgattaatgttattttgtcatccttt

SEQ ID NO: 214 - DNA, 77 bp, Artificial Sequence
  P′ for attP donor cassette
taagttgtatatttaaaatctctttaattatcagtaaattaatgtaagtaggtcattattagtcaaaataaaatcat

SEQ ID NO: 215 - DNA, 3014 bp, Artificial Sequence
  CF Native replacement sequence for exon 3 mutations recovery using CF10 and CF12
ctacttttaaaaacaaagtctaacagatttttctcatgttaaatcacagaaaaagccacctgacattttaacttgtttttgatttgacagtgaaatcttataaatctgccacagttctaaaccaataaagatcaaggtataagggaaaaatgtagaatgtttgtgtgtttattttttccaccttgttctaagcacagcaatgagcattcgtaaaagccttactttatttgtccacccttttcattgttttttagaagcccaacacttttctttaacacatacaatgtggccttttcatgaaatcaattccctgcacagtgatatatggcagagcattgaattctgccaaatatctggctgagtgtttggtgttgtatggtctccatgagattttgtctctataatacttgggttaatctccttggatatacttgtgtgaatcaaactatgttaagggaaataggacaactaaaatatttgcacatgcaacttattggtcccactttttattcttttgcagagaatgggatagagagctggcttcaaagaaaaatcctaaactcattaatgcccttcggcgatgttttttctggagatttatgttctatggaatctttttatatttaggggtaaggatctcatttgtacattcattatgtatcacataactatattcatttttgtgattatgaaaagactacgaaatctggtgaataggtgtaaaaatataaaggatgaatccaactccaaacactaagaaaccacctaaaactctagtaaggataagtaaaaatcctttggaactaaaatgtcctggaacacgggtggcaatttacaatctcaatgggctcagcaaaataaattgcttgcttaaaaaattattttctgttatgattccaaatcacattatcttactagtacatgagattactggtgcctttattttgctgtattcaacaggagagtgtcaggagacaatgtcagcagaattaggtcaaatgcagctaattacatatatgaatgtttgtaatattttgaaatcatatctgcatggtgaattgtttcaaagaaaaacactaaaaatttaaagtatagcagctttaaatactaaataaataatactaaaaatttaaagttctcttgcaatatattttcttaatatcttacatctcatcagtgtgaaaagttgcacatctgaaaatccaggctttgtggtgtttaagtgccttgtatgttccccagttgctgtccaatgtgactctgatttattattttctacatcatgaaagcattatttgaatccttggttgtaacctataaaaggagacagattcaagacttgtttaatcttcttgttaaagctgtgcacaatatttgctttggggcgtttacttatcatatggattgacttgtgtttatattggtctttatgcctcagggagttaaacagtgtctcccagagaaatgccatttgtgttacattgcttgaaaaatttcagttcatacacccccatgaaaaatacatttaaaacttatcttaacaaagatgagtacacttaggcccagaatgttctctaatgctcttgataatttcctagaagaaatttttctgacttttgaaataatagatccataatatatattcttatggaaatctgaaaccatttgggcatttgggggtaaaaagtattttattagtaaatttaaatgaggtagctggataattaaattacttttaagttacctttgagatgatttttctcaatcagagcaccacccagagctttgagaaacaattttattcacagcttctgattctatttgatgtaatttttagaaaataagttttgctggttgctttgaatcagggtatggagtacagttcactctgatcctatcatataaatcatgtaagtatataacattttcaataagtgattgttggattgaagtgaatgatatttcaagtaattgttatgtcatggccaagatttcagtgaaactcaaaatttctcctggttgtgttctccattgcatgctgcttctattgattaacctaagcactactgagtagaagctggaagaggggtctaattagaaggcccctttctatgctctgcttggcttgtaaaataatttatttctctagatcccaccaacatagtagtttcatgtatgcaaaaacacccacctaaatgtcaaagtttgtatgatacatggacatatctatagaattttttttggtctggtgcatgccaaaaaataaacatgatatagaagaatttaatatttattgagtacctaatctgttccagttcaatatgaaggtctttatgcagattattttacttaattttcctagtaactccatggagcaaaaattatctctaatttatataacaggaagttgagcgtgaggcaaattaagtaactttcccaaagttacacatatggtaagtttgagagatatcccagtctctttagctccaaagcctttgaccctttcaccataccagattatgattgctattaatatataattataattataatgattgtatttaggtactcaacagaatggtgactctagtaaccagccttggttctgctgagcttctctgcgtcttctcaggagacacaggctacagagcttgaaggctgaggattcttccagggtcacttcaggggcaaatctgaaactttcttcaggacaggaatcaacgagatcttctcacttacttatacctgggggaggaactgtatgaaatccacccaagaaccagtcatgctaagggccaaacctatagacaaaaaaagggataggagaatggagtatgtatggagaaagactaaattgttcttaaacttctcaagcttaaaaatatcccagcaaaagagatcgtaaaagcccttcatggcgtattaattatccatgcatgggggtgagtggaaaggtactcctgagcccgaggctacagctttggaactagcagcacctttgaaggggaaagcgt

SEQ ID NO: 216 - DNA, 4443 bp, Artificial Sequence
  CF Universal replacement cassette with cDNA for any mutations recovery
atgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttag

SEQ ID NO: 217 - DNA, 22964 bp, Artificial Sequence
  DMD Native replacement sequence for exon 44 mutations recovery using DMD2 and DMD3
taccttttctccattaagcaatttcctatccttcgcccccatcccaccctctcgcccttctgagtctccagtgtctattattccacactctgtgcgcatgtgtacacattatttagcttccacttgtaagtgagaacatgcaatatttgactttctgtttttgagttattccacttaagatgaccaccagttccatccatgttgctgcaaaagacatgatttcattctttactatggctttgtagtatttttcattgtgtatatgaaattgtttattccatacgcaatttgtgtgtgtgtacatatatatatatatatatatatatatatatatatatatatatatatatgcttagacttagaagctaggatagacacacaatggaatactacacaatggaatacattcattcacacacatataaataaaagaatatgtggagatatatctccacatattctttatccaatcatctgtttttaaataatgctattgacttctttagggtgaattttatcaatattgttttggtttaaaacactcaccttaaaagagtcacagtccctaaatgtgcatcctcatatttaaattaggtctcagtaaatttgtgcaaagtgtattctttttaggatggtgttgaacttgctaaattatttatctttaagaatcatcattttgtgtcttttattaatgaaaacaacaattatgtgattgctgatatatttggaaaatgatttctgatgtagattgatttttttattctaaattctgtgtcggtattaaaaatttatagattactaactgtattaatatcgataatactaaattttattgctatttataacttggagtgtactttcatcctcctgaaaaagctgaatgaggtaggcagtattattctgggtttatgtgtgagataactgagactcagaggtaaaatagtgtatccaagcattcatggctcttaaatggaagatataaggggtttgtgaaattactcatggacttttttattcattcattcagttattaaaatgtattcaacatttatcatgtaccaggaacagcgcttagtaccaggaattcaaaggtgcataaaacatcttccttattctaagaggtacatagtgtactggaacaaacagccttgtaaatacataattagaacatgaagtagtatgttaatagaggttttcacaaagctgtggaagcttgtcttatgaagtaactaattccaagggagagaagccttatggaatagtgacattttagatagggtgtcattctaaaatacagcaaaaggcccacagtaaaaaaggaattttggttgttatgaaaattttcagattttctatgttttcagtacagtatacatggtgggctatgtgaatgtttgtatagggaccaaagtaggaagtgaggttgtctgttagagagcgctgagaaaccgaaaatagggagagatgagttggaatatgctgaggaaaagttattaggagttttcaagaaaggccacgacagtggggctagagagaagaggctaaattaaagagtcatttctggtttagaattgataaaatatagagacaagcatgataagaaagaagtcgagaagtaaacgatggtctcaagatttctagcttggaaatcattgactaaaattaaaactaaggactggattaggccattcttgcattgctataaagaaatacctgagactgggtgtttataaagtaaagaggtttaattggctgacgattctgcaggctctacaggaagcatagcaacatctgtttctggggaggcctcagggagcttttactcatggtggaaggcagagcaggtgtaggcatttcacatggcgaaagcagagagagagagttggtggtgggggtgggtggctacctacttttaaacaaccagatcttggagaactcactcattttcatgaggacagtaccaagaggatggtattaaaccgtgagaaaccaccctgatgatccagtcacctctcaccaggccccacctccaacattggggattacaatttaatatgagatttgggtggggacacagatccaaatcatatcaaagacttgcatgggaaaataaggaattgttgacataacatctttgaggttcacatcaaatgttctgatgaggatagtccaagtagcagttggctatatacctcagataagggctgaaatttggagctatgtcataatcagcctagattaagagtcaataatctcctgcccatgggccaattacacccaccacttgtttttgtaaagtagtattgaatcccagccatatccatttgcttatgctccatgtataccttttttttgaacttcaaggcagagttgagtagttgtaacaaaaaccatacggcccacaaagcctgaaatatttgttctcaagatctttatctataaagtttgccaatacctgctgtagatgttagttgaagctttgaaagcaaatgaggtttcataaggcagtgtccatacaagacatttaacaagtttacctataaaaactagaattcctttgaggggaacacatcctagtctccattaagcacagtagaagagtcccctataatgggaaagaggtcactttaggtgttgatgttggtggtacaggtcaaagaaaatttatctttgctgtttattcagaatgcaataagtgaagttatgagaaataagggaaaaaatgtgtagaatttcaacagcgaagagaggggataaaggcatgagaatgagttcctaagctcaagtattataaacactgtgagaaacttaaaatcaaagtatgactccaaacgtatttgaagcctgagaacaaggctcacaacctagggaggattagggatcaataaaatagagtgttacaaagtataatgtcaatccagagttgtaaaaatatcagcattgaatatattgaaagcagtaaaactgaatgaggagactatcattttatatcactgtgtttatttctttgccttgttctataaatatttaaaattataaaatttttattaacagtgagagcagaactaccagagtgagcagatcaaaattgggacagatgcttttcactgcacacacttttatttttctgctgttcatgcattatcttgtacagtgcacatgttttacctaaaaaattaaaatggagtctcctgcttaggaaaaaagtatatattctgtttcaaactatatacaaaaataaaatcccaggtgactaaaaactgacatgagaaaaaaacaaattgataaagcttttacagtaaaatagaggagaatatgttaattaatatagggtaagaaaaaattgcttacacaaatgatgaagcactaatcatgaataaaaataataaagtggactaccttgtatattaataacatctatacatcaaaagacagcactgagagagtaaaaatgaaacccacagagtaggataaattatttggaatacacacataatggatgaaatgtgtgtattcataattataaagaattcctacaaatctttcagaaaagaacagataatccaatagaaaaatgggaaaagttcttgaaaagtgaaccatggcacaaaaagggcttgtggcctgctggcaatattctgtatcttgacctggatggcatttttaaggtgatcactttatagtaaataactaatgtgttttatgcatcatagtaacgttaagatttttgtcatctttacaaaataagaaatccaaacggccaataaatatataaagaatttctaagtcccattaatggtccaggccatgcaaattaaaactaaaatgaaatatcactgcttaccaaccagaatcattgaaatttataagtctgacaattccatgtggtggtgagaatatacagcaattagaaatttcacacaatgttacttggtctgtgaattgtaaatagaagtgtaaaattacactactgcttcttggagtgaaatccatttggcactatttagtaaattcaaagatctgcataacctatagcccaccaatttcacttctatatatacactctacagaaatgcatatgttcatattccaggagacatgtttgggaatgtcatagcagcatagtaatagccccaaaccaaaactacttcagtatttattaatagtaaaatttgctatagtttgaatgtgtctctttccaaattcaggtgtcgataatgtgctagtactaagaggtagggtgtttaagtggtgattaggccatgagggctccttctttgttaataaaaataagacccttataaacaaggcttcacgcagcattcagtcagcttgctctcttgcccttctaccttctgccttgtgaagatacagcaggaaggccctcaccagacaccaaatgccagagcctttatcttggacttcccagcctccagaactgtgagtgaatacattggtattatttgtaaattacccagtctcaggcattttgttataacagcacaaacagactaagacaatcatacagtgagaaattaatcaacaactaataagcaaagaggtagattaatcttgaaactatgatatagagtgttccatttggctgctggaagttttatttcttggtctgggtgatggtcaccatgggtttatatgaatggttccctatattatgtttcacaacaaaaagcatttaaaaagtaaatatatgtaatgtactcagggataggcatggccaaccatggattctatgctgaaataatgattcagatttcatcagcaggctaatgacactgcctatttaaatactttaagtcctgaaattaaagaaggtaatttctcaagaaggaatttctaatttatgggtgggtctattccccaccagagagacactagcatggctcagattctatgttggtcattttatttgcatttaaagtcttaagccaaatagaggtacactaataatgacaacaactactactactcatacttgtggaacactgccagatgctgttttaagaaatttgcattttcatttgtaactgagcttacttgaatcttctctctttttttcttggttaatctaactactggtctatcaattttacttatcttttcaaagaatcaacattttgtttcattgatcttttatatttttgtttcaatttcatttagttctgctctgatctttgttatttcttttcttctggagctttgtgttggctttgttgttgattctctagttccttcaggtgtgatgttaggtagtcagactgtgaactttcaggctctttgatgtaggcatttggtgctagaaaatttcctcttagccttgcttttgctgtatcccagaggttttgaatagattttgttgtgaatgtgatgaaaacggaacatttgtacactgctggtgattgtaaattagtacaacctacatggaaaacagtatgaagatttcttaaagaactaaaagtagatctaacatttgatctggaaatctcactaccgattatgtacctagaggaagagaattcattatatcaaaaagacacttgcacgcatatgtttatagcagcacaattcacagttgcaaagatatggaaccatcctaagtgccagccgaccaatgagtggataaagaaaatgtggcatatattttcatataccgtgaaatactattcagccacataccatgcaatactactcagccgtagaaaataatgaaataatgtcttttgcagcaactttgatggagctggatgccattattctaagtgaagtaattcaggaatggaaaaccaaatactgtatgttctcacttataagtgggagctacgctgtaggtacacaaaggcagacagagtggtagaatggactttgaagactcagaaggggcagagtgggaaggtagtgagggataaaaaattacctttggggtgtaatgtacactacttgggtgacacgtgcactaaaatatctgattttacttctatacaattcattcatgtaaccaaaaatcacttgtattccaaagactattgaatttgaattttttaaaaacattaataaaataaaagatgtaaaaaaagaaatttatatatactcatttattgagctcccacaattaaccttaggaggtaagtacttcataattggtagtatacttatcttttactaaatatttgtattacttgggaagttgagggttggggagaagtagcaaggtactatgatttggggcagataactaacttatttattcgcacatacagtttggaccatgagacacgagctcaggtccctcctcctcacctaatcaaagatgaaatatgtgggatgggatgaaataatcagcagtccaatgctgagtttccagaccgaagtataaagcaacaatggatatgtcagaagtctactagggtgttatttatttaaatctatttcatggaatttactaccaccttaatggcccgaaagtgttaaagtatgccccagagtaccgaattactccctaaatgtaatttatgcttgagaataatctgactaacttgatttagaacatcagaaaataagttatgctgcacataaatgaagcagcagtgtaattttaaataccggttgcacggtgaatgagaattttaatatttgcaaaattctaaaatcacttgatttattatccttatgtttatactgacatttttttgccctttgttaagttccatccatatttcttcttactgccaagaaaaaaaactttttttcctagaaatattacagaaggcaaaaattatatttgtttccctgaatgctatttttgatgtctctacttgtttctcattgttaccatttgcttcattcatgggcagcccaattaatggagcgagacaaatttagggagcacagtgactaattagatattaaattggtaaatctaactttgtaaaaccagaaaaaatatatatatatttttttcatttggaattttccttggtggaaaagagtttaaaagtagtcatgataaaaaatgtaattttacgtagtaaattcaagaatagatttagactgtgctattaacagcacctattaaatactgaaaagtgtattttaaaattttatgtgaggcttgaaatggagtctaaagtattattactcacattaagtgtcatcacatgtaaagcccatgattttattctttaatattttgtttgaatagttacttatttcaacagtaatttcaataataaaattaaatcaactttacagttttcaaaggtttagcagttgcatgctgtaataaatacttcatatttatatatttataaagtgacagcataagtcatttttattaggtccttgaggatgcaaaagtttggattatacgaggagacgagagaaaaagggaagaagggcatttcagaaatatgctaccgatatgcaaattcacaagtcctaagacagtagcaggggtcgggcagaaagtccatcctgcctccctcttgtgggcctggaacaatggtgtaagtggaaggcctgttccccttctcttcctacctccagctctgtcttacagagctacggataccatgagcaagtgtatgaacccttacggttttcttctcttgggagaatgtaaaggaaagataacttgtagaaacttgtagataacttgtaaaaaggaaaagaattcagggtgagagggggatttgttgaatttgatagaggatggcaattaccaatatgatgagtgattgagaaacaagtctgtgcaacaggtttgaaatcgaaaatctttgaggtgtacaggatcctgaaatgaagaatgggcatttatagcagtatgtcagagaaacagtcacctcctagtagctaaaagtgttggcaaaagtatagttcaagtgattgggtaggaaaaacagcaaaccaagagtggagactgatggttgctacaaaggtggagtggtaagtcgtgaccaactggtacttctctgtgctctggttagctgctgactgtttctcagactgtggtagcaggaggagggttggagttagcagtcatttgcatatgagactgccatttaaaaaaaaattttaaattatttcatttttctgactctcaatatgaaaagcacattgtagacaaattgaaaaatatagaaaaattatataagaaaatatagtctcaccagtatggaacaatgctaactatgttgcatagatttttagattctcattcaaaagcaactctttgactccagtgatgcaaatgcatgtaacatatgcaatgtgcaattcatttttaaagggaataaacttacgatatattcataggtcatttattgtgtgttatataccattgaaaatatatgaatgctaaattattagtaaacatgcaaaaacattggcaagatcattttgttgtggaaggatatattgtatctgaataactctagaataccataaatcatcaaaggcaacattcttatttttcactaactacagttagagaatacctcttcggctaccttcggttgccttttttatgctaccaaaatgctgtctgttttacaagattttaaaggttaagcatataattattcattaaatacaatgagtgcaatgtacatgtagatacattattaaattttgggtagttaataaaaataaggggaaaaaacctctagaactatcacttttaattgtttaactgataaagtgaagcttcatcttggaaaaataatttcacaagagagcatgtgcactggtagaaaagtgccattgaaacaagagatatttgggttagaagcctctctctactatttaataccattttcaccttttggcaaattacttggcctctgttttctccaatggaaaatgggaataataattgttatgctgcagggttattgtaggtgtcaatgaaatgatgtgtctggcactataaaagcacagagcccggtgcctggctattagtaactgtttaataaatgttaattcctttctctgcccaggacatcagtaggcagatgtagcaatttaaaacttctagtgttactttaaattcctgaatgaaggtagaggactgaaaagatatcatggtattcaaaagtatgatccattgcttcttaagaatagagttcagaaaagcttgacagattcctgtactctgaggcagcaccatagccggtaatctgtaggatggctattggttttgtgctcacaaatgcttgcttgggcaggccccaggaaatctggtagactgtaagcccagtaagatttcaaatcttactttacggcagtgtttttcaccttgactgtacattgaaatcacctggatgctttgaaaaataacagcgtcagtgtccaacctccagaaatactgattaagttggtctggaatggagccccaggatcactgtttggttattgttgttgctgtgttttaaatgccccagttgattcttatgtgcaactgtcttaggtaaacatacagccctggttcatattatttctgcctcagtctcttttatgactggaaggtgaccaaatgcttgtttcctaatattctttccatgtgtagtattaacacatttgacttgtactaagttcctgcagtattccaatctaaaattttagtgactacaataaaataagaaggattaaagaaggcatcgcatagtttagtatatcggttatttaatgcttacatgtgagcctacaatatgaattatatctgtcatcttattttaaatattgacagaatctttaatgatagtgacgaattattgatttattggtgtgataatggtattttagttatatttttaaagttttatttgtaataactatatgtatttatggggtacagtgtgacgtttcagtgtaatgtttcattgtgtaatgatcaaatcaggtttcttggcagatccatagcctcaaacatttataatttctctgtggtgagaaaatttaaaattctctttcactattttgaaatatacagcacaatattggtaactttgttcatattactatgcaatagaacactagaacttattactcctttcagttgatgaacaggcagttttggatcaagaataatattgaaagtgatagaatttatgaagtaatttttatccaaaaatattttgaaagggaatatattgcttccaaataatttattacaatgttaagatatttgtaaatttctagaattaaaaaaatatatttttaggaaagaaaatgccaatagtccaaaatagttgctttatctttcttttaatcaataaatatattcattttaaagggaaaaattgcaaccttccatttaaaatcagcttttatattgagtatttttttaaaatgttgtgtgtacatgctaggtgtgtatattaatttttatttgttacttgaaactaaactctgcaaatgcaggaaactatcagagtgatatctttgtcagtataaccaaaaaatatacgctatatctctataatctgttttacataatccatctatttttcttgatccatatgcttttacctgcaggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggtaagtctttgatttgttttttcgaaattgtatttatcttcagcacatctggactctttaacttcttaaagatcaggttctgaagggtgatggaaattacttttgactgttgttgtcatcattatattactagaaagaaaattatcataatgataatattagagcacggtgctatggactttttgtgtcaggatgagagagtttgcctggacggagctggtttatctgataaactgcaaaatataattgaatctgtgacagagggaagcatcgtaacagcaaggtgttttgtggctttggggcagtgtgtatttcggctttatgttggaacctttccagaaggagaacttgtggcatacttagctaaaatgaagttgctagaaatatccatcatgataaaattacagttctgttttcctaaagacaattttgtagtgctgtagcaatatttctatatattctattgacaaaatgccttctgaaatagtccagaggccaaaacaatgcagagttaattgttggtacttattgacattttatggtttatgttaatagggaaacagcatatggatgataaccagtgtgtagtttaatttcaacttgtggtgtcctttgaatatgcaggtaaagatagattagattgtccaggatataatttggttgctaaattacatagtttaggcataagaaacactgtgtttattacacgaagacttaattatttttgcatcttttttagctcaaattgttcatgttgcaatagtcaatcaagtggatttgaattgtagccaatttttaatgccagaaaatactgattaagacagatgagggcaaaaaacacccagtagtttattaaatactttagatatttcaaaatgctggattcacaaaagcagtatcacatttgactttacaagtcttcattctcaaatatgtttccatagtaaatatgccctttaatattaaggagttaagcatttaaacacctatttatatgataagctatttaaacacagaaaatatttttaaaaccttgtgtaattatatgtgtatcaatcaaacttgcatgcacaccagcgttggcatttgtatagagaggaaatgtatggattcccaatctgctttaatatagaagatacattttaaaaatagcactgaagtgaattttgggctaatgtagcataatggggtttctgcctgagaggcagaaacatattagagttatataaaatgttttggggtagatatagaaaccacttgccattttcaatgatatccaacccaaggtagttatatatttcaatttatattttattatcaaattagtacttattgtgaaaaaaatcaagtaacatagaaatttgtaaaagtacctccattctactctttggaggatagttgttcagtatgaattttgctacatatttcaggctgggtttcttggaaagccattgtaaaatggagatttgtatgtagaaggttaactagggagtacttttacgatgaagcaatttgttttgatgtaacttggtgtagttttcttcatgtttcttgttcttgaagtcagttaagctcttgaatctgtgcatttaacatttcatcaaatttagaaacctttcaaccatttttttaaaaaaaatggaactccaattgtacatttattaggctccttaaagtgccccactactcactgatgttatgttcattgtctgtttggtctctcttttctctgtaatttgttttatataatctctattgtcaaattgactaatctttttcaaagtctaatctatggctaatcccatgtagtatatatttttaacatcagacattttcatctcttagaagtaaaagttgggtctttttatttcttccatgtgtctactcaacatgttcagtctttactttcttgactatatggaatacagatataataactgttagaatattcttctctactaattttatcatctgtgtctattctgggttaatttaaattgatttatttttctcctcattaagtgtgttgtttaactgcttctttggatgactggtaatttttgactatatgccagacattgtgaattttaacttagcgcgtgcttgatacttcaaataaattcaaatatattgaaataaatattctcaaacctcgttctggaacacagttaattcacttggaaacaatttgatcttttgagaatcttccttttatgctttgttatgaccagaacagtgtaagtttagggctactttttccccactactgaggcaaaacccttctgagtactctctctgatgtcctgtgaatgataaaatttttcactggggctcgtgggaacaggtggtattactagccacgtgtgagctctggtgattgtttcctttaattcttttgtgaagttctttccttagctttgagtggttttcttgcatacatgaactgatcaagactcagatgaagaataaaataaagctttctacaaatctccaaaatttcctctgtgtatatatcacctctctggtattttgccctgtgatcactagtcagccttgggctgctgaaactctcagcttcatcttttaacaaaagcctcctggcaaggatcactgtccttcaatgtctgatgttcaatgtgttgaaaaccgttgtagcatatattttgtctttttttttttttttttttttttaagtgtttcaggtgtttcaggcaggagattaagttcagcctcctttactccaacttgaaaacaagtccaaaacaaactattttgatgtaatttgatcttttaatacattaacattacacaattttgtgaatatatcataatttaaaattttcagagaatgtctaatggtcctcatttcttgacagtgtggtttagttgaaactgatgaacattttatcaaaacttttcccctcaattggatacttttttttttttgagatggaattttgcttttgtcacccaggctggagtggcatgatctcagctcactgcaacctctgcctccaggcttcaagcaattctcctgccttagcctcccgagtagctgggattacaggtgcccacccccacacctggctaatttttgtatttttagtagagacgagatttcaccatgttggtcaggctggtctagatctccgacctcaggtggtctgcctgtctcagcctcccaaagtgctgggattgcagacgtgagccaccatgcctggccaactggataattttaaaaagaccattttatttagtctattttttctcaatctatagatgagataagaaaaatcattctagatgtccaaggaaaaattctttcagaaaagagctgtgaatgatatcacaaaccccccaaacagttaaggtatttctttcctggttattttatgtccaaaatcatgcatatgaacatgtgcacacacatgagcgtgcacacacacatgaatacatatacacgcacataatgtaccttaggttatctttccattctgagtaattatcgtaaaatgggtaaaatcaaccccgtaagataccttcatcgataaggcaaatcaaagctttggtaatttctgctatcttggcctttgttgattgactaataatgaataagagaatgagtttcaatatttactatgaaattattttagaagacaggatgtagacagtggctgttagcaggcaattgtttggcatgagccagtaatggttactgtgaaaaaaatcaaccaagcagcccatatattaaacaaacacacgcagaagcacgttggagtctgaagcctcatatgtacaattttcagtaaagaaataacttttagatatgaaataaacaaatagatatatgttgtaaacttgtccctatgtattttgatcaaattgcatcatatttttttcactttaaagaagagaatttagtgctttaactgagacttagtgttatcattcaaaatatactgactgccaatagcagtagaaagataatctggttccatgcaactctattttttttcctctgtcgcaagtaaaagacaaaattaagtacatgaattagtgctttttgaagatattccagagcaatataccatgccactatggagaacctctctaaaaatatcccatttttttacctgagaaaaatattgatcatgttatatgccactcaaattggtttattaaattcgttgaatgatatcagcatctcttaatgcattcactaaacaagcagtaattgagtgcatatacaaagttttatcatccaccaaaacagtgacaatccacatgaggctctaatagaagtttagaaagggggttaagtggttaaatgctggactcagaaagattggattcaaatcccaggtcctttagcttaatagttgtagaatcttgtgaaaatatcttaattcttttcatgtctctgatttctcttctctaaaatggaaatataaatgagatgtgtataaagccacttggaatagcattttgcacaaaataattactcattaaatgtaagcccctattataactaatcactctttataagtgattagttcatatcaatacaaactaagacttatttactgaattatcgtctctaaacatccacactgcagaaaaaccaacctggaaatttcataaaaccttatttttatgtagtataatttcttctcaaagcataagggctcttggattaggaattgaggaaaattccaattcagccaaacgcatctgtttcagatagctgacacttctgcctactcatttcctagctaacaagaagaaatgttaatgggagttttcaaaggaaaagctgaacaccatgaaggaaagtgacacaaataatgttagctcatatattgacagggtgaatttgtgtgctttcaagtcccttcagtgaaaataggaaagtagaaattataaaatgccctaacatttaaagctagcatgttcttggagactaggaaaaaataagttttaaaacatgggctatgatagaatgagatggaaaatgtttgtagttgccagtagaaacaataacaattaccattagattaagtatttaaaccagctgaatatttttattaatggaaatggcatctgttttatgaaataatgctgctgaatgaaccatattaaaaatgaccagtatttcctgcagaacgttgtcgcagacatacaagcctgagaccctaaaatcttaaggtattccatttgaaatcgaccttaagacattaacagtagtggtattgtttagatgaaattttttaggctttaaatcaacaaatgttaagcagacatggggagcgaaacaccagtgtgttattctgacatgaataaactgctgtttttagggaaaaaatatagtcttgttaaggttaagctaattggttttctggtatcttttgcaatgttagtgtgttttactgctccataacctatgttatatggtaaatgtgcaatatatttatatatgttgctgtaaagaaatgtaataaaaaactgtttactttgtgatatgaaagtaaaaatttattcattgtcattgagcatacagaagtaaatatggattacatatgtcatattttaatgttcacatggtcccaccatcaaatgttgaaaaacttatagtttaacgtcatattctattgaagaaaaatacactcccttttctcaaatgtgaaatgtccagagagaatggaaaattacatataaagcatgtagttatagcatggtgaccctgctgtgatctctcagatgaggaacaaaagggagaaagaaagagcacactggtgctttggagttgagagaaggcaaaaaaagagtacaaaaatgtcaaagccaagtttagctgctcttcagctctccctttagctgctcttcagctttaccttaccatggttattagtgattgaagaaaattctaaagcactttttaaaggacccaattctgaagagtttagattcagagagcacaatggagttggagtgactcctgctcaaaagtttgagacaagcgagtccatgaaaagaccgtcctcctcttaatggaaatacccaggttttctcattcttctcgccttgctttcagcactcgcagcccagaaagcccttatctaacaggtactgccgttgaaaggtcattgacttgtacaaaaatgatgagtgctgaatagatgtgcataggtcactgacagtatctgctacagagaatgagttttcgtatttttattaggatacacctaacatggcaatctactgcctcaaagaactctataggaggtaagtgaatttatattaatacagattgaattaaaggataatctagaaaaaggcatatgatgtaaaaaaatcagacacaagtatattttctgtatagtcagtttttacattgtgatttcaccagctggctgctgagtttgacggcttcttaacagccacactgctgagattcaaatgctgatagaaactttgatggaaaaatcactggagtaaatatttctaccatctgttgcccttcactgggaccctaacgttaagaataattcataccattgcttgtcctttatatttccccagcagtaataaaatttcataagattttgttttgtggtcacaaagctatcctggtttctgtaactagaagacatacactagcataagggaatcagccggaaaatttactgctaagagaatttgtctctagtcacttactttaaggttacagcaatgtgtaagtgtgggaatacattttaaaatgagcttttcaaagttattagctggtagtggcatgagagttaagtctcttaatacagttaaacagttgggcacttcatccttgcgtaaatattgttacccttttattgctgcttggaaactcctctgcaactttttggcccctatccatcttttcagaagtagtaaataaccaatttactgggagtgtggtaccaggcagaaattccgagaggggctttcaatccttgcccatcaagtgtatctttcagaaataagtatattaaaataattggataatttcagtggcttgttattagacttccgttgtccagcatggcatgtttaagaagatgacagattttcatacattattggaaagaagcaagaacaaaaaaacataacttactgtagtaaccacggtaaagaactgcttaaaatgcaggataaacatgtcatccctaagggattcccattcttagagcatgaaattatcaagagagtaagagactacaaaaaatgagaagaatgctgattgcaaattccaaatagaaaaaatcaaaacaaaactgcgcaccatcattctggaagcaatgagaagcagaaattgtcatttaatgaaatgtaagattaaagttaatagaagtaattttcatgaaataatattttgcaaggacgatgttccagccatattgatcttcgtgttttcttttcacatcccttcttactgttccctagaatgcttgtttctacctttaaatttgcttttctctctaccagagggctctaccctatctccagtttctcaccatgtcccaatctactccctctcagaatttttgtacacttccctttatatatatttgtgctctaattttatattcacagatatgccttttgtaactcccccatcttaaagaaagcacacacgtacgcacacatgcacacacacaaaattgaactctttctgggagatctgcttaactttcttcataactctgtcacttgctgaaactgtagtatgtgttttcatgtttattatcttttccattagaatgaacatattttgggtacttggtctttctcgatcaccaatatacctcggtacgtagaaaaattgattcatatattgaaaatgtaatattcagtagaacgaataaatacataaataaatttaaaaatgatacttttattgtattacctgagacaaatgatccccaagtttgtccttgcttttcatagccaaaacattctctcttacattgagcttccttcacctcttctgtgtacagagcacttaaaattttcacattgcctgatactttaacaatatgatggccctgttctcttacccattggagcatatgttaaataccagaacccatgtaacaaacatatattgtgatcctactgtgtgcaaagcagatactgcttgctgctaggaatacagagctgactaagagctccttttctctttatgagctcacagtctcatgagttcaacgtcttaaggcacaacgtctaaagcaaagggcagtaagtaaacactccagaaagtactggatctggcctaggacaaatggtgggttgtttttccagctgttatttttcctgccccctaattgacagtcctccattacacctctgggatacctagtctgacttgggaaaacctgactttgggaatcagaggcagtctctcttgcttatatatgaggaactctaatggatacttactgtcattagagaaactctgcttctagcctggctccttttgtaaagaaggttgagtccccttggagagcctgcagaacataaccatttgcatgtaatgaacagtttgtaatactttgagattgatgtgcaatttctatttgacaagggaaaaacaattaggattaaccgtggtcgtatatcccagaataccaacgttgtttccacactctaagtgttgttgggtcattatatgagattcataattttgtcctgttgtacccacgtttgcattaccattcagtcttaatttattataccctattaaaagtttttttggtaatttgttcttattgctactcaggcattaaaatgtctgcaggctgtgaaaatgaataaatttaatgtggcagcatagttctcaaaatcctggctttacaactcatagtacaggcttgtattgtaaatcctagttaacatggatttatttgaaaatccaattttactgctaatcttaaataacacatttttcaaacattttatccttgaatttctatttttttataatttatggctgttgtatgtatttacaaaaggacaatgtgtgtacttttaaatactagtaatggattgctgaaacaactgtaactttaaaacaatgcaattgttaaaaaaataaactgtgcagcctggcttaatggaggcttatgaacatatgattaagatatatgctataataagcaaattcactcaactgatagttcataggaactttcaaatttaatctcataaccagtgctatccttcaaagaatggtcagggcaatttaacgagtacatgaccacgcaagataatttcattgaagagtggctgaactgttgaaatattttctagtctccttgggatatcattaagagcagaaattttgaaatggaattgtaatgatgttcagaaaagataagtaggtaactctcttaatacgttttgtgctgctgtaacaaagtacctaagactaggtaataatttgtaatgaacaaaaatgtattggctcacagttctggagactaggaagtctaacattaaggtgtcagcctctggcgagggcctacttgatatgtcatcacatgatggacgattagagggcaagaaagatcaaaagggggctgaactcccacttttataagggaaccaaacccactcgtgagggtggagccctcaatccttaatcacctcctaaagctcccaccccttaatactgtcacaatggcaattaaatttcaacatcagttttggagggaaaaacattgaaaccatagtagtgatactgactactaccacacagggcttgggaggctaccctagctgttgcacccaagagatgaatcttctaatgtgattacctttatcattttttttactttattaaaatacttttattttacatgtatacttttgtctacccaccatttccatgtctgaccactgctactactatgtcctagcataacattccatacatccttaaaaccaagcaaagggtggagttccatctttaaaaactaaacaggcattttggacaacacattcttggcaatggaatctggacaacatttatcaaacatggtagggaaggttctcactctgcattatcaaaacgacagccagatatcaactgttacagaaacgaaatcagatggaaaatttttaacaaattgtttaaactattttcttagagagacttcctccactgccagagatcttgaatagcctctggtcagtcatctggaagcaattcttcacataattcatgaacttggcttccactttaggaagagaaccacctttttctatacttgcttgcatttttgctttaatgtcttctacagaactaggtcctttgggtgttttaggagtttttccttgttttgaaggattcttgtccttttgatcttggtgttgacggttttgagtcttttccattccgatttgacttttgtgcatttttggctggagtatctcatatagatttcttcactggcgctttttcttcagtttcctcatcatcaaaatcatcatcatcatcaaaatcatcatcttcatcagcagcaagttttacttttttctgtggaaccttgctaccacctccaggagcagatcgctttccagatatacttatgagtttcacatcctcctcctgttcgtcttctgactctgtatcttcctccccagctactaaatgctgtccactcacatgcactggccctgaaccacacttcaaccgtaagaccactgatggtgttatttcaaagccctcaagggaaaccatgggctgtacagacattttcaaagctgccagtgttactttaattggactgcctttgtaactcattgcctctgcttcaacaatgtgcaatttatcctttgccccagcccctaaactgaccgttcttaaagataactgttgctcaatttcattattatccaccttaaagtgatcatctttgtcggcctttagttcacaaccaaaaagatagttttggggcctcagaggactcatgtccatcatcgtccatcaggtggcaggacgcacttaggtgggagagaaggcagatgatgataaaggaccactgctcaagagaacagctgtgcaggacagaatcacaccagggagattacctttatcttagaaaacctgaacatcttgtgtactttgacacttctctacatttcacctaacctttaacatcaacacatttattcagaaaacttttacttttggagctgctctgtgtcaggctctatgctaggtgctcaggatattgaaattgatacaatcctaacctattcacatataatccaaggtttgctgaaattgatggacatttaaacaattgaaacatttaagtggtataattagcaaatggacatttaagccataaaaatagcatctaatagatataatagaggtcggtacaccattgatgagtcagagcagaggcaacccaaagagtaactagccagaagaattgggaaagcttcatagagagagcgatatgaaaataagggagagaattgtaaatccatgaaaatgagaaaaagttgaaaagtgatggtgtcagaaaaacttgtggtatgataatgacaagatgagaggaactcttggtaagcgtgttggatgcatggaaagaaatggcacaaaataatgctgaggacattttttattttattgttggttttgttttggttaatttcattttttaaatctagtatgctagtgttcattgtccaaactgtgaatcataaactcagtttgtggatcaacaccggcctttgatttttagtgaaacaaaatagaaaatatcagcattcatcacaaatagatgtttcacagattttttgttttaattgcgactgtgtgtgtgtgggtgtgtgtgtgtgtgtgtgtgtgtgtgtgtgtatgtgagagagagagagagagagagagagagagatggcttggatgtttatcacctccgaatcttatattgaaatgtgatttccaatgttggaggcagggcctggtaggtgtgattggatcatgtgggtggatccttcatgaatgatccctttggtgacaagttagttcatgctatatgtggttgtttaaaagagtatgagacctcaacccccacctgtttcctgctctcccctttgccttccaccatggttggttgtaaacttcctgaggctctcaccagaagtagatgccagtgacatgcttcctgtacagcctgcagaaccgtaagtcaaaagaaaaccccttttctttttaaagcac

SEQ ID NO: 218 - DNA, 11058 bp, Artificial Sequence
  DMD Universal replacement cassette with cDNA for any mutations recovery
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcagatcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagcgagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtag

SEQ ID NO: 219 - DNA, 7607 bp, Artificial Sequence
  CTNS Native replacement sequence for Promoter, exons 1-3 mutations recovery using CTNS4 and CTNS1
atacttatgagtgaaaagtatgaacttgaggaaagaacacagccagcagatattacttttttttttttttttttttttttggagacagagtcttactctgttgcccaggctggagtgcagtggtatgatctgggctcactgcaacctctgcctcccgagttcaagcaattctcctgcctcagcctcccaagtagctgggattacaagcacgcatcaccacgcccggctaatttttgttattttgtagtagagacagggtttcaccatgttggccaggctggtctcgaactcctgacctcaagtgatccacccacctccgcctcccaaagtgctgggattacaggcaagagccaccgcgcccggccacagatatgactatagatcactggttcctactcggggtggtcttgtcacctagggaacatttggcaacatggagacatttttggttgtcacatctggggaagaggggcaagcgtggctggcatctagtgggccagagatgttgctaaacattctacaacatgcaggacacccctcacacaacaaaaactatgcagcccaaaatgtcagcagcaccaaggttgagaaaccctgctatatagactaactcacagcagtgctgtttgtcccagagcacgattcatatgtggtgtgggggggttaatgactggcctccgctaagcacttcattaaataggtgtgacacactgggtgagcctgtaagcacagaacagcctgctgaaagctggggagggagggcagaaaagttttcaagaagtggccgtgctgccgcccctactgggaagtgaggagcccctctgcccggccaccaccccgtctgggtagtgtacccaacagctcattgagaatgggccatgatgacaatggcggttttgtggaatagaaaagggggaaaggtggggaaaagattgagaaatcggatggttgctgtgtctgtgtagaaagaagtagacatgggagacttttcattttgttccgtactaagaaaaattcttctgccttgggatcctgttgatctgtgaccttacccccaaccctgtgctctctcaaacatgtgctgtgtccactcagggttaaatggattaagggcggtgcaagatgtgctttgttaaacagatgcttgaaggcagcatgcccgttaagagtcatcaccactccctaatctcaagtacccagggacacaaacactgcggaaggccgcagggtcctctgcctaggaaaaccagagacctttgttcacttgtttatctgctgtccttccctccactattgtcctatgaccctgccaaatccccctctgcgagaaacacccaagagtgatcaattaaaaaaaaaaaaaaagtggccatgctgggtgcggtggctcacacctgtaatcccagcactttgggaaaccgaggcaggcagatcagttgaggtcaggagtttgagaccagccttgccaacatggtgaaaccccatctctaccaaaaatacaaaaaaattctccaagcatggtggcgcacacctgtaatcccagctactcgggaaactgaggcacgaaaatcacttgaacccgggaggcagaggtttcagtgagcagagattgcaccactgcactccagcctgggtgacagagcgagaccctgtctcaaaaaaaaaaaaaaaaaaaaaagaagtgctctatttcaggagaaactggcactttctgagcctactctcccctaatgccagctctcctgctcaccccaccagggtcagagccaactttgcctccaattcatagtcctttaagtaagaatccttttaatatgccctaatgtcccaaccaaactaatcttgaaagcttctatgtagatacaaagtgctcctgaaatccctatcctcagaaatgcttctgagccaaatgggctctgaaccctaaacaaccgtgtccatgtatgtggcaagagcttgtgaaaaacaaagctgggccaggcgcagtgactcacaactgtaatcctagcactttgggaggctgaagtgggcagatcacttgaggtcaggagttcaagaccagtctggcgaacatggcgaaaccctgtctctactaaaaatacaaaaagtagccgggcgcggtggctcacacctgtagtcccagctactcgggaggctgaagcaggagaatcacttgaatccagttggcggaggttgcagtgagcccagatcacgccactgtactccagcctgggcaacagagcgagacttggtaagaaagagaaagaaaggaaagaatgaaggaaggaaggaaggaaggaaggaaggaaggaaggaaggaaggaaggaagggaaggaagggaaggagtctcgctctgtcacccaggctggagtgcaacggagcgatctcgactcactgcaagctccgcctcccgggttcgcgccattctcctgcctcagcctcccgagtagctgggactacaggcgcccgccaccacgccccgctaattttttgtatttttagtacagacggggtttcaccgtgttagccaggatggtctcgatctcctgacctcgtgatccgcccgcctcggcctcccaaagcgctgggattacaggcgtgagccaccgcgcccggctgaccaaaggtttcttggtccgcattctgcttctgtggaatgagccaggagccagttaggcctgatttgacatctgatttccggaggaaaacccagactctgccctgggcaacaaactgaatcctgaacttgaggtcacagggcaggtgtgaggagcggagagcagcaagagtgaaagggaggcctgtggtcattccatacacacaagagatcagttcctccaaggtcaggggacagagagcacagggatccagcgccaagcgcaaggcccccagaagaagccagagagtcggggagggggcgggggggaatcggtcccagcaggtgggaaggattctgggaccagacctaagggatcatgagcacagctgctgcaggcagacgggcccctggagaagctggggacaagctggaatagagacttcattgcgggaagggctgtcagggaggcctcctggggtggaaaagggtggtcaggaggctcctggaggcggcgcggccccgggggtccaactcacctggggcccggccaccgcgctctcgaccgccgcctctgcccgcgcagcacgggcacagctcgccagcactgcgaacccggatgggtcgtcgggcgcggccctcagcagagctgccttcacagatgtggtgcccaggtcaatgccgagggtgatcggccgcgcagccattatctccctgacccgcgcagctccagtctgcagccagcggccccacaagtccgcgctcttcgcccaggggggcggggcaggggcggggagtcgcctgccaatctttcagccacacccaacatggaggcttctcgtcttcccactggccggggaaggcgagcttccacgcaacctctcggcgggccccggctataggcggagaggcggcggaaggcgggacctaaagggggccccgccccacgggctctgatttccgcccaatggagggcggtctgagcttcgctcacgaaaggagccgggaggcgctggcggctccaagagtctctgtgtccctggcagcggacctcatcttccctcacgccggagccccgatctctgcgccccggcccgacccagctgcgctctgtccgtctaagacgcgcggaaactacaactcccagagctcatctcgccgagatccggccccacgagtcaggtggcggaggtcaggtgacagcggacccgcctctcccaaagtctagccgggcaggggaacgcggtgcattcctgaccggcacctggcgaggctcatgcgtcccgtgagggcggttcctcgagcctgggggcgctcaggtgagagcggacgcggcctcccctgtttcccaggcggaccccttgaggcacagcaggtcagcggggcagcctgccgggggtccagcgccctcagccgcggcgggctcctttccccgccaccagtgctggcctcgcgacacgggacaacccccgggtggaagggcccgagcggtggtcagccgaggcaggggcagcgggctgccggggtgggtgccgttcccagccccttaccttctgctcagttgccgcctgggtctcggttggggaatttgcagattgctttggagacgctgagagaacctttgcgagagcgccggttgacgtgcggagtgcggggctccgggggactgagcagcacgagaccccatcctcccctccgggttttcacactgggcgaagggaggactcctgagctctgcctcttccagtaacattgaggattactgtgttttgtgagagctcgctaggcgccctaagcaacagaggtaaccactttatatccttgtttctcaacctcgttattcctacctacccccttcccataaaatttaataccactagtacgctgtgtatttgtttctgtggccacaaaccattgtaatagctagatttcttcactaccaccccaagccaattttttttttttttttgagatggagtctgcagcctctgtcacccaggctggagtgcagtggcgcgatctcggctcactgcaacctccgcctccggggttcaagcgattctcctacctcagccttccgagtagctgggactacaggcctgagccaccatgcccagctaatttttgtatttttagtagagatggggattcaccatgttggccaggctggtctcgaactcctgacctcaggtgatgcgctcacctcggcctcccaaagtgctgggatgacaggcgtgagccaccgcgcccagcctacccccagccaattttagtcccacttgacaatgcgtgctttacatctcctcatttaagtcctgtgaggtagttaccacctccttgtttggcaccacaaggtcgcataagtaataaataggtcaagcctgtctccagtgcacacagcccttgccactatttgtgtaccctctccaaaagcaggagacccagggagttccaggtcgtagaacagaggacaggaccaactcatacctggcagacaggagctgccacactagacccctagccccaggttgctcctgggaagggactgaatgggtgaggagccttcttgaaacatgtgacatctgaatgaggcctggacaatagttagaacttacataggaagggcacgccagacagagcccattgtcaggagatacttcatttctatcttgtagctttcacaagccactagttgtatgtaattatcaatctggttttttttttgttttttttttttaatttgagacggagtttcactcttatcactcaggctggagtgcaatggtgcaatctcggctcactgcaacctccacctcccgggttcaagcgattctcctgcctcagcctcctgagtagctgggactacaggcacatgccaccacgcctggctaatttttgtatttttagtagagacggggattcaccatgttggccaggctggtctcgaactcctgacttcaagtgatccaactgcctcggcctcccaaagtgctggaattacacacacgagccactgcgctcagcctaatctgatgttttttaacattttaattgacttacctctcaatgtcgttttgtctctgctggcatcgttcctccaggggtctcagcctttgaggcttgggaatgtttgctgaccaagtctgtgagtttgagaagctggttaggcctgattctgcatctaatttctggagaaaaaccagactctgtcctgggcaacaaactgaatcctgaacttgaggccacagggcaggtgtgaggagcggagggcagcaagagtgagagggaggcctgtggtcattccatacacgcaggagggcaattcctccaaggtcaggggacagagcacagggatccagcgccaagagcaaggcccccagaggaggccagagagtaggtacggggtcattcccggccggtgagaagggtctcagatgaggcagacctgcagcaggcaaagagagaaccctggaggagacgggccaacagaggtcagacagctggagcagccagggagacttcttgaggagtgtgtaagggagatgtccggagatgctggaggccttggggaaactgaaatcagagtgggaacagggatgtctccacacagaccttacccagagctccccacagtctgcaggaggcccgtgagactgtgtactgaggcagcacggagaccaagctacagaaatccatgccggcctggctgctcttgacccactgttcacctgctgtgtcttgggtttacaggaatgcagctccccatcttccacactaaaccaaggacttgctctggggctcatccctccccgagtcctccttgtgaatgaccccagccagtcctggaatggtgacacttgtcaaataaagtcttgacaggcgcggtggctcctacctgtaaccccagcactttgggaggctgaggcgggcggatcactcgaggtcaggagtttgagaccaggctggccaacatggtgaaaccccatctctactaaaaatacaaaagttagccgggcatggtggggggcacctgtaatcccagctactcaggaggctgaggcacaagaattgcttgaacccagggggtggaggtttcagtgaacagagttcgcaccactgcactccagcctgggcaacagagcaagactctgtctcaaaaaaaaaaaaatttaaatatgtatattaaaaaaaaatgtttttttaagtcttaagggtcagttggtgtcatcagcccttagactcttatcccaggacaggaaaggaaattaatttccttgaggtttataggttcacaatgtcaaatatctgaccacagttttaacaacttttggagaaaaagaatctcaagccagtaaaattgcattctttctttctgctaactaagtttttacaaaaagcaattgaagagggaaaaattctggtctttgttcacttcctcaggggggcactttacacaacccatttatctgctcggagcccgtttcccctgtatatcaaagaaagataagtcctctctagggtgtccctctgaggccgtgatgcaaagccctgaggtcacagctgtcaggtggcagtcctttatgagccatccatgctccagagggcagattgtctacagggagctgagctgattcaacattcccctgaacttctctcttgctgtttttcttcctagttctgagaaatcgagaaacatgataaggaattggctgactatttttatcctttttcccctgaagctcgtagagaaatgtggtaagtttagaaatgacacgtcaactttgtaaagagggaaatggtggctagaggaaggagtaatctgatctgtttgttgccaagggtttagaatcattcagaccacatgtctctgtctgcctcttggccatgtggccactggggtggtggagcagacccaggtctgggatccaggtgttctgcaaagagccagatagttccacatataattggccttctgccctggtatctctgtacctttctgtaccaaagtga

SEQ ID NO: 220 - DNA, 1899 bp, Artificial Sequence
  CTNS Universal replacement cassette with Promoter-cDNA for any mutations recovery
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatcctgagctctgcctcttccagtaacattgaggattactgtgttttgtgagagctcgctaggcgccctaagcaacagagttctgagaaatcgagaaacatgataaggaattggctgactatttttatcctttttcccctgaagctcgtagagaaatgtgagtcaagcgtcagcctcactgttcctcctgtcgtaaagctggagaacggcagctcgaccaacgtcagcctcaccctgcggccaccattaaatgcaaccctggtgatcacttttgaaatcacatttcgttccaaaaatattactatccttgagctccccgatgaagttgtggtgcctcctggagtgacaaactcctcttttcaagtgacatctcaaaatgttggacaacttactgtttatctacatggaaatcactccaatcagaccggcccgaggatacgctttcttgtgatccgcagcagcgccattagcatcataaaccaggtgattggctggatctactttgtggcctggtccatctccttctaccctcaggtgatcatgaattggaggcggaaaagtgtcattggtctgagcttcgacttcgtggctctgaacctgacgggcttcgtggcctacagtgtattcaacatcggcctcctctgggtgccctacatcaaggagcagtttctcctcaaataccccaacggagtgaaccccgtgaacagcaacgacgtcttcttcagcctgcacgcggttgtcctcacgctgatcatcatcgtgcagtgctgcctgtatgagcgcggtggccagcgcgtgtcctggcctgccatcggcttcctggtgctcgcgtggctcttcgcatttgtcaccatgatcgtggctgcagtgggagtgatcacgtggctgcagtttctcttctgcttctcctacatcaagctcgcagtcacgctggtcaagtattttccacaggcctacatgaacttttactacaaaagcactgagggctggagcattggcaacgtgctcctggacttcaccgggggcagcttcagcctcctgcagatgttcctccagtcctacaacaacgaccagtggacgctgatcttcggagacccaaccaagtttggactcggggtcttctccatcgtcttcgacgtcgtcttcttcatccagcacttctgtttgtacagaaagagaccggggtatgaccagctgaactag

SEQ ID NO: 221 - DNA, 96 bp, Artificial Sequence
  SCN1A Native replacement sequence for intron 6 mutations recovery using SCN1A3 and SCN1A4
atactttgcactgtaaagtgtctaaagtatctttgcactgtatctaatctaatgtcatttcttcataatgaagaaatactttgcactgtaaagtat

SEQ ID NO: 222 - DNA, 5997 bp, Artificial Sequence
  SCN1A Universal replacement cassette with cDNA for any mutations recovery
atggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataa

SEQ ID NO: 223 - protein, 357 aa, Artificial Sequence
  N303K mutant of the HK022 integrase
MGRRRSHERRDLPPNLYIRNNGYYCYRDPRTGKEFGLGRDRRIAITEAIQANIELLSGNRRESLIDRIKGADAITLHAWLDRYETILSERGIRPKTLLDYASKIRAIRRKLPDKPLADISTKEVAAMLNTYVAEGKSASAKLIRSTLVDVFREAIAEGHVATNPVTATRTAKSEVRRSRLTANEYVAIYHAAEPLPIWLRLAMDLAVVTGQRVGDLCRMKWSDINDNHLHIEQSKTGAKLAIPLTLTIDALNISLADTLQQCREASSSETIIASKHHDPLSPKTVSKYFTKARNASGLSFDGKPPTFHELRSLSARLYRNQIGDKFAQRLLGHKSDSMAARYRDSRGREWDKIEIDK

SEQ ID NO: 224 - DNA, 1071 bp, Artificial Sequence
  N303K mutant of the HK022 integrase
atgggcaggcggcggagccacgagcggagagacctgccccccaacctgtacatccggaacaacggctactactgctaccgggacccccggaccggcaaagagttcggcctgggccgggacaggcggatcgccatcaccgaggccatccaggccaacatcgagctgctgtccggcaaccggcgggagagcctgatcgaccggatcaagggcgccgacgccatcaccctgcacgcctggctggacagatacgagaccatcctgagcgagcggggcatccggcccaagaccctgctggactacgcctctaagatccgggccatcagacggaagctgcccgacaagcccctggccgacatcagcaccaaagaagtggccgccatgctgaacacctacgtggccgagggcaagagcgccagcgccaagctgatccggtccaccctggtggacgtgttccgggaggccatcgccgagggccacgtcgccaccaaccccgtgaccgccacccggaccgccaagagcgaagtgcggcggagcaggctgaccgccaacgagtacgtggccatctaccatgccgctgagcccctgcccatctggctgcggctggccatggacctggccgtggtgaccggccagagagtgggcgacctgtgccggatgaagtggagcgacatcaacgacaaccacctgcacatcgagcagagcaagaccggcgccaaactggccatccccctgaccctgaccatcgacgccctgaacatcagcctggccgataccctgcagcagtgcagagaggccagcagcagcgagaccatcatcgccagcaagcaccacgaccccctgagccccaagaccgtgagcaagtacttcaccaaggcccggaacgccagcggcctgagcttcgacggcaaaccccccaccttccacgagctgcggagcctgtctgccaggctgtaccggaaccagatcggcgacaagttcgctcagcggctcctgggccacaagagcgacagcatggccgccagataccgggacagccggggacgggagtgggacaagatcgagatcgacaag

SEQ ID NO: 225 - DNA, 28 bp, Artificial Sequence
  primer 1288
gctctctcccagcttgatttccaatggg

SEQ ID NO: 226 - protein, 3685 aa, Homo sapiens
  MISC_FEATURE: dystrophin (DMD), transcript variant Dp427m, isoform Dp427m, accession number NP_003997.2
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSDLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLRELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 227 - protein, 1480 aa, Homo sapiens
  MISC_FEATURE: cystic fibrosis transmembrane conductance regulator (CFTR), accession number NP_000483.3
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 228 - protein, 367 aa, Homo sapiens
  MISC_FEATURE: cystinosin lysosomal cystine transporter (CTNS) isoform 2 precursor, accession number NP_004928.2
MIRNWLTIFILFPLKLVEKCESSVSLTVPPVVKLENGSSTNVSLTLRPPLNATLVITFEITFRSKNITILELPDEVVVPPGVTNSSFQVTSQNVGQLTVYLHGNHSNQTGPRIRFLVIRSSAISIINQVIGWIYFVAWSISFYPQVIMNWRRKSVIGLSFDFVALNLTGFVAYSVFNIGLLWVPYIKEQFLLKYPNGVNPVNSNDVFFSLHAVVLTLIIIVQCCLYERGGQRVSWPAIGFLVLAWLFAFVTMIVAAVGVTTWLQFLFCFSYIKLAVTLVKYFPQAYMNFYYKSTEGWSIGNVLLDFTGGSFSLLQMFLQSYNNDQWTLIFGDPTKFGLGVFSIVFDVVFFIQHFCLYRKRPGYDQLN

SEQ ID NO: 229 - protein, 529 aa, Homo sapiens
  MISC_FEATURE: beta-hexosaminidase subunit alpha (HEXA) isoform 2 preproprotein, accession number NP_000511.2
MTSSRLWFSLLLAAAFAGRATALWPWPQNFQTSDQRYVLYPNNFQFQYDVSSAAQPGCSVLDEAFQRYRDLLFGSGSWPRPYLTGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYIVEPLAFEGTPEQKALVIGGEACMWGEYVDNTNLVPRLWPRAGAVAERLWSNKLTSDLTFAYERLSHFRCELLRRGVQAQPLNVGFCEQEFEQT

SEQ ID NO: 230 - protein, 3056 aa, Homo sapiens
  MISC_FEATURE: serine/threonine kinase (ATM) isoform a, accession number NP_000042.3
MSLVLNDLLICCRQLEHDRATERKKEVEKFKRLIRDPETIKHLDRHSDSKQGKYLNWDAVFRFLQKYIQKETECLRIAKPNVSASTQASRQKKMQEISSLVKYFIKCANRRAPRLKCQELLNYIMDTVKDSSNGAIYGADCSNILLKDILSVRKYWCEISQQQWLELFSVYFRLYLKPSQDVHRVLVARIIHAVTKGCCSQTDGLNSKFLDFFSKAIQCARQEKSSSGLNHILAALTIFLKTLAVNFRIRVCELGDEILPTLLYIWTQHRLNDSLKEVIIELFQLQIYIHHPKGAKTQEKGAYESTKWRSILYNLYDLLVNEISHIGSRGKYSSGFRNIAVKENLIELMADICHQVFNEDTRSLEISQSYTTTQRESSDYSVPCKRKKIELGWEVIKDHLQKSQNDFDLVPWLQIATQLISKYPASLPNCELSPLLMILSQLLPQQRHGERTPYVLRCLTEVALCQDKRSNLESSQKSDLLKLWNKIWCITFRGISSEQIQAENFGLLGAIIQGSLVEVDREFWKLFTGSACRPSCPAVCCLTLALTTSIVPGTVKMGIEQNMCEVNRSFSLKESIMKWLLFYQLEGDLENSTEVPPILHSNFPHLVLEKILVSLTMKNCKAAMNFFQSVPECEHHQKDKEELSFSEVEELFLQTTFDKMDFLTIVRECGIEKHQSSIGFSVHQNLKESLDRCLLGLSEQLLNNYSSEITNSETLVRCSRLLVGVLGCYCYMGVIAEEEAYKSELFQKAKSLMQCAGESITLFKNKTNEEFRIGSLRNMMQLCTRCLSNCTKKSPNKIASGFFLRLLTSKLMNDIADICKSLASFIKKPFDRGEVESMEDDTNGNLMEVEDQSSMNLFNDYPDSSVSDANEPGESQSTIGAINPLAEEYLSKQDLLFLDMLKFLCLCVTTAQTNTVSFRAADIRRKLLMLIDSSTLEPTKSLHLHMYLMLLKELPGEEYPLPMEDVLELLKPLSNVCSLYRRDQDVCKTILNHVLHVVKNLGQSNMDSENTRDAQGQFLTVIGAFWHLTKERKYIFSVRMALVNCLKTLLEADPYSKWAILNVMGKDFPVNEVFTQFLADNHHQVRMLAAESINRLFQDTKGDSSRLLKALPLKLQQTAFENAYLKAQEGMREMSHSAENPETLDEIYNRKSVLLTLIAVVLSCSPICEKQALFALCKSVKENGLEPHLVKKVLEKVSETFGYRRLEDFMASHLDYLVLEWLNLQDTEYNLSSFPFILLNYTNIEDFYRSCYKVLIPHLVIRSHFDEVKSIANQIQEDWKSLLTDCFPKILVNILPYFAYEGTRDSGMAQQRETATKVYDMLKSENLLGKQIDHLFISNLPEIVVELLMTLHEPANSSASQSTDLCDFSGDLDPAPNPPHFPSHVIKATFAYISNCHKTKLKSILEILSKSPDSYQKILLAICEQAAETNNVYKKHRILKIYHLFVSLLLKDIKSGLGGAWAFVLRDVIYTLIHYINQRPSCIMDVSLRSFSLCCDLLSQVCQTAVTYCKDALENHLHVIVGTLIPLVYEQVEVQKQVLDLLKYLVIDNKDNENLYITIKLLDPFPDHVVFKDLRITQQKIKYSRGPFSLLEEINHFLSVSVYDALPLTRLEGLKDLRRQLELHKDQMVDIMRASQDNPQDGIMVKLVVNLLQLSKMAINHTGEKEVLEAVGSCLGEVGPIDFSTIAIQHSKDASYTKALKLFEDKELQWTFIMLTYLNNTLVEDCVKVRSAAVTCLKNILATKTGHSFWEIYKMTTDPMLAYLQPFRTSRKKFLEVPRFDKENPFEGLDDINLWIPLSENHDIWIKTLTCAFLDSGGTKCEILQLLKPMCEVKTDFCQTVLPYLIHDILLQDTNESWRNLLSTHVQGFFTSCLRHFSQTSRSTTPANLDSESEHFFRCCLDKKSQRTMLAVVDYMRRQKRPSSGTIFNDAFWLDLNYLEVAKVAQSCAAHFTALLYAEIYADKKSMDDQEKRSLAFEEGSQSTTISSLSEKSKEETGISLQDLLLEIYRSIGEPDSLYGCGGGKMLQPITRLRTYEHEAMWGKALVTYDLETAIPSSTRQAGIIQALQNLGLCHILSVYLKGLDYENKDWCPELEELHYQAAWRNMQWDHCTSVSKEVEGTSYHESLYNALQSLRDREFSTFYESLKYARVKEVEEMCKRSLESVYSLYPTLSRLQAIGELESIGELFSRSVTHRQLSEVYIKWQKHSQLLKDSDFSFQEPIMALRTVILEILMEKEMDNSQRECIKDILTKHLVELSILARTFKNTQLPERAIFQIKQYNSVSCGVSEWQLEEAQVFWAKKEQSLALSILKQMIKKLDASCAANNPSLKLTYTECLRVCGNWLAETCLENPAVIMQTYLEKAVEVAGNYDGESSDELRNGKMKAFLSLARFSDTQYQRIENYMKSSEFENKQALLKRAKEEVGLLREHKIQTNRYTVKVQRELELDELALRALKEDRKRFLCKAVENYINCLLSGEEHDMWVFRLCSLWLENSGVSEVNGMMKRDGMKIPTYKFLPLMYQLAARMGTKMMGGLGFHEVLNNLISRISMDHPHHTLFIILALANANRDEFLTKPEVARRSRITKNVPKQSSQLDEDRTEAANRIICTIRSRRPQMVRSVEALCDAYIILANLDATQWKTQRKGINIPADQPITKLKNLEDVVVPTMEIKVDHTGEYGNLVTIQSFKAEFRLAGGVNLPKIIDCVGSDGKERRQLVKGRDDLRQDAVMQQVFQMCNTLLQRNTETRKRKLTICTYKVVPLSQRSGVLEWCTGTVPIGEFLVNNEDGAHKRYRPNDFSAFQCQKKMMEVQKKSFEEKYEVFMDVCQNFQPVFRYFCMEKFLDPAIWFEKRLAYTRSVATSSIVGYILGLGDRHVQNILINEQSAELVHIDLGVAFEQGKILPTPETVPFRLTRDIVDGMGITGVEGVFRRCCEKTMEVMRNSQETLLTIVEVLLYDPLFDWTMNPLKALYLQQRPEDETELHPTLNADDQECKRNLSDIDQSFNKVAERVLMRLQEKLKGVEEGTVLSVGGQVNLLIQQAIDPKNLSRLFPGWKAWV

SEQ ID NO: 231 - protein, 218 aa, Homo sapiens
  MISC_FEATURE: hypoxanthine-guanine phosphoribosyltransferase 1 (HPRT1), accession number NP_000185.1
MATRSPGVVISDDEPGYDLDLFCIPNHYAEDLERVFIPHGLIMDRTERLARDVMKEMGGHHIVALCVLKGGYKFFADLLDYIKALNRNSDRSIPMTVDFIRLKSYCNDQSTGDIKVIGGDDLSTLTGKNVLIVEDIIDTGKTMQTLLSLVRQYNPKMVKVASLLVKRTPRSVGYKPDFVGFEIPDKFVVGYALDYNEYFRDLNHVCVISETGKAKYKA

SEQ ID NO: 232 - protein, 154 aa, Homo sapiens [sequence omitted]

SEQ ID NO: 233 - protein, 414 aa, Homo sapiens
  MISC_FEATURE: TAR DNA-binding protein (TARDBP) 43, accession number NP_031401.1
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 234 - protein, 243 aa, Homo sapiens
  MISC_FEATURE: vesicle-associated membrane protein-associated protein B/C (VAPB), accession number NP_004729.1
MAKVEQVLSLEPQHELKFRGPFTDVVTTNLKLGNPTDRNVCFKVKTTAPRRYCVRPNSGIIDAGASINVSVMLQPFDYDPNEKSKHKFMVQSMFAPTDTSDMEAVWKEAKPEDLMDSKLRCVFELPAENDKPHDVEINKIISTTASKTETPIVSKSLSSSLDDTEVKKVMEECKRLQGEVQRLREENKQFKEEDGLRMRKTVQSNSPISALAPTGKEEGLSTRLLALVVLFFIVGVIIGKIAL

SEQ ID NO: 235 - protein, 1278 aa, Homo sapiens
  MISC_FEATURE: NPC intracellular cholesterol transporter 1 precursor (NPC1), accession number NP_000262.2
MTARGLALGLLLLLLCPAQVFSQSCVWYGECGIAYGDKRYNCEYSGPPKPLPKDGYDLVQELCPGFFFGNVSLCCDVRQLQTLKDNLQLPLQFLSRCPSCFYNLLNLFCELTCSPRQSQFLNVTATEDYVDPVTNQTKTNVKELQYYVGQSFANAMYNACRDVEAPSSNDKALGLLCGKDADACNATNWIEYMFNKDNGQAPFTITPVFSDFPVHGMEPMNNATKGCDESVDEVTAPCSCQDCSIVCGPKPQPPPPPAPWTILGLDAMYVIMWITYMAFLLVFFGAFFAVWCYRKRYFVSEYTPIDSNIAFSVNASDKGEASCCDPVSAAFEGCLRRLFTRWGSFCVRNPGCVIFFSLVFITACSSGLVFVRVTTNPVDLWSAPSSQARLEKEYFDQHFGPFFRTEQLIIRAPLTDKHIYQPYPSGADVPFGPPLDIQILHQVLDLQIAIENITASYDNETVTLQDICLAPLSPYNTNCTILSVLNYFQNSHSVLDHKKGDDFFVYADYHTHFLYCVRAPASLNDTSLLHDPCLGTFGGPVFPWLVLGGYDDQNYNNATALVITFPVNNYYNDTEKLQRAQAWEKEFINFVKNYKNPNLTISFTAERSIEDELNRESDSDVFTVVISYAIMFLYISLALGHMKSCRRLLVDSKVSLGIAGILIVLSSVACSLGVFSYIGLPLTLIVIEVIPFLVLAVGVDNIFILVQAYQRDERLQGETLDQQLGRVLGEVAPSMFLSSFSETVAFFLGALSVMPAVHTFSLFAGLAVFIDFLLQITCFVSLLGLDIKRQEKNRLDIFCCVRGAEDGTSVQASESCLFRFFKNSYSPLLLKDWMRPIVIAIFVGVLSFSIAVLNKVDIGLDQSLSMPDDSYMVDYFKSISQYLHAGPPVYFVLEEGHDYTSSKGQNMVCGGMGCNNDSLVQQIFNAAQLDNYTRIGFAPSSWIDDYFDWVKPQSSCCRVDNITDQFCNASVVDPACVRCRPLTPEGKQRPQGGDFMRFLPMFLSDNPNPKCGKGGHAAYSSAVNILLGHGTRVGATYFMTYHTVLQTSADFIDALKKARLIASNVTETMGINGSAYRVFPYSVFYVFYEQYLTIIDDTIFNLGVSLGAIFLVTMVLLGCELWSAVIMCATIAMVLVNMFGVMWLWGISLNAVSLVNLVMSCGISVEFCSHITRAFTVSMKGSRVERAEEALAHMGSSVFSGITLTKFGGIVVLAFAKSQIFQIFYFRMYLAMVLLGATHGLIFLPVLLSYIGPSVNKAKSCATEERYKGTERERLLNF

SEQ ID NO: 236 - protein, 1998 aa, Homo sapiens
  MISC_FEATURE: sodium channel protein type 1 subunit alpha 1 (SCN1A), isoform 2, accession number NP_008851.3
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 237 - protein, 1466 aa, Homo sapiens
  MISC_FEATURE: collagen type III alpha 1 chain preproprotein, (COL3A1) accession number NP_000081.2
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVHLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 238 - protein, 170 aa, Homo sapiens
  MISC_FEATURE: transmembrane protein 252 (TMEM252), also known as c9ORF 71-1, accession number NP_694969.1
MQNRTGLILCALALLMGFLMVCLGAFFISWGSIFDCQGSLIAAYLLLPLGFVILLSGIFWSNYRQVTESKGVLRHMLRQHLAHGALPVATVDRPDFYPPAYEESLEVEKQSCPAEREASGIPPPLYTETGLEFQDGNDSHPEAPPSYRESIAGLVVTAISEDAQRRGQEC

SEQ ID NO: 239 - protein, 147 aa, Homo sapiens
  MISC_FEATURE: Hemoglobin subunit beta PROTEIN (HBB), accession number NP_000509.1
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 240 - DNA, 140 bp, Artificial Sequence
  P variant (tenP1) for attP donor cassette
agggtcctaatactatctaagtagttgattcatagtgactggatatgttgcgttttgtcgcattatgtagtctatcatttaaccacagattagtgtaatgcgatgatttttaagtgattaatgttattttgtcatccttt

SEQ ID NO: 241 - DNA, 140 bp, Artificial Sequence
  P variant (tenP2) for attP donor cassette
aggtcactaatactatctaagtagttgattcataggacctggatatgttgcgttttgtcgcattatgtagtctatcatttaaccacagattagtgtaatgcgatgatttttaagtgattaatgttattttgtcatccttt

SEQ ID NO: 242 - DNA, 77 bp, Artificial Sequence
  P′ variant (tenP′1) for attP donor cassette
taagttgtatatttaaaatctctttaattatcagtaaattaatgtaagtagggtcttattagtcaaaataaaatcat

SEQ ID NO: 243 - DNA, 77 bp, Artificial Sequence
  P′ variant (tenP′2) for attP donor cassette
taagttgtatatttaaaatctctttaattatcagtaaattaatgtaagtaggtcattattaggtcaaataaaatcat

SEQ ID NO: 244 - DNA, 77 bp, Artificial Sequence
  P′ variant (tenP′3) for attP donor cassette
taagttgtatatttaaaatctctttaattatcagtaaattaatgtaagtaggtcattattagtcaaaataaaagtct

(19) United States
(12) Patent Application Publication
(10) Pub. No.: US 2022/0154221 A1
(43) Pub. Date: May 19, 2022

(54) SITE SPECIFIC RECOMBINASE INTEGRASE VARIANTS AND USES THEREOF IN GENE EDITING IN EUKARYOTIC CELLS

(71) Applicant: RAMOT AT TEL-AVIV UNIVERSITY LTD., Tel Aviv (IL)

(72) Inventors: Mikhail KOLOT, Tel Aviv (IL); Amer ELIAS, Kfar-Yasif (IL)

(21) Appl. No.: 17/430,085

(22) PCT Filed: Feb. 11, 2020
(86) PCT No.: PCT/IL2020/050164

§ 371 Date: Aug. 11, 2021

Related U.S. Application Data
(60) Provisional application No. 62/803,634, filed on Feb. 11, 2019.
(60) Provisional application No. 62/803,637, filed on Feb. 11, 2019.
(60) Provisional application No. 62/803,640, filed on Feb. 11, 2019.

(51) Int. Cl. C12N 15/90; C07K 14/47; C12N 9/14

(52) U.S. Cl. CPC C12N 15/90; C12N 2795/10022; C12N 9/14; C07K 14/4712

(57) ABSTRACT

The invention relates to novel variants and mutants of HK022 bacteriophage integrase (HK-Int), systems, kits, compositions, methods and uses thereof for gene therapy using site-specific recombination. More specifically, the invention further provides donor cassettes comprising replacement sequences for targeted replacement of target nucleic acid sequences using the HK-Int variants of the invention.

### FIELD OF THE INVENTION

The invention relates to gene editing in eukaryotic cells. More specifically, the invention provides novel mutants of a specific integrase, compositions, methods and uses thereof for gene therapy using site-specific recombination.

### BACKGROUND REFERENCES

References considered to be relevant as background to the presently disclosed subject matter are listed below:
1. Jarmin, S., Kymalainen, H., Popplewell, L. and Dickson, G. (2014) New developments in the use of gene therapy to treat Duchenne muscular dystrophy. Expert. Opin. Biol. Ther., 14, 209-230.
2. Zhao, C., Farruggio, A. P., Bjornson, C. R., Chavez, C. L., Geisinger, J. M., Neal, T. L., Karow, M. and Calos, M. P. (2014) Recombinase-mediated reprogramming and dystrophin gene addition in mdx mouse induced pluripotent stem cells. PLoS. ONE., 9, e96279.
3. Turan, S., Zehe, C., Kuehle, J., Qiao, J. and Bode, J. (2013) Recombinase-mediated cassette exchange (RMCE)—a rapidly-expanding toolbox for targeted genomic modifications. Gene, 515, 1-27.
4. Azaro, M. A. and Landy, A. (2002) Integrase and the λ int family. In Craig, N. L., Craigie, R., Gellert, M. and Lambowitz, A. (eds.), Mobile DNAII. ASM Press, Washington D. C., pp. 118-148.
5. Biswas, T., Aihara, H., Radman-Livaja, M., Filman, D., Landy, A. and Ellenberger, T. (2005) A structural basis for allosteric control of DNA recombination by lambda integrase. Nature, 435, 1059-1066.
6. Weisberg, R. A., Gottesmann, M. E., Hendrix, R. W. and Little, J. W. (1999) Family values in the age of genomics: comparative analyses of temperate bacteriophage HK022. Annu. Rev. Genet., 33, 565-602.
7. Harel-Levy G., Goltsman J., Tuby C. N. J. H., Yagil E. and Kolot, M. (2008) Human genomic site-specific recombination catalyzed by coliphge HK022 integrase. J. Biotechnol., 134, 45-54.
8. Kolot, M., Malchin, N., Elias, A., Gritsenko, N. and Yagil, E. (2015) Site promiscuity of coliphage HK022 integrase as tool for gene therapy. Gene Ther., 22, 602.
9. Malchin, N., Goltsman, J., Dabool, L., Gorovits, R., Bao, Q., Droge, P., Yagil, E. and Kolot, M. (2009) Optimization of coliphage HK022 Integrase activity in human cells. Gene, 437, 9-13.
10. Voziyanova, E., Malchin, N., Anderson, R. P., Yagil, E., Kolot, M. and Voziyanov, Y. (2013) Efficient Flp-Int HK022 dual RMCE in mammalian cells. Nucleic Acids Res., 41, e125.
11. Kolot, M., Meroz, A. and Yagil, E. (2003) Site-specific recombination in human cells catalyzed by the wild-type integrase protein of coliphage HK022. Biotechnol. Bioeng., 84, 56-60.
12. Malchin, N., Molotsky, T., Yagil, E., Kotlyar, A. B. and Kolot, M. (2008) Molecular analysis of recombinase-mediated cassette exchange reactions catalyzed by integrase of coliphage HK022. Res. in Microbiol., 159, 663-670.
13. Bolusani, S., Ma, C. H., Paek, A., Konieczka, J. H., Jayaram, M. and Voziyanov, Y. (2006) Evolution of variants of yeast site-specific recombinase Flp that utilize native genomic sequences as recombination target sites. Nucleic Acids Research, 34, 5259-5269.
14. Malchin, N., Tuby, C. N., Yagil, E. and Kolot, M. (2011) Arm site independence of coliphage HK022 integrase in human cells. Mol. Genet. Genomics, 285, 403-413.
15. Kolot, M., Silberstein, N. and Yagil, E. (1999) Site-specific recombination in mammalian cells expressing the Int recombinase of bacteriophage HK022. Molec. Biol. Reports, 26, 207-213. Acknowledgement of the above references herein is not to be inferred as meaning that these are in any way relevant to the patentability of the presently disclosed subject matter.

### BACKGROUND OF THE INVENTION

Gene therapy is one of the most promising approaches for basic science, industrial biotechnology and medicine. These manipulations are carried out by using different gene-editing endonucleases: Zing finger nucleases, Transcription activator-like effector nucleases (TALENs), Clustered Regularly Interspaced Short Palindromic Repeats-CRISPR-associated protein-9 nuclease (CRISPR-Cas9) system and site-specific recombinases.

Nevertheless, several hurdles still need to be overcome, specifically, low efficiency of correction and potential off-target effects of the endonucleases. Potential off-target cutting can lead to oncogenic mutations and is especially relevant for cells with high proliferative potential such as human Induced pluripotent stem cells (hIPSCs) (1).

Site-specific recombinases (SSRs) are widely used in developmental, synthetic biology, genome manipulations and gene therapy (2). SSRs catalyze the site-specific recombination reaction between two specific short DNA sequences—recombination sites (RSs), resulting in integration, excision, inversion and translocation, depending on the location and relative orientation of the RSs. The efficient approach for genome manipulations by SSRs, named recombinase mediated cassette exchange (RMCE) overcomes the inefficiency of integration in trans reaction due to more favorable excision in cis reaction. This technology based on using one or two different recombinases allows replacing a genomic sequence carried a harmful mutation or deletion flanked by two incompatible RSs with a plasmid-borne normal sequence flanked by matching RSs (3). RMCE has expanded substantial input in various research areas in recent years: generation of induced pluripotent stem (iPS) cells, production of therapeutic monoclonal antibodies and combination with other genome-editing approaches, as TALENs and CRISPR/Cas. The site-specific recombinase Integrase (HK-Int) of the HK022 bacteriophage belongs to the tyrosine family of SSRs and catalyzes phage integration into the E. coli chromosome as well as prophage excision. The mechanism of these site-specific recombination reactions have some similarity with the Integrase of coliphage Lambda (4). The Integrase of the Lambda includes three different domains may act both in cis and in trans and facilitate functional assembly of a higher order tetrameric complex with DNA substrate known as an intasome. The N-terminal DNA binding domain (ND) (residues 1-63) recognizes the ‘arm-type’ DNA sequences adjacent to the attP core site. The binding results in allosteric modifications allowing the function of the core-binding (CB) domain (residues 75-175) and C-terminal catalytic domain (CD) (residues 176-356) function. The CB domain recognizes the attP (C and C′)×attB (B and B′) core DNA sequences and is associated to the CD domain responsible for DNA cleavage and rejoining (5).

HK022 bacterial recombination site attB (BOB′) is 21 bp long comprising a central 7 bp overlap region (O, the site of DNA exchange) flanked by two 7 bp incomplete inverted repeats (B and B′) that serve as weak binding sites for Int. The phage attP recombination site is over 200 bp long. It is composed of a similar 21 bp core (COC′) flanked by two long arms (P and P′). The phage integration reaction takes place between attP and attB sites and leads to generation of two new recombination attL (BOP′) and attR (POB′) sites flanking the integrated prophage. The reverse excision reaction of the prophage takes place between the attL (BOP′) and attR (POB′) sites and restores the attP and attB sites (6).

The inventors have previously reported that the wild type Integrase was active in human cells without any of the prokaryotic accessory proteins (7). Still further, the inventors have previously identified several native active secondary attB sites that flank variety of human deleterious mutations associated with genetic disorders, raising the prospect of using such sites to cure the ‘attB’-flanked mutations by Wild type Int-catalyzed RMCE (8). However, the inventors have shown that Wild type Tnt exhibits low efficiency in catalyzing RMCE reaction in human cells.

The gene of the wild type Int from the HK022 coliphage was also adapted to the human codon usage (9) and exploited for genomic manipulation in plants, Cyanobacteria, mice and human cells (7-10). It was previously shown for the Integrase of the Lambda coliphage, only Integration host factor (IHF)-independent mutants of Int can catalyze the recombination reactions in mammalian cells.

However, there is an unmet need to produce an optimized Integrase enzyme with enhanced activity that would not exhibit off-target effects. Such effective Integrase variants are required for gene therapy and open the way of performing RMCE reactions for gene editing in human cells.

### SUMMARY OF THE INVENTION

In a first aspect, the invention relates to a HK022 bacteriophage site-specific recombinase Integrase (HK-Int) variant and/or mutated molecule or any functional fragments or peptides thereof. In some embodiments, the HK-Int variant/mutated molecule comprise at least one substituted amino acid residue in at least one of the core-binding domain (CB), the N-terminal DNA binding domain (ND) and the C-terminal catalytic domain (CD) of the Wild type HK-Int molecule.

In a further aspect, the invention relates to a nucleic acid molecule comprising a nucleic acid sequence encoding a HK-Int variant and/or mutated molecule or any functional fragments or peptides thereof.

In yet a further aspect, the invention relates to a host cell comprising at least one HK-Int variant/mutated molecule or any functional fragments or peptides thereof, any nucleic acid molecule comprising a nucleic acid sequence encoding at least one HK-Int variant/mutated molecule or any functional fragments or peptides thereof, any combinations thereof, or with any vector, vehicle, matrix, nano- or micro-particle comprising the same.

In another aspect, the invention relates to a system and/or kit may comprise at least one of: As a first component (a), at least one nucleic acid molecule comprising a replacement-sequence flanked by a first and a second Int recognition sites. In some embodiments, the first site attP1 may comprise a first overlap sequence O1 and the second site attP2 may comprise a second overlap sequence O2. In some further embodiments, the first O1 and the second O2 overlap sequences may be different, each consisting of seven nucleotides, the O1 may be identical to an overlap sequence O1 comprised within a first Int recognition site attE1 in a eukaryotic cell and the O2 may be identical to an overlap sequence O2 comprised within a second Int recognition site attE2 in the eukaryotic cell. In some embodiments, the eukaryotic recognition sites attE1 and attE2 may flank a target nucleic acid sequence of interest or any fragment thereof in the eukaryotic cell; and/or As a second component (b), at least one HK-Int variant/mutated molecule or any functional fragments or peptides thereof, any nucleic acid molecule comprising a sequence encoding the HK-Int variant/mutated molecule or any vector, vehicle, matrix, nano- or micro-particle comprising the same. In some embodiments, the HK-Int variant/mutated molecule comprise at least one substituted amino acid residue in at least one of the CB, ND and the CD of the Wild type HK-Int molecule. Another aspect of the invention relates to a nucleic acid molecule or any nucleic acid cassette or vector thereof, comprising a replacement-sequence flanked by a first and a second Int recognition sites. The first site attP1, comprises a first overlap sequence O1 and the second site attP2, comprises a second overlap sequence O2, wherein the first O1 and said second O2 overlap sequences are different, each consisting of seven nucleotides. The O1 is identical to an overlap sequence O1 comprised within a first Int recognition site attE1 in a eukaryotic cell and said O2 is identical to an overlap sequence O2 comprised within a second Int recognition site attE2 in said eukaryotic cell, said eukaryotic recognition sites attE1 and attE2 flank a target nucleic acid sequence of interest or any fragment thereof in said eukaryotic cell.

In another aspect, the invention relates to a composition comprising as an active ingredient an effective amount of (a) at least one HK-Int variant/mutated molecule or any functional fragments or peptides thereof, any nucleic acid molecule comprising a sequence encoding the HK-Int variant/mutated molecule or any vector, vehicle, matrix, nano- or micro-particle comprising the same, or any host cell comprising the HK-Int variants of the invention or any nucleic acid sequence encoding these variants. In some embodiments, the HK-Int variant/mutated molecule comprise at least one substituted amino acid residue in at least one of the CB, ND and the CD of the Wild type HK-Int molecule. In some further embodiments, the composition of the invention may optionally further comprise as an additional component (b), at least one nucleic acid molecule comprising a replacement-sequence flanked by a first and a second Int recognition sites. In some embodiments, the first site attP1 may comprise a first overlap sequence O1 and the second site attP2 may comprise a second overlap sequence O2. In yet another embodiment, the first O1 and the second O2 overlap sequences may be different, each consisting of seven nucleotides, the O1 may be identical to an overlap sequence O1 comprised within a first Int recognition site attE1 in a eukaryotic cell and the O2 may be identical to an overlap sequence O2 comprised within a second Int recognition site attE2 in the eukaryotic cell. In some embodiments, the eukaryotic recognition sites attE1 and attE2 may flank a target nucleic acid sequence of interest or any fragment thereof in the eukaryotic cell, or a kit or system comprising (a) and (b).

In yet another aspect, the invention relates to a method for replacing at least one nucleic acid sequence in a target nucleic acid sequence of interest or any fragment thereof with at least one a replacement-sequence, by site specific recombination of DNA in at least one eukaryotic cell, the method comprising the step of contacting said cell with: (a), at least one nucleic acid molecule comprising a replacement-sequence flanked by a first and a second Int recognition sites. In some embodiments, the first site attP1 may comprise a first overlap sequence O1 and the second site attP2 may comprise a second overlap sequence O2. In yet another embodiment, the first O1 and the second O2 overlap sequences may be different, each consisting of seven nucleotides, the O1 may be identical to an overlap sequence O1 comprised within a first Int recognition site attE1 in a eukaryotic cell and the O2 may be identical to an overlap sequence O2 comprised within a second Int recognition site attE2 in the eukaryotic cell. In other embodiments, the eukaryotic recognition sites attE1 and attE2 may flank a target nucleic acid sequence of interest or any fragment thereof in the eukaryotic cell. The cells are further contacted with (b), at least one HK-Int variant/mutated molecule or any functional fragments or peptides thereof, any nucleic acid molecule comprising a sequence encoding said HK-Int variant/mutated molecule or any vector, vehicle, matrix, nano- or micro-particle comprising the same. The method may thereby allow replacement of the target nucleic acid sequence of interest or any fragment thereof flanked by the attE1 and attE2 recognition sites in the eukaryotic cell, with the replacement sequence provided by the invention.

In yet another aspect, the invention relates to a method of curing or treating, preventing, inhibiting, reducing, eliminating, protecting or delaying the onset of a genetic disorder or condition in a subject in need thereof by administering to the subject an effective amount of at least one of: In a first option (i) (a) at least one nucleic acid molecule comprising a replacement-sequence flanked by a first and a second Int recognition sites. In some embodiments, the first site attP₁ may comprise a first overlap sequence O₁ and the second site attP₂ may comprise a second overlap sequence O₂. In another embodiment, the first O₁ and the second O₂ overlap sequences may be different, each consisting of seven nucleotides, the O₁ may be identical to an overlap sequence O₁ comprised within a first Int recognition site attE₁ in a cell of the subject and the O₂ may be identical to an overlap sequence O₂ comprised within a second Int recognition site attE₂ in the cell. In other embodiment, the recognition sites attE₁ and attE₂ may flank a target nucleic acid sequence of interest or any fragment thereof in the cell; and (b) at least one HK-Int mutated molecule or any functional fragments or peptides thereof at least one HK-Int variant/mutated molecule or any functional fragments or peptides thereof, any nucleic acid molecule comprising a sequence encoding the HK-Tnt variant/mutated molecule or any vector, vehicle, matrix, nano- or micro-particle comprising the same. In some embodiments, the HK-Int variant/mutated molecule comprise at least one substituted amino acid residue in at least one of the CB, ND and the CD of the Wild type HK-Int molecule.

In another option (ii), the method may involve administering to the subject an effective amount of at least one kit/system or composition comprising (a) and (b).

In an option (iii), the method may comprise the step of administering to the subject an effective amount of a cell comprising the nucleic acid molecule of (a) and a HK-Int variant/mutated molecule or nucleic acid molecule encoding such HK Int variants of (b). It should be understood that the invention further encompasses, in some embodiments thereof, the option of administering any combination of options (i), (ii) and (iii).

The method of the invention may thereby allow replacement of the target nucleic acid sequence of interest or any fragment thereof flanked by the attE₁ and attE₂ sites in the subject or in at least one cell of the subject, with the replacement gene.

In another aspect, the invention relates to an HK-Int variant/mutated molecule or any functional fragments or peptides thereof, any nucleic acid molecule comprising a sequence encoding the HK-Int variant/mutated molecule or any vector, vehicle, matrix, nano- or micro-particle comprising the same, any composition thereof or any cell transduced or transfected with the HK-Int variant/mutated molecule for use in a method for curing or treating, preventing, inhibiting, reducing, eliminating, protecting or delaying the onset of a genetic disorder or condition a genetic disorder in a subject in need thereof. The invention further relates to at least one nucleic acid molecule or any nucleic acid cassette or vector according to the invention, for use in a method for curing or treating, preventing, inhibiting, reducing, eliminating, protecting or delaying the onset of a genetic disorder or condition a genetic disorder in a subject in need thereof.

These and other aspects of the invention will become apparent by the hand of the following figures.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1D: Schemes of recombinase mediated cassette exchange (RMCE) mechanism

FIG. 1A: Incoming plasmid with sequence of interest (√) flanked by a compatible attP1 and attP2 sites.

FIG. 1B: Genomic DNA mutated Sequence (M) flanked by two incompatible site-specific RSs, attB1 and attB2 (triangles).

FIG. 1C: Result of RMCE of the Incoming plasmid of 1A with the genomic DNA of 1B, producing a recombinant genomic sequence.

FIG. 1D: Schematic representation of the lysogenic cycle of coliphage HK022. In phage HK022 infected E. coli, the phage circularized DNA integrates into the host genome via an Int-catalyzed attP×attB recombination forming a lysogenic host, in which the inserted prophage is flanked by the recombinant attL and attR sites. O is the overlap, P, B and C are Int binding sites.

FIG. 2A-2D: Comparative analysis of Int mutants integration activity using attP and attB w.t.

FIG. 2A: HK022 Integrase protein sequence as denoted by SEQ ID NO. 13. Substituting Mutational AA's are in bold presented under the w.t. original AA's. The N-terminal DNA binding domain (ND) (residues 1-63), as denoted by SEQ ID NO: 177, core-binding (CB) domain (residues 75-175), as denoted by SEQ ID NO: 178 and C-terminal catalytic domain (CD) (residues 176-356), as denoted by SEQ ID NO: 179.

FIG. 2B: Scheme of transient in trans w.t. attB×attP integration reaction using a promoter-GFP trap assay. Stop—transcription terminator.

FIG. 2C: FACS Quantitative data of Int variants recombination activity. Each plotted as percent of cells transfected with the o-nt (100%). The bars show the mean values of three independent experiments each with three repeats; the error bars indicate standard deviation.

FIG. 2D: FACS Quantitative data of Int variants recombination activity. Each plotted as percent of cells transfected with the oInt (100%). The bars show the mean values of three independent experiments each with three repeats; the error bars indicate standard deviation.

FIG. 3A-3C: Comparative analysis of Int mutants integration activity using “attP” and “attB” HEXA3, ATM4, DMD2, DMD3, CTNS1, CTNS4, CF10, CF12, SCN1A-3 and SCN1A-4 sites FIG. 3A: Scheme of transient in trans HEXA3, ATM4, DMD2, DMD3, CTNS1, CTNS4, CF10, CF12, SCN1A-3 and SCN1A-4 “attP”×“attB” integration reaction using a promoter-EGFP trap assay in human HEK293T cells. Stop—transcription terminator.

FIG. 3B: FACS data of Int variants relative recombination activity compare to oInt with HEXA3 and ATM4 sites.

FIG. 3C: FACS data of Int variants relative recombination activity compare to oInt with DMD2, DMD3, CTNS1, CTNS4, CF10, CF12, SCN1A-3 and SCN1A-4 sites. The bars show the mean values of three independent experiments each with three repeats; the error bars indicate standard deviation.

FIG. 4A-4H: Int-catalyzed transient RMCE reaction in HEK293 cells

FIG. 4A: Docking plasmid coding EF1α promoter-“attB”1-“attB”2-mCherry (ORF) cassette.

FIG. 4B: Incoming plasmid coding EGFP (ORF)-CMV promoter cassette flanked by “attP”1 and “attP”2.

FIG. 4C: Int-catalyzed RMCE product co-express GFP and mCherry from the EF1alfa and CMV promoters, respectively.

FIG. 4D: Representative FACS analysis of GFP-mCherry co-expressing cells (gated region) confirming Int-catalyzed transient RMCE reaction.

FIG. 4E: Bar graph shows the FACS quantification mean values of three independent experiments each with three repeats. More than 6% of the gated cells are GFP-mCherry positive.

FIG. 4F: PCR analysis of EF1α-GFP junction by primer 635 as denoted by SEQ ID NO:200 and primer 206 as denoted by SEQ ID NO: 160.

FIG. 4G: PCR analysis of CMV-mCherry junction by primer 204 as denoted by SEQ ID NO: 1 and primer 1185 as denoted by SEQ ID NO:201.

FIG. 4H: PCR analysis of RMCE full exchanged cassette by primer 635 as denoted by SEQ ID NO:200 and primer 1185 as denoted by SEQ ID NO:201. attB/P/L1-HEXA3 “att” sites. attB/P/L2-ATM4 “att” sites. Arrows—primers used for PCR analysis. L—appropriate fragments of 1 kb ladder.

FIG. 5A-5K: Int-catalyzed genome RMCE reaction in HEK293-Flp-in cells model FIG. 5A: Docking plasmid to be inserted in the genomic frt-integration site by Flp recombinase, coding EF1α promoter, “attB” 1, “attB”2 sites and mCherry (ORF).

FIG. 5B: HEK293-Flp-in genomic SV40 promoter-frt cassette.

FIG. 5C: Flp mediated integration product of the docking plasmid resulting Hygromycin resistant cells.

FIG. 5D: Incoming plasmid coding EGFP (ORF) upstream to CMV promoter flanked by “attP” 1 and “attP” 2 sites.

FIG. 5E: Int-RMCE product co-express GFP and mCherry.

FIG. 5F: Representative FACS analysis of GFP-mCherry co-expressing cells (gated region) confirming Int-catalyzed genomic RMCE reaction.

FIG. 5G: Bar graph show the FACS quantification mean values of three independent experiments each with three repeats. More than 1% of the gated cells are GFP-mCherry positive.

FIG. 5H: PCR analysis of SV40-HygR junction by primer 421 as denoted by SEQ ID NO:202 and primer 1016 as denoted by SEQ ID NO:203.

FIG. 5I: PCR analysis of EF1α-GFP junction by primer 635 as denoted by SEQ ID NO:200 and primer 206 as denoted by SEQ ID NO: 160.

FIG. 5J: PCR analysis of CMV-mCherry junction by primer 834 as denoted by SEQ ID NO:204 and primer 1191 as denoted by SEQ ID NO:205.

FIG. 5K: PCR analysis of RMCE full exchanged cassette by primer 635 as denoted by SEQ ID NO:200 and primer 1191 as denoted by SEQ ID NO:205. The figure further shows PCR analysis of Nested PCRs of EF1α-GFP junction (635+206) and CMV-mCherry junction (834+1191) on the recombinant cassette PCR. attB/P/L1-HEXA3 “att” sites. attB/P/L2-ATM4 “att” sites. Arrows—primers used for PCR analysis. L—appropriate fragments of 1 kb or 100 bp ladders.

FIG. 6A-6D. Schematic representation of the two steps assay for off-target Int activity analysis in E. coli

FIG. 6A: Step 1: KmR gene PCR analysis of ApR+KmR colonies obtained by Int-expressing cells transformation with KmR pSSK10 plasmid that carries the attP site wild type. Negative PCR in step 1 would indicate a false-positive phenotype.

Step 2: KmR gene PCR positive colonies obtained on the first step were used for the Int-catalyzed integration activity analysis.

FIG. 6B. KmR gene PCR analysis of ApR+KmR colonies obtained by Int-expressing cells transformation with KmR pSSK10 plasmid that carries human “attP”s (HEXA 5 and 10 or ATM 2 and 4). Positive PCR would indicate off-target activity while negative PCR would indicate a false-positive phenotype.

FIG. 6C. Quantification data of Int w.t. integration activity.

FIG. 6D. Quantification data of Int E174K mutant integration activity (HEXA5 and HEXA10 in the table correspond to sites HEXA3 and HEXA7, respectively, as referred to herein by the invention).

FIG. 7A-7D. Sequence alignment of the relevant attB sites

Figure shows attB of coliphage HK022. B and B′ are binding sites for Int. O—overlap (site of genetic exchange with attP).

FIG. 7A—attB of coliphage HK022 (SEQ ID NO. 161), having the o as denoted by SEQ ID NO. 162.

FIG. 7B (lines 1-6), the active human attBs that flank the mutation in exons 44 (DMD2 SEQ ID NO. 92 and DMD3 SEQ ID NO.93), exon 45 (DMD4 SEQ ID NO. 108 and DMD5 SEQ ID NO. 110) and exon 52 (DMD6 SEQ ID NO. 112 and DMD7 SEQ ID NO.114) of Dystrophin gene.

FIG. 7C (lines 1-2), the active human attBs that flank the mutation in exon 3 (CTNS4 SEQ ID NO.72 and CTNS1 SEQ ID NO. 116) of CTNS gene.

FIG. 7D. consensus sequence of an active attB. Arrows—CTTnnnnnnnAAG conserved palindrome (SEQ ID NO. 163).

FIG. 8. Scheme of the relevant human attB sites (“attB”), DMD

Schematic representation of the human attB sites that flank the mutations in exons 44, 45 and 52 of the dystrophin gene (DMD2 and DMD3, are indicated as D2 and D3).

FIG. 9. Scheme of the relevant human attB sites (“attB”), CTNS

Scheme of the relevant human attB sites (“attB”) that flank the mutation in exon 3 (b, c, henceforth CTNS4 and CTNS1, respectively) of CTNS gene (marked in grey) and a 57 kb deletion (marked by red line) (a, d) that extended outside the gene (CTNS A and CTNS D, as denoted by SEQ ID NO. 129 and SEQ ID NO. 130, respectively).

FIG. 10A-10D. Human attB sites (“attB”) activity assay in E. coli

FIG. 10A. Scheme of recombination substrate plasmid. Stop—transcription terminator. Arrows depict PCR primers.

FIG. 10B. Recombination products. Arrows depict PCR primers.

FIG. 10C. Colonies showing an active and an inactive “attB” site.

FIG. 10D. PCR analysis from a blue (b) and a white (w) colony. Black arrows depict the location of the primers used for PCR analysis as well as the PCR products.

FIG. 11A-11I: Scheme of Int catalyzed RMCE using “attB”s in the CTNS gene

FIG. 11A: Scheme EGFP-poly A trap assay: Incoming plasmid coding CMV-EGFP (ORF) lake of poly A, 2A, SD all flanked by “attP” CTNS4 and “attP” CTNS1 sites.

FIG. 11B: Scheme EGFP-poly A trap assay: Genomic CTNS locus with active “attB” CTNS4 and “attB” CTNS1 sites that flanks the CTNS promoter-exon 1-3 cassette.

FIG. 11C: Scheme EGFP-poly A trap assay: The RMCE reaction product at the genomic CTNS locus.

FIG. 11D: mRNA product of the RMCE produced incoming cassette (EGFP-P2A) fused to exons 4-11.

FIG. 11E: Representative FACS analysis of GFP expressing cells (gated regions) confirming Int-catalyzed genomic RMCE reaction.

FIG. 11F: Bar graph show the FACS quantification mean values of three independent experiments each with three repeats. More than 0.6% of the gated cells are GFP positive.

FIG. 11G: PCR analysis of CTNS locus-CMV junction by primer 1298 as denoted by SEQ ID NO:207 and primer 432 as denoted by SEQ ID NO:206.

FIG. 11H: PCR analysis of EGFP-exon 4 junction by primer 1015 as denoted by SEQ ID NO:208 and primer 1300 as denoted by SEQ ID NO:209.

FIG. 11I: PCR analysis of EGFP-exon 4 mRNA junction by primer 1015 as denoted by SEQ ID NO:208 and primer 1279 as denoted by SEQ ID NO:210. SD—Splicing donor. 2A—2a peptide ribosome skipping. Stop—transcription terminator. L—appropriate fragments of 100 bp ladder.

FIG. 12A-12I: Scheme of Int catalyzed RMCE in the DMD gene using exon 44 flanking “attB”s

FIG. 12A: Scheme EGFP-promoter trap assay: Incoming plasmid coding promoter-less EGFP-ORF, SA, 2A and Poly A all flanked by “attP” DMD2 and “attP” DMD3 sites.

FIG. 12B: Scheme EGFP-promoter trap assay: Genomic DMD locus with active “attB” DMD2 and “attB” DMD3 sites in introns 43 and 44 respectively that flanks exon 44.

FIG. 12C: Scheme EGFP-promoter trap assay: The RMCE reaction product at the genomic DMD locus.

FIG. 12D: Scheme EGFP-promoter trap assay: mRNA product of the RMCE produced incoming cassette (EGFP-P2A) fused to exons 1-43.

FIG. 12E: Representative FACS analysis of GFP expressing cells (gated regions) confirming Int-catalyzed genomic RMCE reaction.

FIG. 12F: Bar graph shows the FACS quantification mean values of three independent experiments each with three repeats. More than 0.4% of the gated cells are GFP positive.

FIG. 12G: PCR analysis of Exon 43-EGFP junction by primer 1232 as denoted by SEQ ID NO:211 and primer 1243 as denoted by SEQ ID NO: 152.

FIG. 12H: PCR analysis of EGFP-exon 45 junction by primer 1015 as denoted by SEQ ID NO:208 and primer 1236 as denoted by SEQ ID NO:212.

FIG. 12I: PCR analysis of Exon 43-EGFP mRNA junction by primer 1288 as denoted by SEQ ID NO:225 and primer 206 as denoted by SEQ ID NO:160.

SA—Splicing acceptor 0.2A—2a peptide ribosome skipping. Stop—transcription terminator. L—appropriate fragments of 1 kb or 100 bp ladders.

FIG. 13A-13C: Sequence alignment of the relevant human CFTR “attB” sites.

FIG. 13A—attB of coliphage HK022. O, as denoted by SEQ ID NO. 161, and the overlap sequence as denoted by SEQ ID NO. 162—overlap (site of genetic exchange with attP). B and B′ are binding sites for Int.

FIG. 13B—the active human “attB”s that flank the exon3 of CFTR gene. Specifically, CFTR10, CFTR12, CFTR13, CFTR14, as denoted by SEQ ID NO. 96, 97, 125, 126, respectively.

FIG. 13C—consensus sequence of an active attB as denoted by SEQ ID NO. 163. Arrows—CTTnnnnnnnAAG conserved palindrome.

FIG. 14: The “attB” sites location in human CFTR/

Figure shows scheme of the “attB” sites location in human CFTR gene suitable for integration of CFTR cDNA by Int-catalyzed RMCE reaction.

### DETAILED DESCRIPTION OF THE INVENTION

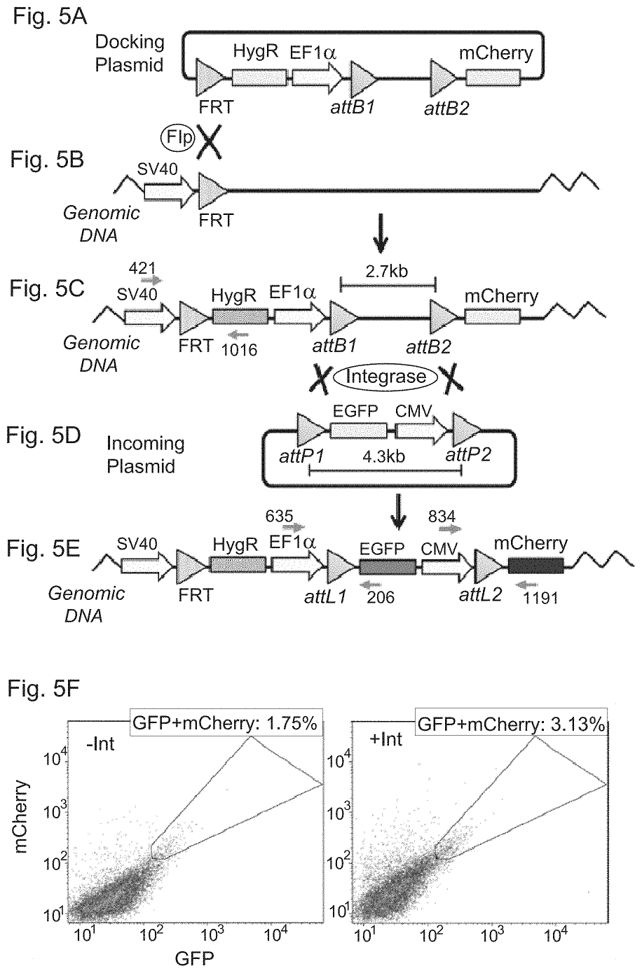

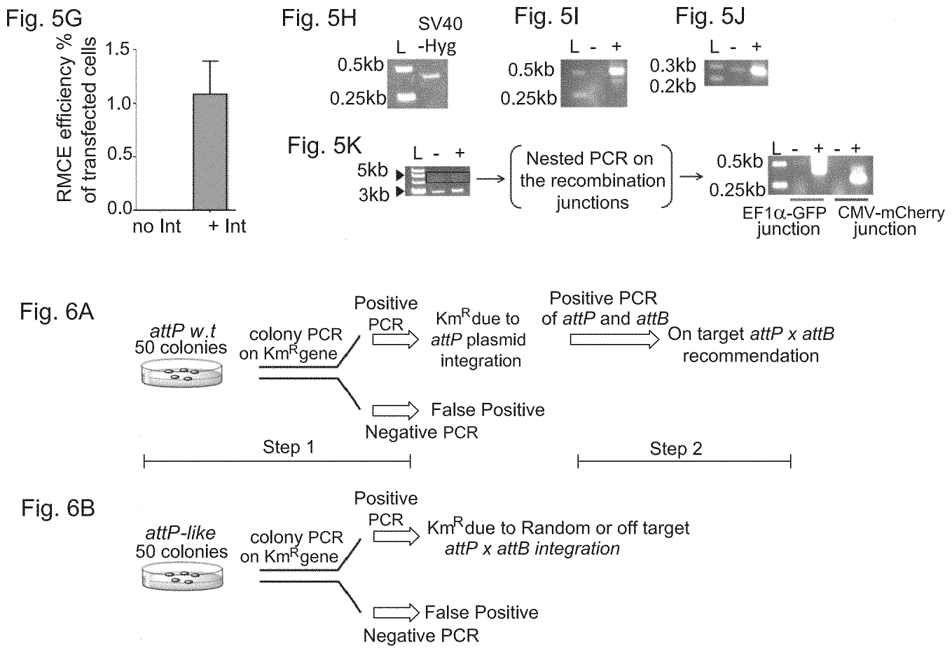

The present invention relates to novel mutants of the E. coli HK022 bacteriophage site specific recombinase Integrase (HK-Int) for gene editing in eukaryotic cells. The Inventors have identified eleven different mutants of HK-Int obtained by site-directed mutagenesis, as well as combinations thereof. More specifically, the E174K, E134K, D149K mutants located at the CB domain (the core-binding (CB) domain), the I43F mutant located at the ND domain (N-terminal DNA binding domain (ND), and the E264G, R319G, D336V, D215K, D278K, E309K, N303K mutants that carry substitutions located at the CD domain (C-terminal catalytic domain). The invention further encompasses mutants combining at least two of these substitutions, for example, the double mutants E174K/I43F, E174K/D278K, E174K/R319G, E174K/E264G, E174K/D336V, or the triple mutant E174K/I43F/R319G. The activity of these mutants was compared to the Wild type integrase in a trans integrative recombination between two plasmids. The results surprisingly revealed that the E174K and the D278K mutants exhibit a significantly enhanced activity over the WT Integrase enzyme. To demonstrate that Int is a potential tool for human genome manipulations, the inventors utilized the most RMCE transiently successive Int variant (Int E174K) to achieved stable genomic RMCE in the human model cell-line Flp-In-293 using GFP-mCherry co-expression promoter trap assay, showing over 1%, without any selection enrichment (FIG. 5G). The inventors have further exemplified Recombinase-Mediated Cassette Exchange reaction (RMCE) catalyzed by the HK-Int mutant of the invention using human native attB sites in human cells. Native attB sites flanking the human dystrophin gene (DMD), the human Cystinosin CTNS gene, as well as the cystic fibrosis transmembrane conductance regulator (CFTR) gene and the Sodium voltage-gated channel alpha subunit 1 (SCN1A) gene revealed by the inventors, allow the use of the novel HK-Int mutants of the invention in the treatment of Duchenne muscular dystrophy (DMD), Cystinosis, Cystic Fibrosis, and Dravet syndrome respectively, using the site specific recombination disclosed herein.

These findings have great implications on facilitating genetic manipulation of specific sites within the eukaryotic genome, for purposes of genetically modifying properties or traits as well of correcting DNA mutations that are associated with genetic disorders and diseases.

“Site-specific recombination” as used herein (also known as sequence-specific or conservative site-specific recombination), is a genetic recombination process in which DNA strand exchange takes place between segments possessing only a limited degree of sequence homology. As a non-limited example, site-specific-recombination occurs between specific sites on bacteriophage genome, such as λ or the coliphage HK022 and bacterial DNA molecules (e.g. E. coli) (6). Site-specific-recombination is guided primarily by proteins that recognize particular DNA sequences, which include site-specific recombinases or integrases. Improved integrases that recognize the eukaryotic sites and efficiently mediate recombination in eukaryotic cells are therefore desired for eukaryotic applications of gene editing, specifically, in gene therapy.

Therefore, in a first aspect the invention relates to a HK022 bacteriophage site specific recombinase Integrase (HK-Int) variant and/or mutated molecule or any functional fragments or peptides thereof.

Most site-specific recombinases are grouped into one of the two families, namely the tyrosine recombinase family and the serine recombinase family, based on the active amino acid and recombination mechanism. The names stem from the conserved nucleophilic amino acid residue that they use to attack the DNA and which becomes covalently linked to it during strand exchange. Among the known members of the tyrosine recombinases, are lambda (λ) integrase (Gene ID: 6065335), Cre (from the P1 phage, Gene ID: 2777477), including its derivative and FLP (from yeast S. Cerevisiae, having the accession number BBa_K313002). The serine recombinases include enzymes such as gamma-delta resolvase (from the Tn1000 transposon), the Tn3 resolvase (from the Tn3 transposon) and the φC31 integrase (from the φC31 phage, Gene ID: 2715866) or similar ones.

The HK022 integrase, as used herein, is a 357 amino acid protein (accession number P16407) as denoted by SEQ ID NO. 13. The gene encoding the Integrase (Int) recombinase of coliphage HK022, also termed “HK022p28 lambda family integrase, gp29” or “Enterobacteria phage HK022” consists of the nucleic acid sequence as denoted by Gene ID 1262484.

The Integrase (Int) recombinase of coliphage HK022 naturally mediates integration and excision of the bacteriophage into and out of the chromosome of its Escherichia coli host, using a mechanism that is similar to that used by coliphage λ integrase. In both phages, site-specific recombination reactions occur between two defined pairs of DNA attachment (att) sites. In nature, integration results from recombination between the phage attP site and the bacterial host attB, and excision occurs between the recombinant attR and attL sites that flank the integrated prophage. In addition to Int, these reactions require DNA-bending accessory proteins. Integrative recombination generally requires the host-encoded integration host factor (IHF) and excisive recombination requires IHF and the phage-encoded excisionase (Xis) (6). In a heterologous human cells environment, Int-HK022 accomplishes site-specific recombination even in the absence of the accessory proteins, namely, integration host factor (IHF) and Excisionase (Xis) that are required in the natural E. coli host (6) nevertheless the accessory proteins alleviate the efficiency of the reactions (9). The Integrase of the coliphage HK022 includes three different domains may act both in cis and in trans and facilitate functional assembly of a higher order tetrameric complex with DNA substrate known as an intasome. The N-terminal DNA binding domain (ND) (residues 1-63, also denoted by SEQ ID NO. 177) recognizes the ‘arm-type’ DNA sequences adjacent to the attP core site. The binding results in allosteric modifications allowing the function of the core-binding (CB) domain (residues 75-175, also denoted by SEQ ID NO. 178) and C-terminal catalytic domain (CD) (residues 176-356, also denoted by SEQ ID NO. 179) function. The CB domain recognizes the attP (C and C′)×attB (B and B′) core DNA sequences and is associated to the CD domain responsible for DNA cleavage and rejoining.

Still further, the present invention relates to HK Int variants, mutants, and mutated molecules, that are used herein interchangeably. A mutated molecule, or mutant as used herein refers to a mutated protein, specifically the integrase of the invention that carry at least one mutation in its encoding nucleic acid sequence. More specifically, a mutation as used herein is the permanent alteration of the nucleotide sequence encoding for the integrase of the invention. Mutations in accordance with the invention may comprise small scale mutations or large scale mutations (e.g., duplications, rearrangement, translocation or deletions or insertions of large fragments). More specifically, in accordance with the invention the mutants of the invention were prepared by performing small scale mutations, specifically, change that affect one or a few nucleotides, also indicated herein as a point mutation. It should be understood that mutation includes insertion or deletions of one nucleotide or more that may cause to a shift in the reading frame (frameshift), or substitutions of one nucleotide or more. Most common is the transition that exchanges a purine for a purine (A↔G) or a pyrimidine for a pyrimidine, (C↔T). In some embodiments, the mutants of the invention are created by point mutations, specifically, substitutions that alter the protein product (e.g., activity and/or stability), and more specifically, improves the recognition of eukaryotic sites and the efficiency of recombination in eukaryotic cells.

In some specific embodiments, the HK-Int variant and/or mutated molecule of the invention may comprise at least one substituted amino acid residue in at least one of the core-binding domain (CB), the N-terminal DNA binding domain (ND) and the C-terminal catalytic domain (CD) of the Wild type HK-Int molecule. It should be however appreciated that the Int variant and/or mutated molecule of the invention may comprise at least one mutation in at least one nucleotide of the nucleic acid sequence encoding the HK Int, that results in, point mutation, deletion, insertion causing deletion, insertion or substitution of any amino acid reside of the Wild type Int molecule. It should be noted that such mutations may involve one or more nucleotides, specifically, 1, 2, 3, 4, 5, 6, 7, 8, 9, 10, 15, 20, 25, 30, 35, 40, 45, 50 nucleotides or more, for example between 50-100, specifically, 60, 70, 80, 90, 100 or more, for example, 100-500 or more, specifically, 100, 150, 200, 250, 300, 350, 400, 450, 500 or more, for example, 500-1000 or more, 1000 (1 kb) to 10000 (10 kb) or more, for example, 10 kb to 100 kb or more, specifically, 100 kb to 1000 kb or more and 10000 kb to 100000 kb or more nucleotides. More specifically, the variant and/or mutated molecule of the invention may comprise in some embodiments mutation/s causing deletion/s, insertion/s and/or substitution/s of at least 1, 2, 3, 4, 5, 6, 7, 8, 9, 10, 15, 20, 25, 30, 35, 40, 45, 50, 60, 70, 80, 90, 100 or more, for example, 100-500 or more, specifically, 100, 150, 200, 250, 300, 350, 400, 450, 500 and more amino acid residues.

In some embodiment, the HK-Int mutated molecule may exhibit an improved activity in comparison with the activity of the Wild type Integrase, i.e. the ability to perform RMCE, specifically, RMCE in a particular eukaryotic target site. In more specific embodiments, the HK-Int mutated molecule of the invention may exhibit at least about 10-200% higher activity in comparison with the Wild type integrase, more specifically about 10%, 15%, 20%, 25%, 30%, 35%, 40%, 45%, 50%, 55%, 60%, 65%, 70%, 75%, 80%, 85%, 90%, 95%, 99%, 100%, 110%, 115%, 120%, 125%, 130%, 135%, 140%, 145%, 150%, 155%, 160%, 165%, 170%, 175%, 180%, 185%, 190%, 195% and even 200% or more increased, enhanced, improved, elevated, enlarged and higher activity in comparison with the Wild type integrase. With regards to the above, it is to be understood that, where provided, percentage values such as, for example, 10%, 50%, 100%, 120%, 500%, etc., are interchangeable with “fold change” values, i.e., 2, 3, 4, 5, 6, 7, 8, 9, 10, 15, 20, 25, 30, 40, 50 or more, etc., respectively.

In some further specific embodiments, the HK-Int variant and/or mutated molecule of the invention may comprise at least one substitution at any position of residues 174, 278, 43, 319, 134, 149, 215, 264, 303, 309 and 336, and any combinations thereof of the amino acid sequence of the Wild type HK-Int molecule. In some specific embodiments, the wild type HK-Int comprises the amino acid sequence as denoted by SEQ ID NO. 13.

In some specific embodiments, the HK-Int variant and/or mutated molecule may comprise at least one substitution at the CB domain, of the amino acid sequence of the Wild type HK-Int molecule as denoted by SEQ ID NO. 13. In some optional embodiments, the HK-Int variant or mutated molecule comprises at least one substitution in at least one of residues 174, 134, 149 and any combinations thereof.

In more specific embodiments, the HK-Int variant and/or mutated molecule of the invention may comprise at least one substitution at position 174 of the Wild type HK-Int molecule as denoted by SEQ ID NO. 13, and any functional fragments, variants, fusion proteins or derivatives thereof. In yet some further specific embodiments, the HK-Int variant and/or mutated molecule may comprise at least one substitution replacing glutamic acid (E) with lysine (K) at position 174 of the Wild type HK-Int molecule as denoted by SEQ ID NO. 13, and any functional fragments, variants, fusion proteins or derivatives thereof.

In some particular embodiments, the HK-Int variant and/or mutated molecule may be designated E174K. In more specific embodiments, the E174K variant of the invention may comprise the amino acid sequence as denoted by SEQ ID NO. 14, or any functional fragments, variants, fusion proteins or derivatives thereof. In some embodiments, the E174K mutant of the invention may be encoded by a nucleic acid sequence comprising the sequence as denoted by SEQ ID NO. 15, or any functional fragments, variants, or derivatives thereof. Still further, in some embodiments, the HK-Int variant and/or mutated molecule of the invention may comprise at least one substitution in other residues of the CB domain of the Int molecule, for example, at positions 134 and/or 149. In more specific embodiments, such variant may comprise a substituted amino acid residue at position 134. In more specific embodiments, the variant may comprise a substitution of E at position 134 to K, specifically, the E134K variant that comprises in some embodiments the amino acid sequence as denoted by SEQ ID NO. 180, or any functional fragments, variants, fusion proteins or derivatives thereof. In some embodiments, the E134K mutant of the invention may be encoded by a nucleic acid sequence comprising the sequence as denoted by SEQ ID NO. 181, or any functional fragments, variants, or derivatives thereof. In more specific embodiments, such variant may comprise a substituted amino acid residue at position 149. In more specific embodiments, the variant may comprise a substitution of D at position 149 to K, specifically, the D149K variant that comprises in some embodiments the amino acid sequence as denoted by SEQ ID NO. 188, or any functional fragments, variants, fusion proteins or derivatives thereof. In some embodiments, the D149K mutant of the invention may be encoded by a nucleic acid sequence comprising the sequence as denoted by SEQ ID NO. 189, or any functional fragments, variants, or derivatives thereof.

In some specific embodiments, the HK-Int variant and/or mutated molecule of the invention may comprise at least one substitution in the N-terminal DNA binding domain (ND) of the Int molecule.

In more specific embodiments, such variant may comprise a substituted amino acid residue at position 43. In more specific embodiments, the variant may comprise a substitution of Isoleucine with Phenylalanine at position 43, specifically, the I43F variant that comprises in some embodiments the amino acid sequence as denoted by SEQ ID NO. 42, or any functional fragments, variants, fusion proteins or derivatives thereof. In yet some further embodiments, such mutant may be encoded by a nucleic acid sequence comprising SEQ ID NO. 43.

In yet some further specific embodiments, the HK-Int variant and/or mutated molecule of the invention may comprise at least one substitution in the C-terminal catalytic domain (CD) of the Wild type HK-Int molecule. In more specific embodiments, such variant may comprise a substituted amino acid residue at any one of positions 278, 215, 264, 303, 309, 319, 336. In more specific embodiments, the variant may comprise a substitution of Glutamic acid with Glycine at position 264, specifically, the E264G variant that comprises in some embodiments the amino acid sequence as denoted by SEQ ID NO. 44, or any functional fragments. In yet some further embodiments, such mutant may be encoded by a nucleic acid sequence comprising SEQ ID NO. 45. In yet some further embodiments the variant may comprise a substitution of Glutamic acid with Glycine at position 319, specifically, the R319G variant that comprises in some embodiments the amino acid sequence as denoted by SEQ ID NO. 46, or any functional fragments. In yet some further embodiments, such mutant may be encoded by a nucleic acid sequence comprising SEQ ID NO. 47. In yet some further specific embodiments, the variant may comprise a substitution of Aspartic acid with Valine at position 336, specifically, the D336V variant that comprises in some embodiments the amino acid sequence as denoted by SEQ ID NO. 48, or any functional fragments.

In yet some further embodiments, such mutant may be encoded by a nucleic acid sequence comprising SEQ ID NO. 49. Still further in some embodiments the variant may comprise a substitution of aspartic acid with lysine at position 215, specifically, the D215K variant that comprises in some embodiments the amino acid sequence as denoted by SEQ ID NO. 190, or any functional fragments. In yet some further embodiments, such mutant may be encoded by a nucleic acid sequence comprising SEQ ID NO. 191.

In some additional embodiments, the variant may comprise a substitution of asparagine (N) with lysine at position 303, specifically, the N303K variant that comprises in some embodiments the amino acid sequence as denoted by SEQ ID NO. 223, or any functional fragments. In yet some further embodiments, such mutant may be encoded by a nucleic acid sequence comprising SEQ ID NO. 224.

In some further embodiments the variant may comprise a substitution of aspartic acid with lysine at position 309, specifically, the D309K variant that comprises in some embodiments the amino acid sequence as denoted by SEQ ID NO. 192, or any functional fragments. In yet some further embodiments, such mutant may be encoded by a nucleic acid sequence comprising SEQ ID NO. 193.

Still further, the mutant or variant of the invention may comprise at least two substituted amino acid residues. In yet some further embodiments, such double or triple mutants may carry at least one, at least two, at least three, at least four, at least five, at least six, at least seven, at least eight, at least nine, at least ten, at least eleven or more of any of the substitutions disclosed by the invention.

Thus, in some specific embodiments, the HK-Int variant and/or mutated molecule of the invention may be a double mutant.

In some specific embodiments, such mutant may comprise a substitution of glutamic acid (E) with lysine (K) at position 174 of the Wild type HK-Int molecule as denoted by SEQ ID NO. 13, and in addition at least one of the following substitutions: a substitution replacing Aspartic acid (D) with Lysine (K) at position 278, a substitution replacing Isoleucine (I) with Phenyl alanine (F) at position 43, a substitution replacing glutamic acid (E) with Glycine (G) at position 319, a substitution replacing glutamic acid (E) with Glycine (G) at position 264, and a substitution replacing Aspartic acid (D) with Valine (V) at position 336 of the Wild type HK-Int molecule as denoted by SEQ ID NO. 13, and any variants, homologs or derivatives thereof. In some specific embodiments, the HK-Int variant and/or mutated molecule of the invention may comprise a substitution of glutamic acid (E) with lysine (K) at position 174 of the Wild type HK-Int molecule as denoted by SEQ ID NO. 13, and in addition a substitution replacing D with K at position 278.

In some specific embodiments, such mutant is designated E174K/D278K mutant or variant.

In some further specific embodiments, the HK-Int variant and/or mutated molecule of the invention may comprise a substitution of E with K at position 174 of the Wild type HK-Int molecule as denoted by SEQ ID NO. 13, and in addition a substitution replacing Isoleucine (I) with Phenyl alanine (F) at position 43. In some specific embodiments, such mutant is designated E174K/I43F mutant or variant. In yet some further specific embodiments, the HK-Int variant and/or mutated molecule of the invention may comprise a substitution of E with K at position 174 of the Wild type HK-Int molecule as denoted by SEQ ID NO. 13, and in addition a substitution replacing E with G at position 319, such mutant is designated E174K/E319G mutant or variant. In some further specific embodiments, the HK-Int variant and/or mutated molecule of the invention may comprise a substitution of E with K at position 174 of the Wild type HK-Int molecule as denoted by SEQ ID NO. 13, and in addition a substitution replacing E with G at position 264 (mutant E174K/E264G), or in another embodiments, replacing D with V at position 336 (mutant E174K/D336V).

In some particular embodiments, the E174K/I43F mutant may comprise the amino acid sequence as denoted by SEQ ID NO. 83, or any derivatives, homologs, fusion proteins or variants thereof.

In yet some further embodiments, such mutant may be encoded by the nucleic acids sequence that comprises SEQ ID NO. 82.

In yet some further embodiments, the double mutant of the invention may comprise a substitution of E K at position 174 of the Wild type HK-Int molecule as denoted by SEQ ID NO. 13, and in addition a substitution replacing E with G at position 319 of the Wild type HK-Int molecule as denoted by SEQ ID NO. 13. In some specific embodiments, such mutant is designated E174K/R319G mutant.

In some particular embodiments, such mutant may comprise the amino acid sequence as denoted by SEQ ID NO. 85, or any derivatives, homologs, fusion proteins or variants thereof. In yet some further embodiments, such mutant may be encoded by the nucleic acids sequence that comprises SEQ ID NO. 84.

In yet some further embodiments, the double mutant of the invention may comprise a substitution E with K at position 174 of the Wild type HK-Int molecule as denoted by SEQ ID NO. 13, and in addition a substitution replacing D with K at position 278 of the Wild type HK-Int molecule as denoted by SEQ ID NO. 13. In some specific embodiments, such mutant is designated E174K/D278K mutant. In some particular embodiments, such mutant may comprise the amino acid sequence as denoted by SEQ ID NO. 184, or any derivatives, homologs, fusion proteins or variants thereof. In yet some further embodiments, such mutant may be encoded by the nucleic acids sequence that comprises SEQ ID NO. 186.

Further embodiments for double mutants include the mutants HK-Int molecule E174K/E264G and E174K/D336V that comprise in some embodiments the amino acid sequence as denoted by SEQ ID NO. 87 and SEQ ID NO. 89, respectively. In yet some further embodiments, such mutants are encoded by a nucleic acid sequence comprising the nucleic acid sequence as denoted by SEQ ID NO. 86 and 88, respectively.

In yet some further embodiments, the HK-Int variant of the invention may be a triple mutant that comprise three substitutions, specifically, three of the substitution disclosed by the invention. In some non-limiting example for such triple mutant, the HK-Int molecule E174K/I43F/R319G that comprise in some embodiments the amino acid sequence as denoted by SEQ ID NO. 185. In yet some further embodiments, such mutant is encoded by a nucleic acid sequence comprising the nucleic acid sequence as denoted by SEQ ID NO. 187, and any functional fragments, variants, fusion proteins or derivatives thereof.

In yet some further embodiments, the HK-Int variant/s, mutant/s and/or mutated molecule/s of the invention may comprise at least one substitution at the CD domain of the amino acid sequence of the Wild type HK-Int molecule as denoted by SEQ ID NO. 13. In yet some further embodiments, such HK-Int variant or mutated molecule may comprise at least one substitution in at least one of residues 278, 215, 264, 303, 309, 319, 336, and any combinations thereof.

In more specific embodiments, the HK-Int variant and/or mutated molecule of the invention comprises at least one substitution at position 278 of the Wild type HK-Int molecule as denoted by SEQ ID NO. 13 and any functional fragments, variants, fusion proteins or derivatives thereof.

In some particular embodiments, such HK-Int variant comprises at least one substitution replacing D with K at position 278 of the Wild type HK-Int molecule.

In some specific and non-limiting embodiments the HK-Int mutated molecule is designated D278K. More specifically, in some embodiments this mutant may comprise the amino acid sequence as denoted by SEQ ID NO. 182, or any functional fragments, variants, fusion proteins or derivatives thereof.

Still further, it must be understood that the invention further encompasses the option of triple mutants comprising for example E174K/E264G/D336V, or E174K/I43F/R319G, a mutant comprising four of the discussed mutations, for example, E174K/I43F/R319G/E264G or E174K/I43F/R319G/R319G, and any other possible combinations of all mutants discussed herein, or a mutant comprising six mutations, for example, E174K/D278K/I43F/R319G/E264G/R309K, or mutants comprising all eleven mutations, for example, E174K/D278K/I43F/R319G/E264G/R309K/E134K/D149K/N303K/D336V/D215K, or even additional substitutions.

It should be noted that “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide. Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, manosylation, amidation, carboxylation, sulfhydryl bond formation, cleavage and the like.

By “fragments or peptides” it is meant a fraction of said HK-Int variant, mutated molecule or mutant. A “fragment” of a molecule, such as any of the amino acid sequences of the present invention, is meant to refer to any amino acid subset of the HK-Int mutated molecule. For example, any peptide comprising 10 amino acid residues or more, specifically, 15, 20, 25, 30, 35, 40, 45, 50, 55, 60, 65, 70, 75, 80, 85, 90, 95, 100 or more, specifically, 150, 200, 250, 300, 350, 400, 450, 500 amino acid residues or more. This may also include “variants” or “derivatives” thereof. A “peptide” is meant to refer to a particular amino acid subset having functional activity. By “functional” is meant having the same biological function, for example, having the ability to perform RMCE, as described by the invention.

Integrase activity, as used herein refers to recombination between short sequences of DNA, the phage attachment site (attP), and a short sequence of target DNA, that may be either the bacterial attachment site (attB), or the site in the target eukaryotic nucleic acid sequence (attE). Integrases that catalyze the recombination are categorized as tyrosine or serine integrases, according to their mode of catalysis. More specifically, bacteriophage integrases are site-specific recombinases whose natural purpose is to insert and excise the viral genome during the establishment of lysogeny and the transition from lysogenic to lytic life cycle. Thus, as used herein, integrase activity refers to at least one of, the integration and/or the excision activity. The integration process is highly specific and is executed solely by the activity of the integrase enzyme. The enzyme binds to the two recombination substrates attB, found in the bacterial target genome (or the eukaryotic target genome, attE, as used herein), and attP, found in the phage genome and brings them together. DNA cleavage and strand exchange follow resulting in Holliday junction intermediate, which is resolved to form a recombinant molecule that comprise an insertion of the phage genome into the bacterial chromosome. The phage genome is flanked by two recombinant sites, each containing half of attB (or attE) and attP recombination substrates. The site on the left of the inserted phage is designated as attL, whereas, the one on the right as attR. A cellular protein, IHF (integration host factor), facilitates recombination by bending DNA and thus bringing the participating DNA strands in close proximity. The excision reaction takes place via similar steps and requires two additional accessory factors: Xis and Fis. Int, IHF, Xis, and Fis form a complex, which specifically binds to the P region of attR and promotes DNA cleavage and strand exchange recovering the original attB and attP sites, thus effectively executing clean and scarless removal of the phage.

RMCE (recombinase-mediated cassette exchange) is a procedure in reverse genetics allowing the systematic, repeated modification of higher eukaryotic genomes by targeted integration, based on the features of site-specific recombination processes (SSRs). For RMCE, this is achieved by the clean exchange of a preexisting gene cassette, or target genomic sequence, for an analogous cassette (e.g., compatible donor gene cassette) carrying the “replacement sequence”. More specifically, one or two relevant site-specific recombinases catalyze the exchange of an introduced DNA fragment located on an incoming plasmid with a genomic DNA fragment, both flanked by two relevant site-specific recombination sites. With this technology, the most abundant site-specific recombinases used in RMCE reactions are Cre of coliphage P1, Flp of yeast, and Integrase (Int) of the Streptomyces phage ΦC31. After “gene swapping” the donor cassette is safely locked in, but can nevertheless be re-mobilized in case other compatible donor cassettes are provided (“serial RMCE”). These features considerably expand the options for systematic, stepwise genome modifications.

It should be appreciated that the invention encompasses any variant or derivative of the HK-Int mutated molecules of the invention and any polypeptides that are substantially identical or homologue. The term “derivative” is used to define amino acid sequences (polypeptide), with any insertions, deletions, substitutions and modifications to the amino acid sequences (polypeptide) that do not alter the activity of the original polypeptides. In this connection, a derivative or fragment of the variant and/or mutated molecule of the invention may be any derivative or fragment of the variant and/or mutated molecule, specifically as denoted by SEQ ID NO. 14, 182, 184, 42, 44, 46, 48, 83, 85, 87, 89, 180, 185, 188, 190, 192, 223, that do not reduce or alter the activity of the variant of the invention. By the term “derivative” it is also referred to homologues, variants and analogues thereof. Proteins orthologs or homologues having a sequence homology or identity to the proteins of interest in accordance with the invention, specifically that may share at least 50%, at least 60% and specifically 65%, 70%, 75%, 80%, 85%, 90%, 91%, 92%, 93%, 94%, 95%, 96%, 97%, 98%, 99%, 100%, or higher, specifically as compared to the entire sequence of the proteins of interest in accordance with the invention, for example, any of the proteins that comprise the amino acid sequence as denoted by SEQ ID NO. 14, 182, 184, 42, 44, 46, 48, 83, 85, 87, 89, 180, 185, 188, 190, 192, 223. Specifically, homologs that comprise or consists of an amino acid sequence that is identical in at least 50%, at least 60% and specifically 65%, 70%, 75%, 80%, 85%, 90%, 91%, 92%, 93%, 94%, 95%, 96%, 97%, 98%, 99%, or higher to SEQ ID NO. 14, 182, 184, 42, 44, 46, 48, 83, 85, 87, 89, 180, 185, 188, 190, 192, 223, specifically, the entire sequence as denoted by SEQ ID NO. 14, 182, 184, 42, 44, 46, 48, 83, 85, 87, 89, 180, 188, 190, 192, 223.

It should be understood that the invention encompasses any HK-Int molecule for any of the aspects of the invention as disclosed herein after, with the proviso that such HK-Int is not the wild type molecule, specifically as denoted by SEQ ID NO. 13. In some embodiments thereof, the invention encompasses any of the of the HK-Int variants of the invention and any combinations thereof.

In some embodiments, derivatives refer to polypeptides, which differ from the polypeptides specifically defined in the present invention by insertions, deletions or substitutions of amino acid residues. It should be appreciated that by the terms “insertion/s”, “deletion/s” or “substitution/s”, as used herein it is meant any addition, deletion or replacement, respectively, of amino acid residues to the polypeptides disclosed by the invention, of between 1 to 50 amino acid residues, between 20 to 1 amino acid residues, and specifically, between 1 to 10 amino acid residues. More particularly, insertion/s, deletion/s or substitution/s may be of any one of 1, 2, 3, 4, 5, 6, 7, 8, 9, or 10 amino acids. It should be noted that the insertion/s, deletion/s or substitution/s encompassed by the invention may occur in any position of the modified peptide, as well as in any of the N′ or C′ termini thereof. With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention. For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M). Thus, in some embodiments, the invention encompasses HK-Int mutated molecules or any derivatives thereof, specifically a derivative that comprise at least 1, 2, 3, 4, 5, 6, 7, 8, 9, 10 or more conservative substitutions to the amino acid sequences as denoted by any one of SEQ ID NO. 14, 182, 184, 42, 44, 46, 48, 83, 85, 87, 89, 180, 185, 188, 190, 192, 223. More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q). Variants of the polypeptides of the invention may have at least 80% sequence similarity or identity, often at least 85% sequence similarity or identity, 90% sequence similarity or identity, or at least 95%, 96%, 97%, 98%, or 99% sequence similarity or identity at the amino acid level, with the protein of interest, such as the various polypeptides of the invention.

In a further aspect, the invention relates to a nucleic acid molecule comprising a nucleic acid sequence encoding a HK-Int mutated molecule and/or variant or any functional fragments or peptides thereof. Specifically, the invention relates to any nucleic acid sequence encoding any of the HK-Int mutated molecules of the invention, as well as to any nucleic acid cassette or vector comprising such nucleic acid sequence that encodes the mutants of the invention.

In some further embodiments, the nucleic acid sequence of the invention may comprise a nucleic acid sequence encoding a HK-Int mutated molecule and/or variant, wherein said variant comprise at least one substituted amino acid residue in at least one of the CB, the ND and the CD domains of the Wild type HK-Int molecule. In some specific embodiments, the HK-Int mutated molecule/s, mutants/s and/or variant/s encoded by the nucleic acid molecules of the invention may comprise at least one substitution at any position of residues 174, 278, 43, 319, 134, 149, 215, 264, 303, 309 and 336, of the amino acid sequence of the Wild type HK-Int molecule as denoted by SEQ ID NO. 13, and any combinations thereof (e.g., having double, triple, 4, 5, 6, 7, 8, 9, 10, 11 substitutions, mutations or more). In some particular embodiments, the HK-Int mutated molecule and/or variant encoded by the nucleic acid molecules of the invention may comprise at least one substitution at the CB domain. In some embodiments, the HK-Int mutated molecule and/or variant encoded by the nucleic acid molecules of the invention may comprise at least one substitution at positions 174, 134, 149, specifically, at position 174 of the Wild type HK-Int molecule as denoted by SEQ ID NO. 13, and any functional fragments, variants, fusion proteins or derivatives thereof. In yet some further embodiments, the HK-Int mutated molecule and/or variant encoded by the nucleic acid molecules of the invention may comprise at least one substitution replacing glutamic acid (E) with lysine (K) at position 174 of the Wild type HK-Int molecule as denoted by SEQ ID NO. 13.

In more specific embodiments, the HK-Int variant and/or mutated molecule encoded by the nucleic acid molecules of the invention may be designated E174K and may comprise the amino acid sequence as denoted by SEQ ID NO. 14 or any functional fragments, variants or derivatives thereof. In some embodiments, the HK-Int mutated molecule and/or variant encoded by the nucleic acid molecules of the invention may comprise at least one substitution at position 278 of the Wild type HK-Int molecule as denoted by SEQ ID NO. 13. In yet some further embodiments, the HK-Int mutated molecule and/or variant encoded by the nucleic acid molecules of the invention may comprise at least one substitution replacing D with K at position 278 of the Wild type HK-Int molecule as denoted by SEQ ID NO. 13.

In more specific embodiments, the HK-Int variant and/or mutated molecule encoded by the nucleic acid molecules of the invention may be designated D278K and may comprise the amino acid sequence as denoted by SEQ ID NO. 182 or any functional fragments, variants or derivatives thereof. Other alternative embodiments relate to the HK-Int mutated molecule and/or variant that comprise the amino acid sequence as denoted by any one of SEQ ID NO. 42, 44, 46 and 48, or the double mutants of the invention as denoted by SEQ ID NO. 184, 83, 85, 87, 89, or the triple mutants of SEQ ID NO.185, and any functional fragments, variants, fusion proteins or derivatives thereof.

In some particular embodiments, the nucleic acid molecules of the invention may comprise the nucleic acid sequence as denoted by SEQ ID NO. 15 (E174K) or any variants, derivatives, homologs or any fusion proteins thereof. In yet some other particular embodiments, the nucleic acid molecules of the invention may comprise the nucleic acid sequence as denoted by SEQ ID NO. 183 (D278K) or variants, derivatives, homologs or any fusion proteins thereof.

In yet some further particular alternative embodiments, nucleic acid molecules provided by the invention may comprise nucleic acid sequence encoding any of the Int variant and/or mutated molecule according to the invention. Non limiting examples may include the nucleic acid molecules that comprise at least one of the nucleic acid sequence as denoted by any one of SEQ ID NO. 43, SEQ ID NO. 45, SEQ ID NO. 47, SEQ ID NO.49, SEQ ID NO.181, SEQ ID NO.189, SEQ ID NO. 191, SEQ ID NO.193, and SEQ ID NO.224. Still further, the nucleic acid sequences provided by the invention include also nucleic acid sequences encoding the double mutants of the invention, for example, the nucleic acid sequences as denoted by any one of SEQ ID NO. SEQ ID NO. 82, SEQ ID NO. 84, SEQ ID NO. 86, SEQ ID NO. 88, SEQ ID NO. 186 and of the triple variant of SEQ ID NO. 187, and any functional fragments, variants, or derivatives thereof.

The term “nucleic acid”, “nucleic acid sequence”, or “polynucleotide” and “nucleic acid molecule” refers to polymers of nucleotides, and includes but is not limited to deoxyribonucleic acid (DNA), ribonucleic acid (RNA), DNA/RNA hybrids including polynucleotide chains of regularly and/or irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides, wherein the attachment of various entities or moieties to the nucleotide units at any position are included. The terms should also be understood to include, as equivalents, analogs of either RNA or DNA made from nucleotide analogs, and, as applicable to the embodiment being described, single-stranded (such as sense or antisense) and double-stranded polynucleotides. Preparation of nucleic acids is well known in the art.

It should be noted that the nucleic acid molecules (or polynucleotides) according to the invention can be produced synthetically, or by recombinant DNA technology. Methods for producing nucleic acid molecules are well known in the art.

The nucleic acid molecule according to the invention may be of a variable nucleotide length. For example, in some embodiments, the nucleic acid molecule according to the invention comprises 1-100 nucleotides, e.g., about 10, 20, 30, 40, 50, 60, 70, 80, 90 or 100 nucleotides. In other embodiments the nucleic acid molecule according to the invention comprises 100-1,000 nucleotides, e.g., about 100, 200, 300, 400, 500, 600, 700, 800, 900 or 1000 nucleotides. In further embodiments the nucleic acid molecule according to the invention comprises 1,000-10,000 nucleotides, e.g., about 1,000, 2,000, 3,000, 4,000, 5,000, 6,000, 7,000, 8,000, 9,000 or 10,000 nucleotides. In yet further embodiments the nucleic acid molecule according to the invention comprises more than 10,000 nucleotides, for example, 20,000, 30,000, 40,000, 50,000, 60,000, 70,000, 80,000, 90,000, 100,000 or more nucleotides.

The invention relates to nucleic acid sequences as well as to any variants, derivatives, fragments and homologs thereof. The term “homologues” is used to define nucleic acid sequences (oligonucleotide) which maintain a minimal homology to the nucleic acid sequences defined by the invention, e.g. preferably have at least about 65%, more preferably at least about 70%, at least about 75%, even more preferably at least about 80%, at least about 85%, most preferably at least about 90%, at least about 95% overall sequence homology, specifically, with the entire nucleic acid sequence of any of the nucleic acid sequences of the invention as structurally defined above, e.g. of a specified sequence, more specifically, the nucleic acid sequences that encode any of the HK-Int variants of the invention, specifically, any one of SEQ ID NO. SEQ ID NO. 15, 43, 45, 47, 49, 82, 84, 86, 88, 183, 186, 187, 181, 189, 191, 193, 224, any nucleic acid sequence comprising any combination of these sequences and any variants and derivatives thereof. It should be noted however that the invention relates to any homologs, derivative or variants of any of the nucleic acid sequences of any of the cassettes disclosed herein after in connection with other aspects of the invention, for example, any of the replacement sequences discussed herein after (e.g., of SEQ ID NO. 215, 216, 217, 218, 219, 220, 221, 222), or any of the ate sites disclosed by the invention, and any variants and derivatives thereof. The term “derivative” or “variant” is used to define nucleic acid sequences (oligonucleotide), with any insertions, deletions, substitutions and modifications of between about 1 to 100 bases, to the nucleic acid sequences that do not alter the activity of the original nucleotide sequences (specifically, to encode the functional HK-Int variants of the invention, as well as any of the nucleic acid replacement sequences). More specifically, 1, 2, 3, 4, 5, 6, 7, 8, 9, 10, 15, 20, 25, 30, 35, 40, 45, 50, 55, 60, 65, 70, 75, 80, 85, 90, 95, 100 or more nucleotides, more specifically, 1 to 10 nucleotides.

In some specific embodiments, the nucleic acid molecule of the invention may be any vector, nucleic acid cassette or vehicle comprising a nucleic acid sequence encoding a HK-Int mutated molecule and/or variant of the invention or any functional fragments, variants, derivatives or peptides thereof.

In some embodiments, the vector of the invention may comprise a nucleic acid sequence encoding any of the HK-Int mutated molecules and/or variants as defined above by the invention. Vectors, as used herein, are nucleic acid molecules of particular sequence that can be introduced into a host cell, thereby producing a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression. Many vectors, e.g. plasmids, cosmids, minicircles, phage, viruses, (as detailed below) useful for transferring nucleic acids into target cells may be applicable in the present invention. The vectors comprising the nucleic acid(s) may be maintained episomally, e.g. as plasmids, minicircle DNAs, viruses such cytomegalovirus, adenovirus, or they may be integrated into the target cell genome, through homologous recombination or random integration, e.g. retrovirus-derived vectors such as AAV, MMLV, HIV-1, ALV, etc. Other vectors that may be applicable for the nucleic acid sequence of the invention are those disclosed herein after in connection with other aspects of the invention.

In some specific embodiments, the HK-Int variant and/or mutated molecules or any functional fragments or peptides thereof or any nucleic acid molecules of the invention may be present in a host cell.

Thus, in yet a further aspect, the invention relates to a host cell transformed or transfected with at least one nucleic acid molecule comprising a nucleic acid sequence encoding at least one HK-Int variant and/or mutated molecule or any functional fragments or peptides thereof, any combinations thereof, or with any vector, vehicle, matrix, nano- or micro-particle comprising the same. In yet some further embodiments, the Int variant and/or mutated molecules expressed by the host cells of the invention may comprise at least one mutation causing at least one of substitution, deletion or insertion of one or more, two or more, three or more, five or more, six or more, seven or more, eight or more, nine or more, and ten or more amino acid residues.

In yet some embodiments, the host cell of the invention comprise, or may be transformed or transfected with at least one nucleic acid molecule comprising a nucleic acid sequence encoding at least one HK-Int variant and/or mutated molecule comprising at least one substituted amino acid residue in at least one of the CB, the ND and the CD domains of the Wild type HK-Int molecule.

In some particular embodiments, the HK-Int mutated molecule and/or variant may comprise at least one substitution at any position of residues 174, 278, 43, 319, 134, 149, 215, 264, 303, 309, 336 of the amino acid sequence of the Wild type HK-Int molecule as denoted by SEQ ID NO. 13 and any combinations thereof. In some other embodiments, the HK-Int mutated molecule and/or variant may comprise at least one substitution at the CB domain. In yet some specific embodiments, the HK-Int mutated molecule and/or variant may comprise at least one substitution at position 174 of the Wild type HK-Int molecule as denoted by SEQ ID NO. 13. In yet some further specific embodiments, the HK-Int mutated molecule and/or variant may comprise at least one substitution replacing E with K at position 174 of the Wild type HK-Int molecule as denoted by SEQ ID NO. 13. In some embodiments, the HK-Int mutated molecule and/or variant may comprise at least one substitution at position 278 of the Wild type HK-Int molecule as denoted by SEQ ID NO. 13. In yet some further embodiments, the HK-Int mutated molecule and/or variant encoded by the nucleic acid molecules of the host cells of the invention may comprise at least one substitution replacing D with K at position 278 of the Wild type HK-Int molecule as denoted by SEQ ID NO. 13.

In more specific embodiments, the HK-Int variant and/or mutated molecule of the host cells of invention may be designated D278K and may comprise the amino acid sequence as denoted by SEQ ID NO. 182 or any functional fragments, variants or derivatives thereof. Still further, in some specific embodiments, HK-Int variant or mutant of the host cells of the invention may comprise a substitution of E with K at position 174 of the Wild type HK-Int molecule as denoted by SEQ ID NO. 13, and in addition at least one of a substitution replacing D with K at position 278, a substitution replacing I with F at position 43, a substitution replacing E with G at position 319, a substitution replacing E with G at position 264 and a substitution replacing D with V at position 336 of the Wild type HK-Int molecule as denoted by SEQ ID NO. 13, and any functional fragments, variants, fusion proteins or derivatives thereof. In some specific embodiments, the HK-Int variant and/or mutated molecule of the invention may comprise a substitution of E with K at position 174 of the Wild type HK-Int molecule as denoted by SEQ ID NO. 13, and in addition a substitution replacing D with K at position 278. In some specific embodiments, such mutant is designated E174K/D278K mutant or variant. In some particular embodiments, such mutant may comprise the amino acid sequence as denoted by SEQ ID NO. 184, or any derivatives, homologs, fusion proteins or variants thereof. In yet some further embodiments, such mutant may be encoded by the nucleic acids sequence that comprises SEQ ID NO. 186, or any fragments, derivatives and homologs thereof. In yet some further embodiments the HK-Int mutated molecule and/or variant may comprise may comprise a substitution of E with K at position 174 and in addition a substitution replacing I with F at position 43. In some specific embodiments, such mutant is designated E174K/I43F mutant. In some particular embodiments, such mutant may comprise the amino acid sequence as denoted by SEQ ID NO. 83. In yet some further embodiments, such mutant may be encoded by the nucleic acids sequence that comprises SEQ ID NO. 82 or any fragments, derivatives and homologs thereof. In yet some further embodiments, the double mutant of the invention may comprise a substitution of E with K at position 174 of the Wild type HK-Int molecule and in addition a substitution replacing E with G at position 319 of the Wild type HK-Int molecule as denoted by SEQ ID NO. 13. In some specific embodiments, such mutant is designated E174K/R319G mutant. In some particular embodiments, such mutant may comprise the amino acid sequence as denoted by SEQ ID NO. 85. In yet some further embodiments, such mutant may be encoded by the nucleic acids sequence that comprises SEQ ID NO. 84 or any fragments, derivatives and homologs thereof. Still further, in some embodiments, the mutant expressed by the host cells of the invention may comprise the amino acid sequence as denoted by SEQ ID NO. 87 or 89. In yet some further embodiments, the host cells of the invention may comprise and express HK Int mutants comprising three substitutions, for example, the triple mutant that may comprise the amino acid sequence as denoted by SEQ ID NO. 185. In yet some further embodiments, the mutant of the host cells of the invention may comprise four, five, six, seven, eight, nine, ten, eleven or more of the point mutations discussed herein in any possible combinations thereof, or alternatively, all eleven mutations discussed herein.

In yet some further embodiments, the HK-Int variant and/or mutated molecule of the host cells of the invention may comprise at least one substitution at the CD domain of the amino acid sequence of the Wild type HK-Int molecule as denoted by SEQ ID NO. 13. In yet some further embodiments, such variant or mutated molecule may comprise at least one substitution in at least one of residues 278, 215, 264, 303, 309, 319, 336, and any combinations thereof.

In some specific embodiments, the host cell of the invention may comprise (e.g., transformed or transfected with) at least one nucleic acid molecule comprising a nucleic acid sequence encoding at least one HK-Int variant and/or mutated molecule comprising the amino acid sequence as denoted by SEQ ID NO. 14 (E174K), or any fragments, derivatives, homologs, fusion proteins or variants thereof. In some specific embodiments, the host cell of the invention may be transformed or transfected with at least one nucleic acid molecule comprising a nucleic acid sequence encoding at least one HK-Int variant and/or mutated molecule comprising the amino acid sequence as denoted by SEQ ID NO. 182 (E278K), or any fragments, derivatives, homologs, fusion proteins or variants thereof. It should be noted that the invention further encompasses any host cells transformed or transfected with at least one nucleic acid molecule encoding any of the Int variants of the invention as denoted by SEQ ID NO. 42, 44, 46, 48, 83, 85, 87, 89, 184, 185, 180, 188, 190, 192, 223 or any functional fragments, variants, fusion proteins or derivatives thereof. These HK-Int mutants or variants of the invention may be encoded according to some embodiments with the nucleic acid sequence as denoted by any one of SEQ ID NO. 15, 43, 45, 47, 49, 82, 84, 86, 88, 183, 186, 187, 181, 189, 191, 193, 224, or any functional fragments, variants, or derivatives thereof.

The term “host cell” includes a cell into which a heterologous (e.g., exogenous) nucleic acid or protein has been introduced. Persons of skill upon reading this disclosure will understand that such terms refer not only to the particular subject cell, but also is used to refer to the progeny of such a cell, as well as any population of cells comprising the host cell/s of the invention. Because certain modifications may occur in succeeding generations due to either mutation or environmental influences, such progeny may not, in fact, be identical to the parent cell, but are still included within the scope of the term “host cell”.

The term “host cells” as used herein refers to any cell known to a skilled person wherein the HK-Int variant and/or mutated molecule or any functional fragments or peptides thereof or any nucleic acid molecule according to the invention may be introduced. For example, a host cell may be eukaryotic or prokaryotic cell of a unicellular or multi-cellular organism. More specifically, a host cell may include, but is not limited to a yeast, fungi, an insect cell, an invertebrate cell, vertebrate cell, mammalian cell and the like.

The “host cell” as used herein refers also to cells that comprise, and/or express any of the HK Int variant/s, mutant/s of the invention, which can be transformed or transfected with naked DNA, any plasmid or expression vectors constructed using recombinant DNA techniques, as disclosed herein before. A drug resistance or other selectable marker carried on the transforming or transfecting plasmid is intended in part to facilitate the selection of the transformants. Additionally, the presence of a selectable marker, such as drug resistance marker may be of use in keeping contaminating microorganisms from multiplying in the culture medium. Such a pure culture of the transformed host cell would be obtained by culturing the cells under conditions which require the phenotype for survival. It should be understood that the term “host cells” as used herein also encompasses cells of an autologous source, allogenic source or a syngeneic source that are discussed herein after, in connection with the therapeutic methods provided by the invention.

It should be noted that in some embodiments, the presence in the host cell of at least one of any of the HK-Int variant and/or mutated molecules or any functional fragments or peptides thereof or any nucleic acid molecules of the invention may enable a process of directed and targeted manipulation or replacement of a target sequences comprised within the host cell, specifically, within the genome of the host cells of the invention, with a replacement sequence, using directed recombination mediated by the Int variant of the invention comprised within the host cell of the invention. Thus, a host cell in accordance with some embodiments of the invention, that expresses the HK Int variant/s and mutant/s of the invention together with a relevant nucleic acid sequence comprising a replacement sequence may enable and support the process of RMCE as described by the invention.

Phage DNA and bacteria served as a classical model system for studying such recombination reactions and hence recombination terminology was based thereon. The attachment site for a recombinase in bacteria is generally referred to as “attB” and the base sequence thereof is symbolized B-O-B′ (B for “bacterial”). Respectively, the specific attachment site for a recombinase on phage DNA is termed “attP” and the base sequence thereof is termed P-O-P′ (P for “phage”).

The terms attP and attB have become known in the art to generally refer to a donor DNA and a recipient DNA, respectively. In some embodiments, the recipient DNA is of a eukaryotic cell and therefore it is referred to herein as ate. As some non-limiting examples, while the donor DNA may be carried by a plasmid, a nucleic acid cassette, a vector or a virus, or any vehicle as disclosed by the invention, the recipient DNA usually refers to the host cell, for example, a bacterial or a eukaryotic cell.

The letter “O” in the terms B-O-B′ and P-O-P′ denotes the overlap core sequence, which consists of identical nucleic acid sequence in both DNA sequences to be recombined (e.g. on both the donor and the recipient DNA). After all four chains are cut, B joins P′ and P joins B′ to form one DNA molecule comprising sequences from both origins, namely, forming BOP′ (attL) and POB′ (attR) structures.

While some site-specific recombination systems only require a recombinase enzyme and the adequate recombination sites for performing site-specific-recombination, in other systems a number of accessory proteins and/or accessory sites are also required. For example, insertion of phage (for example, HK022 or lambda) DNA into bacterial DNA, mediated by an integrase, may also involve the accessory proteins “integration host factor” (IHF) and excisionase (Xis), which are required in for recombination in the natural E. coli host.

Recombination sites (i.e. attP and attB, or attP and attE) are typically between 30 and 200 nucleotides-long and consist of two motifs, namely P and P′ and B and B′, respectively. As detailed above, the motifs P and P′ as well as B and B′, or E and E′, to which the recombinase binds, share a partial inverted-repeat symmetry. It should be noted that this partial inverted symmetry is limited for the B and B′ or E and E′ sites, and does not include the Int binding sites on the P and P′ arms.

To facilitate the RMCE by the Int variant/s and/or mutant/s of the invention expressed by the host cell, the host cell must be provided also with a “donor” nucleic acid molecule, e.g., a plasmid that comprises a replacement nucleic acid sequence that is suitable for replacing a target nucleic acid sequence within the genome of the host cell. “Donor nucleic acid” is defined here as any nucleic acid supplied to an organism or receptacle to be inserted or recombined wholly or partially into the target sequence by recombination mediated by the Int variant/s and/or mutants of the invention. For example, in case that the target sequence, that may comprise or comprised within a target nucleic acid sequence or any fragment thereof, that should be replaced may be a mutated sequence, for example, a gene that carry at least one mutation causing a congenital disease, the host cells must be provided with a replacing nucleic acid sequence that is an un-mutated version of the same gene or fragment of gene. The replacement sequence should be provided with a sequence that enables or facilitates recombination and replacement of the target sequence in the target cell.

Thus, in yet some other embodiments, the host cell of the invention may further comprise, or may be transformed or transfected by at least one nucleic acid molecule or any nucleic acid cassette or vector thereof. In some embodiments, such nucleic acid molecule/s comprises at least one replacement-sequence flanked by a first and a second Int recognition sites. More specifically, the first site attP1 may comprise a first overlap sequence O1 and the second site attP2 may comprise a second overlap sequence O2. In some embodiments, the first O1 and the second O2 overlap sequences are different, each consisting of seven nucleotides, the O1 may be identical to an overlap sequence O1 comprised within a first Int recognition site attE1 in a eukaryotic cell and the O2 may be identical to an overlap sequence O2 comprised within a second Int recognition site attE2 in said eukaryotic cell. It should be noted that in some embodiments, the eukaryotic recognition sites attE1 and attE2 flank a target nucleic acid sequence of interest or any fragment thereof in the eukaryotic cell, wherein the O1 and O2 overlap sequences are each flanked by a first E and a second E′ Int binding sites. In some embodiments, the first binding sites E may comprise the sequence of C1-T2-T3-W4, as denoted by SEQ ID NO. 16, and the second binding sites E′ may comprise the sequence of A12-A13-A14-G15, as denoted by SEQ ID NO. 17.

In more specific embodiments, the first and second Int sites comprised within the nucleic acid molecule of the invention that comprise the replacement sequence, comprise the native attP sites, with the non-native “O” sequence. In some embodiments, the first attP₁ sequence comprises a first overlap nucleic acid sequence O₁ flanked by a wild type P₁ and P′₁ arms of attP. It should be noted that in some embodiments, in addition to Int recognition sites these arms may also include recognition sites for IHF and XIS proteins. The second attP₂ sequence may comprise a second overlap O₂ nucleic acid sequence likewise flanked by the wild type P₂ and P′₂ arms. In some embodiments, the native arms of attP are identical in both, attP₁ and attP₂. It should be therefore understood that, as used herein throughout the specification, the nucleic acid sequence of the native P₁ may be identical to the sequence of P₂ and the sequence of P′₁ may be identical to P′₂. As mentioned above, the first O₁ and the second O₂ overlap nucleic acid sequences are random sequences that must be identical to the overlap nucleic acid sequence in the Int sites of the host eukaryotic cell (attE).

By the terms “a first” and “a second” as used herein, it is referred to different positions of the nucleotide sequences, in a 5′ to 3′ direction along the nucleic acid molecule, specifically, the target nucleic acid molecule (acceptor) or the donor cassette that comprise the replacement sequence. For example, as indicated above, the present invention provides a nucleic acid molecule comprising a replacement-sequence flanked by a first and a second Int attP nucleic acid sequences. Accordingly, the first Int-attP nucleic acid sequence is located 5′ (or upstream) to the second Int attP nucleic acid sequence. Similarly, the first Int attE₁ nucleic acid sequence that flank the target sequence in a eukaryotic cell is located 5′ (or upstream) to the second Int attE₂ nucleic acid sequence in the eukaryotic cell.

In a similar fashion, by the terms “first overlap nucleic acid sequence O₁” and “second overlap nucleic acid sequence O₂” it is referred to the nucleic acid sequence O₁ being located 5′ (or upstream) to the nucleic acid sequence O₂. As indicated above, the overlap “O” sequence, or element of the attP, and/or ate sites of the invention comprise, and in some embodiments is composed of seven nucleotides or bases. However, it should be understood that the invention further encompasses in some embodiments thereof the option of the overlap “O” sequence that comprise more than 7 nucleotides or less than 7 nucleotides, for example, at least 3, 4, 5, 6 nucleotides or less, or alternatively, at least 8, 9, 10 nucleotides or more.

Still further, as noted above, the overlap “O” sequence, element or segment of the attP site, is identical to it's corresponding “O” element in the ate site. More specifically, for O1 of the attP1 is identical to an overlap sequence O1 comprised within a first Int recognition site attE1, and O2 of the attP2 is identical to an overlap sequence O2 comprised within a second Int recognition site attE2. It means that O1 of the attP1 and O1 of the attE1 consists of the same sequence, the same seven nucleotides as they are identical, and that O2 of the attP2 and O2 of the attE2, are identical, and consist of the same sequence. However, it should be understood that the invention in some embodiments thereof, further encompasses the option that the “O” sequences in the attP and the “O” sequence in the corresponding attE sites, are not completely identical. For example, these “o” elements may differ in one nucleotide or more. In yet some further embodiments, the “O” sequences in the attP and the “O” sequence in the corresponding attE sites display 80%, 81%, 82%, 83%, 84%, 85%, 86%, 87%, 88%, 89%, 90%, 91%, 92%, 93%, 94%, 95%, 96%, 97%, 98%, 99% identity, and preferably, 100 identity.

The term “flanked” as used herein refers to a nucleic acid sequence positioned between two defined regions. For example, as indicated above, the replacement-sequence is flanked by a first and a second Int attP nucleic acid sequences, where the first Tnt attP nucleic acid sequence is positioned 5′ (or upstream) to the replacement-gene and the second Int attP nucleic acid sequence is positioned 3′ (or downstream) to the replacement-sequence.

The invention provides, as indicated above, at least one nucleic acid molecule comprising a replacement-sequence flanked by a first and a second Int attP nucleic acid sequences or any vector or nucleic acid cassette comprising such sequence. The invention further provides host cells comprising and/or transformed or transfected with such nucleic acid sequences. As used herein, the term “replacement-sequence” refers to a nucleic acid sequence that is positioned between two different Int-attP nucleic acid sequences, specifically, the natural sites of the phage except for their overlap sequences, and is intended for replacing a nucleic acid fragment in the host DNA (i.e. the target nucleic acid sequence of interest or any fragment thereof) which is positioned between two corresponding different Int attE nucleic acid sequences. In some embodiments, such replacement sequence may comprise at least one nucleic acid sequence encoding a product (e.g., protein and/or RNA) that is directly or indirectly essential, beneficial or advantageous for the expressing cell. In some embodiments, such replacement sequence may comprise the native, non-mutated version of a gene or any nucleic acid sequence that should replace the mutated version in the target cell. It should be however understood that this method further enables manipulation of genes or gene fragments that do not necessarily comprise any mutation. The replacement gene may be in some embodiment, a gene or fragment thereof that may comprise mutation or any manipulation that may improve and/or change the native nucleic acid sequence within the target cell, or even modulate the expression of a target nucleic acid sequence, e.g., at least one gene or any fragments thereof. In some embodiments, the length of such replacement nucleic acid sequence provided by the cassette of the invention may range between about 100,000 nucleotides or more, to about 10 nucleotides or less. More specifically, the length of the nucleic acid sequence of interest may be about 100,000 nucleotides in length, or less than 75,000 nucleotides in length or less than 50,000 nucleotides in length, or less than 40,000 nucleotides in length, or less than 30,000 nucleotides in length, or less than 20,000 nucleotides in length, or less than 15,000 nucleotides in length, or less than 10,000 nucleotides in length, or less than 5000 nucleotides in length, or less than 1000 nucleotides in length, or less than 900 nucleotides in length, or less than 800 nucleotides in length, or less than 700 nucleotides in length, or less than 600 nucleotides in length, or less than 500 nucleotides in length, or less than 450 nucleotides in length, or less than 400 nucleotides in length, or less than 300 nucleotides in length, or less than 200 nucleotides in length, or less than 100 nucleotides in length, or less than 50 nucleotides in length, or less than 40 nucleotides in length, or less than 30 nucleotides in length, or less than 20 nucleotides in length, or less than 10 nucleotides in length. In some embodiments, the replacement nucleic acid sequence provided by the cassette of the invention may be in the length of 20,000 (20 Kb) nucleotides or more.

In some embodiments, the replacement sequence comprise a sequence that differs from the target nucleic acid sequence in at least 1, 2, 3, 4, 5, 6, 7, 8, 9, 10 or more, 20, 30, 40, 50, 60, 70, 80, 90, 100 or more, 200, 300, 400, 500 nucleotides or more. It should be understood that the replacement sequence differs from the target sequence that is replaced, and display in some embodiments only 50% to 99% identity, for example, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identity. It should be noted that the described replacement sequence is relevant to all aspects of the invention. As noted above, the attE sites that flank the target nucleic acid sequence of interest (or any fragment thereof) in the target eukaryotic cell, comprise random O1 and O2 sequences each flanked by E and E′ sites having a consensus sequence as denoted by SEQ ID NO.16 and 17 (for E and E′, respectively). It should be understood that “A” refers to adenosine, “T” refers to thymidine, “C” relates to cytidine, “G” refers to guanosine and “W” as used herein may be any one of “A” (adenosine) or “T” (thymidine).

In more specific embodiments, the HK-Int variant and/or mutated molecules of the invention may use the recognition sites comprising the nucleotide sequence of SEQ ID NO. 100 and SEQ ID NO. 101, as the P and P′ arm sites, respectively. These molecules are preceded by 7 nucleotides of the “O” sequence, specifically, at positions 5, 6, 7, 8, 9, 10, 11, that are followed by the E′ element as denoted by SEQ ID NO. 17, that includes nucleotides 12, 13, 14, and 15.

In some embodiments, the first overlap sequence O1 and the second overlap sequence O2 of the transfected or transformed host cell of the invention may comprise a nucleic acid sequence as denoted by any one of SEQ ID NO: 94 (DMD2 atggaga), SEQ ID NO: 95 (DMD3 aaaaaga), SEQ ID NO: 109 (DMD4, ttGcctA), SEQ ID NO: 111 (DMD5, tGtaaAc), SEQ ID NO: 113 (DMD6, AtGTttt), SEQ ID NO: 115 (DMD7, cctgacA), SEQ ID NO: 98 (CFTR10 taaaaac), SEQ ID NO: 99 (CFTR12 ccccttc), SEQ ID NO: 102 (NPC1 agatgcc), SEQ ID NO: 127 (CFTR13, tctTaAt), SEQ ID NO: 128 (CFTR14, gttaGcA), SEQ ID 70 (Cystinosis CTNS2, ctaagca), SEQ ID 71 Cystinosis CTNS3 tactaca), SEQ ID 73 (Cystinosis CTNS4 tgagtga), SEQ ID NO:117 (CTNS1, gGtacAg), SEQ ID NO: 131 (CTNS A, AGccccg), SEQ ID NO: 132 (CTNS D, AGGcaAA), SEQ ID NO: 18 (Tay-Sachs Hexa3: accaatg), SEQ ID NO: 19 (Tay-Sachs Hexa7 taaaaat), SEQ ID NO: 104 (SCN1A4 gcactgt), SEQ ID NO: 105 (SCN1A3, acagtgc). It should be noted that O1 and said O2 are different.

In some further embodiments, the first overlap sequence O1 and the second overlap sequence O2 of the transfected or transformed host cell of the invention may comprise a nucleic acid sequence as denoted by any one of SEQ ID NO: 18 (Tay-Sachs Hexa3: accaatg), SEQ ID NO: 19 (Tay-Sachs Hexa7 taaaaat), SEQ ID NO: 20 (Ataxia ATM4 gactcag), SEQ ID NO: 21 (Ataxia ATM8 gtgaggt), SEQ ID 51 (Ataxia ATM2 taccacg), SEQ ID NO: 22 (Sickle cell anemia HBB tctgaac), SEQ ID NO: 23 (Sickle cell anemia haem13: gactagg), SEQ ID NO: 24 (Lesch-Nyhan syndrome hgprt1 tatccct), SEQ ID NO: 25 (hgprt13 cttttag), SEQ ID 54 (ALS SOD-1 catgctg), SEQ ID 55 (ALS SOD-2 actgata), SEQ ID 58 (ALS TARDBP4 gcctccc), SEQ ID 59 (ALS TARDBP5 gtaggaa), SEQ ID 62 (ALS VAPB5 ctcttcc), SEQ ID 63 (ALS VAPB6 gtgggag), SEQ ID 66 (ALS c90RF 71-1 gagagtg), SEQ ID 67 (ALS c90RF 71-2, catctgc), SEQ ID NO: 102 (NPC1, agatgcc), SEQ ID NO: 103 (NPC1, acactgg), SEQ ID NO: 106 (COL3A1, aaaacag), SEQ ID NO: 107 (COL3A1, tttaaaa).

It should be noted that these overlap sequences may comprise any random sequence and specifically, any of the sequences indicated herein, provided that O₁ and said O₂ are different. The fact that both overlap sequences are different ensures an oriented recombination and prevents undesired recombination between the attE sites.

As indicated above, it should be appreciated that the invention further provides at least one nucleic acid molecule comprising a replacement-sequence to replace a target nucleic acid sequence of interest or any fragment thereof in at least one eukaryotic cell. In some embodiments, these target nucleic acid molecule that will be described in more detail herein after, are comprised within the host cell/s of the invention. Eukaryotic cells may be mammalian cells, plant cells, fungi or cells of any organism. As used herein, the term “eukaryotic cell” refers to any cell type known to a person skilled in the art which is suitable for gene therapy. More specifically, any cell derived from any vertebrate organism, specifically, an organism derived from any of the vertebrates groups that include Fish, Amphibians, Reptiles, Birds and Mammals (e.g., Marsupials, Primates, Rodents and Cetaceans). More specifically, a cell of a mammal (specifically, at least one of a human, Cattle, rodent, domestic pig (swine, hog), sheep, horse, goat, alpaca, lama and Camels), preferably, human cells. It should be noted that the term “eukaryotic cells” as used herein, further encompasses the autologous cells or allogeneic cells used by the methods of the invention via adoptive transfer, as discussed herein after in connection with other aspects of the invention.

In some embodiments, the replacement-sequence flanked by a first and a second Int recognition sites of the transfected or transformed host cell of the invention, may comprise a nucleic acid sequence that differs in at least one nucleotide from said target nucleic acid sequence of interest or any fragments thereof.

The terms “gene of interest”, “a target gene of interest”, a target gene”, “a target nucleic acid sequence”, are used interchangeably, and refer in some embodiments to a nucleic acid sequence that may comprise or comprised within a gene or any fragment or derivative thereof that is comprised by the target cell (or host cell) of the invention and is intended to be replaced. The target nucleic acid sequence or gene of interest may comprise coding or non-coding DNA regions, or any combination thereof.

In some embodiments, the gene of interest may comprise coding sequences and thus may comprise exons or fragments thereof that encode any product, for example, a protein or an enzyme (or fragments thereof). In other embodiments, the target nucleic acid sequence of interest may comprise non-coding sequences, as for example start codons, 5′ un-translated regions (5′ UTR), 3′ un-translated regions (3′ UTR), or other regulatory sequences, in particular regulatory sequences that are capable of increasing or decreasing the expression of specific genes within an organism. By way of example, regulatory sequences may be selected from, but are not limited to, transcription factors, activators, repressors and promoters. In further embodiments, the target nucleic acid sequence or gene of interest may comprise a combination of coding and non-coding regions.

Still further, the term “target gene of interest” or “target nucleic acid sequence of interest” as used herein refers to a gene in a eukaryotic cell or any fragment thereof to be replaced by the replacement sequence according to the invention. The target nucleic acid sequence of interest may be either identical or otherwise different, e.g., mutated with respect to the sequence of a normal target nucleic acid sequence in a healthy individual, or with respect to a frequent allele (major allele in case of polymorphism).

In some embodiments, the target gene or nucleic acid sequence of interest may be any nucleic acid sequence or gene or fragments thereof that display aberrant expression, stability, activity or function in a mammalian subject, as compared to normal and/or healthy subject. Such target gene or any fragments thereof or any target nucleic acid sequence may be in some embodiments, associated, linked or connected, directly or indirectly with at least one pathologic condition. Thus, the target nucleic acid sequence or gene of interest in some embodiments may be a nucleic acid sequence or gene that carry at least one of: (a) at least one point mutation; (b) deletion; (c) insertion; (d) rearrangement of at least one nucleotide or more, in at least one of its coding regions or non-coding regions. In some embodiments, the target nucleic acid sequence or gene of interest may comprise a sequence that differs in at least one nucleotide, from the normal and/or healthy, and/or frequent counterpart. More specifically, a target sequence that carry a mutation in its coding sequence that may be associated with a pathologic disorder.

In yet some further embodiments, the replacing sequence, that may be the corresponding gene or fragment, as containing a non-mutated form of the gene of interest or fragments thereof, replaces the mutated target sequence of interest or fragment thereof, thereby resolving the undesired effects of the mutation.

In some particular embodiments, the target nucleic acid sequence of interest of the eukaryotic cell may comprise or is comprised within the human dystrophin (DMD) gene or any fragment thereof. Such target nucleic acid sequence may be flanked by a first Int recognition site attE1 (also referred to herein as DMD2) comprising the nucleic acid sequence as denoted by SEQ ID NO. 92 and a second Int recognition site attE2 (also referred to herein as DMD3) comprising the nucleic acid sequence as denoted by SEQ ID NO. 93. In some embodiments, the O1 of the Int recognition site may comprise the nucleic acid sequence as denoted by SEQ ID NO. 94 and O2 may comprise the nucleic acid sequence as denoted by SEQ ID NO. 95. It should be noted that mutated forms of the DMD gene are associated with Duchenne muscular dystrophy (DMD). Still further, in some embodiments, other DMD fragments that should be replaced, may be flanked by any of the attE sequence designated herein as DMD4, having the sequence of SEQ ID NO. 108 (with an O sequence as denoted by SEQ ID NO. 109), DMD5, having the sequence of SEQ ID NO. 110 (with an O sequence as denoted by SEQ ID NO. 111), DMD6, having the sequence of SEQ ID NO. 112 (with an O sequence as denoted by SEQ ID NO. 113) or DMD7, having the sequence of SEQ ID NO. 114 (with an O sequence as denoted by SEQ ID NO. 115). As indicated above, in more specific embodiments, the target gene or nucleic acid sequence of interest may be the human DMD gene also named DMD gene, having the accession number ENSG00000198947 and encoding for the protein having the accession number NP_003997.2. In some further embodiments, the human DMD gene may encode a protein comprising an amino acid sequence as denoted by SEQ ID NO: 226. In some embodiments, the In some embodiments the DMD2 site is located at nucleotides 1111828-1111848, the DMD3 site is located at nucleotides 1134771-1134791, the DMD4 site is located at nucleotides 1340410-1340430, the DMD5 site is located at nucleotides 1381532-1381552, the DMD6 site is located at nucleotides 1561051-1561071, and the DMD7 site is located at nucleotides 1619335-1619355, of the DMD gene, having the accession number ENSG00000198947. In some embodiments, the DMD gene applicable in the present invention is located at Chromosome X: 31,097,677 to 33,339,441.

In some particular embodiments, a replacement sequence provided with the nucleic acid cassette or molecule of the invention, may be a sequence that may replace any mutation in exon 44 of the DMD gene. In some embodiments, the replacement sequence may be targeted at attE sites that comprise the sequence of DMD2 and DMD3 sites (of SEQ ID NO. 92 and 93, respectively), specifically, the O sequences of these sites comprise SEQ ID NO. 94 and 95, respectively. In some embodiments a suitable replacement sequence may comprise the nucleic acid sequence as denoted by SEQ ID NO. 217, or any derivatives or homologs thereof. In yet some further embodiments, any of the DMD sites, specifically those disclosed by the invention (e.g., DMD2, DMD3, DMD4, DMD5, DMD6, DMD7, and any combinations thereof), may be used for a replacement. In such case, a suitable replacement sequence, also referred to herein as universal sequence may be used. In some embodiments, such universal replacement sequence may comprise the cDNA of the normal non-mutated DMD gene. Integration of such nucleic acid sequence to any of the specified attE sites, replaces any mutation in the DMD gene. Thus, in some embodiments, a replacement sequence that may be used comprise the nucleic acid sequence as denoted by

In yet some further particular embodiments, the target nucleic acid sequence of interest of the eukaryotic cell may comprise or is comprised within the human cystic fibrosis transmembrane conductance regulator (CFTR) gene or any fragment thereof. More specifically, the nucleic acid sequence of interest is flanked by a first Int recognition site attE1 (also referred to herein as CFTR10) comprising the nucleic acid sequence as denoted by SEQ ID NO. 96 and a second Int recognition site attE2 (also referred to herein as CFTR12) comprising the nucleic acid sequence as denoted by SEQ ID NO. 97. In some embodiments, the O1 of the recognition site may comprise the nucleic acid sequence as denoted by SEQ ID NO. 98 and O2 may comprise the nucleic acid sequence as denoted by SEQ ID NO. 99. It should be noted that mutated forms of the CFTR gene are associated with cystic fibrosis. Still further, in some embodiments, other CFTR fragments that should be replaced, may be flanked by any of the attE sequence designated herein as CFTR13, having the sequence of SEQ ID NO. 125 (with an O sequence as denoted by SEQ ID NO. 127) and CFTR14, having the sequence of SEQ ID NO. 126 (with an O sequence as denoted by SEQ ID NO. 128). As indicated above, in more specific embodiments, the target gene or nucleic acid sequence of interest may be the human CFTR gene, having the accession number NM_000492.4 and encoding for the protein having the accession number NP_000483.3. In some further embodiments, the human CFTR gene may encode a protein comprising an amino acid sequence as denoted by SEQ ID NO: 227. In some embodiments the CF10 site is located at nucleotides 142731-142751, the CF12 site is located at nucleotides 145724-145744, the CF13 site is located at nucleotides 192958-192978, the CF14 site is located at nucleotides 197886-197906 of the CFTR gene, having the accession number. NM_000492.4. In some embodiments, the CFTR gene applicable in the present invention is located at Chromosome 7: 117,287,120 to 117,715,971.

In some particular embodiments, a replacement sequence provided with the nucleic acid cassette or molecule of the invention, may be a sequence that may replace any mutation in exon 3 of the CFTR gene. In some embodiments, the replacement sequence may be targeted at attE sites that comprise the sequences of CFTR10 and CFTR12 sites (of SEQ ID NO. 96 and 97, respectively). Specifically, the O sequences of these sites comprise SEQ ID NO. 98 and 99, respectively. In some embodiments a suitable replacement sequence may comprise the nucleic acid sequence as denoted by SEQ ID NO. 215, or any derivatives or homologs thereof. In yet some further embodiments, any of the CFTR sites, specifically those disclosed by the invention (e.g., CFTR10, CFTR 12, CFTR13, CFTR14, and any combinations thereof), may be used for a replacement using a universal sequence that may comprise the cDNA of the normal non-mutated CFTR gene. Integration of such nucleic acid sequence to any of the specified ate sites, replaces any mutation in the CFTR gene. Thus, in some embodiments, a replacement sequence that may be used comprise the nucleic acid sequence as denoted by SEQ ID NO. 216.

It should be noted that the invention further provides attE sequences for the mouse CFTR gene. More specifically, such attE sequences may comprise the mCF1, mCF2, mCF3, that comprise the nucleic acid sequence as denoted by SEQ ID NO. 194, 195, 196, respectively, and comprise the ‘O’ sequences as denoted by SEQ ID NO. 195, 197, 199, respectively. These sites are useful for mouse model for cystic fibrosis, and are applicable for any aspect of the invention.

In some other embodiments, the target nucleic acid sequence of interest of the eukaryotic cell may comprise or is comprised within the human cystinosin (CTNS) gene or any fragment thereof. Such nucleic acid sequence of interest is flanked by a first Int recognition site attE₁ comprising the nucleic acid sequence as denoted by SEQ ID NO. 68 (CTNS2) and a second Int recognition site attE₂ comprising the nucleic acid sequence as denoted by SEQ ID NO. 69 (CTNS3). In some embodiments, the O₁ may comprise the nucleic acid sequence as denoted by SEQ ID NO. 70 and O₂ may comprise the nucleic acid sequence as denoted by SEQ ID NO. 71. It should be noted that mutated forms of the CTNS gene are associated with Cystinosis.

In some other embodiments, the target nucleic acid sequence of interest of the eukaryotic cell may comprise or is comprised within the human CTNS gene or any fragment thereof. Such nucleic acid sequence of interest is flanked by a first Int recognition site attE₁ comprising the nucleic acid sequence as denoted by SEQ ID NO. 68 (CTNS2) and a second Int recognition site attE₂ comprising the nucleic acid sequence as denoted by SEQ ID NO. 72 (CTNS4). In some embodiments, the O₁ may comprise the nucleic acid sequence as denoted by SEQ ID NO. 70 and O₂ may comprise the nucleic acid sequence as denoted by SEQ ID NO. 73. In some other embodiments, the target nucleic acid sequence of interest of the eukaryotic cell may comprise or is comprised within the human CTNS gene or any fragment thereof, Such nucleic acid sequence of interest is flanked by a first Int recognition site attE₁ comprising the nucleic acid sequence as denoted by SEQ ID NO. 69 (CTNS3) and a second Int recognition site attE₂ comprising the nucleic acid sequence as denoted by SEQ ID NO. 72 (CTNS4). In some embodiments, the O₁ may comprise the nucleic acid sequence as denoted by SEQ ID NO. 71 and O₂ may comprise the nucleic acid sequence as denoted by SEQ ID NO. 73.

In yet some further embodiments, the target nucleic acid sequence of interest of the eukaryotic cell may comprise or is comprised within the human CTNS gene or any fragment thereof. Such nucleic acid sequence of interest is flanked by a first Int recognition site attE₁ comprising the nucleic acid sequence as denoted by SEQ ID NO. 72 (CTNS4) and a second Int recognition site attE₂ comprising the nucleic acid sequence as denoted by SEQ ID NO. 116 (CTNS1). In some embodiments, the O₁ may comprise the nucleic acid sequence as denoted by SEQ ID NO. 73 and O₂ may comprise the nucleic acid sequence as denoted by SEQ ID NO. 117. In some other embodiments, the target nucleic acid of interest in the target eukaryotic cell may comprise or comprised within the human CTNS gene or any fragment thereof, flanked by a first Int recognition site attE₁ comprising the nucleic acid sequence as denoted by SEQ ID NO. 129 (CTNS A) and a second Int recognition site attE₂ comprising the nucleic acid sequence as denoted by SEQ ID NO. 130 (CTNS D). In some embodiments, the O₁ may comprise the nucleic acid sequence as denoted by SEQ ID NO. 131 and O₂ may comprise the nucleic acid sequence as denoted by SEQ ID NO. 132. In more specific embodiments, the target gene or nucleic acid sequence of interest may be the human CTNS gene, having the accession number ENSG00000040531

and encoding for the protein having the accession number NP_004928.2. In some embodiments, the human CTNS gene may encode a protein comprising an amino acid sequence as denoted by SEQ ID NO: 228. In some embodiments the CTNS4 site is located at nucleotides 71449-71469, and the CTNS1 site is located at nucleotides 79035-79055 of the CTNS gene, having the accession number ENSG00000040531. In some embodiments, the CTNS gene applicable in the present invention is located at Chromosome 17: 3,636,459 to 3,661,542.

In some particular embodiments, a replacement sequence provided with the nucleic acid cassette or molecule of the invention, may comprise at least one sequence that may replace any mutation in exons 1 to 3 of the CTNS gene. In some embodiments, the replacement sequence may be targeted at attE sites that comprise the sequence of CTNS4 and CTNS1 sites (of SEQ ID NO. 72 and 116, respectively). These sites comprise the O sites of SEQ ID NO. 73 and 117, respectively.

In some embodiments a suitable replacement sequence may comprise the nucleic acid sequence as denoted by SEQ ID NO. 219, or any derivatives or homologs thereof. In yet some further embodiments, any of the CTNS sites, specifically those disclosed by the invention, may be used for a replacement using a universal sequence that may comprise the cDNA of the normal non-mutated CTNS gene. Integration of such nucleic acid sequence to any of the specified ate sites, replaces any mutation in the CTNS gene. Thus, in some embodiments, a replacement sequence that may be used comprise the nucleic acid sequence as denoted by SEQ ID NO. 220.

In some additional embodiments, the target nucleic acid sequence of interest in the eukaryotic cell may comprise or comprised within the human sodium channel, voltage-gated, type I, alpha subunit (SCN1A) gene or any fragment thereof. Such nucleic acid sequence of interest is flanked by a first Int recognition site attE₁ comprising the nucleic acid sequence as denoted by SEQ ID NO. 120 (SCN1A 4) and a second Int recognition site attE₂ comprising the nucleic acid sequence as denoted by SEQ ID NO. 121 (SCN1A3), and wherein said O₁ comprises the nucleic acid sequence as denoted by SEQ ID NO. 104 and said O₂ comprises the nucleic acid sequence as denoted by SEQ ID NO. 105. In more specific embodiments, the target gene or nucleic acid sequence of interest may comprise or comprised within the human SCN1A gene or any fragments or parts thereof, having the accession number ENSG00000144285 and encoding for the protein having the accession number NP_008851.3. In some further embodiments, the human SCN1A gene may encode a protein comprising an amino acid sequence as denoted by SEQ ID NO: 236. In some embodiments the SCN1A3 site is located at nucleotides 99997-100017, and the SCN1A4 site is located at nucleotides 100072-100092 of the SCN1A gene, having the accession number ENSG00000144285. In some embodiments, the SCN1A gene applicable in the present invention is located at Chromosome 2: 165,984,641 to 166,149,214.

In some particular embodiments, a replacement sequence provided with the nucleic acid cassette or molecule of the invention, may comprise at least one sequence that may replace any mutation in intron 6 of the SCN1A gene. In some embodiments, the replacement sequence may be targeted at attE sites that comprise the sequence of SCN1A3 and SCN1A4 sites (of SEQ ID NO. 121 and 120, respectively). These sites comprise the O sites of SEQ ID NO. 105 and 104, respectively. In some embodiments a suitable replacement sequence may comprise the nucleic acid sequence as denoted by SEQ ID NO. 221, or any derivatives or homologs thereof. In yet some further embodiments, any of the SCN1A sites, specifically those disclosed by the invention, may be used for a replacement using a universal sequence that may comprise the cDNA of the normal non-mutated SCN1A gene. Integration of such nucleic acid sequence to any of the specified ate sites, replaces any mutation in the SCN1A gene. Thus, in some embodiments, a replacement sequence that may be used comprise the nucleic acid sequence as denoted by SEQ ID NO. 222.

In some other specific embodiments, the target nucleic acid sequence of interest of the eukaryotic cell may comprise or comprised within the human Hexosaminidase A (alpha polypeptide), also known as HEXA gene or any fragment thereof. Such target nucleic acid sequence of interest is flanked by a first Int recognition site AttE₁ comprising the nucleic acid sequence as denoted by SEQ ID NO. 26 and a second Int recognition site attE₂ comprising the nucleic acid sequence as denoted by SEQ ID NO. 27. In some embodiments, the O₁ of the Int recognition site may comprise the nucleic acid sequence as denoted by SEQ ID NO. 18 and O₂ may comprise the nucleic acid sequence as denoted by SEQ ID NO. 19. It should be noted that mutated forms of the HEXA gene are associated with Tay-Sachs. In more specific embodiments, the target nucleic acid sequence or nucleic acid sequence of interest may be the human HEXA gene, having the accession number ENSG00000213614 and encoding for the protein having the accession number NP_000511.2. In some further embodiments, the human HEXA gene may encode a protein comprising an amino acid sequence as denoted by SEQ ID NO: 229.

In some other embodiments, the target nucleic acid sequence of interest of the eukaryotic cell may comprise or comprised within the human ATM serine/threonine kinase (ATM) gene or any fragment thereof. Such target nucleic acid sequence of interest is flanked by a first Int recognition site attE₁ comprising the nucleic acid sequence as denoted by SEQ ID NO. 28 and a second Int recognition site attE₂ comprising the nucleic acid sequence as denoted by SEQ ID NO. 29. In some embodiments, the O₁ may comprise the nucleic acid sequence as denoted by SEQ ID NO. 20 and O₂ may comprise the nucleic acid sequence as denoted by SEQ ID NO. 21. It should be noted that mutated forms of the ATM gene are associated with Ataxia telangiectasia.

In some other embodiments, the target nucleic acid sequence of interest of the eukaryotic cell may comprise or comprised within the human ATM gene or any fragment thereof. The target nucleic acid sequence of interest is flanked by a first Int recognition site attE₁ comprising the nucleic acid sequence as denoted by SEQ ID NO. 50 and a second Int recognition site attE₂ comprising the nucleic acid sequence as denoted by SEQ ID NO. 28, and O₁ may comprise the nucleic acid sequence as denoted by SEQ ID NO. 51 and O₂ may comprise the nucleic acid sequence as denoted by SEQ ID NO. 20.

In yet some other alternative embodiments, the target nucleic acid sequence of interest of interest of the eukaryotic cell may comprise or comprised within the human ATM gene or any fragment thereof. Such target nucleic acid sequence of interest is flanked by a first Int recognition site attE₁ comprising the nucleic acid sequence as denoted by SEQ ID NO. 50 and a second Int recognition site attE₂ comprising the nucleic acid sequence as denoted by SEQ ID NO. 29. In some embodiments, the O₁ may comprise the nucleic acid sequence as denoted by SEQ ID NO. 51 and O₂ may comprise the nucleic acid sequence as denoted by SEQ ID NO. 21. In more specific embodiments, the target nucleic acid sequence of interest or nucleic acid sequence of interest may comprise or comprised within the human ATM gene, having the accession number ENSG00000149311 and encoding for the protein having the accession number NP_000042.3. In some further embodiments, the human ATM gene may encode a protein comprising an amino acid sequence as denoted by SEQ ID NO: 230.

In some other embodiments, the target nucleic acid sequence of interest of the eukaryotic cell may comprise or comprised within the human Hemoglobinase (HAEM) gene or any fragment thereof. Such target nucleic acid sequence of interest is flanked by a first Int recognition site attE₁ comprising the nucleic acid sequence as denoted by SEQ ID NO. 30 and a second Int recognition site attE₂ comprising the nucleic acid sequence as denoted by SEQ ID NO. 31. In some embodiments, the O₁ may comprise the nucleic acid sequence as denoted by SEQ ID NO. 22 and O₂ may comprise the nucleic acid sequence as denoted by SEQ ID NO. 23. It should be noted that mutated forms of the HAEM gene are associated with Sickle cell anemia. In more specific embodiments, the target gene or nucleic acid sequence of interest may comprise or comprised within the human HBB gene or any fragments or parts thereof, having the accession number NM_000518.5 and encoding for the protein having the accession number NP_000509.1. In some further embodiments, the human HBB gene may encode a protein comprising an amino acid sequence as denoted by SEQ ID NO: 239.

In some other embodiments, the target nucleic acid sequence of interest of the eukaryotic cell may comprise or comprised within the human HGPRT gene or any fragment thereof. Such target nucleic acid sequence of interest is flanked by a first Int recognition site attE₁ comprising the nucleic acid sequence as denoted by SEQ ID NO. 32 and a second Int recognition site attE₂ comprising the nucleic acid sequence as denoted by SEQ ID NO. 33. In some embodiments, the O₁ may comprise the nucleic acid sequence as denoted by SEQ ID NO. 24 and O₂ may comprise the nucleic acid sequence as denoted by SEQ ID NO. 25. It should be noted that mutated forms of the HGPRT gene are associated with Lesch-Nyhan syndrome. In more specific embodiments, the target gene or nucleic acid sequence of interest may comprise or comprised within the human HGPRT also named HGPRT1 gene, or any fragments or parts thereof having the accession number HPRT1 ENSG00000165704 and encoding for the protein having the accession number NP_000185.1. In some further embodiments, the human HGPRT gene may encode a protein comprising an amino acid sequence as denoted by SEQ ID NO: 231.

In yet some other embodiments, the target nucleic acid sequence of interest of the eukaryotic cell may comprise or comprised within the human superoxide dismutase 1(SOD1) gene or any fragment thereof. Such target nucleic acid sequence of interest is flanked by a first Int recognition site attE₁ comprising the nucleic acid sequence as denoted by SEQ ID NO. 52 and a second Int recognition site attE₂ comprising the nucleic acid sequence as denoted by SEQ ID NO. 53. In some embodiments, the O₁ may comprise the nucleic acid sequence as denoted by SEQ ID NO. 54 and O₂ may comprise the nucleic acid sequence as denoted by SEQ ID NO. 55. It should be noted that mutated forms of the SOD1 gene are associated with amyotrophic lateral sclerosis (ALS). In more specific embodiments, the target gene or nucleic acid sequence of interest may comprise or comprised within the human SOD1 gene, or any fragments or parts thereof having the accession number ENSG00000142168 and encoding for the protein having the accession number NP_000445.1. In some further embodiments, the human SOD1 gene may encode a protein comprising an amino acid sequence as denoted by SEQ ID NO: 232.

In some other embodiments, the target nucleic acid sequence of interest of the eukaryotic cell may comprise or comprised within the human trans-active response DNA binding protein (TARDBP) gene or any fragment thereof. Such target nucleic acid sequence of interest is flanked by a first Int recognition site attE₁ comprising the nucleic acid sequence as denoted by SEQ ID NO. 56 and a second Int recognition site attE₂ comprising the nucleic acid sequence as denoted by SEQ ID NO. 57. In some embodiments, the O₁ may comprise the nucleic acid sequence as denoted by SEQ ID NO. 58 and O₂ may comprise the nucleic acid sequence as denoted by SEQ ID NO. 59. It should be noted that mutated forms of the TARDBP gene are associated with familial forms of ALS. In more specific embodiments, the target gene or nucleic acid sequence of interest may comprise or comprised within the human TARDBP gene, or any fragments or parts thereof having the accession number ENSG00000120948 and encoding for the protein having the accession number NP_031401.1. In some further embodiments, the human TARDBP gene may encode a protein comprising an amino acid sequence as denoted by SEQ ID NO: 233.

In some other embodiments, the target nucleic acid sequence of interest of the eukaryotic cell may comprise or comprised within the human vesicle-associated membrane protein (VAPB) gene or any fragment thereof. Such target nucleic acid sequence of interest is flanked by a first Int recognition site attE₁ comprising the nucleic acid sequence as denoted by SEQ ID NO. 60 and a second Int recognition site attE₂ comprising the nucleic acid sequence as denoted by SEQ ID NO. 61. In some embodiments, the O₁ may comprise the nucleic acid sequence as denoted by SEQ ID NO. 62 and O₂ may comprise the nucleic acid sequence as denoted by SEQ ID NO. 63. It should be noted that mutated forms of the VAPB gene are associated with ALS. In more specific embodiments, the target gene or nucleic acid sequence of interest may comprise or comprised within the human VAPB gene or any fragments or parts thereof, having the accession number ENSG00000124164 and encoding for the protein having the accession number NP_004729.1. In some further embodiments, the human VAPB gene may encode a protein comprising an amino acid sequence as denoted by SEQ ID NO: 234.

In some other embodiments, the target nucleic acid sequence of interest of the eukaryotic cell may comprise or comprised within the human C9ORF71 gene or any fragment thereof. Such target nucleic acid sequence of interest is flanked by a first Int recognition site attE₁ comprising the nucleic acid sequence as denoted by SEQ ID NO. 64 and a second Int recognition site attE₂ comprising the nucleic acid sequence as denoted by SEQ ID NO. 65. In some embodiments, the O₁ may comprise the nucleic acid sequence as denoted by SEQ ID NO. 66 and O₂ may comprise the nucleic acid sequence as denoted by SEQ ID NO. 67. It should be noted that mutated forms of the C9ORF71 gene are associated with Amyotrophic lateral sclerosis (ALS). In more specific embodiments, the target gene or nucleic acid sequence of interest may comprise or comprised within the human C9ORF71 gene or any fragments or parts thereof also named transmembrane protein 252 (TMEM252), having the accession number NM_153237.2 and encoding for the protein having the accession number NP_694969.1. In some further embodiments, the human TMEM252 gene may encode a protein comprising an amino acid sequence as denoted by SEQ ID NO: 238.

In some other embodiments, the target nucleic acid sequence of interest of the eukaryotic cell may comprise or comprised within the human NPC1 gene or any fragment thereof. Such target nucleic acid sequence of interest is flanked by a first Int recognition site attE1 comprising the nucleic acid sequence as denoted by SEQ ID NO. 118 and a second Int recognition site attE2 comprising the nucleic acid sequence as denoted by SEQ ID NO. 119. In some embodiments, the O1 of the Int recognition site may comprise the nucleic acid sequence as denoted by SEQ ID NO. 102 and O2 may comprise the nucleic acid sequence as denoted by SEQ ID NO. 103. It should be noted that mutated forms of the NPC1 gene are associated with Niemann-Pick disease. In more specific embodiments, the target gene or nucleic acid sequence of interest may comprise or comprised within the human NPC1 gene or any fragments or parts thereof, having the accession number ENSG00000141458 and encoding for the protein having the accession number NP_000262.2. In some further embodiments, the human NPC1 gene may encode a protein comprising an amino acid sequence as denoted by SEQ ID NO: 235.

In some other embodiments, the target nucleic acid sequence of interest of the eukaryotic cell may comprise or comprised within the human COL3A gene or any fragment thereof. Such target nucleic acid sequence of interest is flanked by a first Int recognition site attE1 comprising the nucleic acid sequence as denoted by SEQ ID NO. 122 and a second Int recognition site attE2 comprising the nucleic acid sequence as denoted by SEQ ID NO. 123. In some embodiments, the O1 of the Int recognition site may comprise the nucleic acid sequence as denoted by SEQ ID NO. 106 and O2 may comprise the nucleic acid sequence as denoted by SEQ ID NO. 107. It should be noted that mutated forms of the COL3A gene are associated with type III and IV Ehlers-Danlos syndrome and with aortic and arterial aneurysms. In more specific embodiments, the target gene or nucleic acid sequence of interest may comprise or comprised within the human COL3A1 gene or any fragments or parts thereof, having the accession number ENSG00000168542 and encoding for the protein having the accession number NP_000081.2. In some further embodiments, the human COL3A1 gene may encode a protein comprising an amino acid sequence as denoted by SEQ ID NO: 237.

As indicated above, the host cell of the invention may comprise in addition to the Int variant discussed herein or any nucleic acid sequence encoding the Int variants of the invention, also at least one nucleic acid molecule that comprise at least one nucleic acid sequence that should replace a target sequence within the cell, referred to herein as “replacement sequence”. Said nucleic acid molecule may be comprised within a cassette and referred to herein as a recombination cassette. It should be therefore noted that the invention further pertains to any of the recombination cassettes disclosed herein and therefore, in certain embodiments, the nucleic acid molecules provided by the invention may comprise any of the recombination cassettes described by the invention. More specifically, the term “recombination cassette” as used herein refers to a modular DNA sequence composed of fragments of DNA enabling RMCE.

In another aspect, the invention relates to a system and/or kit may comprise at least one of: As a first component (a), at least one nucleic acid molecule or any nucleic acid cassette or vector comprising said nucleic acid molecule, wherein the nucleic acid molecule or cassette comprising a replacement-sequence flanked by a first and a second Int recognition sites. In some embodiments, the first site attP1 may comprise a first overlap sequence O1 and the second site attP2 may comprise a second overlap sequence O2. In some further embodiments, the first O1 and the second O2 overlap sequences may be different, each consisting of seven nucleotides, the O1 may be identical to an overlap sequence O1 comprised within a first Int recognition site attE1 in a eukaryotic cell and the O2 may be identical to an overlap sequence O2 comprised within a second Int recognition site attE2 in the eukaryotic cell. In some embodiments, the eukaryotic recognition sites attE1 and attE2 may flank a target nucleic acid sequence of interest or any fragment thereof in the eukaryotic cell. In some embodiments, the first binding sites E may comprise the sequence of C1-T2-T3-W4, as denoted by SEQ ID NO. 16, and the second binding sites E′ may comprise the sequence of A12-A13-A14-G15, as denoted by SEQ ID NO. 17.

As a second component (b), at least one HK-Int variant and/or mutated molecule or any functional fragments or peptides thereof, any nucleic acid molecule comprising a sequence encoding the HK-Int variant and/or mutated molecule or any vector, vehicle, matrix, nano- or micro-particle comprising the same.

In some embodiments, the HK-Int variant and/or mutated molecule of the system/kit of the invention may comprise at least one substituted amino acid residue in at least one of the CB, ND and the CD domains of the Wild type HK-Int molecule. In some specific embodiments, the HK-Int mutated molecule and/or variant of the system/kit of the invention may comprise at least one substitution at any position of residues 174, 278, 43, 319, 134, 149, 215, 264, 303, 309, 336, and any combinations thereof, of the amino acid sequence of the Wild type HK-Int molecule as denoted by SEQ ID NO. 13. In some particular embodiments, the HK-Int mutated molecule and/or variant of the system/kit of the invention may comprise at least one substitution at the CB domain. Examples for such variant/s may be any HK-Int variant comprising a substitution in at least one of residues 174, 134, 149, and any combinations thereof. In some specific embodiments, the HK-Int mutated molecule and/or variant of the system/kit of the invention may comprise at least one substitution at position 174 of the Wild type HK-Int molecule as denoted by SEQ ID NO. 13. In more particular embodiments, the HK-Int mutated molecule and/or variant of the system/kit of the invention may comprise at least one substitution replacing E with K at position 174 of the Wild type HK-Int molecule as denoted by SEQ ID NO. 13.

In some specific embodiments, the HK-Int variant and/or mutated molecule of the system/kit of the invention may comprise a substitution of glutamic acid to glycine, at position 174, as designated by the E174K mutant of the invention.

In yet some further specific embodiments, said Int variant or mutated molecule used by the system/kit of the invention may comprise the amino acid sequence as denoted by SEQ ID NO. 14, or any derivatives, homologs, fusion proteins or variants thereof. In some embodiments, the nucleic acid sequence encoding the E174K variant may comprise the nucleic acid sequence as denoted by SEQ ID NO. 15, and any functional fragments, variants, or derivatives thereof.

In yet some further embodiments, the double mutant used by the system/kit of the invention may comprise a substitution E with K at position 174 of the Wild type HK-Int molecule as denoted by SEQ ID NO. 13, and in addition a substitution replacing D with K at position 278 of the Wild type HK-Int molecule as denoted by SEQ ID NO. 13. In some specific embodiments, such mutant is designated E174K/D278K mutant. In some particular embodiments, such mutant may comprise the amino acid sequence as denoted by SEQ ID NO. 184, or any derivatives, homologs, fusion proteins or variants thereof. In yet some further embodiments, such mutant may be encoded by the nucleic acids sequence that comprises SEQ ID NO. 186, and any functional fragments, variants, or derivatives thereof. In some specific embodiments, the HK-Int variant and/or mutated molecule of the system/kit of the invention may comprise a substitution of E with K at position 174 and in addition a substitution replacing I with F at position 43. In some specific embodiments, such mutant is designated E174K/I43F mutant. In some particular embodiments, such mutant may comprise the amino acid sequence as denoted by SEQ ID NO. 83, and any functional fragments, variants, fusion proteins or derivatives thereof. In yet some further embodiments, such mutant may be encoded by the nucleic acids sequence that comprises SEQ ID NO. 82, and any functional fragments, variants, or derivatives thereof.

In yet some further embodiments, the double mutant of the system/kit of the invention may comprise a substitution of glutamic acid (E) with lysine (K) at position 174 and in addition a substitution replacing glutamic acid (E) with Glycine (G) at position 319 of the Wild type HK-Int molecule as denoted by SEQ ID NO. 13. In some specific embodiments, such mutant is designated E174K/R319G mutant. In some particular embodiments, such mutant may comprise the amino acid sequence as denoted by SEQ ID NO. 85, and any functional fragments, variants, fusion proteins or derivatives thereof. In yet some further embodiments, such mutant may be encoded by the nucleic acids sequence that comprises SEQ ID NO. 84, and any functional fragments, variants, or derivatives thereof.

In yet some further embodiments, the HK-Int variant and/or mutated molecule of the system/kit may comprise at least one substitution at the CD domain of the amino acid sequence of the Wild type HK-Int molecule as denoted by SEQ ID NO. 13. In yet some further embodiments, such variant or mutated molecule may comprise at least one substitution in at least one of residues 278, 215, 264, 303, 309, 319, 336, and any combinations thereof. In more specific embodiments, the HK-Int variant and/or mutated molecule of the system/kit comprises at least one substitution at position 278 of the Wild type HK-Int molecule as denoted by SEQ ID NO. 13 and any variants, homologs or derivatives thereof. In some particular embodiments, such variant comprises at least one substitution replacing D with K at position 278 of the Wild type HK-Int molecule. In some specific and non-limiting embodiments the HK-Int mutated molecule is designated D278K. More specifically, in some embodiments this mutant may comprise the amino acid sequence as denoted by SEQ ID NO. 182, or any functional fragments, variants, fusion proteins or derivatives thereof.

It should be understood that the invention further encompasses systems or kits using any of the other HK-Int variants of the invention, specifically, any of the variants comprising the amino acid sequence as denoted by any one of SEQ ID NO. 14, 42, 44, 46, 48, 83, 85, 87, 89, 184, 185, 180, 188, 190, 192, 223 or any functional fragments, variants, fusion proteins or derivatives thereof. In yet some further embodiments, the nucleic acid sequences encoding the HK-mutants of the invention that are applicable in the kits and systems of the invention may comprise the nucleic acid sequence as denoted by any one of SEQ ID NO. 15, 43, 45, 47, 49, 82, 84, 86, 88, 186, 187, 181, 189, 191, 193, 224 and any functional fragments, variants, or derivatives thereof.

In other embodiments, the first overlap sequence O1 and second overlap sequence O2 of the system and/or kit of the invention may comprise a nucleic acid sequence as denoted by any one of SEQ ID NO. 94, SEQ ID NO. 95 (DMD), SEQ ID NO. 98, SEQ ID NO. 99, SEQ ID NO. 127 and SEQ ID NO. 128 (CFTR), as well as the nucleic acid sequences as denoted by SEQ ID NO. 109, 111, 113, 115 (DMD), and SEQ ID NO. 117, 70, 71, 73, 131, 132 (CTNS), SEQ ID NO. 104, SEQ ID NO. 105 (SCN1A). It should be noted that O1 and O2 are different.

In some further embodiments, the first overlap sequence O1 and second overlap sequence O2 of the system/kit of the invention may comprise a nucleic acid sequence as denoted by any one of SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 54, SEQ ID NO. 55, SEQ ID NO. 58, SEQ ID NO.59, SEQ ID NO. 62, SEQ ID NO.63, SEQ ID NO.66, SEQ ID NO. 67, SEQ ID NO. 102, SEQ ID NO. 103, SEQ ID NO. 106 and SEQ ID NO. 107, any functional fragments, variants, or derivatives thereof.

In yet some further embodiments, the replacement sequence of the nucleic acid molecule or nucleic acid cassette relevant to the system/kit of the invention, may comprise a nucleic acid sequence that differs in at least one nucleotide from the at least one sequence to be replaced in a target nucleic acid sequence of interest or any fragments thereof. In more specific embodiments, such replacement sequence may be a nucleic acid sequence or any fragments thereof, that may replace a target nucleic acid sequence or any fragments thereof, that display an abnormal expression, stability or function in a mammalian subject. Such abnormal or unusual expression (either reduced or alternatively, over expression) or function (impaired or different), or stability (either reduced or alternatively, enhanced) of the target nucleic acid sequence as compared to the expression, stability or activity in the corresponding target sequence in healthy or normal subjects (or subjects displaying a major allele), may be associated either directly or indirectly with a pathologic condition or disorder in the subject.

In some specific embodiments of the kits and systems of the invention, the target nucleic acid sequence of interest in the eukaryotic cell may comprise or comprised within the human DMD gene or any fragment thereof that relates to the Duchenne disease, this target nucleic acid sequence is flanked by a first Int recognition site attE1 comprising the nucleic acid sequence as denoted by SEQ ID NO. 92 and a second Int recognition site attE2 comprising the nucleic acid sequence as denoted by SEQ ID NO. 93. In some embodiments, the O1 of the Int recognition site may comprise the nucleic acid sequence as denoted by SEQ ID NO. 94 and O2 may comprise the nucleic acid sequence as denoted by SEQ ID NO. 95. Still further, in some embodiments, other DMD fragments that should be replaced, may be flanked by any of the attE sequence designated herein as DMD4, having the sequence of SEQ ID NO. 108 (with an O sequence as denoted by SEQ ID NO. 109), DMD5, having the sequence of SEQ ID NO. 110 (with an O sequence as denoted by SEQ ID NO. 111), DMD6, having the sequence of SEQ ID NO. 112 (with an O sequence as denoted by SEQ ID NO. 113) or DMD7, having the sequence of SEQ ID NO. 114 (with an O sequence as denoted by SEQ ID NO. 115). In some specific and non-limiting embodiments, a suitable replacement sequence in the nucleic acid molecule or cassette provided by the kit/s or systems of the invention may comprise the nucleic acid sequence as denoted by SEQ ID NO. 217, or any functional fragments, variants, or derivatives thereof, specifically, when attE sites comprising the nucleic acid sequence as denoted by SEQ ID NO. 92 and 93 (DMD2 and DMD3) that flank exon 44 in the DMD gene, are targeted. In such embodiments, the replacement sequence in the nucleic acid cassette used by the kits and systems of the invention, is flanked by attP sites that comprise the 0 (overlap sequence) as denoted by SEQ ID NO. 94 and 95. In yet some further embodiments, a suitable replacement sequence may comprise the nucleic acid sequence as denoted by SEQ ID NO. 218, or any functional fragments, variants, or derivatives thereof. Such universal replacement sequence may be used when any other DMD site, specifically, as disclosed above, is used. It should be further appreciated that in some embodiments, P and P′ sequences that flank the replacement sequence comprise the nucleic acid sequences as denoted by SEQ ID NO. 213 and 214, respectively, or any derivatives, fragments or variants thereof. Still further, in some embodiments, the first attP1 and a second attP2 sites that flank the replacement sequence in the nucleic acid cassette of the invention may comprise P and P′ sequences that flank the “o” sequence. Such P sequence may comprise the sequence of any one of SEQ ID NO. 100, 213, 240 or 241, and the P′ sequence may comprise the sequence of any one of SEQ ID NO. 101, 214, 242, 243 or 244. It should be noted that any combination of the P and P′ sequences in an attP sites is encompassed by the invention.

In some further alternative embodiments, the target nucleic acid sequence of interest in the eukaryotic cell may comprise or comprised within the human CFTR gene or any fragments thereof that is associated with cystic fibrosis. In some embodiments, the target nucleic acid sequence is flanked by a first Int recognition site attE1 comprising the nucleic acid sequence as denoted by SEQ ID NO. 96 and a second Int recognition site attE2 comprising the nucleic acid sequence as denoted by SEQ ID NO. 97. The O1 of the recognition site may comprise the nucleic acid sequence as denoted by SEQ ID NO. 98 and O2 may comprise the nucleic acid sequence as denoted by SEQ ID NO. 99. Still further, in some embodiments, the first attP₁ site may comprise a first overlap sequence O₁ as denoted by SEQ ID NO. 127 and the second attP₂ site may comprise a second overlap O₂ sequence as denoted by SEQ ID NO. 128. In yet more specific embodiments the attE₁ may comprise a nucleic acid sequence as denoted by SEQ ID NO. 125 and the attE₂ may comprise a nucleic acid sequence as denoted by SEQ ID NO. 126. In some specific and non-limiting embodiments, a suitable replacement sequence in the nucleic acid molecule or cassette provided by the kit/s or systems of the invention may comprise the nucleic acid sequence as denoted by SEQ ID NO. 215, or any functional fragments, variants, or derivatives thereof, specifically, when attE sites comprising the nucleic acid sequence as denoted by SEQ ID NO. 96 and 97 (CFTR10 and CFTR12) that flank exon 3 in the CFTR gene, are targeted. In such embodiments, the replacement sequence in the nucleic acid cassette used by the kits and systems of the invention, is flanked by attP sites that comprise the O (overlap sequence) as denoted by SEQ ID NO. 98 and 99. In yet some further embodiments, a suitable replacement sequence may comprise the nucleic acid sequence as denoted by SEQ ID NO. 216, or any functional fragments, variants, or derivatives thereof. Such universal replacement sequence may be used when any other CFTR site, specifically, as disclosed by the invention (CF10, CF12, CF13, CF14), is used. It should be further appreciated that in some embodiments, P and P′ sequences that flank the replacement sequence comprise the nucleic acid sequences as denoted by SEQ ID NO. 213 and 214, respectively. Still further, in some embodiments, the first attP1 and a second attP2 sites that flank the replacement sequence in the nucleic acid cassette of the invention may comprise P and P′ sequences that flank the “o” sequence. Such P sequence may comprise the sequence of any one of SEQ ID NO. 100, 213, 240 or 241, and the P′ sequence may comprise the sequence of any one of SEQ ID NO. 101, 214, 242, 243 or 244. It should be noted that any combination of the P and P′ sequences in an attP sites is encompassed by the invention.

In some other embodiments, the target nucleic acid sequence of interest of the eukaryotic cell may comprise or comprised within the human cystinosin (CTNS) gene or any fragment thereof. Such nucleic acid sequence of interest is flanked by a first Int recognition site attE₁ comprising the nucleic acid sequence as denoted by SEQ ID NO. 68 and a second Int recognition site attE₂ comprising the nucleic acid sequence as denoted by SEQ ID NO. 69. In some embodiments, the O₁ may comprise the nucleic acid sequence as denoted by SEQ ID NO. 70 and O₂ may comprise the nucleic acid sequence as denoted by SEQ ID NO. 71. It should be noted that mutated forms of the CTNS gene are associated with Cystinosis.

In some other embodiments, the target nucleic acid sequence of interest of the eukaryotic cell may comprise or comprised within the human CTNS gene or any fragment thereof, flanked by a first Int recognition site attE₁ comprising the nucleic acid sequence as denoted by SEQ ID NO. 68 and a second Int recognition site attE₂ comprising the nucleic acid sequence as denoted by SEQ ID NO. 72. In some embodiments, the O₁ may comprise the nucleic acid sequence as denoted by SEQ ID NO. 70 and O₂ may comprise the nucleic acid sequence as denoted by SEQ ID NO. 73.

In some other embodiments, the target nucleic acid sequence of interest of the eukaryotic cell may comprise or comprised within the human CTNS gene or any fragment thereof, flanked by a first Int recognition site attE₁ comprising the nucleic acid sequence as denoted by SEQ ID NO. 69 and a second Int recognition site attE₂ comprising the nucleic acid sequence as denoted by SEQ ID NO. 72. In some embodiments, the O₁ may comprise the nucleic acid sequence as denoted by SEQ ID NO. 71 and O₂ may comprise the nucleic acid sequence as denoted by SEQ ID NO. 73.

In yet some further embodiments, the target nucleic acid sequence of interest may comprise or comprised within the human CTNS gene or any fragment thereof. Such nucleic acid sequence of interest is flanked by a first Int recognition site attE₁ comprising the nucleic acid sequence as denoted by SEQ ID NO. 72 (CTNS4) and a second Int recognition site attE₂ comprising the nucleic acid sequence as denoted by SEQ ID NO. 116 (CTNS1). In some embodiments, the O₁ may comprise the nucleic acid sequence as denoted by SEQ ID NO. 73 and O₂ may comprise the nucleic acid sequence as denoted by SEQ ID NO. 117. In some other embodiments, the target nucleic acid of interest in the target eukaryotic cell may comprise or comprised within the human CTNS gene or any fragment thereof, flanked by a first Int recognition site attE₁ comprising the nucleic acid sequence as denoted by SEQ ID NO. 129 (CTNS A) and a second Int recognition site attE₂ comprising the nucleic acid sequence as denoted by SEQ ID NO. 130 (CTNS D). In some embodiments, the O₁ may comprise the nucleic acid sequence as denoted by SEQ ID NO. 131 and O₂ may comprise the nucleic acid sequence as denoted by SEQ ID NO. 132.

In some specific and non-limiting embodiments, a suitable replacement sequence in the nucleic acid molecule or cassette provided by the kit/s or systems of the invention may comprise the nucleic acid sequence as denoted by SEQ ID NO. 219, or any functional fragments, variants, or derivatives thereof, specifically, when attE sites comprising the nucleic acid sequence as denoted by SEQ ID NO. 72 and 116 (CTNS4 and CTNS1) that flank exons 1 to 3 in the CTNS gene, are targeted. In such embodiments, the replacement sequence in the nucleic acid cassette used by the kits and systems of the invention, is flanked by attP sites that comprise the O (overlap sequence) as denoted by SEQ ID NO. 73 and 117, respectively. In yet some further embodiments, a suitable replacement sequence may comprise the nucleic acid sequence as denoted by SEQ ID NO. 220, or any functional fragments, variants, or derivatives thereof. Such universal replacement sequence may be used when any other CTNS site, specifically, as disclosed above, is used. It should be further appreciated that in some embodiments, P and P′ sequences that flank the replacement sequence comprise the nucleic acid sequences as denoted by SEQ ID NO. 213 and 214, respectively. Still further, in some embodiments, the first attP1 and a second attP2 sites that flank the replacement sequence in the nucleic acid cassette of the invention may comprise P and P′ sequences that flank the “o” sequence. Such P sequence may comprise the sequence of any one of SEQ ID NO. 100, 213, 240 or 241, and the P′ sequence may comprise the sequence of any one of SEQ ID NO. 101, 214, 242, 243 or 244. It should be noted that any combination of the P and P′ sequences in an attP sites is encompassed by the invention.

Still further, in some embodiments, the target nucleic acid sequence of interest in said eukaryotic cell comprises, or is comprised within the human SCN1A gene or any fragment thereof. Such nucleic acid sequence of interest is flanked by a first Int recognition site attE₁ comprising the nucleic acid sequence as denoted by SEQ ID NO. 120 and a second Int recognition site attE₂ comprising the nucleic acid sequence as denoted by any one of SEQ ID NO. 121, and wherein said O₁ comprises the nucleic acid sequence as denoted by SEQ ID NO. 104 and said O₂ comprises the nucleic acid sequence as denoted by SEQ ID NO. 105. In some specific and non-limiting embodiments, a suitable replacement sequence in the nucleic acid molecule or cassette provided by the kit/s or systems of the invention may comprise the nucleic acid sequence as denoted by SEQ ID NO. 221, or any functional fragments, variants, or derivatives thereof, specifically, when attE sites comprising the nucleic acid sequence as denoted by SEQ ID NO. 121 and 120 (SCN1A3 and SCN1A4) that flank intron 6 in the SCN1A gene, are targeted. In such embodiments, the replacement sequence in the nucleic acid cassette used by the kits and systems of the invention, is flanked by attP sites that comprise the O (overlap sequence) as denoted by SEQ ID NO. 105 and 104, respectively. In yet some further embodiments, a suitable replacement sequence may comprise the nucleic acid sequence as denoted by SEQ ID NO. 222, or any functional fragments, variants, or derivatives thereof. Such universal replacement sequence may be used when any other SCN1A sites are used (ctns1, 2, 3, 4, a and d). It should be further appreciated that in some embodiments, P and P′ sequences that flank the replacement sequence comprise the nucleic acid sequences as denoted by SEQ ID NO. 213 and 214, respectively. Still further, in some embodiments, the first attP1 and a second attP2 sites that flank the replacement sequence in the nucleic acid cassette of the invention may comprise P and P′ sequences that flank the “o” sequence. Such P sequence may comprise the sequence of any one of SEQ ID NO. 100, 213, 240 or 241, and the P′ sequence may comprise the sequence of any one of SEQ ID NO. 101, 214, 242, 243 or 244. It should be noted that any combination of the P and P′ sequences in an attP sites is encompassed by the invention.

In yet some other embodiments of the kit/s and systems of the invention, the target nucleic acid sequence of interest of the eukaryotic cell may comprise or comprised within the human HEXA gene or any fragments thereof, flanked by a first Int recognition site AttE₁ comprising the nucleic acid sequence as denoted by SEQ ID NO. 26 and a second Int recognition site attE₂ comprising the nucleic acid sequence as denoted by SEQ ID NO. 27. In some embodiments, the O₁ may comprise the nucleic acid sequence as denoted by SEQ ID NO. 18 and O₂ may comprise the nucleic acid sequence as denoted by SEQ ID NO. 19.

In some other embodiments of the kit/s and systems of the invention, the target nucleic acid sequence of interest of the eukaryotic cell may comprise or comprised within the human ATM gene or any fragments thereof. Such target nucleic acid sequence of interest is flanked by a first Int recognition site attE₁ comprising the nucleic acid sequence as denoted by SEQ ID NO. 28 and a second Int recognition site attE₂ comprising the nucleic acid sequence as denoted by SEQ ID NO. 29, and O₁ may comprise the nucleic acid sequence as denoted by SEQ ID NO. 20 and O₂ may comprise the nucleic acid sequence as denoted by SEQ ID NO. 21.

In some further embodiments of the kit/s and systems of the invention, the target nucleic acid sequence of interest of the eukaryotic cell may comprise or comprised within the human ATM gene or any fragments thereof, flanked by a first Int recognition site attE₁ comprising the nucleic acid sequence as denoted by SEQ ID NO. 50 and a second Int recognition site attE₂ comprising the nucleic acid sequence as denoted by SEQ ID NO. 28, and O₁ may comprise the nucleic acid sequence as denoted by SEQ ID NO. 51 and O₂ may comprise the nucleic acid sequence as denoted by SEQ ID NO. 20.

In some other alternative embodiments of the kit/s and systems of the invention, the target nucleic acid sequence of interest of the eukaryotic cell may comprise or comprised within the human ATM gene. Such nucleic acid sequence is flanked by a first Int recognition site attE₁ comprising the nucleic acid sequence as denoted by SEQ ID NO. 50 and a second Int recognition site attE₂ comprising the nucleic acid sequence as denoted by SEQ ID NO. 29, and O₁ may comprise the nucleic acid sequence as denoted by SEQ ID NO. 51 and O₂ may comprise the nucleic acid sequence as denoted by SEQ ID NO. 21.

In some other embodiments of the kit/s and systems of the invention, the target nucleic acid sequence of interest of the eukaryotic cell may comprise or comprised within the human HAEM gene or any fragments thereof, flanked by a first Int recognition site attE₁ comprising the nucleic acid sequence as denoted by SEQ ID NO. 30 and a second Int recognition site attE₂ comprising the nucleic acid sequence as denoted by SEQ ID NO. 31, and wherein O₁ may comprise the nucleic acid sequence as denoted by SEQ ID NO. 22 and O₂ may comprise the nucleic acid sequence as denoted by SEQ ID NO. 23.

In some other embodiments of the kit/s and systems of the invention, the target nucleic acid sequence of interest of the eukaryotic cell may comprise or comprised within the human HGPRT gene or any fragments thereof, flanked by a first Int recognition site attE₁ comprising the nucleic acid sequence as denoted by SEQ ID NO. 32 and a second Int recognition site attE₂ comprising the nucleic acid sequence as denoted by SEQ ID NO. 33, and O₁ may comprise the nucleic acid sequence as denoted by SEQ ID NO. 24 and O₂ may comprise the nucleic acid sequence as denoted by SEQ ID NO. 25.

In some other embodiments of the kit/s and systems of the invention, the target nucleic acid sequence of interest of the eukaryotic cell may comprise or comprised within the human SOD1 gene or any fragments thereof, flanked by a first Int recognition site attE₁ comprising the nucleic acid sequence as denoted by SEQ ID NO. 52 and a second Int recognition site attE₂ comprising the nucleic acid sequence as denoted by SEQ ID NO. 53, and O₁ may comprise the nucleic acid sequence as denoted by SEQ ID NO. 54 and O₂ may comprise the nucleic acid sequence as denoted by SEQ ID NO. 55.

In some other embodiments of the kit/s and systems of the invention, the target nucleic acid sequence of interest of the eukaryotic cell may comprise or comprised within the human TARDBP gene or any fragments thereof, flanked by a first Int recognition site attE₁ comprising the nucleic acid sequence as denoted by SEQ ID NO. 56 and a second Int recognition site attE₂ comprising the nucleic acid sequence as denoted by SEQ ID NO. 57, and O₁ may comprise the nucleic acid sequence as denoted by SEQ ID NO. 58 and O₂ may comprise the nucleic acid sequence as denoted by SEQ ID NO. 59.

In some other embodiments of the kit/s and systems of the invention, the target nucleic acid sequence of interest of the eukaryotic cell may comprise or comprised within the human VABP gene or any fragments thereof, flanked by a first Int recognition site attE₁ comprising the nucleic acid sequence as denoted by SEQ ID NO. 60 and a second Int recognition site attE₂ comprising the nucleic acid sequence as denoted by SEQ ID NO. 61, and O₁ may comprise the nucleic acid sequence as denoted by SEQ ID NO. 62 and O₂ may comprise the nucleic acid sequence as denoted by SEQ ID NO. 63.

In some other embodiments of the kit/s and systems of the invention, the target nucleic acid sequence of interest of the eukaryotic cell may comprise or comprised within the human C9ORF71 gene or any fragments thereof, flanked by a first Int recognition site attE₁ comprising the nucleic acid sequence as denoted by SEQ ID NO. 64 and a second Int recognition site attE₂ comprising the nucleic acid sequence as denoted by SEQ ID NO. 65, and O₁ may comprise the nucleic acid sequence as denoted by SEQ ID NO. 66 and O₂ may comprise the nucleic acid sequence as denoted by SEQ ID NO. 67.

In some other embodiments of the kits and/or systems of the invention, the target gene or nucleic acid sequence of interest of the eukaryotic cell may be, may comprise or may comprised within the human COL3A1 gene or any fragment thereof, flanked by a first Int recognition site attE₁ comprising the nucleic acid sequence as denoted by SEQ ID NO. 122 and a second Int recognition site attE₂ comprising the nucleic acid sequence as denoted by SEQ ID NO. 123. In some embodiments, the O₁ may comprise the nucleic acid sequence as denoted by SEQ ID NO. 106 and O₂ may comprise the nucleic acid sequence as denoted by SEQ ID NO. 107.

In some other embodiments kits and/or systems of the invention, the target gene or nucleic acid sequence of interest of the eukaryotic cell may be, may comprise or may comprised within the human NPC1 gene or any fragment thereof, flanked by a first Tnt recognition site attE₁ comprising the nucleic acid sequence as denoted by SEQ ID NO. 118 and a second Int recognition site attE₂ comprising the nucleic acid sequence as denoted by SEQ ID NO. 119. In some embodiments, the O₁ may comprise the nucleic acid sequence as denoted by SEQ ID NO. 102 and O₂ may comprise the nucleic acid sequence as denoted by SEQ ID NO. 103.

Another aspect of the invention relates to nucleic acid molecule or any nucleic acid cassette or vector thereof. The nucleic acid molecule or cassette in accordance with the invention comprises a replacement-sequence flanked by a first and a second Int recognition sites. The first site attP1, comprises a first overlap sequence O1 and the second site attP2, comprises a second overlap sequence O2. It should be noted that the first O1 and the second O2 overlap sequences are different, each consisting of seven nucleotides. The O1 is identical to an overlap sequence O1 comprised within a first Int recognition site attE1 in a eukaryotic cell and the O2 is identical to an overlap sequence O2 comprised within a second Int recognition site attE2 in the eukaryotic cell. It should be noted that the said eukaryotic recognition sites attE1 and attE2 flank a target nucleic acid sequence of interest or any fragment thereof in said eukaryotic cell. In some embodiments, the first binding sites E may comprise the sequence of C1-T2-T3-W4, as denoted by SEQ ID NO. 16, and the second binding sites E′ may comprise the sequence of A12-A13-A14-G15, as denoted by SEQ ID NO. 17.

In some embodiments, the nucleic acid molecule or cassette of the invention comprise replacement sequence for target nucleic acid sequence of interest in the eukaryotic cell.

In some embodiments such target nucleic acid sequence comprises, or is comprised within the human CFTR gene, specifically, the nucleic acid sequence of interest is flanked by a first Int recognition site attE1 comprising the nucleic acid sequence as denoted by SEQ ID NO. 96 and a second Int recognition site attE2 comprising the nucleic acid sequence as denoted by SEQ ID NO. 97. The O1 comprises the nucleic acid sequence as denoted by SEQ ID NO. 98 and the O2 comprises the nucleic acid sequence as denoted by SEQ ID NO. 99.

In some specific and non-limiting embodiments, a suitable replacement sequence in the nucleic acid molecule or cassette of the invention may comprise the nucleic acid sequence as denoted by SEQ ID NO. 215, or any functional fragments, variants, or derivatives thereof, specifically, when attE sites comprising the nucleic acid sequence as denoted by SEQ ID NO. 96 and 97 (CFTR10 and CFTR12) that flank exon 3 in the CFTR gene, are targeted. In such embodiments, the replacement sequence in the nucleic acid cassette of the invention, is flanked by attP sites that comprise the O as denoted by SEQ ID NO. 98 and 99. In yet some further embodiments, a suitable replacement sequence may comprise the nucleic acid sequence as denoted by SEQ ID NO. 216, or any functional fragments, variants, or derivatives thereof. Such universal replacement sequence may be used when any other CFTR site, specifically, as disclosed above in connection with other aspects of the invention, are used. It should be further appreciated that in some embodiments, P and P′ sequences that flank the replacement sequence in the nucleic acid cassette of the invention comprise the nucleic acid sequences as denoted by SEQ ID NO. 213 and 214, respectively. Accordingly, a donor cassette may comprise the replacement sequence as flanked by attP1 and attP2 sites that comprise the O1 and O2 sequences, respectively. These O1 and O2 are different from each other, and are identical to O1 and O2 sites in the target sequence in the target eukaryotic cell. Still further, in some embodiments, the first attP1 and a second attP2 sites that flank the replacement sequence in the nucleic acid cassette of the invention may comprise P and P′ sequences that flank the “o” sequence. Such P sequence may comprise the sequence of any one of SEQ ID NO. 100, 213, 240 or 241, and the P′ sequence may comprise the sequence of any one of SEQ ID NO. 101, 214, 242, 243 or 244. It should be noted that any combination of the P and P′ sequences in an attP sites is encompassed by the invention. Thus, the cassette of the invention may comprise in some embodiments P and P′ sequences that flank any of the CFTR O sequences discussed by the invention, forming the POP′ sites that flank the suitable replacement sequences, for example, the replacement sequence of SEQ ID NO. 215 when O sequences of CFTR10 and CFTR12 are used, or the universal replacement sequence as denoted by SEQ ID NO. 216, when any other CFTR O sequences are used.

In yet some further embodiments, target nucleic acid sequence comprises, or is comprised within the human CTNS gene or any fragment thereof. Such nucleic acid sequence of interest is flanked by a first Int recognition site attE₁ comprising the nucleic acid sequence as denoted by SEQ ID NO. 116 and a second Int recognition site attE₂ comprising the nucleic acid sequence as denoted by any one of SEQ ID NO. 72, and wherein said O₁ comprises the nucleic acid sequence as denoted by SEQ ID NO. 117 and said O₂ comprises the nucleic acid sequence as denoted by SEQ ID NO. 73. In some specific and non-limiting embodiments, a suitable replacement sequence in the nucleic acid molecule or cassette of the invention may comprise the nucleic acid sequence as denoted by SEQ ID NO. 219, or any functional fragments, variants, or derivatives thereof, specifically, when attE sites comprising the nucleic acid sequence as denoted by SEQ ID NO. 72 and 116 (CTNS4 and CTNS1) that flank exons 1 to 3 in the CTNS gene, are targeted. In such embodiments, the replacement sequence in the nucleic acid cassette of the invention, is flanked by attP sites that comprise the O (overlap sequence) as denoted by SEQ ID NO. 73 and 117, respectively. In yet some further embodiments, a suitable replacement sequence may comprise the nucleic acid sequence as denoted by SEQ ID NO. 220, or any functional fragments, variants, or derivatives thereof. Such universal replacement sequence may be used when any other CTNS site, specifically, as disclosed above, is used. It should be further appreciated that in some embodiments, P and P′ sequences that flank the replacement sequence comprise the nucleic acid sequences as denoted by SEQ ID NO. 213 and 214, respectively. Accordingly, a donor cassette may comprise the replacement sequence as flanked by attP1 and attP2 sites that comprise the O1 and O2 sequences, respectively. These O1 and O2 are different from each other, and are identical to O1 and O2 sites in the target sequence in the target eukaryotic cell. Still further, in some embodiments, the first attP1 and a second attP2 sites that flank the replacement sequence in the nucleic acid cassette of the invention may comprise P and P′ sequences that flank the “o” sequence. Such P sequence may comprise the sequence of any one of SEQ ID NO. 100, 213, 240 or 241, and the P′ sequence may comprise the sequence of any one of SEQ ID NO. 101, 214, 242, 243 or 244. It should be noted that any combination of the P and P′ sequences in an attP sites is encompassed by the invention. Thus, the cassette of the invention may comprise in some embodiments P and P′ sequences that flank any of the CTNS O sequences discussed by the invention, forming the POP′ sites that flank the suitable replacement sequences, for example, the replacement sequence of SEQ ID NO. 219 when O sequences of CTNS4 and CTNS1 are used, or the universal replacement sequence as denoted by SEQ ID NO. 220, when any other CTNS O sequences are used.

In some embodiments such target nucleic acid sequence comprises, or is comprised within the human SCN1A gene or any fragment thereof. such nucleic acid sequence of interest is flanked by a first Int recognition site attE₁ comprising the nucleic acid sequence as denoted by SEQ ID NO. 120 and a second Int recognition site attE₂ comprising the nucleic acid sequence as denoted by any one of SEQ ID NO. 121, and wherein said O₁ comprises the nucleic acid sequence as denoted by SEQ ID NO. 104 and said O₂ comprises the nucleic acid sequence as denoted by SEQ ID NO. 105.

In some specific and non-limiting embodiments, a suitable replacement sequence in the nucleic acid molecule or cassette of the invention may comprise the nucleic acid sequence as denoted by SEQ ID NO. 221, or any functional fragments, variants, or derivatives thereof, specifically, when attE sites comprising the nucleic acid sequence as denoted by SEQ ID NO. 121 and 120 (SCN1A3 and SCN1A4) that flank intron 6 in the SCN1A gene, are targeted. In such embodiments, the replacement sequence in the nucleic acid cassette of the invention, is flanked by attP sites that comprise the O (overlap sequence) as denoted by SEQ ID NO. 105 and 104, respectively. In yet some further embodiments, a suitable replacement sequence may comprise the nucleic acid sequence as denoted by SEQ ID NO. 222, or any functional fragments, variants, or derivatives thereof. Such universal replacement sequence may be used when any other SCN1A sites are used. It should be further appreciated that in some embodiments, P and P′ sequences that flank the replacement sequence comprise the nucleic acid sequences as denoted by SEQ ID NO. 213 and 214, respectively. Accordingly, a donor cassette may comprise the replacement sequence as flanked by attP1 and attP2 sites that comprise the O1 and O2 sequences, respectively. These O1 and O2 are different from each other, and are identical to O1 and O2 sites in the target sequence in the target eukaryotic cell. Still further, in some embodiments, the first attP1 and a second attP2 sites that flank the replacement sequence in the nucleic acid cassette of the invention may comprise P and P′ sequences that flank the “o” sequence. Such P sequence may comprise the sequence of any one of SEQ ID NO. 100, 213, 240 or 241, and the P′ sequence may comprise the sequence of any one of SEQ ID NO. 101, 214, 242, 243 or 244. It should be noted that any combination of the P and P′ sequences in an attP sites is encompassed by the invention.

Thus, the cassette of the invention may comprise in some embodiments P and P′ sequences that flank any of the SCN1A O sequences discussed by the invention, forming the POP′ sites that flank the suitable replacement sequences, for example, the replacement sequence of SEQ ID NO. 221 when O sequences of SCN1A3 and SCN1A4 are used, or the universal replacement sequence as denoted by SEQ ID NO. 222, when any other SCN1A O sequences are used.

Still further, in some embodiments, such target nucleic acid sequence comprises, or is comprised within the human DMD gene or any fragment thereof. Such nucleic acid sequence of interest is flanked by a first Int recognition site attE1 comprising the nucleic acid sequence as denoted by SEQ ID NO. 92 and a second Int recognition site attE2 comprising the nucleic acid sequence as denoted by any one of SEQ ID NO. 93, and wherein said O1 comprises the nucleic acid sequence as denoted by SEQ ID NO. 94 and said O2 comprises the nucleic acid sequence as denoted by SEQ ID NO. 95. In some specific and non-limiting embodiments, a suitable replacement sequence in the nucleic acid molecule or cassette of the invention may comprise the nucleic acid sequence as denoted by SEQ ID NO. 217, or any functional fragments, variants, or derivatives thereof, specifically, when attE sites comprising the nucleic acid sequence as denoted by SEQ ID NO. 92 and 93 (DMD2 and DMD3) that flank exon 44 in the DMD gene, are targeted. In such embodiments, the replacement sequence in the nucleic acid cassette of the invention, is flanked by attP sites that comprise the O (overlap sequence) as denoted by SEQ ID NO. 94 and 95. In yet some further embodiments, a suitable replacement sequence may comprise the nucleic acid sequence as denoted by SEQ ID NO. 218, or any functional fragments, variants, or derivatives thereof. Such universal replacement sequence may be used when any other DMD sites, specifically, as disclosed above, are used. It should be further appreciated that in some embodiments, P and P′ sequences that flank the replacement sequence comprise the nucleic acid sequences as denoted by SEQ ID NO. 213 and 214, respectively. Accordingly, a donor cassette may comprise the replacement sequence as flanked by attP1 and attP2 sites that comprise the O1 and O2 sequences, respectively. These O1 and O2 are different from each other, and are identical to O1 and O2 sites in the target sequence in the target eukaryotic cell. Still further, in some embodiments, the first attP1 and a second attP2 sites that flank the replacement sequence in the nucleic acid cassette of the invention may comprise P and P′ sequences that flank the “o” sequence. Such P sequence may comprise the sequence of any one of SEQ ID NO. 100, 213, 240 or 241, and the P′ sequence may comprise the sequence of any one of SEQ ID NO. 101, 214, 242, 243 or 244. It should be noted that any combination of the P and P′ sequences in an attP sites is encompassed by the invention. Thus, the cassette of the invention may comprise in some embodiments P and P′ sequences that flank any of the DMD O sequences discussed by the invention, forming the POP′ sites that flank the suitable replacement sequences, for example, the replacement sequence of SEQ ID NO. 217 when O sequences of DMD2 and DMD3 are used, or the universal replacement sequence as denoted by SEQ ID NO. 218, when any other DMD O sequences are used.

It should be understood that the invention further encompasses any nucleic acid molecule and nucleic acid cassette that comprise any replacement sequence suitable for replacing any target nucleic acid sequence, specifically, any of the target nucleic acid sequences disclosed by the invention in connection with other aspects of the invention. Still further, these cassettes comprise the suitable replacement sequence flanked by POP and P′OP′ (forming the appropriate attP1 and attP2 that flank the replacement sequences) that comprise the P sequence as denoted by SEQ ID NO. 213, and the P′ sequence as denoted by SEQ ID NO. 214, and any of the suitable overlap “O” sequences disclosed by the invention. In some embodiments, the replacement sequence in the nucleic acid molecule or cassette provided by the invention (also referred to herein as donor cassette) is flanked by a first attP1 and a second attP2 recognition sites that comprise “O” sequences that are identical to the “O” sequences that flank the target nucleic acid sequence in the eukaryotic cell. In some embodiments, the recognitions sites are composed of only the “o” sequences that flank the replacement sequences. In yet some further embodiments, these “o” sequences in the first and second recognition sites are flanked by P and P′ arms that may comprise between 0 to 500 or more nucleotides. In some further embodiments, the P and P′ arms may comprise a nucleic acid sequence of between about 1 to 500 nucleotides or more, about 1 to 450, 400, 350, 300, 250, 200, 150, 100, 50, 40, 30, 20, 15, 10, 9, 8, 7, 6, 5, 4, 3, 2 nucleotides. In some specific and non-limiting embodiments these first and second recognition sites may comprise P and P′ sequences of the wild type Int-HK022 attP sites. In some embodiments, the P sequence may comprise the nucleic acid sequence as denoted by SEQ ID NO. 100 or any fragments or derivatives thereof. In yet some further embodiments, the P′ may comprise the Int-HK022 attP′ as denoted by SEQ ID NO. 101 or any fragments or derivatives thereof. It should be further appreciated that in some embodiments, P and P′ sequences that flank the replacement sequence comprise the nucleic acid sequences as denoted by SEQ ID NO. 213 and 214, respectively, or any derivatives, fragments or variants thereof. Still further, in some embodiments, the first attP1 and a second attP2 sites that flank the replacement sequence in the nucleic acid cassette of the invention may comprise P and P′ sequences that flank the “o” sequence. Such P sequence may comprise the sequence of any one of SEQ ID NO. 100, 213, 240 or 241, and the P′ sequence may comprise the sequence of any one of SEQ ID NO. 101, 214, 242, 243 or 244. It should be noted that any combination of the P and P′ sequences in an attP sites is encompassed by the invention. It should be understood that any of the nucleic acid sequences that comprise the at least one replacement sequence flanked by the appropriate attP1 and attP2 sites, as disclosed by the invention in connection with other aspects of the invention, are also applicable in the present aspect as well and each forms an independent embodiment of the invention.

The term “nucleic acid cassette” refers to a polynucleotide sequence comprising at least one regulatory sequence operably linked to a sequence encoding the nucleic acid sequence encoding any of the HK-Int variants and or mutants of the invention. It should be understood that the term “cassette” as used by the invention further encompasses any cassette or vector comprising any replacement sequence as will be described in more detail in connection with other aspects of the invention. All elements comprised within the cassette of the invention are operably linked together. The term “operably linked”, as used in reference to a regulatory sequence and a structural nucleotide sequence, means that the nucleic acid sequences are linked in a manner that enables regulated expression of the linked structural nucleotide sequence. In some embodiments, the cassette of the invention may further comprise at least one genetic element. In some specific embodiments, such genetic element may be at least one of: at least one splice acceptor (SA), and/or splice donor (SD), internal ribosome entry sequences (IRES), a 2A peptide coding sequence, a promoter or any functional fragments thereof (e.g., a minimal promoter, constitutive, inducible, endogenous or heterologous promoter), degron sequence, Signal peptide leader, mRNA stabilizing sequence, stop codon, 3-frame stop codon sequence, at least one polyadenylation sequence and a transcription enhancer.

In another aspect, the invention relates to a composition comprising as an active ingredient an effective amount of

(a) at least one HK-Int variant and/or mutated molecule or any functional fragments or peptides thereof, any nucleic acid molecule comprising a sequence encoding the HK-Int variant and/or mutated molecule or any vector, vehicle, matrix, nano- or micro-particle comprising the same, or any host cell comprising the HK-Int variant or nucleic acid sequence encoding the HK-Int variant.

In some embodiments, the variant HK-Int variant and/or mutated molecule of the composition of the invention comprise at least one substituted amino acid residue in at least one of the CB, ND and the CD of the Wild type HK-Int molecule.

In some further embodiments, the composition of the invention may optionally further comprise as an additional component (b), at least one nucleic acid molecule or nucleic acid cassette comprising a replacement-sequence flanked by a first and a second Int recognition sites. In some embodiments, the first site attP1 may comprise a first overlap sequence O1 and the second site attP2 may comprise a second overlap sequence O2. In yet another embodiment, the first O1 and the second O2 overlap sequences may be different, each consisting of seven nucleotides, the O1 may be identical to an overlap sequence O1 comprised within a first Int recognition site attE1 in a eukaryotic cell and the O2 may be identical to an overlap sequence O2 comprised within a second Int recognition site attE2 in the eukaryotic cell. In some embodiments, the eukaryotic recognition sites attE1 and attE2 may flank a target nucleic acid sequence of interest or any fragment thereof in the eukaryotic cell, or a kit or system comprising (a) and (b). In some embodiments, the first binding sites E may comprise the sequence of C1-T2-T3-W4, as denoted by SEQ ID NO. 16, and the second binding sites E′ may comprise the sequence of A12-A13-A14-G15, as denoted by SEQ ID NO. 17. It should be appreciated that the invention further encompasses compositions comprising host cell/s that comprise the Int variants of the invention, or any nucleic acid sequence encoding said variants and in addition, at least one nucleic acid molecule that comprise the replacement sequence as discussed above.

In some further embodiments, the HK-Int mutated molecule and/or variant of the composition of the invention may be as the HK-Int mutated molecules/variants as defined by the invention. More specifically, at least one HK-Int variant and/or mutated molecule/s that may be used in the composition of the invention may comprise at least one substituted amino acid residue in at least one of the CB, the ND and the CD domains of the Wild type HK-Int molecule. In some particular embodiments, the HK-Int mutated molecule and/or variant may comprise at least one substitution at any position of residues 174, 278, 43, 319, 134, 149, 215, 264, 303, 309, 336 of the amino acid sequence of the Wild type HK-Int molecule as denoted by SEQ ID NO. 13 and any combinations thereof. In some other embodiments, the HK-Int mutated molecule and/or variant may comprise at least one substitution at the CB domain. In yet some specific embodiments, the HK-Int mutated molecule and/or variant may comprise at least one substitution at position 174 of the Wild type HK-Int molecule as denoted by SEQ ID NO. 13. In yet some further specific embodiments, the HK-Int mutated molecule and/or variant or the composition of the invention may comprise the E174K, specifically of the amino acid sequence as denoted by SEQ ID NO. 14. In yet some further embodiments, the composition of the invention may comprise an Int mutant or variant that comprise a substitution of amino acid residue at position 278, specifically, replacing D278 with K. In some embodiments, such mutant comprise the amino acid sequence as denoted by SEQ ID NO.182, or any derivatives, homologs, fusion proteins or variants thereof. It should be further appreciated that any of the HK-Int variants of the invention as denoted by SEQ ID NO. 14, 182, 42, 44, 46, 48, 180, 188, 190, 192, 223 or the double mutants having the amino acid sequence as denoted by any one of SEQ ID NO. 83, 85, 87, 89, 184, or the triple mutant of SEQ ID NO. 185, and any functional fragments, variants, fusion proteins or derivatives thereof, may be used by any of the compositions of the invention.

In some other embodiment, the composition of the invention may comprise a nucleic acid molecule comprising a nucleic acid sequence encoding a HK-Int mutated molecule and/or variant or any functional fragments or peptides thereof. In some embodiments, the nucleic acid molecules of the composition of the invention may comprise a nucleic acid sequence encoding for any of the HK-Int mutated molecules and/or variants as defined by the invention. In yet some further embodiments, the composition of the invention may comprise at least one nucleic acid molecule comprising the nucleic acid molecules as denote by any one of SEQ ID NO. 15, 43, 45, 47, 49, 82, 84, 86, 88, 186, 183, 187, 181, 189, 191, 193, 224, or any derivatives, homologs or variants thereof.

In some further embodiments, the composition of the invention may comprise a host cell comprising (for example, transformed or transfected with) at least one nucleic acid molecule comprising a nucleic acid sequence encoding at least one HK-Int variant and/or mutated molecule or any functional fragments or peptides thereof, any combinations thereof, or with any vector, vehicle, matrix, nano- or micro-particle comprising the same, or encoding any of HK-Int mutated molecules and/or variants as defined by the invention.

In yet another embodiments, the host cell comprised within the composition of the invention may further comprise at least one nucleic acid molecule comprising a replacement-sequence flanked by a first and a second Int recognition sites, said first site attP1 comprises a first overlap sequence O1 and said second site attP2 comprises a second overlap sequence O2, wherein said first O1 and said second O2 overlap sequences are different, each consisting of seven nucleotides, said O1 is identical to an overlap sequence O1 comprised within a first Int recognition site attE1 in a eukaryotic cell and said O2 is identical to an overlap sequence O2 comprised within a second Int recognition site attE2 in said eukaryotic cell, said eukaryotic recognition sites attE1 and attE2 flank a target nucleic acid sequence of interest or any fragment thereof in said eukaryotic cell, wherein said O1 and O2 overlap sequences are each flanked by a first E and a second E′ Int binding sites, wherein said first binding sites E comprise the sequence of C1-T2-T3-W4, as denoted by SEQ ID NO. 16, and said second binding sites E′ comprise the sequence of A12-A13-A14-G15, as denoted by SEQ ID NO. 17.

In some embodiments, the replacement-sequence flanked by a first and a second Int recognition sites of the host cell comprised within the composition of the invention, may comprise at least one nucleic acid sequence that differs in at least one nucleotide from the at least one sequence to be replaced in the target nucleic acid sequence. It should be understood that the replacement nucleic acid sequence comprised within the composition of the invention may replace a target nucleic acid sequence of interest in a target eukaryotic cell.

In some specific embodiments, the target nucleic acid sequence of interest of the eukaryotic cell may comprise or comprised within the human DMD gene or any fragment thereof. Such target nucleic acid sequence is flanked by a first Int recognition site attE1 comprising the nucleic acid sequence as denoted by SEQ ID NO. 92 and a second Int recognition site attE2 comprising the nucleic acid sequence as denoted by SEQ ID NO. 93, and O1 may comprise the nucleic acid sequence as denoted by SEQ ID NO. 94 and O2 may comprise the nucleic acid sequence as denoted by SEQ ID NO. 95. Still further, in some embodiments, other DMD fragments that should be replaced, may be flanked by any of the attE sequence designated herein as DMD4, having the sequence of SEQ ID NO. 108 (with an O sequence as denoted by SEQ ID NO. 109), DMD5, having the sequence of SEQ ID NO. 110 (with an O sequence as denoted by SEQ ID NO. 111), DMD6, having the sequence of SEQ ID NO. 112 (with an O sequence as denoted by SEQ ID NO. 113) or DMD7, having the sequence of SEQ ID NO. 114 (with an O sequence as denoted by SEQ ID NO. 115). Non limiting examples for replacement nucleic acid sequence suitable for DMD, are disclosed herein above in connection with other aspects of the invention, specifically, the replacement sequences that comprise the nucleic acid sequence as denoted by SEQ ID NO. 217 and 218, or any variants or derivatives thereof.

In some other specific embodiments, the target nucleic acid sequence of interest of the eukaryotic cell may comprise or comprised within the human CFTR gene or any fragment thereof, flanked by a first Int recognition site attE1 comprising the nucleic acid sequence as denoted by SEQ ID NO. 96 and a second Int recognition site attE2 comprising the nucleic acid sequence as denoted by SEQ ID NO. 97, and O1 may comprise the nucleic acid sequence as denoted by SEQ ID NO. 98 and O2 may comprise the nucleic acid sequence as denoted by SEQ ID NO. 99. In yet some other specific embodiments, the target nucleic acid sequence of interest of the eukaryotic cell may comprise or comprised within the human CFTR gene or any fragment thereof, flanked by a first Int recognition site attE1 comprising the nucleic acid sequence as denoted by SEQ ID NO. 125 and a second Int recognition site attE2 comprising the nucleic acid sequence as denoted by SEQ ID NO. 126, and O1 may comprise the nucleic acid sequence as denoted by SEQ ID NO. 127 and O2 may comprise the nucleic acid sequence as denoted by SEQ ID NO. 128. Non limiting examples for replacement nucleic acid sequences suitable for CFTR, are disclosed herein above in connection with other aspects of the invention, specifically, the replacement sequence that comprise the nucleic acid sequence as denoted by SEQ ID NO. 215 and 216, or any variants or derivatives thereof.

In some other specific embodiments, the target nucleic acid sequence of interest of the eukaryotic cell may comprise or comprised within the human CTNS gene or any fragment thereof, flanked by a first Int recognition site attE₁ comprising the nucleic acid sequence as denoted by SEQ ID NO. 68 and a second Int recognition site attE₂ comprising the nucleic acid sequence as denoted by SEQ ID NO. 69. In some embodiments, the O₁ may comprise the nucleic acid sequence as denoted by SEQ ID NO. 70 and O₂ may comprise the nucleic acid sequence as denoted by SEQ ID NO. 71. In some other embodiments, the target nucleic acid sequence of interest of the eukaryotic cell may comprise or comprised within the human CTNS gene or any fragment thereof, flanked by a first Int recognition site attE₁ comprising the nucleic acid sequence as denoted by SEQ ID NO. 68 and a second Int recognition site attE₂ comprising the nucleic acid sequence as denoted by SEQ ID NO. 72. In some embodiments, the O₁ may comprise the nucleic acid sequence as denoted by SEQ ID NO. 70 and O₂ may comprise the nucleic acid sequence as denoted by SEQ ID NO. 73. In some other embodiments, the target nucleic acid sequence of interest of the eukaryotic cell may comprise or comprised within the human CTNS gene or any fragment thereof, flanked by a first Int recognition site attE₁ comprising the nucleic acid sequence as denoted by SEQ ID NO. 69 and a second Int recognition site attE₂ comprising the nucleic acid sequence as denoted by SEQ ID NO. 72. In some embodiments, the O₁ may comprise the nucleic acid sequence as denoted by SEQ ID NO. 71 and O₂ may comprise the nucleic acid sequence as denoted by SEQ ID NO. 73. In yet some further embodiments, the target nucleic acid sequence of interest of the eukaryotic cell may comprise or is comprised within the human CTNS gene or any fragment thereof. Such nucleic acid sequence of interest is flanked by a first Int recognition site attE₁ comprising the nucleic acid sequence as denoted by SEQ ID NO. 72 (CTNS4) and a second Int recognition site attE₂ comprising the nucleic acid sequence as denoted by SEQ ID NO. 116 (CTNS1). In some embodiments, the O₁ may comprise the nucleic acid sequence as denoted by SEQ ID NO. 73 and O₂ may comprise the nucleic acid sequence as denoted by SEQ ID NO. 117. In some further embodiments, the target nucleic acid sequence of interest may comprise or comprised within the human CTNS gene or any fragment thereof, flanked by an Int recognition site attE as denoted by SEQ ID NO. 129, with an “o” sequence as denoted by SEQ ID NO. 131. Still further e, the target nucleic acid sequence of interest may be the human CTNS gene or any fragment thereof, flanked by an Int recognition site ate as denoted by SEQ ID NO. 130, with an “o” sequence as denoted by SEQ ID NO. 132. Non limiting examples for replacement nucleic acid sequences suitable for CTNS, are disclosed herein above in connection with other aspects of the invention, specifically, the replacement sequence that comprise the nucleic acid sequence as denoted by SEQ ID NO. 219 and 220, or any variants or derivatives thereof.

In some other specific embodiments, the target nucleic acid sequence of interest of the eukaryotic cell may comprise or comprised within the human SCN1A gene or any fragment thereof. Such nucleic acid sequence of interest is flanked by a first Int recognition site attE₁ comprising the nucleic acid sequence as denoted by SEQ ID NO. 120 and a second Int recognition site attE₂ comprising the nucleic acid sequence as denoted by any one of SEQ ID NO. 121, and wherein said O₁ comprises the nucleic acid sequence as denoted by SEQ ID NO. 104 and said O₂ comprises the nucleic acid sequence as denoted by SEQ ID NO. 105. Non limiting examples for replacement nucleic acid sequences suitable for SCN1A, are disclosed herein above in connection with other aspects of the invention, specifically, the replacement sequence that comprise the nucleic acid sequence as denoted by SEQ ID NO. 221 and 222, or any variants or derivatives thereof.

In yet some alternative embodiments, the composition of the invention may comprise a system/kit comprising at least one nucleic acid molecule (a) and at least one HK-Int variant and/or mutated molecule (b).

In some embodiments, the at least one nucleic acid molecule (a) may comprise a replacement-sequence flanked by a first and a second Int recognition sites. In some further embodiments, the first site attP1 may comprise a first overlap sequence O1 and the second site attP2 may comprise a second overlap sequence O2. In other embodiments, the first O1 and the second O2 overlap sequences may be different, each consisting of seven nucleotides, the O1 may be identical to an overlap sequence O1 comprised within a first Int recognition site attE1 in a eukaryotic cell and the O2 may be identical to an overlap sequence O2 comprised within a second Int recognition site attE2 in the eukaryotic cell. In yet another embodiment, the eukaryotic recognition sites attE1 and attE2 may flank a target nucleic acid sequence of interest or any fragment thereof in the eukaryotic cell. It should be understood that any of the nucleic acid sequences that comprise the at least one replacement sequence flanked by the appropriate attP1 and attP2 sites, as disclosed by the invention in connection with other aspects of the invention, are also applicable in the present aspect as well and each forms an independent embodiment of the invention.

In some further embodiments, the composition may comprise (b) the at least one HK-Int variant and/or mutated molecule or any functional fragments or peptides thereof, any nucleic acid molecule comprising a sequence encoding the HK-Int variant and/or mutated molecule or any vector, vehicle, matrix, nano- or micro-particle comprising the same or any of the HK-Int variant and/or mutated molecules as defined by the invention.

In other embodiments, the composition of the invention may comprise any of the systems/kits as defined by the invention.

The term “effective amount” relates to the amount of an active agent present in a composition, specifically, the HK-Int variant/s or mutants, nucleic acid sequences encoding the HK-Int variant/s or mutants, host cells, nucleic acid molecules and cassettes that comprise the replacement nucleic acid sequences flanked by the appropriate attP and attP′ sites (that comprise any of the o sites disclosed by the invention), kit/s or system/s of the invention as described herein that is needed to provide a desired level of active agent in the bloodstream or at the site of action in an individual to be treated to give an anticipated physiological response when such composition is administered. The precise amount will depend upon numerous factors, e.g., the active agent, the activity of the composition, the delivery device employed, the physical characteristics of the composition, intended patient use (i.e., the number of doses administered per day), patient considerations, and the like, and can readily be determined by one skilled in the art, based upon the information provided herein.

An “effective amount” of the HK-Int mutant, nucleic acid, host cell or system of the invention can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

In yet some further embodiments, the composition of the invention may optionally further comprises at least one of pharmaceutically acceptable carrier/s, excipient/s, additive/s diluent/s and adjuvant/s.

The pharmaceutical compositions of the invention can be administered and dosed by the methods of the invention, in accordance with good medical practice, systemically, for example by parenteral intravenous. It should be noted however that the invention may further encompass additional administration modes. In other examples, the pharmaceutical composition can be introduced to a site by any suitable route including intraperitoneal, subcutaneous, transcutaneous, topical, intramuscular, intraarticular, subconjunctival, or mucosal, e.g. oral, intranasal, or intraocular administration.

Local administration to the area in need of treatment may be achieved by, for example, by local infusion during surgery, topical application, direct injection into the specific organ. More specifically, the compositions used in any of the methods of the invention, described herein before, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

In yet some further embodiments, the composition of the invention may optionally further comprises at least one of pharmaceutically acceptable carrier/s, excipient/s, additive/s diluent/s and adjuvant/s.

More specifically, pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients, specifically, the HK-Int variant/s or mutants, nucleic acid sequences encoding the HK-Int variant/s or mutants, host cells, nucleic acid molecules and cassettes that comprise the replacement nucleic acid sequences flanked by the appropriate attP and attP′ sites (that comprise any of the o sites disclosed by the invention), kit/s or system/s of the invention with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

It should be understood that in addition to the ingredients particularly mentioned above, the formulations may also include other agents conventional in the art having regard to the type of formulation in question.

Still further, pharmaceutical preparations are compositions that include the HK-Int variant/s or mutants, nucleic acid sequences encoding the HK-Int variant/s or mutants, host cells, nucleic acid molecules and cassettes that comprise the replacement nucleic acid sequences flanked by the appropriate attP and attP′ sites (that comprise any of the o sites disclosed by the invention), kit/s or system/s of the invention present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of the HK-Int mutant, nucleic acid, host cell or system of the invention can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation.

The active agent may be formulated for immediate activity or it may be formulated for sustained release.

Still further, the composition/s of the invention and any components thereof may be applied as a single daily dose or multiple daily doses, preferably, every 1 to 7 days. It is specifically contemplated that such application may be carried out once, twice, thrice, four times, five times or six times daily, or may be performed once daily, once every 2 days, once every 3 days, once every 4 days, once every 5 days, once every 6 days, once every week, two weeks, three weeks, four weeks or even a month. The application of the combination/s, composition/s and kit/s of the invention or of any component thereof may last up to a day, two days, three days, four days, five days, six days, a week, two weeks, three weeks, four weeks, a month, two months three months or even more. Specifically, application may last from one day to one month. Most specifically, application may last from one day to 7 days.

Typical delivery routes for the compositions of the invention include parenteral administration, e.g., intradermal, intramuscular or subcutaneous delivery. Other routes include oral administration, intranasal, intramuscular and mucosal administration (such as intranasal, oral, intratracheal, and ocular).

The pharmaceutical compositions of the invention can be administered and dosed by the methods of the invention, in accordance with good medical practice, systemically, for example by parenteral, e.g. intravenous, intraperitoneal or intramuscular injection. In another example, the pharmaceutical composition can be introduced to a site by any suitable route including intravenous, subcutaneous, transcutaneous, topical, intramuscular, intraarticular, subconjunctival, or mucosal, e.g. oral, intranasal, or intraocular administration.

Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the active ingredients (e.g., donor cassette and HK-Int variants).

Needle-free injectors are well suited to deliver vaccines to all types of tissues, particularly to skin and mucosa. In some embodiments, a needle-free injector may be used to propel a liquid that contains the vaccine to the surface and into the subject's skin or mucosa. Representative examples of the various types of tissues that can be treated using the invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof. “Parenteral administration” that is also contemplated by the invention includes subcutaneous injections, submucosal injections, intravenous injections, intramuscular injections, intrasternal injections, transcutaneous injections, and infusion. Injectable preparations (e.g., sterile injectable aqueous or oleaginous suspensions) can be formulated according to the known art using suitable excipients, such as vehicles, solvents, dispersing, wetting agents, emulsifying agents, and/or suspending agents. These typically include, for example, water, saline, dextrose, glycerol, ethanol, corn oil, cottonseed oil, peanut oil, sesame oil, benzyl alcohol, benzyl alcohol, 1,3-butanediol, Ringer's solution, isotonic sodium chloride solution, bland fixed oils (e.g., synthetic mono- or diglycerides), fatty acids (e.g., oleic acid), dimethyl acetamide, surfactants (e.g., ionic and non-ionic detergents), propylene glycol, and/or polyethylene glycols. Excipients also may include small amounts of other auxiliary substances, such as pH buffering agents.

In yet another aspect, the invention relates to a method for replacing at least one target nucleic acid sequence of interest with at least one a replacement-sequence, by site specific recombination of DNA in at least one eukaryotic cell, the method comprising the step of contacting the cell with at least the following components (a) and (b). More specifically, contacting the cells with (a) at least one nucleic acid molecule or nucleic acid cassette comprising a replacement-sequence flanked by a first and a second Int recognition sites. In some embodiments, the first site attP1 may comprise a first overlap sequence O1 and the second site attP2 may comprise a second overlap sequence O2. In yet some other embodiments, the first O1 and the second O2 overlap sequences may be different, each consisting of seven nucleotides, the O1 may be identical to an overlap sequence O1 comprised within a first Int recognition site attE1 in a eukaryotic cell and the O2 may be identical to an overlap sequence O2 comprised within a second Int recognition site attE2 in the eukaryotic cell. In other embodiments, the eukaryotic recognition sites attE1 and attE2 flank a target nucleic acid sequence of interest or any fragment thereof in the eukaryotic cell. The O1 and O2 overlap sequences are each flanked by a first E and a second E′ Int binding sites. In some embodiments, the first binding sites E may comprise the sequence of C1-T2-T3-W4, as denoted by SEQ ID NO. 16, and the second binding sites E′ may comprise the sequence of A12-A13-A14-G15, as denoted by SEQ ID NO. 17. The cells are further contacted with (b), at least one HK-Int variant and/or mutated molecule or any functional fragments or peptides thereof, any nucleic acid molecule comprising a sequence encoding said HK-Int variant and/or mutated molecule or any vector, vehicle, matrix, nano- or micro-particle comprising the same. In some embodiments, the HK-Int variant and/or mutated molecule comprise at least one substituted amino acid residue in at least one of the CB, ND and CD domains of the HK-Int.

It should be understood that the cells may be contacted by the methods of the invention with the components (a) and (b) or with any composition or kit/s or system/s comprising the components of (a) and (b).

In yet some further embodiments, the sequence encoding the at least one HK-Int variants of the invention is used as component (b). In such case, it should be appreciated that the nucleic acid molecule (e.g., donor cassette) of (a), that comprise the replacement sequence, and the nucleic acid sequence of component (b), that encodes the HK-Int variant, may be provided either in separate vectors or cassettes, or alternatively, in one vector, plasmid or cassette. Specifically, in one cassette or construct that comprises nucleic acid sequence that encodes the HK-Int variant of the invention, and further comprises the replacement sequence flanked by the appropriate attP1 and attP2 sites, as discussed above.

The method may thereby allow replacement of the target nucleic acid sequence of interest that may be any target gene or any fragment thereof flanked by the attE1 and attE2 recognition sites in the eukaryotic cell, with the replacement sequence provided by the invention, specifically, by the donor nucleic acid cassettes of the invention.

In some particular embodiments, the HK-Int mutated molecule and/or variant of the method of the invention may comprise at least one substitution at any position of residues 174, 278, 43, 319, 134, 149, 215, 264, 303, 309, 336 of the amino acid sequence of the Wild type HK-Int molecule as denoted by SEQ ID NO. 13 and any combinations thereof. In some specific embodiments, the HK-Int mutated molecule and/or variant of the method of the invention may comprise at least one substitution at the CB domain. In more specific embodiments, the HK-Int mutated molecule and/or variant may comprise at least one substitution at position 174 of the Wild type HK-Int molecule as denoted by SEQ ID NO. 13. In further specific embodiments, the HK-Int mutated molecule and/or variant of the method of the invention may comprise at least one substitution replacing E with K at position 174 of the Wild type HK-Int molecule as denoted by SEQ ID NO. 13.

In some particular embodiments, the HK-Int mutated molecule of the method of the invention may comprise a the amino acid sequence as denoted by SEQ ID NO. 14 or any functional fragments, variants, fusion proteins or derivatives. In yet some further embodiments, the Int mutant or variant of the methods of the invention may comprise a substitution of amino acid residue at position 278, specifically, replacing D278 with K. In some embodiments, such mutant comprise the amino acid sequence as denoted by SEQ ID NO.182, or any derivatives, homologs, fusion proteins or variants thereof.

In some particular embodiments, the HK-Int variants or mutated molecules used by the methods of the invention may comprise a the amino acid sequence as denoted by any one of SEQ ID NO. 14, 42, 44, 46, 48, 83, 85, 87, 89, 182, 184, 185, 180, 188, 190, 192, 223 or any functional fragments, variants, fusion proteins or derivatives thereof. In yet some further embodiments, the nucleic acid sequence encoding the HK-Int variant used by the methods of the invention may comprise the nucleic acid sequence as denoted by any one of SEQ ID NO. SEQ ID NO. 15, 43, 45, 47, 49, 82, 84, 86, 88, 186, 187, 181, 183, 189, 191, 193, 224, or any functional fragments, variants, or derivatives thereof.

Site-specific recombination reaction is based on the integrase specific recognition sites located both on the first plasmid and in the eukaryotic cell, namely, the first Int attP₁ and the second attP₂ sequences flanking the replacement-sequence carried on the first plasmid and the first and second Int attE₁ and attE₂ nucleic acid sequences flanking the target nucleic acid sequence of interest or any fragment thereof in a eukaryotic cell.

The site-specific recombination reaction mediated by the integrase, specifically, any one of the HK-Int variant and/or mutated molecule of the invention, used by any of the methods of the invention, results in the replacement of the target nucleic acid sequence of interest in a eukaryotic cell by the replacement-sequence carried on the first plasmid (also indicated herein as a nucleic acid cassette, or donor cassette), forming the product schematically represented by E₁-O₁-P′₁-replacement-gene-E₂-O₂-P′₂ (where O₁ and O₂ are different, each is identical to the corresponding O sequence in the target eukaryotic genome). As indicated above, the nucleic acid sequences denoted by P₁ and P₂ (as denoted by the nucleic acid sequences SEQ ID NO. 100) and P′₁ and P′₂ (as denoted by SEQ ID NO. 101) originate from the nucleic acid molecule of (a), while the nucleic acid sequences denoted by E₁, E₂ and E′₁ and E′₂ (as denoted by the nucleic acid sequences SEQ ID NO. 16 and SEQ ID NO. 17, respectively) originate from the eukaryotic cell. Still further, it should be noted that in some embodiments, the P and P′ sequences that may be used by the invention may comprise the nucleic acid sequences as denoted by SEQ ID NO. 213 and 214, respectively. Accordingly, a donor cassette contacted by the methods of the invention with the target cells comprise the replacement sequence as flanked by attP1 and attP2 sites that comprise the O1 and O2 sequences, respectively. These O1 and O2 are different from each other, and are identical to O1 and O2 sites in the target sequence in the target eukaryotic cell. Still further, in some embodiments, the first attP1 and a second attP2 sites that flank the replacement sequence in the nucleic acid cassette of the invention may comprise P and P′ sequences that flank the “o” sequence. Such P sequence may comprise the sequence of any one of SEQ ID NO. 100, 213, 240 or 241, and the P′ sequence may comprise the sequence of any one of SEQ ID NO. 101, 214, 242, 243 or 244. It should be noted that any combination of the P and P′ sequences in an attP sites is encompassed by the invention.

As indicated above, the method of the invention involves contacting or introducing the nucleic acid molecule/s of (a) and the Int variant or nucleic acid sequence encoding said variant, in accordance with (b) within at least one eukaryotic cell. This step therefore may involve contacting the cell at least with the elements or components of (a) and (b). The term “contacting” means to bring, put, incubate or mix together. More specifically, in the context of the present invention, the term “contacting” includes all measures or steps, which allow the HK-Int mutant, or nucleic acid molecules, vectors, vehicles, compositions or systems of the invention such that they are in direct or indirect contact with the target cell/s.

To induced DNA integration either in vitro or in vivo, the nucleic acid molecules of the invention may be provided to and/or contacted with the target cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which may be repeated with a frequency of about every day to about every 4 days, e.g., every 1.5 days, every 2 days, every 3 days, or any other frequency from about every day to about every four days. The nucleic acid molecules may be provided to the target cells one or more times, e.g. one time, twice, three times, or more than three times, and the cells allowed to incubate with the nucleic acid molecules for some amount of time following each contacting event e.g. 16-24 hours.

As noted above, in some embodiments, the nucleic acid molecule as well as systems/kits and compositions thereof used by the methods of the invention may be comprised within a nucleic acid cassette or vector, specifically, any of the nucleic acid cassettes disclosed by the invention. Vectors may be provided directly to the subject cells thereby being contacted with the cell/s. In other words, the cells are contacted with vectors comprising the nucleic acid molecules of the invention that comprise the nucleic acid sequence of interest such that the vectors are taken up by the cells. Methods for contacting cells with nucleic acid vectors that are plasmids, such as electroporation, calcium chloride transfection, and lipofection, are well known in the art. DNA can be introduced as naked nucleic acid, as nucleic acid complexed with an agent such as a liposome or poloxamer, or can be delivered by viruses (e.g., adenovirus, AAV).

As used herein, the term “introducing the DNA molecules of (a) and (b) (in case nucleic acid sequence encoding the HK-Int variant is used as component (b)) into said eukaryotic cell” may refer in some embodiments, to a transfection procedure, meaning the introduction of a nucleic acid, e.g., an expression vector, or a replicating vector, into recipient cells by nucleic acid-mediated gene transfer. Transfection of eukaryotic cells may be either transient or stable, and is accomplished by various ways known in the art.

For example, transfection of eukaryotic cells may be chemical, e.g. via a cationic polymer (such as DEAE-dextran, polyethyleneimine, dendrimer, polybrene, calcium), calcium phosphate (e.g. phosphate, lipofectin, DOTAP, lipofectamine, CTAB/DOPE, DOTMA) or via a cationic lipid. Transfection of eukaryotic cells may also be physical, e.g. via a direct injection (for example, by Micro-needle, AFM tip, Gene Gun, Amaxa Nucleofector), via biolistic particle delivery (for example, phototransfection, Magnetofection), or via electroporation, laser-irradiation, sonoporation or a magnetic nanoparticle.

In some specific embodiments, the first overlap sequence O1 and the second overlap sequence of the target sequence in accordance with the method of the invention may comprise a nucleic acid sequence as denoted by any one of SEQ ID NO. 94, SEQ ID NO. 95 (DMD), SEQ ID NO. 98 and SEQ ID NO. 99, SEQ ID NO. 127 and SEQ ID NO. 128 (CFTR), as well as the nucleic acid sequences as denoted by SEQ ID NO. 109, 111, 113, 115 (DMD), SEQ ID NO. 117, 70, 71, 73, 131, 132 (CTNS), and SEQ ID NO. 104, SEQ ID NO. 105 (SCN1A). In some embodiments, the O1 and the O2 may be different.

In some further embodiments, the first overlap sequence O1 and the second overlap sequence of the method of the invention may comprise a nucleic acid sequence as denoted by any one of SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 54, SEQ ID NO. 55, SEQ ID NO. 58, SEQ ID NO.59, SEQ ID NO. 62, SEQ ID NO.63, SEQ ID NO.66, SEQ ID NO. 67, SEQ ID NO. 102, SEQ ID NO. 103, SEQ ID NO. 106, SEQ ID NO. 107, SEQ ID NO. 18, SEQ ID NO. 19, SEQ ID NO. 20, SEQ ID NO. 21, SEQ ID NO. 104, SEQ ID NO. 105 and SEQ ID NO. 181. It should be understood that any of the nucleic acid sequences that comprise the at least one replacement sequence flanked by the appropriate attP1 and attP2 sites, as disclosed by the invention in connection with other aspects of the invention, are also applicable in the present aspect as well and each forms an independent embodiment of the invention. Accordingly, a donor cassette contacted by the methods of the invention with the target cells comprise the replacement sequence as flanked by attP1 and attP2 sites that comprise the O1 and O2 sequences, respectively. These O1 and O2 are different from each other, and are identical to O1 and O2 sites in the target sequence in the target eukaryotic cell. More specifically, attP sites that comprise the P sequence as denoted by SEQ ID NO. 213 and the P′ sequence as denoted by SEQ ID NO. 214, that flank any of the overlap “O” sequences disclosed by the invention. Still further, in some embodiments, the first attP1 and a second attP2 sites that flank the replacement sequence in the nucleic acid cassette of the invention may comprise P and P′ sequences that flank the “o” sequence. Such P sequence may comprise the sequence of any one of SEQ ID NO. 100, 213, 240 or 241, and the P′ sequence may comprise the sequence of any one of SEQ ID NO. 101, 214, 242, 243 or 244. It should be noted that any combination of the P and P′ sequences in an attP sites is encompassed by the invention.

In yet some embodiments, the replacement sequence relevant to the method of the invention may comprises a nucleic acid sequence that differs in at least one nucleotide from the target nucleic acid sequence of interest or any fragments thereof. As noted above, such replacement nucleic acid sequence provided by the method of the invention may replace a corresponding target nucleic acid sequence in a eukaryotic cell. Such target nucleic acid sequence may comprise at least one coding and/or non-coding sequences, or alternatively, may comprise or may be comprised within a target nucleic acid sequence of interest or ay fragment thereof. In some embodiments, the target nucleic acid sequence may comprise a target gene or any fragment thereof that may display aberrant expression or function that may be associated directly or indirectly with at least one pathologic condition. In more particular embodiments, the target nucleic acid sequence may comprise at least one mutation that is connected or associated with a pathologic disorder. Thus, in some embodiments, replacement of such target sequence (a gene or fragment thereof), or any non-coding sequence with the replacement nucleic acid sequence encompassed by the invention (e.g., a corresponding gene or fragments thereof that differs in at least one nucleotide from the target nucleic acid sequence and display normal expression and function) provided by the method of the invention using RCME.

In some further embodiments, the target nucleic acid sequence of interest in the eukaryotic cell replaced by the replacement sequence provided by the methods of the invention may comprise or comprised within the DMD gene or any fragments thereof. Such target nucleic acid sequence is flanked by a first Int recognition site attE1 comprising the nucleic acid sequence as denoted by SEQ ID NO. 92 (DMD2) and a second Int recognition site attE2 comprising the nucleic acid sequence as denoted by SEQ ID NO. 93 (DMD3). In some embodiments, the O1 comprises the nucleic acid sequence as denoted by SEQ ID NO. 94 and said O2 comprises the nucleic acid sequence as denoted by SEQ ID NO. 95. Still further, in some embodiments, other DMD fragments that should be replaced by the methods of the invention, may be flanked by any of the attE sequence designated herein as DMD4, having the sequence of SEQ ID NO. 108 (with an O sequence as denoted by SEQ ID NO. 109), DMD5, having the sequence of SEQ ID NO. 110 (with an O sequence as denoted by SEQ ID NO. 111), DMD6, having the sequence of SEQ ID NO. 112 (with an O sequence as denoted by SEQ ID NO. 113) or DMD7, having the sequence of SEQ ID NO. 114 (with an O sequence as denoted by SEQ ID NO. 115). Non limiting examples for replacement nucleic acid sequence suitable for DMD, are disclosed herein above in connection with other aspects of the invention, specifically, the replacement sequences that comprise the nucleic acid sequence as denoted by SEQ ID NO. 217 and 218, or any variants or derivatives thereof. Accordingly, a donor cassette contacted by the methods of the invention with the target cells comprise the replacement sequence as flanked by attP1 and attP2 sites that comprise the O1 and O2 sequences, respectively. These O1 and O2 are different from each other, and are identical to O1 and O2 sites in the target sequence in the target eukaryotic cell. Still further, in some embodiments, the first attP1 and a second attP2 sites that flank the replacement sequence in the nucleic acid cassette (donor cassette) of the invention may comprise P and P′ sequences that flank the “o” sequence. Such P sequence may comprise the sequence of any one of SEQ ID NO. 100, 213, 240 or 241, and the P′ sequence may comprise the sequence of any one of SEQ ID NO. 101, 214, 242, 243 or 244. It should be noted that any combination of the P and P′ sequences in an attP sites is encompassed by the invention.

In some further embodiments, the target nucleic acid sequence of interest in the eukaryotic cell replaced by the method of the invention may comprise or comprised within the CFTR gene or any fragments thereof, flanked by a first Int recognition site attE1 comprising the nucleic acid sequence as denoted by SEQ ID NO. 96 and a second Int recognition site attE2 comprising the nucleic acid sequence as denoted by SEQ ID NO. 97. The O1 comprises the nucleic acid sequence as denoted by SEQ ID NO. 98 and said O2 comprises the nucleic acid sequence as denoted by SEQ ID NO. 99. Still further, in some embodiments, other CFTR fragments that should be replaced, may be flanked by any of the attE sequence designated herein as CFTR3, having the sequence of SEQ ID NO. 125 (with an O sequence as denoted by SEQ ID NO. 127) and CFTR 4, having the sequence of SEQ ID NO. 126 (with an O sequence as denoted by SEQ ID NO. 128). Non limiting examples for replacement nucleic acid sequence suitable for CFTR, are disclosed herein above in connection with other aspects of the invention, specifically, the replacement sequence that comprise the nucleic acid sequence as denoted by SEQ ID NO. 215 and 216, or any variants or derivatives thereof. Accordingly, a donor cassette contacted by the methods of the invention with the target cells comprise the replacement sequence as flanked by attP1 and attP2 sites that comprise the O1 and O2 sequences, respectively. These O1 and O2 are different from each other, and are identical to O1 and O2 sites in the target sequence in the target eukaryotic cell. Still further, in some embodiments, the first attP1 and a second attP2 sites that flank the replacement sequence in the nucleic acid cassette (donor cassette) of the invention may comprise P and P′ sequences that flank the “o” sequence. Such P sequence may comprise the sequence of any one of SEQ ID NO. 100, 213, 240 or 241, and the P′ sequence may comprise the sequence of any one of SEQ ID NO. 101, 214, 242, 243 or 244. It should be noted that any combination of the P and P′ sequences in an attP sites is encompassed by the invention.

In some other embodiments, the target nucleic acid sequence of interest of the eukaryotic cell replaced by the method of the invention may comprise or comprised within the human CTNS nucleic acid sequence or any fragment thereof, flanked by a first Int recognition site attE₁ comprising the nucleic acid sequence as denoted by SEQ ID NO. 68 and a second Int recognition site attE₂ comprising the nucleic acid sequence as denoted by SEQ ID NO. 69. In some embodiments, the O₁ may comprise the nucleic acid sequence as denoted by SEQ ID NO. 70 and O₂ may comprise the nucleic acid sequence as denoted by SEQ ID NO. 71. It should be noted that mutated forms of the CTNS gene are associated with Cystinosis.

In some other embodiments, the target nucleic acid sequence of interest of the eukaryotic cell replaced by the method of the invention may comprise or comprised within the human CTNS gene or any fragment thereof, flanked by a first Int recognition site attE₁ comprising the nucleic acid sequence as denoted by SEQ ID NO. 68 and a second Int recognition site attE₂ comprising the nucleic acid sequence as denoted by SEQ ID NO. 72. In some embodiments, the O₁ may comprise the nucleic acid sequence as denoted by SEQ ID NO. 70 and O₂ may comprise the nucleic acid sequence as denoted by SEQ ID NO. 73.

In some other embodiments, the target nucleic acid sequence of interest of the eukaryotic cell replaced by the method of the invention may comprise or comprised within the human CTNS gene or any fragment thereof, flanked by a first Int recognition site attE₁ comprising the nucleic acid sequence as denoted by SEQ ID NO. 69 and a second Int recognition site attE₂ comprising the nucleic acid sequence as denoted by SEQ ID NO. 72. In some embodiments, the O₁ may comprise the nucleic acid sequence as denoted by SEQ ID NO. 71 and O₂ may comprise the nucleic acid sequence as denoted by SEQ ID NO. 73.

In yet some further embodiments, the target nucleic acid sequence of interest replaced by the method of the invention may comprise or comprised within the human CTNS gene or any fragment thereof. Such nucleic acid sequence of interest is flanked by a first Int recognition site attE₁ comprising the nucleic acid sequence as denoted by SEQ ID NO. 72 (CTNS4) and a second Int recognition site attE₂ comprising the nucleic acid sequence as denoted by SEQ ID NO. 116 (CTNS1). In some embodiments, the O₁ may comprise the nucleic acid sequence as denoted by SEQ ID NO. 73 and O₂ may comprise the nucleic acid sequence as denoted by SEQ ID NO. 117.

In some other embodiments, the target nucleic acid sequence of interest of the eukaryotic cell replaced by the method of the invention may comprise or comprised within the human CTNS gene or any fragment thereof, flanked by a first Int recognition site attE₁ comprising the nucleic acid sequence as denoted by SEQ ID NO. 129 (CTNS A) and a second Int recognition site attE₂ comprising the nucleic acid sequence as denoted by SEQ ID NO. 130 (CTNS D). In some embodiments, the O₁ may comprise the nucleic acid sequence as denoted by SEQ ID NO. 131 and O₂ may comprise the nucleic acid sequence as denoted by SEQ ID NO. 132.

Non limiting examples for replacement nucleic acid sequence suitable for CTNS, are disclosed herein above in connection with other aspects of the invention, specifically, the replacement sequences that comprise the nucleic acid sequence as denoted by SEQ ID NO. 219 and 220, or any variants or derivatives thereof. Accordingly, a donor cassette contacted by the methods of the invention with the target cells comprise the replacement sequence as flanked by attP1 and attP2 sites that comprise the O1 and O2 sequences, respectively. These O1 and O2 are different from each other, and are identical to O1 and O2 sites in the target sequence in the target eukaryotic cell. Still further, in some embodiments, the first attP1 and a second attP2 sites that flank the replacement sequence in the nucleic acid cassette (donor cassette) of the invention may comprise P and P′ sequences that flank the “o” sequence. Such P sequence may comprise the sequence of any one of SEQ ID NO. 100, 213, 240 or 241, and the P′ sequence may comprise the sequence of any one of SEQ ID NO. 101, 214, 242, 243 or 244. It should be noted that any combination of the P and P′ sequences in an attP sites is encompassed by the invention.

In some other embodiments, the target nucleic acid sequence of interest in the eukaryotic cell replaced by the method of the invention may comprise or comprised within the human SCN1A gene or any fragment thereof. Such nucleic acid sequence of interest is flanked by a first Int recognition site attE₁ comprising the nucleic acid sequence as denoted by SEQ ID NO. 120 and a second Int recognition site attE₂ comprising the nucleic acid sequence as denoted by any one of SEQ ID NO. 121, and wherein said O₁ comprises the nucleic acid sequence as denoted by SEQ ID NO. 104 and said O₂ comprises the nucleic acid sequence as denoted by SEQ ID NO. 105. Non limiting examples for replacement nucleic acid sequences suitable for SCN1A, are disclosed herein above in connection with other aspects of the invention, specifically, the replacement sequence that comprise the nucleic acid sequence as denoted by SEQ ID NO. 221 and 222, or any variants or derivatives thereof. Accordingly, a donor cassette contacted by the methods of the invention with the target cells comprise the replacement sequence as flanked by attP1 and attP2 sites that comprise the O1 and O2 sequences, respectively. These O1 and O2 are different from each other, and are identical to O1 and O2 sites in the target sequence in the target eukaryotic cell. Still further, in some embodiments, the first attP1 and a second attP2 sites that flank the replacement sequence in the nucleic acid cassette (donor cassette) of the invention may comprise P and P′ sequences that flank the “o” sequence. Such P sequence may comprise the sequence of any one of SEQ ID NO. 100, 213, 240 or 241, and the P′ sequence may comprise the sequence of any one of SEQ ID NO. 101, 214, 242, 243 or 244. It should be noted that any combination of the P and P′ sequences in an attP sites is encompassed by the invention.

In some other embodiments, the target nucleic acid sequence of interest in the eukaryotic cell replaced by the method of the invention may comprise or comprised within the human HEXA gene or any fragments thereof, flanked by a first Int recognition site AttE₁ comprising the nucleic acid sequence as denoted by SEQ ID NO. 26 and a second Int recognition site attE₂ comprising the nucleic acid sequence as denoted by SEQ ID NO. 27, and O₁ may comprise the nucleic acid sequence as denoted by SEQ ID NO. 18 and O₂ may comprise the nucleic acid sequence as denoted by SEQ ID NO. 19.

In some other embodiments, the target nucleic acid sequence of interest in the eukaryotic cell replaced by the method of the invention may comprise or comprised within the human ATM gene or any fragments thereof, flanked by a first Int recognition site attE₁ comprising the nucleic acid sequence as denoted by SEQ ID NO. 28 and a second Int recognition site attE₂ comprising the nucleic acid sequence as denoted by any one of SEQ ID NO. 29, and O₁ may comprise the nucleic acid sequence as denoted by SEQ ID NO. 20 and O₂ may comprise the nucleic acid sequence as denoted by SEQ ID NO. 21.

In some other embodiments, the target nucleic acid sequence of interest in the eukaryotic cell replaced by the methods of the invention may comprise or comprised within the human ATM gene or any fragments thereof, flanked by a first Int recognition site attE₁ comprising the nucleic acid sequence as denoted by SEQ ID NO. 50 and a second Int recognition site attE₂ comprising the nucleic acid sequence as denoted by any one of SEQ ID NO. 28, and O₁ may comprise the nucleic acid sequence as denoted by SEQ ID NO. 51 and O₂ may comprise the nucleic acid sequence as denoted by SEQ ID NO. 20.

In some other embodiments, the target nucleic acid sequence of interest in the eukaryotic cell replaced by the method of the invention may be the human ATM gene or any fragments thereof, flanked by a first Int recognition site attE₁ comprising the nucleic acid sequence as denoted by SEQ ID NO. 50 and a second Int recognition site attE₂ comprising the nucleic acid sequence as denoted by any one of SEQ ID NO. 29, and O₁ may comprise the nucleic acid sequence as denoted by SEQ ID NO. 51 and O₂ may comprise the nucleic acid sequence as denoted by SEQ ID NO. 21.

In some other embodiments, the target nucleic acid sequence of interest in the eukaryotic cell replaced by the method of the invention may be the human HAEM gene or any fragments thereof, flanked by a first Int recognition site attE₁ comprising the nucleic acid sequence as denoted by SEQ ID NO. 30 and a second Int recognition site attE₂ comprising the nucleic acid sequence as denoted by any one of SEQ ID NO. 31, and wherein O₁ may comprise the nucleic acid sequence as denoted by SEQ ID NO. 22 and O₂ may comprise the nucleic acid sequence as denoted by SEQ ID NO. 23.

In some other embodiments, the target nucleic acid sequence of interest in the eukaryotic cell replaced by the method of the invention may be the human HGPRT gene or any fragments thereof, flanked by a first Int recognition site attE₁ comprising the nucleic acid sequence as denoted by SEQ ID NO. 32 and a second Int recognition site attE₂ comprising the nucleic acid sequence as denoted by any one of SEQ ID NO. 33, and O₁ may comprise the nucleic acid sequence as denoted by SEQ ID NO. 24 and O₂ may comprise the nucleic acid sequence as denoted by SEQ ID NO. 25.

In some other embodiments, the target nucleic acid sequence of interest in the eukaryotic cell replaced by the method of the invention may be the human SOD1 gene or any fragments thereof, flanked by a first Int recognition site attE₁ comprising the nucleic acid sequence as denoted by SEQ ID NO. 52 and a second Int recognition site attE₂ comprising the nucleic acid sequence as denoted by any one of SEQ ID NO. 53, and O₁ may comprise the nucleic acid sequence as denoted by SEQ ID NO. 54 and O₂ may comprise the nucleic acid sequence as denoted by SEQ ID NO. 55.

In some other embodiments, the target nucleic acid sequence of interest in the eukaryotic cell replaced by the method of the invention may be the human TARDBP gene or any fragments thereof, flanked by a first Int recognition site attE₁ comprising the nucleic acid sequence as denoted by SEQ ID NO. 56 and a second Int recognition site attE₂ comprising the nucleic acid sequence as denoted by any one of SEQ ID NO. 57, and O₁ may comprise the nucleic acid sequence as denoted by SEQ ID NO. 58 and O₂ may comprise the nucleic acid sequence as denoted by SEQ ID NO. 59.

In some other embodiments, the target nucleic acid sequence of interest in the eukaryotic cell replaced by the method of the invention may be the human VABP gene or any fragments thereof, flanked by a first Int recognition site attE₁ comprising the nucleic acid sequence as denoted by SEQ ID NO. 60 and a second Int recognition site attE₂ comprising the nucleic acid sequence as denoted by any one of SEQ ID NO. 61, and O₁ may comprise the nucleic acid sequence as denoted by SEQ ID NO. 62 and O₂ may comprise the nucleic acid sequence as denoted by SEQ ID NO. 63.

In some other embodiments, the target nucleic acid sequence of interest in the eukaryotic cell replaced by the method of the invention may be the human C9ORF71 gene or any fragments thereof, flanked by a first Int recognition site attE₁ comprising the nucleic acid sequence as denoted by SEQ ID NO. 64 and a second Int recognition site attE₂ comprising the nucleic acid sequence as denoted by any one of SEQ ID NO. 65, and O₁ may comprise the nucleic acid sequence as denoted by SEQ ID NO. 66 and O₂ may comprise the nucleic acid sequence as denoted by SEQ ID NO. 67.

In some other embodiments, the target nucleic acid sequence of interest in the eukaryotic cell replaced by the method of the invention may be the human COL3A1 gene or any fragment thereof, flanked by a first Int recognition site attE₁ comprising the nucleic acid sequence as denoted by SEQ ID NO. 122 and a second Int recognition site attE₂ comprising the nucleic acid sequence as denoted by SEQ ID NO. 123. In some embodiments, the O₁ may comprise the nucleic acid sequence as denoted by SEQ ID NO. 106 and O₂ may comprise the nucleic acid sequence as denoted by SEQ ID NO. 107.

In some other embodiments, the target nucleic acid sequence of interest in the eukaryotic cell replaced by the method of the invention may be the human NPC1 gene or any fragment thereof, flanked by a first Int recognition site attE₁ comprising the nucleic acid sequence as denoted by SEQ ID NO. 118 and a second Int recognition site attE₂ comprising the nucleic acid sequence as denoted by SEQ ID NO. 119. In some embodiments, the O₁ may comprise the nucleic acid sequence as denoted by SEQ ID NO. 102 and O₂ may comprise the nucleic acid sequence as denoted by SEQ ID NO. 103.

As indicated above, the Int variants provided by the methods of the invention enable site specific recombination facilitating nucleic acid sequence manipulation of eukaryotic cells. A eukaryote cell or eukaryote cells as herein defined refer to cells within an organism that contain complex structures enclosed within membranes. All large complex organisms are eukaryotes, including animals, plants and fungi. Thus eukaryote cells as herein defined may be derived from animals, plants and fungi, for example, but not limited to, insect cells, yeast cells or mammalian cells.

It should be further noted that the HK-Int mutated molecules or nucleic acid molecules, systems and methods of the invention may also be used for genetically modifying plants for food consumption or other needs (e.g. flowers breeding, or enhancing the activity of certain genes).

In some embodiments the method according to the invention is for replacing a target nucleic acid sequence of interest in a eukaryotic cell by a replacement nucleic acid sequence for modifying, improving or enhancing the functional activity of a normal target nucleic acid sequence in a eukaryotic cell. By way of example, methods provided by the invention may be used for replacing a target nucleic acid sequence of interest in a plant cell, thereby genetically modifying or improving a trait is a plant cell.

The present invention also provides a method for gene therapy or a method of curing or treating genetic disorder or condition in a subject in need using site-specific recombination.

The term “gene therapy” as herein defined, refers to the correction of defective genes. The method of the invention is thus suitable for the treatment of diseases caused by the failure of a single gene, or of multiple genes (also referred to as polygenic or chromosomal), provided that the specific mutations resulting in a defective gene or gene are identified. Theoretically, if the dysfunctional gene is replaced with the corresponding healthy one, a cure can be achieved.

The method of the invention is thus suitable for the treatment of diseases caused by the failure of a single gene, or of multiple genes (also referred to as polygenic or chromosomal), provided that the specific mutations resulting in a defective gene or gene are identified. Theoretically, if the dysfunctional gene is replaced with the corresponding healthy one, a cure can be achieved.

Thus, in yet another aspect, the invention relates to a method of curing or treating, preventing, inhibiting, reducing, eliminating, protecting or delaying the onset of a genetic disorder or condition in a subject in need thereof by administering to the subject an effective amount of at least one of: In a first option (i) (a) at least one nucleic acid molecule or nucleic acid cassette comprising a replacement-sequence for at least one nucleic acid sequence in at least one target nucleic acid sequence of interest. The replacement sequence is flanked by a first and a second Int recognition sites. In some embodiments, the first site attP₁ may comprise a first overlap sequence O₁ and the second site attP2 may comprise a second overlap sequence O₂. In another embodiment, the first O₁ and the second O₂ overlap sequences may be different, each consisting of seven nucleotides, the O₁ may be identical to an overlap sequence O₁ comprised within a first Tnt recognition site attE₁ in a cell of the subject and the O2 may be identical to an overlap sequence O2 comprised within a second Int recognition site attE2 in the cell. In other embodiment, the recognition sites attE1 and attE2 flank a target nucleic acid sequence of interest or any fragment thereof in the target cell in the treated subject. The O1 and O2 overlap sequences are each flanked by a first E and a second E′ Int binding sites. In some embodiments, the first binding sites E may comprise the sequence of C1-T2-T3-W4, as denoted by SEQ ID NO. 16, and the second binding sites E′ may comprise the sequence of A12-A13-A14-G15, as denoted by SEQ ID NO. 17; and

(b) at least one HK-Int mutated molecule or any functional fragments or peptides thereof, any nucleic acid molecule comprising a sequence encoding the HK-Int variant and/or mutated molecule or any vector, vehicle, matrix, nano- or micro-particle comprising the same. In some embodiments, this variant or mutated molecule comprise at least one substituted amino acid residue in at least one of the CB, ND and the CD of the Wild type HK-Int molecule.

In another option (ii), the method may involve administering to the subject an effective amount of at least one kit and/or system or composition comprising (a) and (b).

In an option (iii), the method may comprise the steps of administering to the subject an effective amount of a cell comprising (e.g., transduced or transfected with) the nucleic acid molecule of (a), and a HK-Int variant and/or mutated molecule or nucleic acid molecule of (b). It should be understood that the invention further encompasses, in some embodiments thereof, the option of administering any combination of options (i), (ii) and (iii) or any system, kit or composition thereof. In yet some further embodiments, the sequence encoding the at least one HK-Int variants of the invention is used as component (b). In such case, it should be appreciated that the nucleic acid molecule (e.g., donor cassette) of (a), that comprise the replacement sequence, and the nucleic acid sequence of component (b), that encodes the HK-Int variant, may be administered to the subject either in separate vectors or cassettes, or alternatively, in one vector, plasmid or cassette. Specifically, in one cassette or construct that comprises nucleic acid sequence that encodes the HK-Int variant of the invention, and further comprises the replacement sequence flanked by the appropriate attP1 and attP2 sites, as discussed above.

The method of the invention may thereby allow replacement of the target nucleic acid sequence of interest or any fragment thereof flanked by the attE1 and attE2 sites in the cell of the subject, with the replacement sequence.

In some alternative embodiments, the HK-Int mutated molecule and/or variant of the method of the invention may comprise at least one substitution at any position of residues 174, 278, 43, 319, 134, 149, 215, 264, 303, 309, 336 of the amino acid sequence of the Wild type HK-Int molecule as denoted by SEQ ID NO. 13 and any combinations thereof. In some other embodiments, the HK-Int mutated molecule and/or variant may comprise at least one substitution at the CB domain. In some embodiments, the HK-Int mutated molecule and/or variant may comprise at least one substitution at any one of positions 174, 134, 149, specifically, at position 174 of the Wild type HK-Int molecule as denoted by SEQ ID NO. 13. In other embodiments, the HK-Int mutated molecule and/or variant may comprise at least one substitution replacing E with K at position 174 of the Wild type HK-Int molecule as denoted by SEQ ID NO. 13.

In some further embodiments, the HK-Int mutated molecule used by the methods of the invention may comprise a the amino acid sequence as denoted by SEQ ID NO. 14 and any functional fragments, variants, fusion proteins or derivatives thereof. In yet some further embodiments, the method of the invention may use an Int mutant or variant that comprise a substitution of amino acid residue at position 278, specifically, replacing D278 with K. In some embodiments, such mutant comprise the amino acid sequence as denoted by SEQ ID NO.182, or any derivatives, homologs, fusion proteins or variants thereof.

Non-limiting examples for variants useful in the methods of the invention include the variants of any one of SEQ ID NO. 14, 182, 42, 44, 46, 48, 83, 85, 87, 89, 184, 185, 180, 188, 190, 192, 223 any functional fragments, variants, fusion proteins or derivatives thereof.

In some further embodiments, the nucleic acid sequence encoding the HK-Int variant or mutated molecule used by the methods of the invention may comprise a the nucleic acid sequence as denoted by any one of SEQ ID NO. 15, 183, 43, 45, 47, 49, 82, 84, 86, 88, 186, 187, 181, 189, 191, 193, 224 any functional fragments, variants, fusion proteins or derivatives thereof.

In some embodiments, the first overlap sequence O1 and the second overlap sequence used by the methods of the invention may comprise a nucleic acid sequence as denoted by any one of SEQ ID NO. 94, SEQ ID NO. 95 (DMD), SEQ ID NO. 98 and SEQ ID NO. 99, SEQ ID NO. 127 and SEQ ID NO. 128 (CFTR), as well as the nucleic acid sequences as denoted by SEQ ID NO. 109, 111, 113, 115 (DMD), and SEQ ID NO. 117, 70, 71, 73, 131, 132 (CTNS), and the O1 and the O2 may be different.

In some further embodiments, the first overlap sequence O1 and the second overlap sequence of the method of the invention may comprise a nucleic acid sequence as denoted by any one of SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 54, SEQ ID NO. 55, SEQ ID NO. 58, SEQ ID NO.59, SEQ ID NO. 62, SEQ ID NO.63, SEQ ID NO.66, SEQ ID NO. 67, SEQ ID NO. 102, SEQ ID NO. 103, SEQ ID NO. 104, SEQ ID NO. 105, SEQ ID NO. 106, SEQ ID NO. 107.

In yet some other embodiments, the replacement sequence relevant to the methods of the invention may comprise a nucleic acid sequence that differs in at least one nucleotide from the at least one target nucleic acid sequence to be replaced in the a nucleic acid sequence of interest or any fragments thereof.

In some embodiments, the methods of the invention may be useful in the treatment of Duchenne Muscular Dystrophy (DMD). In such embodiments, the target nucleic acid sequence of interest in at least one cell of the subject replaced by the methods of the invention may comprise or comprised within the DMD gene or any fragments thereof. Such target sequence is flanked by a first Int recognition site attE1 comprising the nucleic acid sequence as denoted by SEQ ID NO. 92 (DMD2) and a second Int recognition site attE2 comprising the nucleic acid sequence as denoted by SEQ ID NO. 93 (DMD3). The O1 comprises the nucleic acid sequence as denoted by SEQ ID NO. 94 and said O2 comprises the nucleic acid sequence as denoted by SEQ ID NO. 95. Still further, in some embodiments, other DMD fragments that should be replaced and targeted by the methods of the invention, may be flanked by any of the attE sequence designated herein as DMD4, having the sequence of SEQ ID NO. 108 (with an O sequence as denoted by SEQ ID NO. 109), DMD5, having the sequence of SEQ ID NO. 110 (with an O sequence as denoted by SEQ ID NO. 111), DMD6, having the sequence of SEQ ID NO. 112 (with an O sequence as denoted by SEQ ID NO. 113) or DMD7, having the sequence of SEQ ID NO. 114 (with an O sequence as denoted by SEQ ID NO. 115). In some specific and non-limiting embodiments, a suitable replacement sequence in the nucleic acid molecule or cassette used by the methods of the invention may comprise the nucleic acid sequence as denoted by SEQ ID NO. 217, or any functional fragments, variants, or derivatives thereof. Such replacement sequence is appropriate specifically, when attE1 and attE2 sites comprising the nucleic acid sequence as denoted by SEQ ID NO. 92 and 93 (DMD2 and DMD3) that flank exon 44 in the DMD gene, are targeted in the treated subject or in any cell thereof. In such embodiments, the replacement sequence in the nucleic acid cassette used by methods of the invention, is flanked by attP1 and attP2 sites that comprise the O (overlap sequence) as denoted by SEQ ID NO. 94 and 95, respectively. In yet some further embodiments, a suitable replacement sequence may comprise the nucleic acid sequence as denoted by SEQ ID NO. 218, or any functional fragments, variants, or derivatives thereof. Such universal replacement sequence may be used when any other DMD site, specifically, as disclosed above (e.g., DMD2, DMD3, DMD4, DMD5, DMD6, DMD7), is used. It should be further appreciated that in some embodiments, P and P′ sequences that flank the replacement sequence comprise the nucleic acid sequences as denoted by SEQ ID NO. 213 and 214, respectively. Accordingly, a donor cassette contacted by the methods of the invention with the target cells comprise the replacement sequence as flanked by attP1 and attP2 sites that comprise the O1 and O2 sequences, respectively. These O1 and O2 are different from each other, and are identical to O1 and O2 sites in the target sequence in the target eukaryotic cell. Still further, in some embodiments, the first attP1 and a second attP2 sites that flank the replacement sequence in the nucleic acid cassette of the invention may comprise P and P′ sequences that flank the “o” sequence. Such P sequence may comprise the sequence of any one of SEQ ID NO. 100, 213, 240 or 241, and the P′ sequence may comprise the sequence of any one of SEQ ID NO. 101, 214, 242, 243 or 244. It should be noted that any combination of the P and P′ sequences in an attP sites is encompassed by the invention.

In some embodiments, the methods of the invention may be useful in the treatment of Cystic Fibrosis (CF). In such case, the target nucleic acid sequence of interest in at least one cell of the treated subject targeted by the method of the invention may comprise or comprised within the CFTR gene or any fragments thereof, flanked by a first Int recognition site attE1 comprising the nucleic acid sequence as denoted by SEQ ID NO. 96 (CFTR10) and a second Int recognition site attE2 comprising the nucleic acid sequence as denoted by SEQ ID NO. 97 (CFTR12). The O1 comprises the nucleic acid sequence as denoted by SEQ ID NO. 98 and said O2 comprises the nucleic acid sequence as denoted by SEQ ID NO. 99. Still further, in some embodiments the attE sequence designated herein as CFTR3, having the sequence of SEQ ID NO. 125 (with an O sequence as denoted by SEQ ID NO. 127) and CFTR4, having the sequence of SEQ ID NO. 126 (with an O sequence as denoted by SEQ ID NO. 128). In some specific and non-limiting embodiments, a suitable replacement sequence in the nucleic acid molecule or cassette used by the methods of the invention may comprise the nucleic acid sequence as denoted by SEQ ID NO. 215, or any functional fragments, variants, or derivatives thereof. Such replacement sequence is suitable specifically, when attE1 and attE2 sites comprising the nucleic acid sequence as denoted by SEQ ID NO. 96 and 97 (CFTR10 and CFTR12, respectively) that flank exon 3 in the CFTR gene, are targeted in the treated subject. In such embodiments, the replacement sequence in the nucleic acid cassette used by the methods of the invention, is flanked by attP1 and attP2 sites that comprise the O (overlap sequence) as denoted by SEQ ID NO. 98 and 99. In yet some further embodiments, a suitable replacement sequence may comprise the nucleic acid sequence as denoted by SEQ ID NO. 216, or any functional fragments, variants, or derivatives thereof. Such universal replacement sequence may be used when any other CFTR site, specifically, as disclosed above, is used. It should be further appreciated that in some embodiments, P and P′ sequences that flank the replacement sequence comprise the nucleic acid sequences as denoted by SEQ ID NO. 213 and 214, respectively. Accordingly, a donor cassette contacted by the methods of the invention with the target cells comprise the replacement sequence as flanked by attP1 and attP2 sites that comprise the O1 and O2 sequences, respectively. These O1 and O2 are different from each other, and are identical to O1 and O2 sites in the target sequence in the target eukaryotic cell. Still further, in some embodiments, the first attP1 and a second attP2 sites that flank the replacement sequence in the nucleic acid cassette of the invention may comprise P and P′ sequences that flank the “o” sequence. Such P sequence may comprise the sequence of any one of SEQ ID NO. 100, 213, 240 or 241, and the P′ sequence may comprise the sequence of any one of SEQ ID NO. 101, 214, 242, 243 or 244. It should be noted that any combination of the P and P′ sequences in an attP sites is encompassed by the invention.

In some embodiments, the methods of the invention may be useful in the treatment of Cystinosis. In such case, the target nucleic acid sequence of interest comprises or is comprised within the human CTNS gene or any fragment thereof in at least one cell of the treated subject. The target nucleic acid sequence of interest is flanked by a first Int recognition site attE₁ comprising the nucleic acid sequence as denoted by SEQ ID NO. 116 and a second Int recognition site attE₂ comprising the nucleic acid sequence as denoted by any one of SEQ ID NO. 72, and wherein said O₁ comprises the nucleic acid sequence as denoted by SEQ ID NO. 117 and said O₂ comprises the nucleic acid sequence as denoted by SEQ ID NO. 73. In yet some further alternative embodiments, the target nucleotide sequence is flanked by a first Int recognition site attE₁ comprising the nucleic acid sequence as denoted by SEQ ID NO. 68 and a second Int recognition site attE₂ comprising the nucleic acid sequence as denoted by any one of SEQ ID NO. 72, and O₁ may comprise the nucleic acid sequence as denoted by SEQ ID NO. 70 and O₂ may comprise the nucleic acid sequence as denoted by SEQ ID NO. 73. In some other embodiments, the target nucleic acid sequence of interest in the eukaryotic cell replaced by the method of the invention may be the human CTNS gene or any fragments thereof, flanked by a first Int recognition site attE₁ comprising the nucleic acid sequence as denoted by SEQ ID NO. 69 and a second Int recognition site attE₂ comprising the nucleic acid sequence as denoted by any one of SEQ ID NO. 72, and O₁ may comprise the nucleic acid sequence as denoted by SEQ ID NO. 71 and O₂ may comprise the nucleic acid sequence as denoted by SEQ ID NO. 73. In yet some further embodiments, the target nucleic acid sequence of interest may be the human CTNS gene or any fragment thereof, flanked by a first Int recognition site attE₁ comprising the nucleic acid sequence as denoted by SEQ ID NO. 129 (CTNS A) and a second Int recognition site attE₂ comprising the nucleic acid sequence as denoted by SEQ ID NO. 130 (CTNS D). In some embodiments, the O₁ may comprise the nucleic acid sequence as denoted by SEQ ID NO. 131 and O₂ may comprise the nucleic acid sequence as denoted by SEQ ID NO. 132. In some specific and non-limiting embodiments, a suitable replacement sequence in the nucleic acid molecule or cassette used by the methods of the invention may comprise the nucleic acid sequence as denoted by SEQ ID NO. 219, or any functional fragments, variants, or derivatives thereof. Such replacement sequence is suitable specifically, when attE1 and attE2 sites comprising the nucleic acid sequence as denoted by SEQ ID NO. 72 and 116 (CTNS4 and CTNS1) that flank exons 1 to 3 in the CTNS gene, are targeted in at least one cell of the subject. In such embodiments, the replacement sequence in the nucleic acid cassette used by the methods of the invention, is flanked by attP1 and attP2 sites that comprise the 0 (overlap sequence) as denoted by SEQ ID NO. 73 and 117, respectively. In yet some further embodiments, a suitable replacement sequence may comprise the nucleic acid sequence as denoted by SEQ ID NO. 220, or any functional fragments, variants, or derivatives thereof. Such universal replacement sequence may be used when any other CTNS site, specifically, as disclosed above, is used. It should be further appreciated that in some embodiments, P and P′ sequences that flank the replacement sequence comprise the nucleic acid sequences as denoted by SEQ ID NO. 213 and 214, respectively. Accordingly, a donor cassette contacted by the methods of the invention with the target cells comprise the replacement sequence as flanked by attP1 and attP2 sites that comprise the O1 and O2 sequences, respectively. These O1 and O2 are different from each other, and are identical to O1 and O2 sites in the target sequence in the target eukaryotic cell. Still further, in some embodiments, the first attP1 and a second attP2 sites that flank the replacement sequence in the nucleic acid cassette of the invention may comprise P and P′ sequences that flank the “o” sequence. Such P sequence may comprise the sequence of any one of SEQ ID NO. 100, 213, 240 or 241, and the P′ sequence may comprise the sequence of any one of SEQ ID NO. 101, 214, 242, 243 or 244. It should be noted that any combination of the P and P′ sequences in an attP sites is encompassed by the invention.

In some embodiments, the genetic disorder or condition is SCN1A-related seizure disorder. More specifically, mutated forms of the SCNA1 gene are associated with Dravet Syndrome (DS), Intractable childhood epilepsy with generalized tonic-clonic seizures (ICEGTC), and severe myoclonic epilepsy borderline (SMEB). Thus, in some embodiments, the methods of the invention may be useful in the treatment of at least one of Dravet Syndrome (DS), Intractable childhood epilepsy with generalized tonic-clonic seizures (ICEGTC), and severe myoclonic epilepsy borderline (SMEB).

Accordingly, the target nucleic acid sequence of interest targeted by the method of the invention comprises or is comprised within the human SCN1A gene or any fragment thereof. Such target nucleic acid sequence of interest is flanked by a first Int recognition site attE₁ comprising the nucleic acid sequence as denoted by SEQ ID NO. 120 (SCN1A4) and a second Int recognition site attE₂ comprising the nucleic acid sequence as denoted by any one of SEQ ID NO. 121 (SCN1A1). The O₁ comprises the nucleic acid sequence as denoted by SEQ ID NO. 104 and said O₂ comprises the nucleic acid sequence as denoted by SEQ ID NO. 105, respectively. In some specific and non-limiting embodiments, a suitable replacement sequence in the nucleic acid molecule or cassette used by the methods of the invention may comprise the nucleic acid sequence as denoted by SEQ ID NO. 221, or any functional fragments, variants, or derivatives thereof. Such replacement sequence is suitable specifically, when attE sites comprising the nucleic acid sequence as denoted by SEQ ID NO. 121 and 120 (SCN1A3 and SCN1A4) that flank intron 6 in the SCN1A gene, are targeted in at least one cell of the treated subject. In such embodiments, the replacement sequence in the nucleic acid cassette used by the kits and systems of the invention, is flanked by attP sites that comprise the O (overlap sequence) as denoted by SEQ ID NO. 105 and 104, respectively. In yet some further embodiments, a suitable replacement sequence may comprise the nucleic acid sequence as denoted by SEQ ID NO. 222, or any functional fragments, variants, or derivatives thereof. Such universal replacement sequence may be used when any other SCN1A sites are used. It should be further appreciated that in some embodiments, P and P′ sequences that flank the replacement sequence comprise the nucleic acid sequences as denoted by SEQ ID NO. 213 and 214, respectively. Accordingly, a donor cassette contacted by the methods of the invention with the target cells comprise the replacement sequence as flanked by attP1 and attP2 sites that comprise the O1 and O2 sequences, respectively. These O1 and O2 are different from each other, and are identical to O1 and O2 sites in the target sequence in the target eukaryotic cell. Still further, in some embodiments, the first attP1 and a second attP2 sites that flank the replacement sequence in the nucleic acid cassette of the invention may comprise P and P′ sequences that flank the “o” sequence. Such P sequence may comprise the sequence of any one of SEQ ID NO. 100, 213, 240 or 241, and the P′ sequence may comprise the sequence of any one of SEQ ID NO. 101, 214, 242, 243 or 244. It should be noted that any combination of the P and P′ sequences in an attP sites is encompassed by the invention.

In some other embodiments, the target nucleic acid sequence of interest in at least one cell of the treated subject replaced by the method of the invention may comprise or comprised within the human hexa gene or any fragments thereof, flanked by a first Tnt recognition site AttE₁ comprising the nucleic acid sequence as denoted by SEQ ID NO. 26 and a second Int recognition site attE₂ comprising the nucleic acid sequence as denoted by SEQ ID NO. 27, and O₁ may comprise the nucleic acid sequence as denoted by SEQ ID NO. 18 and O₂ may comprise the nucleic acid sequence as denoted by SEQ ID NO. 19. In some embodiments, such methods may be useful in the treatment of Tay-Sachs disease.

In some other embodiments, the target nucleic acid sequence of interest in the in at least one cell of the treated subject replaced by the method of the invention may comprise or comprised within the human ATM gene or any fragments thereof, flanked by a first Int recognition site attE₁ comprising the nucleic acid sequence as denoted by SEQ ID NO. 28 and a second Int recognition site attE₂ comprising the nucleic acid sequence as denoted by any one of SEQ ID NO. 29, and O₁ may comprise the nucleic acid sequence as denoted by SEQ ID NO. 20 and O₂ may comprise the nucleic acid sequence as denoted by SEQ ID NO. 21.

In some other embodiments, the target nucleic acid sequence of interest in in at least one cell of the treated subject replaced by the method of the invention may be the human ATM gene or any fragments thereof, flanked by a first Int recognition site attE₁ comprising the nucleic acid sequence as denoted by SEQ ID NO. 50 and a second Int recognition site attE₂ comprising the nucleic acid sequence as denoted by any one of SEQ ID NO. 28, and O₁ may comprise the nucleic acid sequence as denoted by SEQ ID NO. 51 and O₂ may comprise the nucleic acid sequence as denoted by SEQ ID NO. 20.

In some other embodiments, the target nucleic acid sequence of interest in the in at least one cell of the treated subject replaced by the method of the invention may be the human ATM gene or any fragments thereof, flanked by a first Int recognition site attE₁ comprising the nucleic acid sequence as denoted by SEQ ID NO. 50 and a second Int recognition site attE₂ comprising the nucleic acid sequence as denoted by any one of SEQ ID NO. 29, and O₁ may comprise the nucleic acid sequence as denoted by SEQ ID NO. 51 and O₂ may comprise the nucleic acid sequence as denoted by SEQ ID NO. 21. In some embodiments, such methods may be useful in the treatment of Ataxia-Telangiectasia (A-T).

In some other embodiments, the target nucleic acid sequence of interest in the in at least one cell of the treated subject replaced by the method of the invention may be the human haem gene or any fragments thereof, flanked by a first Int recognition site attE₁ comprising the nucleic acid sequence as denoted by SEQ ID NO. 30 and a second Int recognition site attE₂ comprising the nucleic acid sequence as denoted by any one of SEQ ID NO. 31, and wherein O₁ may comprise the nucleic acid sequence as denoted by SEQ ID NO. 22 and O₂ may comprise the nucleic acid sequence as denoted by SEQ ID NO. 23. In some embodiments, such methods may be useful in the treatment of Sickle cell anemia.

In some other embodiments, the target nucleic acid sequence of interest in the in at least one cell of the treated subject replaced by the method of the invention may be the human hgprt gene or any fragments thereof, flanked by a first Int recognition site attE₁ comprising the nucleic acid sequence as denoted by SEQ ID NO. 32 and a second Int recognition site attE₂ comprising the nucleic acid sequence as denoted by any one of SEQ ID NO. 33, and O₁ may comprise the nucleic acid sequence as denoted by SEQ ID NO. 24 and O₂ may comprise the nucleic acid sequence as denoted by SEQ ID NO. 25. In some embodiments, such methods may be useful in the treatment of Lesch-Nyhan syndrome (LNS).

In some other embodiments, the target nucleic acid sequence of interest in the in at least one cell of the treated subject replaced by the method of the invention may be the human sod1 gene or any fragments thereof, flanked by a first Int recognition site attE₁ comprising the nucleic acid sequence as denoted by SEQ ID NO. 52 and a second Int recognition site attE₂ comprising the nucleic acid sequence as denoted by any one of SEQ ID NO. 53, and O₁ may comprise the nucleic acid sequence as denoted by SEQ ID NO. 54 and O₂ may comprise the nucleic acid sequence as denoted by SEQ ID NO. 55. In some embodiments, such methods may be useful in the treatment of Amyotrophic lateral sclerosis (ALS).

In some other embodiments, the target nucleic acid sequence of interest in the in at least one cell of the treated subject replaced by the method of the invention may be the human TARDBP gene or any fragments thereof, flanked by a first Int recognition site attE₁ comprising the nucleic acid sequence as denoted by SEQ ID NO. 56 and a second Int recognition site attE₂ comprising the nucleic acid sequence as denoted by any one of SEQ ID NO. 57, and O₁ may comprise the nucleic acid sequence as denoted by SEQ ID NO. 58 and O₂ may comprise the nucleic acid sequence as denoted by SEQ ID NO. 59. In some embodiments, such methods may be useful in the treatment of ALS.

In some other embodiments, the target nucleic acid sequence of interest in in at least one cell of the treated subject replaced by the method of the invention may be the human VABP gene or any fragments thereof, flanked by a first Int recognition site attE₁ comprising the nucleic acid sequence as denoted by SEQ ID NO. 60 and a second Int recognition site attE₂ comprising the nucleic acid sequence as denoted by any one of SEQ ID NO. 61, and O₁ may comprise the nucleic acid sequence as denoted by SEQ ID NO. 62 and O₂ may comprise the nucleic acid sequence as denoted by SEQ ID NO. 63. In some embodiments, such methods may be useful in the treatment of ALS.

In some other embodiments, the target nucleic acid sequence of interest in the in at least one cell of the treated subject replaced by the method of the invention may be the human c9orf71 gene or any fragments thereof, flanked by a first Int recognition site attE₁ comprising the nucleic acid sequence as denoted by SEQ ID NO. 64 and a second Int recognition site attE₂ comprising the nucleic acid sequence as denoted by any one of SEQ ID NO. 65, and O₁ may comprise the nucleic acid sequence as denoted by SEQ ID NO. 66 and O₂ may comprise the nucleic acid sequence as denoted by SEQ ID NO. 67. In some embodiments, such methods may be useful in the treatment of ALS.

In some particular embodiments, the target nucleic acid sequence of interest of the in at least one cell of the treated subject may be the human Niemann-Pick disease, type C1 (NPC1) gene or any fragment thereof. Such fragment may be flanked by a first Int recognition site attE1 comprising the nucleic acid sequence as denoted by SEQ ID NO. 118 and a second Int recognition site attE2 comprising the nucleic acid sequence as denoted by SEQ ID NO. 119. In some embodiments, the O1 of the Int recognition site may comprise the nucleic acid sequence as denoted by SEQ ID NO. 102 and O2 may comprise the nucleic acid sequence as denoted by SEQ ID NO. 103. It should be noted that mutated forms of the NPC1 gene are associated with Niemann-Pick disease. Thus, in some embodiments, such methods may be useful in the treatment of Niemann-Pick disease.

In some other embodiments, the target nucleic acid sequence of interest of the in at least one cell of the treated subject may be the human Collagen alpha-1(III) (COL3A1) gene or any fragment thereof. Such fragment may be flanked by a first Int recognition site attE1 comprising the nucleic acid sequence as denoted by SEQ ID NO. 122 and a second Int recognition site attE2 comprising the nucleic acid sequence as denoted by SEQ ID NO. 123. In some embodiments, the O1 of the Int recognition site may comprise the nucleic acid sequence as denoted by SEQ ID NO. 106 and O2 may comprise the nucleic acid sequence as denoted by SEQ ID NO. 107. It should be noted that mutated forms of the COL3A gene are associated with type III and IV Ehlers-Danlos syndrome and with aortic and arterial aneurysms. Thus, in some embodiments, such methods may be useful in the treatment of type III and IV Ehlers-Danlos syndrome and arterial aneurysms.

It should be appreciated that the methods of the invention enable in vivo insertion of the nucleic acid sequences and/or HK-Int variants of interest into cells of the treated subjects, by administering to the treated subject the HK-Int variant and/or mutated molecules and/or any nucleic acid molecules encoding such variants and in addition the nucleic acid molecules or donor cassettes of the invention that comprise the replacement nucleic acid sequences, as also indicated by options (i) and (ii) above. However, in some alternative embodiments, the insertion of at least one nucleic acid sequences and/or HK-Int variants into a specific locus in cells of the treated subject, may be performed ex vivo, as also illustrated by option (iii). In such option, the targeted insertion of the replacement nucleic acid sequence is performed in cells of an autologous or allogeneic source, that are then administered to the subject.

Still further, in some embodiments, the cells may be of an autologous or allogeneic source.

Thus, in some embodiments, the “host cells” provided herein, specifically, the cells ex vivo and in vivo transduced or transfected with the HK-Int variant and/or mutated molecules and/or the encoding nucleic acid molecules used by the invention, and the donor cassette that comprise the replacement sequence may be cells of an autologous source. The term “autologous” when relating to the source of cells, refers to cells derived or transferred from the same subject that is to be treated by the method of the invention.

In yet some further embodiments, the cells transduced or transfected with the HK-Int variant and/or mutated molecules and/or nucleic acid molecules and the donor cassette that comprise the replacement sequence used by the methods of the invention may be cells of an allogenic source, or even of a syngeneic source.

The term “allogenic” when relating to the source of cells, refers to cells derived or transferred from a different subject, referred to herein as a donor, of the same species. The term “syngeneic” when relating to the source of cells, refers to cells derived or transferred from a genetically identical, or sufficiently identical and immunologically compatible subject (e.g., an identical twin).

The methods of the invention may be useful for replacing a target nucleic acid sequence of interest or any fragment thereof in at least one cell of the treated subject, with a replacement sequence provided by the invention, using recombination. Specifically, recombination mediated by the HK-Int mutants provided by the invention, either in vivo in the treated subject or ex vivo in cells of the subject or of a donor allogeneic subject. There are several types of eukaryotic cells that may be used by the methods of the invention. According to some embodiments, the target cells may be either targeted in vivo, or alternatively, manipulated ex vivo and introduced back to the treated subject. By way of example, target cells may be, but are not limited to, stem cells, e.g. embryonic stem cells, totipotent stem cells, pluripotent stem cells or induced pluripotent stem cells, multipotent progenitor cells and plant cells.

Stem cells are generally known for their three unique characteristics: (i) they have the unique ability to renew themselves continuously; (ii) they have the ability to differentiate into somatic cell types; and (iii) they have the ability to limit their own population into a small number. In mammals, there are two broad types of stem cells, namely embryonic stem cells (ESCs), and adult stem cells. Stem cells may be autologous or heterologous to the subject. In order to avoid rejection of the cells by the subject's immune system, autologous stem cells are usually preferred.

Thus, in some embodiments, the target cells according to the invention may be embryonic stem cells, or human embryonic stem cells (hESCs), that were obtained from self-umbilical cord blood just after birth. Embryonic stem cells are pluripotent stem cells derived from the early embryo that are characterized by the ability to proliferate over prolonged periods of culture while remaining undifferentiated and maintaining a stable karyotype, with the potential to differentiate into derivatives of all three germ layers. hESCs may be also derived from the inner cell mass (ICM) of the blastocyst stage (100-200 cells) of embryos generated by in vitro fertilization. However, methods have been developed to derive hESCs from the late morula stage (30-40 cells) and, recently, from arrested embryos (16-24 cells incapable of further development) and single blastomeres isolated from 8-cell embryos.

In further embodiments, the target cells according to the invention are totipotent stem cells. Totipotent stem cells are versatile stem cells, and have the potential to give rise to any and all human cells, such as brain, liver, blood or heart cells or to an entire functional organism (e.g. the cell resulting from a fertilized egg). The first few cell divisions in embryonic development produce more totipotent cells. After four days of embryonic cell division, the cells begin to specialize into pluripotent stem cells. Embryonic stem cells may also be referred to as totipotent stem cells.

In further embodiments, the target cells according to the invention are pluripotent stem cells. Similar to totipotent stem cells, a pluripotent stem cell refer to a stem cell that has the potential to differentiate into any of the three germ layers: endoderm (interior stomach lining, gastrointestinal tract, the lungs), mesoderm (muscle, bone, blood, urogenital), or ectoderm (epidermal tissues and nervous system). Pluripotent stem cells can give rise to any fetal or adult cell type. However, unlike totipotent stem cells, they cannot give rise to an entire organism. On the fourth day of development, the embryo forms into two layers, an outer layer which will become the placenta, and an inner mass which will form the tissues of the developing human body. These inner cells are referred to as pluripotent cells.

In still further embodiments, the target cells according to the invention are multipotent progenitor cells. Multipotent progenitor cells have the potential to give rise to a limited number of lineages. As a non-limiting example, a multipotent progenitor stem cell may be a hematopoietic cell, which is a blood stem cell that can develop into several types of blood cells, but cannot into other types of cells. Another example is the mesenchymal stem cell, which can differentiate into osteoblasts, chondrocytes, and adipocytes. Multipotent progenitor cells may be obtained by any method known to a person skilled in the art.

In yet further embodiments, the target cells according to the invention are induced pluripotent stem cells. Induced pluripotent stem cells, commonly abbreviated as iPS cells are a type of pluripotent stem cell artificially derived from a non-pluripotent cell, typically an adult somatic cell, even a patient's own. Such cells can be induced to become pluripotent stem cells with apparently all the properties of hESCs. Induction requires only the delivery of four transcription factors found in embryos to reverse years of life as an adult cell back to an embryo-like cell. For example, iPS cells could be used for autologous transplantation in a patient with a rare disease. The mutation or mutations responsible for the patient's disease state could be corrected ex vivo in the iPS cells obtained from the patient as performed by the methods of the invention and the cells may be then implanted back into the patient (i.e. autologous transplantation).

It should be understood that the methods of the invention may replace a target sequence with a replacement sequence in target cells that may be any of the cells disclosed herein. In yet some further embodiments, any of the cells discussed herein may be used by the methods of the invention for ex vivo therapy as disclosed by option (iii) above.

As indicated above, the invention provides methods for curing genetic disorders. Specifically, by replacing a mal functioning or mutated gene or fragment/s thereof that are associated with the genetic condition with a replacement sequence using the methods of the invention. A genetic disorder or condition as herein defined is a disease caused by an abnormality in the DNA sequence of an individual. Abnormalities as used herein refer to a small mutation in a single gene. A genetic disorder or condition may be a heritable disorder and as such may be present from before birth. Other genetic disorders or conditions are caused by new mutations or changes to the DNA.

Based on their genetic contribution, human genetic disorders or conditions can be classified as monogenic (i.e. which involve mutations in a single gene), chromosomal (also referred to as polygenic), or multifactorial genetic diseases. Monogenic diseases are caused by alterations in a single gene.

Proliferative disorders, such as cancer, may also be classified as genetic disorders or conditions, as they may result from a defect in a single or multiple genes. Some non-limiting examples of cancers that are classified as genetic disorders or conditions are FAP (familial adenomatous polyposis) or HNPCC (hereditary non-polyposis colon cancer) and breast or ovarian cancers that are associated with inherited mutations in either the BRCA1 or BRCA2. The latter examples may be classified as polygenic (or chromosomal) genetic disorders. Approximately five to ten percent of cancers are entirely hereditary. Thus, proliferative disorders may also be treated by the method of the invention.

Currently around 4,000 genetic disorders or conditions are known, with more being discovered. Most disorders or conditions are quite rare and affect one person in every several thousands or millions. Interestingly, Cystic fibrosis is one of the most common genetic disorders; around 5% of the population of the United States carry at least one copy of the defective gene.

The method of the invention may also be used for the treatment of orphan diseases. The term “orphan disease” as herein defined refers to a rare disease, which affects a small percentage of the population. Most rare diseases are genetic, and thus are present throughout the person's entire life, even if symptoms do not immediately appear. Many rare diseases appear early in life, and about 30 percent of children with rare diseases will die before reaching their fifth birthday. A disease may be considered rare in one part of the world, or in a particular group of people, but still be common in another. A rare disease was defined in the Orphan Drug Act of 1983 as one that afflicts fewer than 200,000 people in a nation. According to the National Institute of Health, some non-limiting examples of orphan diseases are Cystic fibrosis, Ataxia telangiectasia and Tay-Sachs, to name but few.

In some embodiments, the genetic disorder or condition encompassed by the invention is a monogenic genetic disease, which may be, but is not limited to Duchenne muscular dystrophy, Cystic Fibrosis, Tay-Sachs disease (also known as GM2 gangliosidosis or hexosaminidase A deficiency), Ataxia-Telangiectasia (A-T), Sickle-cell disease (SCD), or sickle-cell anemia (SCA or anemia), Lesch-Nyhan syndrome (LNS, also known as Nyhan's syndrome, Amyotrophic Lateral Sclerosis, Cystinosis, Kelley-Seegmiller syndrome and Juvenile gout), color blindness, Haemochromatosis (or haemosiderosis), Haemophilia, Phenylketonuria (PKU), Phenylalanine Hydroxylase Deficiency disease, Polycystic kidney disease (PKD or PCKD, also known as polycystic kidney syndrome), Alpha-galactosidase A deficiency, Fabry disease, Anderson-Fabry disease, Angiokeratoma Corporis Diffusum, CADASIL (cerebral autosomal dominant arteriopathy with subcortical infarcts and leukoencephalopathy), Cerebral arteriopathy with subcortical infarcts and leukoencephalopathy, Cerebral autosomal dominant ateriopathy with subcortical infarcts and leukoencephalopathy, Carboxylase Deficiency, Multiple (Late-Onset), Cerebroside Lipidosis syndrome, Gaucher's disease, Choreoathetosis self-mutilation hyperuricemia syndrome, Classic Galactosemia, Galactosemia, Crohn's disease, also known as Crohn syndrome and regional enteritis, Incontinentia Pigmenti (also known as “Bloch-Siemens syndrome,” “Bloch-Sulzberger disease,” “Bloch-Sulzberger syndrome” “melanoblastosis cutis,” and “naevus pigmentosus systematicus”), galactosemia Microcephaly, alpha-1 antitrypsin deficiency (Alpha-1), Adenosine deaminase (ADA) deficiency, Severe Combined Immunodeficiency (SCID), neurofibromatosis type 1 (NF1), Wiskott-Aldrich syndrome, Stargardt macular degeneration, Fanconi's anemia, Spinal muscular atrophy (SMA) and Leber's congenital amaurosis (LCA).

According to some embodiments, the method of the invention may be particularly applicable for curing and treating a genetic disorder, that may be a hereditary disease or condition associated with a single gene disorder or with a polygenic disorder.

The term “Hereditary disease” as herein defined refers to a disease or disorder that is caused by defective genes which are inherited from the parents. A hereditary disease may result unexpectedly when two healthy carriers of a defective recessive gene reproduce, but can also happen when the defective gene is dominant. Non-limiting examples of hereditary diseases are Duchenne Muscular Dystrophy (DMD) and Cystic Fibrosis as well as Tay-Sachs, Ataxia-Telangiectasiaand, Lesch-Nyhan syndrome (LNS), Sickle cell anemia, SCN1A related disorders, Amyotrophic lateral sclerosis and Cystinosis.

In some embodiments, the method of the invention may be used for the treatment of a defective gene which is the result of a (sporadic) mutation or mutations. The term “mutation” as herein defined refers to a change in the nucleotide sequence of the genome of an organism. Mutations result from unrepaired damage to DNA or to RNA genomes (typically caused by radiation or chemical mutagens), from errors in the process of replication, or from the insertion or deletion of segments of DNA by mobile genetic elements. Mutations may or may not produce observable (phenotypic) changes in the characteristics of an organism. Mutation can result in several different types of change in the DNA sequence; these changes may have no effect, alter the product of a gene, or prevent the gene from functioning properly or completely. There are generally three types of mutations, namely single base substitutions, insertions and deletions and mutations defined as “chromosomal mutations”.

The term “single base substitutions” as herein defined refers to a single nucleotide base which is replaced by another. These single base changes are also called point mutations. There are two types of base substitutions, namely, “transition” and “transversion”. When a purine base (i.e. Adenosine or Thymine) replaces a purine base or a pyrimidine base (Cytosine, Guanine) replaces a pyrimidine base, the base substitution mutation is termed a “transition”. When a purine base replaces a pyrimidine base or vice-versa, the base substitution is called a “transversion”.

Single base substitutions may be further classified according to their effect on the genome, as follows:

In missense mutations the new base alters a codon, resulting in a different amino acid being incorporated into the protein chain. As a non-limiting example, the disease sickle cell anemia is a result of a single base substitution that is a missense mutation. In sickle cell anemia, the 17th nucleotide of the gene for the beta chain of haemoglobin (haem) is mutated from an ‘a’ to a ‘t’. This changes the codon from ‘gag’ to ‘gtg’, resulting in the 6th amino acid of the chain being changed from glutamic acid to Valine. This alteration to the beta globin gene alters the quaternary structure of haemoglobin, which has a profound influence on the physiology and wellbeing of the individual.

In nonsense mutations the new base changes a codon that specified an amino acid into one of the stop codons (taa, tag, tga). This will cause translation of the mRNA to stop prematurely and a truncated protein to be produced. This truncated protein will be unlikely to function correctly. Nonsense mutations are the molecular basis for between 15% to 30% of all inherited diseases. Some non-limiting examples include Cystic fibrosis, haemophilia, retinitis pigmentosa and duchenne muscular dystrophy.

In silent mutations no change in the final protein product occurs and thus the mutation can only be detected by sequencing the gene. Most amino acids that make up a protein are encoded by several different codons (see genetic code). So, if for example, the third base in the ‘cag’ codon is changed to an ‘a’ to give ‘caa’, a glutamine (Q) would still be incorporated into the protein product, because the mutated codon still codes for the same amino acid. These types of mutations are ‘silent’ and have no detrimental effect.

Mutation may also arise from insertions of nucleic acids into the DNA or from duplication or deletions of nucleic acids therefrom. As herein defined, the term “insertions and deletions” refers to extra base pairs that are added or deleted from the DNA of a gene, respectively. The number of bases can range from a few to thousands. Insertions and deletions of one or two bases or multiples of one or two bases cause, inter alia, frame shift mutations (i.e. these mutations shift the reading frame of the gene). These can have devastating effects because the mRNA is translated in new groups of three nucleotides and the protein being produced may be useless.

Insertions and deletions of three or multiples of three bases may be less serious because they preserve the open reading frame. However, a number of trinucleotide repeat diseases exist including, for example, Huntington's disease and fragile X syndrome.

In Huntington's disease, for example, the repeated trinucleotide is ‘cag’. This adds a string of glutamines to the Huntington protein. The abnormal protein produced interferes with synaptic transmission in parts of the brain leading to involuntary movements and loss of motor control. Genetic disorders (or conditions, diseases) that may be cured by the methods of the invention may be further classified as “recessive” and “dominant” as well as autosomal and X-linked (relating to the position of the gene).

The term “Autosomal dominant disorder” as referred to herein encompasses genetic disorders or diseases, in which only one mutated copy of the gene is required for a person to be affected. Each affected person usually has one affected parent. Some non-limiting examples of autosomal dominant genetic diseases are Huntington's disease, Neurofibromatosis 1, and Marfan syndrome.

The term “autosomal recessive disorder” as referred to herein, encompasses genetic diseases, in which two copies of the gene should be mutated for a person to be affected. An affected person usually has unaffected parents who each carry a single copy of the mutated gene (and are referred to as carriers). Some non-limiting examples of autosomal recessive disorders include Cystic fibrosis, sickle cell anemia, Tay-Sachs disease, spinal muscular atrophy, Sickle-cell disease (SCD) and phenylketonuria (PKU) which is an autosomal recessive metabolic genetic disorder.

The term “X-linked dominant” as herein defined refers to disorders that are caused by mutations in genes on the X chromosome. Males are more frequently affected than females, and the chance of passing on an X-linked dominant disorder differs between men and women. Some X-linked dominant conditions include, but are not limited to Aicardi Syndrome, and Hypophosphatemia. X-linked disorders may also be classified as “recessive X-linked”. Recessive X-linked disorders as herein defined are also caused by mutations in genes on the X chromosome. Males are more frequently affected than females, and the chance of passing on the disorder differs between men and women. Some non-limiting examples of recessive X-linked disorders are Hemophilia A, Duchenne muscular dystrophy, Color blindness, Muscular dystrophy, Androgenetic alopecia and G-6-PD (Glucose-6-phosphate dehydrogenase) deficiency.

Genetic disorders may also be Y-linked. The term “Y-linked disorders” as herein defined refers to genetic diseases that are caused by mutations on the Y chromosome. Only males can get them, and all of the sons of an affected father are affected.

Genetic disorders may also be classified as “Mitochondrial”. The term “Mitochondrial diseases” as herein defined refers to maternal inheritance, and only applies to genes in mitochondrial DNA. Because only egg cells contribute mitochondria to the developing embryo, only females can pass on mitochondrial conditions to their children. A non-limiting example of a mitochondrial genetic disease is Leber's Hereditary Optic Neuropathy (LHON).

In some embodiments, the methods as well as the cells, systems and compositions of the invention may be particularly suitable for curing or treating an hereditary disease or condition such as Duchenne Muscular Dystrophy (DMD), SCN1A-related seizure disorders, cytinosis and Cystic Fibrosis.

According to some specific embodiments, the invention provides a method for curing or treating Duchenne Muscular Dystrophy (DMD) in a subject.

In some embodiments the method of the invention comprises the step of administering to or contacting with at least one cell of the treated subject the following first and second elements or components: (a), at least one nucleic acid molecule or nucleic acid cassette comprising at least one replacement sequence that may comprise a wild type DMD gene or a fragment thereof flanked by a first and a second Int recognition sites. More specifically, the first attP₁ site may comprise a first overlap sequence O₁ as denoted by SEQ ID NO. 94 and the second attP₂ site may comprise a second overlap O₂ sequence as denoted by SEQ ID NO. 95. It should be noted that the first O₁ and second O₂ overlap sequences are different. In more specific embodiments, O₁ is identical to an overlap sequence O₁ comprised within a first Int recognition site attE₁ in at least one cell of said subject and the O₂ is identical to an overlap sequence O₂ comprised within a second Int recognition site attE₂ in this cell. More specifically, attE₁ and attE₂ flank a mutated target sequence comprising or comprised within the DMD gene or a fragment thereof in at least one cell of the subject. In yet more specific embodiments the attE₁ may comprise a nucleic acid sequence as denoted by SEQ ID NO. 92 and the attE₂ may comprise a nucleic acid sequence as denoted by SEQ ID NO. 93. Still further, in some embodiments, other DMD fragments that should be replaced by the methods of the invention, may be flanked by any of the attE sequence designated herein as DMD4, having the sequence of SEQ ID NO. 108 (with an O sequence as denoted by SEQ ID NO. 109), DMD5, having the sequence of SEQ ID NO. 110 (with an O sequence as denoted by SEQ ID NO. 111), DMD6, having the sequence of SEQ ID NO. 112 (with an O sequence as denoted by SEQ ID NO. 113) or DMD7, having the sequence of SEQ ID NO. 114 (with an O sequence as denoted by SEQ ID NO. 115).

The subject is further administered with (b), at least one HK-Int variant and/or mutated molecule or any functional fragments or peptides thereof, any nucleic acid molecule comprising a sequence encoding the HK-Int variant and/or mutated molecule or any vector, vehicle, matrix, nano- or micro-particle comprising the same.

The introduction of both, the nucleic acid cassette that comprise the appropriate replacement sequence and the HK-Int variant of the invention allows replacement of the mutated target sequence that comprise or is comprised within the DMD gene or a fragment thereof in at least one cell of the subject, with at least one replacement sequence that may comprise a wild type DMD gene or a fragment thereof. It should be understood that contacting cells of the treated subject with both (a) and (b) elements may be performed either in vivo, when the first and second elements (a) and (b) are administered to the treated subject, or alternatively, in vitro/ex vivo, where the introduction of the first and second elements (a) and (b), is performed in an autologous or allogeneic cell in vitro. Thus, according to an optionally embodiment, where the recombination is being performed ex-vivo, the method further involves an additional step of re-introducing the at least one cell that was contacted and therefore comprise the replacement sequence and the HK-Int variant, to the subject, thereby curing and treating Duchenne Muscular Dystrophy (DMD).

As used herein, Duchenne muscular dystrophy (DMD) a progressive neuromuscular disorder, is muscle weakness associated with muscle wasting with the voluntary muscles being first affected, especially those of the hips, pelvic area, thighs, shoulders, and calves. Muscle weakness also occurs later, in the arms, neck, and other areas. Calves are often enlarged. Symptoms usually appear before age six and may appear in early infancy.

DMD is caused by a mutation of the dystrophin gene (DMD) at locus Xp21, located on the short arm of the X chromosome. Dystrophin is responsible for connecting the cytoskeleton of each muscle fiber to the underlying basal lamina (extracellular matrix), through a protein complex containing many subunits. The absence of dystrophin permits excess calcium to penetrate the sarcolemma (the cell membrane), leading to mitochondrial dysfunction.

DMD is inherited in an X-linked recessive pattern. Females typically are carriers of the genetic trait while males are affected. Female carriers of an X-linked recessive condition, such as DMD, can show symptoms depending on their pattern of X-inactivation. DMD has an incidence of one in 3,600 male infants. Mutations within the dystrophin gene can either be inherited or occur spontaneously during germline transmission.

According to other specific embodiments, the invention provides methods, as well as mutated integrases, compositions and kits thereof, for curing or treating Cystic Fibrosis in a subject. In some embodiments, the methods of the invention comprises the step of introducing to or contacting with at least one cell of the treated subject the following first and second elements or components: (a), at least one nucleic acid molecule or nucleic acid cassette comprising at least one replacement sequence that may comprise a wild type CFTR gene or a fragment thereof flanked by a first and a second Int recognition sites. More specifically, the first attP₁ site may comprise a first overlap sequence O₁ as denoted by SEQ ID NO. 98 and the second attP2 site may comprise a second overlap O₂ sequence as denoted by SEQ ID NO. 99. It should be noted that the first O₁ and second O₂ overlap sequences are different. In more specific embodiments, O₁ is identical to an overlap sequence O₁ comprised within a first Int recognition site attE₁ in at least one cell of said subject and the O₂ is identical to an overlap sequence O₂ comprised within a second Int recognition site attE₂ in this cell. More specifically, attE₁ and attE₂ flank a target sequence comprising or comprised within a mutated CFTR gene or a fragment thereof in at least one cell of the subject. In yet more specific embodiments the attE₁ may comprise a nucleic acid sequence as denoted by SEQ ID NO. 96 and the attE₂ may comprise a nucleic acid sequence as denoted by SEQ ID NO. 97. Still further, in some embodiments, the first attP₁ site may comprise a first overlap sequence O₁ as denoted by SEQ ID NO. 127 and the second attP2 site may comprise a second overlap O₂ sequence as denoted by SEQ ID NO. 128. In yet more specific embodiments the attE₁ may comprise a nucleic acid sequence as denoted by SEQ ID NO. 125 and the attE₂ may comprise a nucleic acid sequence as denoted by SEQ ID NO. 126. The second element (b), comprise at least one HK-Int variant and/or mutated molecule or any functional fragments or peptides thereof, any nucleic acid molecule comprising a sequence encoding the HK-Int variant and/or mutated molecule or any vector, vehicle, matrix, nano- or micro-particle comprising the same.

The introduction of both, the nucleic acid cassette that comprise the appropriate replacement sequence and the HK-Int variant of the invention allows replacement of the mutated target sequence that comprise or is comprised within the mutated CFTR gene or a fragment thereof in at least one cell of the subject, with a wild type CFTR gene or a fragment thereof. According to an optionally embodiment, where the recombination is being performed ex-vivo, the method further involves an additional step of re-introducing the at least one cell that was contacted and therefore comprise the replacement sequence and the HK-Int variant to the subject, thereby curing and treating Cystic Fibrosis.

Cystic fibrosis (also known as CF or mucoviscidosis, is an autosomal recessive genetic disorder that affects most critically the lungs and also the pancreas, liver, and intestine. It is characterized by abnormal transport of chloride and sodium across an epithelium, leading to thick, viscous secretions. Difficulty in breathing is the most serious symptom and results from frequent lung infections that are treated with antibiotics and other medications.

CF is caused by a mutation in the gene for the protein Cystic fibrosis transmembrane conductance regulator (CFTR). This protein is required to regulate the components of sweat, digestive fluids and mucus. CFTR regulates the movement of chloride and sodium ions across epithelial membranes, such as the alveolar epithelia located in the lungs. Although most people without CF have two working copies of the CFTR gene, only one is needed to prevent Cystic fibrosis due to the disorder's recessive nature. CF develops when neither gene works normally (as a result of mutation) and therefore has autosomal recessive inheritance.

Therefore, in some embodiments, the method of the invention may be used for the treatment of a subject suffering from Cystic fibrosis. The treatment according to the invention may comprise introducing nucleic acid molecules and the Int variants of the invention or any nucleic acid sequence encoding such variants according to the invention to at least one cell of said subject, wherein the nucleic acid molecule provided by the invention comprises a replacement gene which is the desired normal nucleic acid sequence of the CFTR gene or any fragments thereof, and optionally, at least one nucleic acid molecule comprising a sequence encoding at least one HK-Int variant and/or mutated molecule or any functional fragments or peptides thereof, into specific diseased cells in the lungs or the intestine. For example, the nucleic acid molecules as indicated above may be inhaled by the CF patient into the lungs using a nebulizer, where recombination may take place, in vivo, thus enabling translation of a normal CFTR gene.

According to other specific embodiments, the invention provides methods, as well as mutated integrases, compositions and kits thereof, for curing or treating Cytinosis in a subject. In some embodiments, the methods of the invention comprises the step of introducing to or contacting with at least one cell of the treated subject the following first and second elements or components: (a), at least one nucleic acid molecule or nucleic acid cassette comprising a wild type CTNS gene or a fragment thereof flanked by a first and a second Int recognition sites. More specifically, the first attP₁ site may comprise a first overlap sequence O₁ as denoted by SEQ ID NO. 70 and the second attP₂ site may comprise a second overlap O₂ sequence as denoted by SEQ ID NO. 71. It should be noted that the first O₁ and second O₂ overlap sequences are different. In more specific embodiments, O₁ is identical to an overlap sequence O₁ comprised within a first Int recognition site attE₁ in at least one cell of said subject and the O₂ is identical to an overlap sequence O₂ comprised within a second Int recognition site attE₂ in this cell. More specifically, attE₁ and attE₂ flank a mutated CTNS gene or a fragment thereof in at least one cell of the subject. In yet more specific embodiments the attE₁ may comprise a nucleic acid sequence as denoted by SEQ ID NO. 68 and the attE₂ may comprise a nucleic acid sequence as denoted by SEQ ID NO. 69. In other embodiments, the first attP₁ site may comprise a first overlap sequence O₁ as denoted by SEQ ID NO. 70 and the second attP₂ site may comprise a second overlap O₂ sequence as denoted by SEQ ID NO. 73. It should be noted that the first O₁ and second O₂ overlap sequences are different. In more specific embodiments, O₁ is identical to an overlap sequence O₁ comprised within a first Int recognition site attE₁ in at least one cell of said subject and the O₂ is identical to an overlap sequence O₂ comprised within a second Int recognition site attE₂ in this cell. More specifically, attE₁ and attE₂ flank a mutated CTNS gene or a fragment thereof in at least one cell of the subject.

In yet more specific embodiments the attE₁ may comprise a nucleic acid sequence as denoted by SEQ ID NO. 68 and the attE₂ may comprise a nucleic acid sequence as denoted by SEQ ID NO. 72. Still further, the first Int recognition site attE₁ comprising the nucleic acid sequence as denoted by SEQ ID NO. 72 (CTNS4) and a second Int recognition site attE₂ comprising the nucleic acid sequence as denoted by SEQ ID NO. 116 (CTNS1). In some embodiments, the O₁ may comprise the nucleic acid sequence as denoted by SEQ ID NO. 73 and O₂ may comprise the nucleic acid sequence as denoted by SEQ ID NO. 117

The second element (b), comprise at least one HK-Int variant and/or mutated molecule or any functional fragments or peptides thereof, any nucleic acid molecule comprising a sequence encoding the HK-Int variant and/or mutated molecule or any vector, vehicle, matrix, nano- or micro-particle comprising the same.

The introduction of both, the nucleic acid cassette that comprise the appropriate replacement sequence and the HK-Int variant of the invention allows replacement of the mutated target sequence that comprise or is comprised within the mutated target sequence that comprise or is comprised within the CTNS gene or a fragment thereof in at least one cell of the subject, with a wild type CTNS gene or a fragment thereof, provided herein as a replacement sequence. According to an optionally embodiment, where the recombination is being performed ex-vivo, the method further involves an additional step of re-introducing the at least one cell that was contacted and therefore comprise the replacement sequence and the HK-Int variant to the subject, thereby curing and treating Cystinosis.

Cystinosis is a lysosomal storage disease characterized by the abnormal accumulation of the amino acid cystine. It is a genetic disorder that typically follows an autosomal recessive inheritance pattern. It is a rare autosomal recessive disorder resulting from accumulation of free cystine in lysosomes, eventually leading to intracellular crystal formation throughout the body. Cystinosis is the most common cause of Fanconi syndrome in the pediatric age group. Fanconi syndrome occurs when the function of cells in renal tubules is impaired, leading to abnormal amounts of carbohydrates and amino acids in the urine, excessive urination, and low blood levels of potassium and phosphates.

Cystinosis is a genetic disease belonging to the group of lysosomal storage disease disorders. Cystinosis is caused by mutations in the CTNS gene that codes for cystinosin, the lysosomal membrane-specific transporter for cystine. Intracellular metabolism of cystine, as it happens with all amino acids, requires its transport across the cell membrane. After degradation of endocytosed protein to cystine within lysosomes, it is normally transported to the cytosol. But if there is a defect in the carrier protein, cystine is accumulated in lysosomes. As cystine is highly insoluble, when its concentration in tissue lysosomes increases, its solubility is immediately exceeded and crystalline precipitates are formed in almost all organs and tissues.

According to other specific embodiments, the invention provides methods, as well as mutated integrases, compositions and kits thereof, for curing or treating SCN1A-related seizure disorders in a subject. In some embodiments, the methods of the invention comprises the step of introducing to or contacting with at least one cell of the treated subject the following first and second elements or components: (a), at least one nucleic acid molecule or nucleic acid cassette comprising at least one replacement sequence that may comprise a wild type SCN1A gene or a fragment thereof flanked by a first and a second Int recognition sites. More specifically, the first attP₁ site may comprise a first overlap sequence O₁ as denoted by SEQ ID NO. 105 and the second attP₂ site may comprise a second overlap O₂ sequence as denoted by SEQ ID NO. 104. It should be noted that the first O₁ and second O₂ overlap sequences are different. In more specific embodiments, O₁ is identical to an overlap sequence O₁ comprised within a first Int recognition site attE₁ in at least one cell of the subject and the O₂ is identical to an overlap sequence O₂ comprised within a second Int recognition site attE₂ in this cell. More specifically, attE₁ and attE₂ flank a mutated SCN1A gene or a fragment thereof in at least one cell of the subject. In yet more specific embodiments the attE₁ may comprise a nucleic acid sequence as denoted by SEQ ID NO. 121 and the attE₂ may comprise a nucleic acid sequence as denoted by SEQ ID NO. 120. Still further, in some embodiments, the first attP₁ site may comprise a first overlap sequence O₁ as denoted by SEQ ID NO. 105 and the second attP₂ site may comprise a second overlap O₂ sequence as denoted by SEQ ID NO. 104. The second element (b), comprise at least one HK-Int variant and/or mutated molecule or any functional fragments or peptides thereof, any nucleic acid molecule comprising a sequence encoding the HK-Int variant and/or mutated molecule or any vector, vehicle, matrix, nano- or micro-particle comprising the same.

The introduction of both, the nucleic acid cassette that comprise the appropriate replacement sequence and the HK-Int variant of the invention allows replacement of the mutated target sequence that comprise or is comprised within the mutated SCN1A gene or a fragment thereof in at least one cell of the subject, with a wild type SCN1A gene or a fragment thereof. According to an optionally embodiment, where the recombination is being performed ex-vivo, the method further involves an additional step of re-introducing the at least one cell that was contacted and therefore comprise the replacement sequence and the HK-Int variant to the subject, thereby curing and treating SCN1A-related seizure disorders.

SCN1A-related seizure disorders, as used herein are a spectrum that range from simple febrile seizures at the mild end to Dravet syndrome and intractable childhood epilepsy with generalized tonic-clonic seizures that the severe end. A clinical diagnosis of SCN1A-related seizures disorders is difficult because the phenotypes range on a spectrum, even within the same family and many other conditions have epilepsy as a feature. Therefore, a diagnosis relies on molecular testing of the SCN1A gene (2q24). Sequencing of the SCN1A gene detects 73%-92% of mutations. Deletion/duplication analysis of the SCN1A gene detects 8-27% of mutations. Mutations are inherited in an autosomal dominant manner. Phenotypes that are commonly associated with SCN1A-related seizure disorders include febrile seizures (FS), generalized epilepsy with febrile seizures plus (GEFS+), Dravet syndrome, severe myoclonic epilepsy borderline (SMEB), intractable childhood epilepsy with generalized tonic-clonic seizures (ICEGTC), and infantile partial seizures with variable foci. Clinical features associated with SCN1A-related seizure disorders include one or more family members with epilepsy, especially if the epilepsy is of more than one type, febrile seizures, a history of seizures after vaccination, hemiconvulsive seizures, and seizures triggered by environmental factors. SCN1A-related seizure disorders show incomplete penetrance and variable expressivity.

Dravet syndrome or SMEI, previously known as severe myoclonic epilepsy of infancy (SMEI), is a catastrophic type of epilepsy with prolonged seizures that are often triggered by hot temperatures or fever. It is intractable, and hard to treat with anticonvulsant medications. It often begins before 1 year of age. Dravet syndrome has been characterized by prolonged febrile and non-febrile seizures within the first year of a child's life. This disease progresses to other seizure types like myoclonic and partial seizures, psychomotor delay, and ataxia. It is characterized by cognitive impairment, behavioral disorders, and motor deficits. Behavioral deficits often include hyperactivity and impulsiveness, and in more rare cases, autistic-like behaviors. Dravet syndrome is also associated with sleep disorders including somnolence and insomnia. Dravet syndrome is caused by nonsense mutations in the SCN1A gene resulting in a premature stop codon and thus a non-functional protein. This gene normally codes for neuronal voltage-gated sodium channel Na(V)1.1.

The term severe myoclonic epilepsy of infancy borderline (SMEB) is used to designate patients in whom myoclonic seizures or generalized spike and wave activity are absent. It is also used to indicate mild forms of the syndrome.

Intractable childhood epilepsy with generalized tonic-clonic seizures (ICEGTC) is a disorder characterized by generalized tonic-clonic seizures beginning usually in infancy and induced by fever. Seizures are associated with subsequent mental decline, as well as ataxia or hypotonia. Many of the features of ICEGTC overlap those of SMEI, including age at onset, association with fever, intractability, and cognitive decline. Indeed, ICEGTC is considered in the “borderland” of SMEI. However, in ICEGTC, seizures are predominantly generalized tonic-clonic seizures (GTCs) in type, and myoclonic seizures are not present.

Ehlers-Danlos syndromes (EDS) are a group of genetic connective tissue disorders. Symptoms may include loose joints, joint pain, stretchy skin, and abnormal scar formation. These can be noticed at birth or in early childhood. Complications may include aortic dissection, joint dislocations, scoliosis, chronic pain, or early osteoarthritis. EDS occurs due to variations of more than 19 different genes. The specific gene affected determines the type of EDS. Some cases result from a new variation occurring during early development, while others are inherited in an autosomal dominant or recessive manner. Typically, these variations result in defects in the structure or processing of the protein collagen. Diagnosis is often based on symptoms and confirmed with genetic testing or skin biopsy.

Arterial aneurysms are defined as a 50% increase in the normal diameter of the vessel. Clinical symptoms usually arise from the common complications that affect arterial aneurysms-namely, rupture, thrombosis, or distal embolisation. Although the aneurysmal process may affect any large or medium sized artery, the most commonly affected vessels are the aorta and iliac arteries, followed by the popliteal, femoral, and carotid vessels.

In some embodiments, the methods, as well as the mutants, cells, systems, compositions and kits of the invention may be suitable for curing or treating an hereditary disease or condition such as Tay-Sachs disease, Ataxia Telangiectasia (AT) disease, Lesch-Nyhan syndrome, sickle-cell anemia (SCA), Dravet syndrome and Amyotrophic Lateral Sclerosis.

Thus, in some embodiments, the genetic disorder according to the invention is Tay-Sachs disease, also known as GM2 gangliosidosis or hexosaminidase A deficiency. Tay-Sachs is an autosomal recessive genetic disorder. In its most common variant (known as infantile Tay-Sachs disease), it causes a progressive deterioration of mental and physical abilities that commences around six months of age and usually results in death by the age of four. The disease occurs when harmful quantities of cell membrane components (known as gangliosides) accumulate in nerve cells in the brain, eventually leading to the premature death of the cells. There is currently no known cure or treatment for this disease.

Tay-Sachs is caused by a genetic mutation in the hexa gene (hexosaminidase A) on human chromosome 15. A large number of hexa mutations have been discovered to date. hexa mutations are rare and are most seen in genetically isolated populations. Interestingly, these mutations reach significant frequencies in specific populations, e.g. French Canadians of southeastern Quebec and Ashkenazi Jews. Tay-Sachs can occur from the inheritance of either two similar, or two unrelated, causative mutations in the hexa gene.

Thus, in some embodiments, the methods, as well as the mutants, cells, systems, compositions and kits of the invention may be used for the treatment of a subject suffering from Tay-Sachs, thus restoring the normal function of the HEXA gene (i.e. restoring hexosaminidase activity). Since brain cells are able to absorb hexosaminidase from outside the cell, a minimal recovery of functional enzyme in certain cells will have regional beneficial effect on other brain cell as well. Thus, in some embodiments, the genetic disorder according to the invention may be Ataxia-Telangiectasia (A-T), also referred to as Louis-Bar syndrome. A-T is a rare, neurodegenerative inherited disease that causes severe disability. A-T affects many parts of the body, impairs certain areas of the brain, causing difficulty with movement and coordination; weakens the immune system causing a predisposition to infection; and it prevents repair of broken DNA, increasing the risk of cancer. Symptoms of A-T most often first appear in early childhood when children begin to walk. Though they usually start walking at a normal age, they wobble or sway when walking, standing still or sitting. In late pre-school and early school age they develop difficulty moving the eyes in a natural manner from one place to the next. They develop slurred or distorted speech, and swallowing problems. Some have an increased number of respiratory tract infections. Because not all children develop in the same manner or at the same rate, it may be some years before A-T is properly diagnosed, in particular since most children with A-T have stable neurologic symptoms for the first 4-5 years of life. A-T is considered an autosomal recessive human disorder that is a multisystem disease characterized by progressive cerebellar ataxia, oculocutaneous telangiectasia, radio-sensitivity, predisposition to lymphoid malignancies and immunodeficiency, with defects in both cellular and humoral immunity.

The chromosomal instability characteristic of this disease appear to be related to defective activation of cell cycle checkpoints. The ATM gene (Ataxia Telangiectasia Mutated) is related to a family of genes involved in cellular responses to DNA damage and/or cell cycle control. These genes encode large proteins containing a phosphatidylinositol 3-kinase domain, some of which have protein kinase activity. The mutations causing A-T completely inactivate or eliminate the ATM protein. Thus A-T is now realized to be caused by a defect in the ATM gene, which is responsible for managing the cell's response to multiple forms of stress, including double-strand breaks in DNA.

The majority of A-T patients inherit two distinct mutations. More than 500 mutations, spread over the entire coding region have been described for ATM. Most of these changes (80%) in A-T patients are predicted to give rise to truncated proteins, either through nonsense or splicing mutations, or through secondary premature terminations resulting from frame shift mutations. Thus, an attempt to restore normal function to mutant ATM through mutation-targeted therapy would require read-through of the termination codon or concealment of the cryptic splice site. Clearly, taking this approach will necessitate tailoring the plasmids of the invention to the individual mutations causing A-T. Importantly, normal levels of protein should not necessarily be restores, since even low levels of ATM (approximately 5-10%) in some A-T patients result in a considerably milder phenotype. Thus treatment using the plasmids of the invention requires that the ‘corrected’ ATM be induced in the cerebellum where it needs to be effective in restoring normal functioning of Purkinje cells.

Sickle cell anemia also referred to as hemoglobin SS disease (Hb SS) or Sickle cell disease is herein defined as a disorder that affects red blood cells, which utilize hemoglobin to transport oxygen from the lungs to the rest of the body. Hemoglobin molecules comprise two subunits, termed a and (3. Patients with sickle cell disease have a mutation in a gene on chromosome 11 that codes for the R subunit of the hemoglobin protein. As a result, hemoglobin molecules do not form properly, causing red blood cells to be rigid and have a concave shape, while normal red blood cells are round and flexible so they can travel freely through the narrow blood vessels. These fragile, sickle-shaped cells deliver less oxygen to the body's tissues, causing pain and damage to the organs. Sickle cell disease is inherited in an autosomal recessive pattern.

Lesch-Nyhan syndrome (LNS), also known as Nyhan's syndrome, Kelley-Seegmiller syndrome and Juvenile gout. Lesch-Nyhan syndrome (LNS) is a rare inherited disorder caused by a deficiency of the enzyme hypoxanthine-guanine phosphoribosyltransferase (HGPRT), which is produced by mutations in the HPRT gene located on the X chromosome. LNS affects about one in 380,000 live births.

The HGPRT deficiency causes a build-up of uric acid in all body fluids. This results in both hyperuricemia and hyperuricosuria, associated with severe gout and kidney problems.

Neurological signs include poor muscle control and moderate mental retardation. These complications usually appear in the first year of life. Beginning in the second year of life, a particularly striking feature of LNS is self-mutilating behaviors, characterized by lip and finger biting. Neurological symptoms include facial grimacing, involuntary writhing, and repetitive movements of the arms and legs similar to those seen in Huntington disease.

LNS is an X-linked recessive disease. The gene mutation is usually carried by the mother and passed on to her son, although one-third of all cases arise de novo (from new mutations) and do not have a family history. LNS is present at birth in baby boys. Most, but not all, persons with this deficiency have severe mental and physical problems throughout life.

Amyotrophic lateral sclerosis (ALS), also known as motor neurone disease (MND), and Lou Gehrig's disease, is a specific disease which causes the death of neurons controlling voluntary muscles. Some also use the term motor neuron disease for a group of conditions of which ALS is the most common. ALS is characterized by stiff muscles, muscle twitching, and gradually worsening weakness due to muscles decreasing in size. This results in difficulty speaking, swallowing, and eventually breathing.

A defect on chromosome 21, which codes for superoxide dismutase (encoded by the gene SOD1), is associated with about 20% of familial cases of ALS, or about 2% of ALS cases overall. This mutation is believed to be transmitted in an autosomal dominant manner, and has over a hundred different forms of mutation. The most common ALS-causing mutation is a mutant SOD1 gene. A genetic abnormality known as a hexanucleotide repeat was also found in a region called C9orf72, which is associated with ALS combined with frontotemporal dementia (ALS-FTD). TAR DNA-binding protein 43 (TDP-43, transactive response DNA binding protein 43 kDa), is a protein that in humans is encoded by the tardbp gene. A hyper-phosphorylated, ubiquitinated and cleaved form of TDP-43 known as pathologic TDP43 is the major disease protein in Amyotrophic lateral sclerosis (ALS). In addition, mutations in the gene vapb encoding for the Vesicle-associated membrane protein-associated protein B/C may also cause ALS.

In some embodiments, the methods as well as the mutants, cells, systems, compositions and kits of the invention may be applicable for the treatment of alpha-1 antitrypsin deficiency (Alpha-1). The treatment according to the invention comprises delivery of the plasmids of the invention, comprising the desired normal nucleic acid fragment of the SERPINA1 gene, into specific diseased muscle cells, where recombination may take place, in vivo, thus restoring the normal function of the SERPINA1 gene. Alternatively, an Alpha-1 patient's autologous somatic cells may be derived from an Alpha-1 patient and may be induced to become pluripotent stem cells (iPS) and then differentiated into muscle cells.

In yet some further embodiments, the methods as well as the mutants, cells, systems, compositions and kits of the invention may be applicable for the treatment of Leber's congenital amaurosis (LCA). The treatment according to the invention comprises delivery of the nucleic acid molecules of the invention, comprising the desired normal nucleic acid fragment of the any of the genes responsible for the disease (e.g. LCA2), via a sub-retinal injection into the eye, where recombination may take place, in vivo, thereby restoring the normal function of the gene product. In some embodiments, the method of the invention may be applicable for the treatment of Wiskott-Aldrich syndrome. The treatment according to the invention comprises obtaining autologous CD34+ hematopoietic progenitor stem cells (HSCc) from the patient and transfection of said cells with the plasmids of the invention, comprising the desired normal nucleic acid fragment of the WASP gene, to correct the WAS genetic mutation. The treated cells are then re-transplanted into the patient, thereby restoring the normal function of the gene product.

In yet other embodiments, the method of the invention may be applicable for the treatment of Stargardt macular degeneration. The treatment according to the invention comprises delivery of the nucleic acid molecules of the invention comprising the desired normal nucleic acid sequence of the any of the genes that are mutated in Stargardt macular degeneration (e.g. the ABCA4 or ELOVL4 genes), to correct the genetic mutation therein. The delivery may be performed as a subretinal injection, thereby restoring the normal function of the gene product in the patient's eye. In other embodiments, the methods and recombination cassette system of the invention may be used for the treatment of Fanconi's anemia. The treatment according to the invention comprises delivery of the nucleic acid molecules of the invention comprising the desired normal nucleic acid fragment of the any of the genes that are mutated in Fanconi's anemia (e.g. FANCA, FANCB, FANCC, BRCA2, genes), to correct the genetic mutation therein. For example, mutations in FANCC may be corrected by the method of the invention by ex vivo delivery of the plasmids of the invention comprising the desired normal nucleic acid fragment of FANCC to autologous CD34+ hematopoietic progenitor cells obtained from the patient by transfection. The stem cells may then be expanded and re-injected in to the patient's bone marrow, thereby correcting the mutation in the FANCC gene.

Niemann-Pick type C (NPC) disease is an autosomal recessive lipid storage disorder characterized by progressive neurodegeneration. Approximately 95% of cases are caused by mutations in the NPC1 gene, referred to as type C1; 5% are caused by mutations in the NPC2 gene, referred to as type C2. The clinical manifestations of types C1 and C2 are similar because the respective genes are both involved in egress of lipids, particularly cholesterol, from late endosomes or lysosomes. Niemann-Pick disease type C has a highly variable clinical phenotype. Patients with the ‘classic’ childhood onset type C usually appear normal for 1 or 2 years with symptoms appearing between 2 and 4 years. They gradually develop neurologic abnormalities which are initially manifested by ataxia, grand mal seizures, and loss of previously learned speech. Spasticity is striking and seizures, particularly myoclonic jerks, are common. Other features include dystonia, vertical supranuclear gaze palsy, dementia, and psychiatric manifestations. In general, hepatosplenomegaly is less striking than in types A and B, although it can be lethal in some. Cholestatic jaundice occurs in some patients. Foamy Niemann-Pick cells and ‘sea-blue’ histiocytes with distinctive histochemical and ultrastructural appearances are found in the bone marrow.

In further embodiments, the genetic disorder may be a multifactorial genetic disease. Examples of multifactorial genetic diseases include, but are not limited to breast and ovarian cancers that are associated with the BRCA1 or BRCA2 gene, Alzheimer's disease, some forms of colon cancer, e.g. familial adenomatous polyposis (FAP) or hereditary non-polyposis colon cancer (HNPCC) as well as hypothyroidism.

The invention thus provides therapeutic methods for treating variety of genetic and congenital disorders. It is to be understood that the terms “treat”, “treating”, “treatment” or forms thereof, as used herein, mean preventing, ameliorating or delaying the onset of one or more clinical indications of disease activity in a subject having a pathologic disorder. Treatment refers to therapeutic treatment. Those in need of treatment are subjects suffering from a pathologic disorder. Specifically, providing a “preventive treatment” (to prevent) or a “prophylactic treatment” is acting in a protective manner, to defend against or prevent something, especially a condition or disease. The term “treatment or prevention” as used herein, refers to the complete range of therapeutically positive effects of administrating to a subject including inhibition, reduction of, alleviation of, and relief from, a hereditary condition and illness, hereditary condition symptoms or undesired side effects or hereditary disorders. More specifically, treatment or prevention of relapse or recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the immune-related disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described herein.

The terms “delay”, “delaying the onset”, “retard” and all variations thereof are intended to encompass the slowing of the progress and/or exacerbation of a disorder associated with the immune-related disorders and their symptoms slowing their progress, further exacerbation or development, so as to appear later than in the absence of the treatment according to the invention. As indicated above, the methods and compositions provided by the present invention may be used for the treatment of a “pathological disorder” which refers to a condition, in which there is a disturbance of normal functioning, any abnormal condition of the body or mind that causes discomfort, dysfunction, or distress to the person affected or those in contact with that person. It should be noted that the terms “disease”, “disorder”, “condition” and “illness”, are equally used herein.

It should be appreciated that any of the methods and compositions described by the invention may be applicable for treating and/or ameliorating any of the disorders disclosed herein or any condition associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

The present invention relates to the treatment of subjects or patients, in need thereof. By “patient” or “subject in need” it is meant any organism who may be affected by the above-mentioned conditions, and to whom the therapeutic and prophylactic methods herein described are desired, including humans, domestic and non-domestic mammals such as canine and feline subjects, bovine, simian, equine and rodents, specifically, murine subjects. More specifically, the methods of the invention are intended for mammals. By “mammalian subject” is meant any mammal for which the proposed therapy is desired, including human, livestock, equine, canine, and feline subjects, most specifically humans.

It should be noted that any of the administration modes discussed herein in connection with the compositions of the invention, may be applicable for any of the methods of the invention as described in further aspects of the invention. More specifically, administration by parenteral, intraperitoneal, transdermal, pulmonary (for example for CF treatment) (including intranasal), muscular (for example for treating DMD) oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s). In another aspect, the invention relates to an HK-Int variant and/or mutated molecule or any functional fragments or peptides thereof, any nucleic acid molecule comprising a sequence encoding the HK-Int variant and/or mutated molecule or any vector, vehicle, matrix, nano- or micro-particle comprising the same, any composition thereof or any cell transduced or transfected with the HK-Int variant and/or mutated molecule for use in a method for curing or treating, preventing, inhibiting, reducing, eliminating, protecting or delaying the onset of a genetic disorder or condition a genetic disorder in a subject in need thereof.

In some embodiments, the HK-Int variant and/or mutated molecule suitable for use according to the invention may be as the HK-Int variant and/or mutated molecules as defined in the invention, the nucleic acid molecule encoding the HK-Int variant and/or mutated molecule may be as defined in the invention, and the host cell may be as defined according to the invention.

Still further, the invention provides in an additional aspect thereof, nucleic acid molecules comprising at least one replacement-sequence flanked by a first and a second Int recognition sites, said first site attP1 comprises a first overlap sequence O1 and said second site attP2 comprises a second overlap sequence O2, wherein said first O1 and said second O2 overlap sequences are different, each consisting of seven nucleotides, said O1 is identical to an overlap sequence O1 comprised within a first Int recognition site attE1 in a eukaryotic cell and said O2 is identical to an overlap sequence O2 comprised within a second Int recognition site attE2 in said eukaryotic cell, said eukaryotic recognition sites attE1 and attE2 flank a target nucleic acid sequence of interest or any fragment thereof in said eukaryotic cell, wherein said O1 and O2 overlap sequences are each flanked by a first E and a second E′ Int binding sites, wherein said first binding sites E comprise the sequence of C1-T2-T3-W4, as denoted by SEQ ID NO. 16, and said second binding sites E′ comprise the sequence of A12-A13-A14-G15, as denoted by SEQ ID NO. 17. It should be understood, that any of the nucleic acid molecules disclosed by the invention are encompassed by this aspect as well.

The invention encompasses any of the constructs, plasmids, cassettes and vectors disclosed herein by the following examples, each forms a separate embodiment of the invention.

It should be understood that the nucleic acid molecules of the invention may be comprised within any cassette, vehicle or vector as discussed herein before in connection with other nucleic acid molecules provided by the invention.

In some embodiments of the compositions, systems, kits and methods of the invention, the different nucleic acid molecules or cassettes of the invention comprising at least one replacement sequence and are targeted to replace at least one target nucleic acid sequence in the target nucleic acid sequence or fragments thereof, may be combined with any of the HK-Int molecules of the invention and any combinations thereof. More specifically, the compositions, systems, kits and methods of the invention may comprise any of the nucleic acid molecules, and that will replace the target nucleic acid sequence, specifically, any nucleic acid molecules that comprise the 0 sequence of DMD2 site and/or the DMD3 site, may further comprise any HK-Int variant of the invention and any combinations thereof, or any nucleic acid sequence encoding such variant/s. In some specific and non-limiting embodiments when at least one of DMD2 and DMD3 sites are used, suitable HK-Int variants may be any one of E174K/I43F, E174K/R319G, E174K/E278K (specifically for DMD2 sites), and at least one of E174K, E174K/R319G, E174K/E278K, E174K/T43F/R319G variants, specifically when the DMD3 site is used.

Still further, in some embodiments, when at least one of CTNS1 and CTNS4 sites are used, suitable HK-Int variants may be any one of E174K/R319G, E174K/I43F/R319G (specifically for CTNS1), and at least one of E174K, E174K/R319G, E174K/E278K, E174K/I43F (specifically for CTNS4) are used.

In yet some further embodiments, when at least one of CF10 and CF12 sites are used, suitable HK-Int variants may be any one of E174K/I43F, E174K/R319G, E174K/E278K variants (specifically for CF10), and any one of E174K, E174K/R319G, E174K/E278K, E174K/I43F/R319G (specifically for CF12) In further specific embodiments, specifically when SCN1A-3 site is used in the nucleic acid molecules of the invention, the HK-Int variant may be any one of E174K/R319G, E174K/E278K, E174K/I43F/R319G are used.

More specifically, in some embodiments, the vector may be a viral vector. In yet some particular embodiments, such viral vector may be any one of recombinant adeno associated vectors (rAAV), single stranded AAV (ssAAV), self-complementary rAAV (scAAV), Simian vacuolating virus 40 (SV40) vector, Adenovirus vector, helper-dependent Adenoviral vector, retroviral vector and lentiviral vector.

As indicated above, in some embodiments, viral vectors may be applicable in the present invention. The term “viral vector” refers to a replication competent or replication-deficient viral particle which are capable of transferring nucleic acid molecules into a host.

The term “virus” refers to any of the obligate intracellular parasites having no protein-synthesizing or energy-generating mechanism. The viral genome may be RNA or DNA contained with a coated structure of protein of a lipid membrane. Examples of viruses useful in the practice of the present invention include baculoviridiae, parvoviridiae, picornoviridiae, herepesviridiae, poxviridiae, adenoviridiae, picotmaviridiae. The term recombinant virus includes chimeric (or even multimeric) viruses, i.e. vectors constructed using complementary coding sequences from more than one viral subtype.

In some embodiments, the nucleic acid molecules suitable to methods of the invention may be comprised within an Adeno-associated virus (AAV). The term “adenovirus” is synonymous with the term “adenoviral vector”. AAV is a single-stranded DNA virus with a small (˜20 nm) protein capsule that belongs to the family of parvoviridae, and specifically refers to viruses of the genus adenoviridiae. The term adenoviridiae refers collectively to animal adenoviruses of the genus mastadenovirus including but not limited to human, bovine, ovine, equine, canine, porcine, murine and simian adenovirus subgenera. In particular, human adenoviruses includes the A-F subgenera as well as the individual serotypes thereof the individual serotypes and A-F subgenera including but not limited to human adenovirus types 1, 2, 3, 4, 4a, 5, 6, 7, 8, 9, 10, 11 (AdllA and Ad IIP), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91.

Due to its inability to replicate in the absence of helpervirus coinfections (typically Adenovirus or Herpesvirus infections) AAV is often referred to as dependovirus. AAV infections produce only mild immune responses and are considered to be nonpathogenic, a fact that is also reflected by lowered biosafety level requirements for the work with recombinant AAVs (rAAV) compared to other popular viral vector systems. Due to its low immunogenicity and the absence of cytotoxic responses AAV-based expression systems offer the possibility to express genes of interest for months in quiescent cells.

Production systems for rAAV vectors typically consist of a DNA-based vector containing a transgene expression cassette, which is flanked by inverted terminal repeats. Construct sizes are limited to approximately 4.7-5.0 kb, which corresponds to the length of the wild-type AAV genome. rAAVs are produced in cell lines. The expression vector is co-transfected with a helper plasmid that mediates expression of the AAV rep genes which are important for virus replication and cap genes that encode the proteins forming the capsid. Recombinant adeno-associated viral vectors can transduce dividing and non-dividing cells, and different rAAV serotypes may transduce diverse cell types. These single-stranded DNA viral vectors have high transduction rates and have a unique property of stimulating endogenous Homologous Recombination without causing double strand DNA breaks in the host genome.

It should be appreciated that many intermediate steps of the wild-type infection cycle of AAV depend on specific interactions of the capsid proteins with the infected cell. These interactions are crucial determinants of efficient transduction and expression of genes of interest when rAAV is used as gene delivery tool. Indeed, significant differences in transduction efficacy of various serotypes for particular tissues and cell types have been described. Thus, in some embodiments AAV serotype 6 may be suitable for the methods of the invention. In yet some further embodiments, AAV serotype 8 may be suitable for the methods of the invention.

It is believed that a rate-limiting step for the AAV-mediated expression of transgenes is the formation of double-stranded DNA. Recent reports demonstrated the usage of rAAV constructs with a self-complementing structure (scAAV) in which the two halves of the single-stranded AAV genome can form an intra-molecular double-strand. This approach reduces the effective genome size usable for gene delivery to about 2.3 kB, but leads to significantly shortened onsets of expression in comparison with conventional single-stranded AAV expression constructs (ssAAV). Thus, in some embodiments, ssAAV may be applicable as a viral vector by the methods of the invention.

In yet some further embodiments, HDAd vectors may be suitable for the methods of the invention. The Helper-Dependent Adenoviral (HDAd) vectors HDAds have innovative features including the complete absence of viral coding sequences and the ability to mediate high level transgene expression with negligible chronic toxicity. HDAds are constructed by removing all viral sequences from the adenoviral vector genome except the packaging sequence and inverted terminal repeats, thereby eliminating the issue of residual viral gene expression associated with early generation adenoviral vectors. HDAds can mediate high efficiency transduction, do not integrate in the host genome, and have a large cloning capacity of up to 37 kb, which allows for the delivery of multiple transgenes or entire genomic loci, or large cis-acting elements to enhance or regulate tissue-specific transgene expression. One of the most attractive features of HDAd vectors is the long term expression of the transgene.

Still further, in some embodiments, SV40 may be used as a suitable vector by the methods of the invention. SV40 vectors (SV40) are vectors originating from modifications brought to Simian virus-40 an icosahedral papovavirus. Recombinant SV40 vectors are good candidates for gene transfer, as they display some unique features: SV40 is a well-known virus, non-replicative vectors are easy-to-make, and can be produced in titers of 10 (12) IU/ml. They also efficiently transduce both resting and dividing cells, deliver persistent transgene expression to a wide range of cell types, and are non-immunogenic. Present disadvantages of rSV40 vectors for gene therapy are a small cloning capacity and the possible risks related to random integration of the viral genome into the host genome.

In certain embodiments, an appropriate vector that may be used by the invention may be a retroviral vector. A retroviral vector consists of proviral sequences that can accommodate the gene of interest, to allow incorporation of both into the target cells. The vector may also contain viral and cellular gene promoters, to enhance expression of the gene of interest in the target cells. Retroviral vectors stably integrate into the dividing target cell genome so that the introduced gene is passed on and expressed in all daughter cells. They contain a reverse transcriptase that allows integration into the host genome.

In yet some alternative embodiments, lentiviral vectors may be used in the present invention. Lentiviral vectors are derived from lentiviruses which are a subclass of Retroviruses. Commonly used retroviral vectors are “defective”, i.e. unable to produce viral proteins required for productive infection. Rather, replication of the vector requires growth in a packaging cell line. To generate viral particles comprising the nucleic acids sequence of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing the retroviral vectors comprising the nucleic acid molecules of the invention that contains the nucleic acids sequence of interest into packaging cell lines and of collecting the viral particles that are generated by the packaging lines are well known in the art.

In some alternative embodiments, the vector may be a non-viral vector. More specifically, such vector may be in some embodiments any one of plasmid, minicircle and linear DNA.

Nonviral vectors, in accordance with the invention, refer to all the physical and chemical systems except viral systems and generally include either chemical methods, such as cationic liposomes and polymers, or physical methods, such as gene gun, electroporation, particle bombardment, ultrasound utilization, and magnetofection. Efficiency of this system is less than viral systems in gene transduction, but their cost-effectiveness, availability, and more importantly reduced induction of immune system and no limitation in size of transgenic DNA compared with viral system have made them attractive also for gene delivery.

For example, physical methods applied for in vitro and in vivo gene delivery are based on making transient penetration in cell membrane by mechanical, electrical, ultrasonic, hydrodynamic, or laser-based energy so that DNA entrance into the targeted cells is facilitated.

In more specific embodiments, the vector may be a naked DNA vector. More specifically, such vector may be for example, a plasmid, minicircle or linear DNA.

Naked DNA alone may facilitate transfer of a gene (2-19 kb) into skin, thymus, cardiac muscle, and especially skeletal muscle and liver cells when directly injected. It enables also long-term expression. Although naked DNA injection is a safe and simple method, its efficiency for gene delivery is quite low.

Minicircles are modified plasmid in which a bacterial origin of replication (ori) was removed, and therefore they cannot replicate in bacteria.

Linear DNA or Doggybone™ are double-stranded, linear DNA construct that solely encodes an antigen expression cassette, comprising antigen, promoter, polyA tail and telomeric ends.

It should be appreciated that all DNA vectors disclosed herein, may be also applicable for the methods, systems and compositions of the invention.

Still further, it must be appreciated that the invention further provides any vectors or vehicles that comprise any of the nucleic acid molecules disclosed by the invention, as well as any host cell expressing the nucleic acid molecules disclosed by the invention.

It should be understood that any of the viral vectors disclosed herein may be relevant to any of the nucleic acid molecules discussed in other aspects of the invention.

The invention further provides at least one nucleic acid molecule or any nucleic acid cassette or vector thereof for use in a method for curing or treating, preventing, inhibiting, reducing, eliminating, protecting or delaying the onset of a genetic disorder or condition a genetic disorder in a subject in need thereof. In some embodiments, the nucleic acid sequence comprising a replacement-sequence flanked by a first and a second Int recognition sites, said first site attP1, said nucleic acid molecule comprises a first overlap sequence O1 and said second site attP2, comprises a second overlap sequence O2, wherein said first O1 and said second O2 overlap sequences are different, each consisting of seven nucleotides, said O1 is identical to an overlap sequence O1 comprised within a first Int recognition site attE1 in a eukaryotic cell and said O2 is identical to an overlap sequence O2 comprised within a second Int recognition site attE2 in said eukaryotic cell, said eukaryotic recognition sites attE1 and attE2 flank a target nucleic acid sequence of interest or any fragment thereof in said eukaryotic cell. In some embodiments, the first binding sites E may comprise the sequence of C1-T2-T3-W4, as denoted by SEQ ID NO. 16, and the second binding sites E′ may comprise the sequence of A12-A13-A14-G15, as denoted by SEQ ID NO. 17.

In yet some further embodiments, the subject is further administered with at least one HK-Int variant and/or mutated molecule as defined by the invention.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Unless defined otherwise, all technical and scientific terms used herein have the same meaning as commonly understood by one of ordinary skill in the art to which this invention is related. The following terms are defined for purposes of the invention as described herein.

The following Examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Experimental Procedures

Materials and Reagents

Reagents:

Dulbecco's modified Eagle's medium (DMEM) (Biological industries, Beit Haemek, Israel). CalFectin transfection reagent (SignaGen Laboratories, MD, USA)

Plasmids:

Plasmids are listed in Tables 1, 3 and 5.

TABLE 1
List of plasmids

| Plasmid | Relevant Genotype | Use | Source |
|---|---|---|---|
| pcDNA3 | Neo^{R} oriSV40 | Cloning vector | Invitrogen |
| | vector | | |
| pcDNA5/frt | frt/Hygromycin | Cloning vector | Invitrogen |
| pEGFP-N1 | Neo^{R} EGFP-N1 | Cloning vector | Clonthech |
| pOG-Flp | Flp in pOG | Flp expression | Anderson, R.P., et al |
| | | plasmid | (2012) Nucleic |
| | | | Acids Res., 40, e62 |
| pSSK10 | oriR6K, Km^{R} | Off-target | (8) |
| | | assay | |
| pKH70 | Int in pETI1 | Int expression | (15) |
| pMK22 | Int in pKK233-2 | Off-target | present application |
| | | assay | |
| pMK144 | E174K in pKH70 | Off-target | present application |
| | | assay | |
| pMK218 | pCMV-attP-Stop- | Cis reaction | (11) |
| | attB-GFP | | |
| pMK189 | pCMV-attR-Stop- | Cis reaction | (11) |
| | attL | | |
| pMK221 | pCMV-attP on | Trans reaction | (11) |
| | pCDNA3 | | |
| pMK223 | Stop-attB-GFP | Trans reaction | (11) |
| pAM243 | pCMV-attR | Trans reaction | (11) |
| pMK242 | Stop-attL-GFP | Trans reaction | (11) |
| pNA979 | Int in pcDNA3 | Int expression | (9) |
| pNA1285 | attBHEXA5-t1-t2- | pNG1924 | (8) |
| | attPHEXA5 | construction | |
| pNA1328 | attPHEXA5- | pAE1983 | Lab collection |
| | GFP(ORF)-Neo | construction | |
| pNA1344 | pCMV- | Trans reaction | (8) |
| | attB(HEXA3) | | |
| pNA1481 | pCMV-attB(ATM4) | Trans reaction | (8) |
| pNA1483 | attP(ATM4)-GFP | Trans reaction | (8) |
| pNA1608 | attBATM2-t1-t2- | pNG1926 | (7) |
| | attPATM2 | construction | |
| pAE1627 | attB(HEXA5) in | pAE1901 | present application |
| | pcDNA5/frt | construction | |
| pAE1697 | pCMV-attB(CF12) | Trans reaction | present application |
| pAE1752 | attP w.t. in pSSK10 | Off-target | (7) |
| | | assay | |
| pNA1756 | attP(HEXA10) in | Off-target | (7) |
| | pSSK10 | assay | |
| pNA1757 | attP(ATM4) in | Off-target | (7) |
| | pSSK10 | assay | |
| pNG1826 | attP(DMD2)-GFP | Trans reaction | present application |
| pNG1839 | E264G oInt | Int expression | present application |
| pNG1844 | E319G oInt | Int expression | present application |
| pNG1860 | D336V oInt | Int expression | present application |
| pNG1862 | E174K oInt | Int expression | present application |
| pNG1864 | I43F oInt | Int expression | present application |
| pNG1866 | E174K E264G oInt | Int expression | present application |
| pNG1870 | I43F E174K oInt | Int expression | present application |
| pAE1874 | EF1alfa in pAE1627 | pAE1901 | present application |
| | | construction | |
| pAE1881 | Puro^{R} in pAE1874 | pAE1901 | present application |
| | | construction | |
| pAE1883 | mCherry in | pAE1901 | present application |
| | pAE1881 | construction | |
| pAE1901 | EF1alfa- | RMCE | present application |
| | attBHEXA5-Puro^{R}- | “docking” | |
| | attBATM4-mCherry | plasmid SEQ | |
| | | ID NO: 80 | |
| pAE1971 | attPATM4 in | pAE1983 | present application |
| | pNA1328 | construction | |
| pAE1983 | attPHEXA5- | RMCE | present application |
| | GFP(ORF)-NeoR- | “incoming” | |
| | CMV-attPATM4 | plasmid SEQ | |
| | | ID NO: 81 | |
| pNG1924 | attP(HEXA5) in | Off-target | present application |
| | pSSK10 | assay | |
| pNG1926 | attP(ATM2) in | Off-target | present application |
| | Off-p55K10 | assay | |
| | target assay | | |
| pAE2029 | E174K E319G oInt | Int expression | present application |
| pAE2030 | E174K D336V oInt | Int expression | present application |
| pAE2055 | I43F E174K R319G | Int expression | present application |
| | oInt | | |
| pAE2060 | E134K oInt | Int expression | present application |
| pAE2062 | D149K | Int expression | present application |
| pAE2064 | D215K | Int expression | present application |
| pAE2065 | D278K oInt | Int expression | present application |
| pAE2067 | N303K oInt | Int expression | present application |
| pAE2069 | E309K | Int expression | present application |
| pAE2071 | E174K D278K oInt | Int expression | present application |
| pAE2074 | attP(DMD3)-GFP | Trans reaction | present application |
| pAE2076 | attP(CTNS1)-GFP | Trans reaction | present application |
| pAE2077 | attP(CTNS4)-GFP | Trans reaction | present application |

Bacterial Strains

E. coli K12 strain TAP114 (Dorgai, L., et al. (1995) J. Mol. Biol., 252, 178-188)

E. coli S17-1 Lambda pir (Steyert S R, et al. (2007). Appl Environ Microbiol., 73: 4717-4724).

E. coli DH5alfa phi80lacZdeltaM15 delta(lacZYA-argF)U169 deoR recA1 endA1 hsdR17(rk−mk+) phoA supE44 lambda-thi-1 gyrA96 relA1.

Cell Lines:

HEK293T cells (ATCC)

HEK293 Flp-in

Kits:

DNA Spin Plasmid DNA purification Kit (Intron Biotechnology, Korea)

NucleoBond™ Xtra Maxi Plus EF kit (Macherey-Nagel, Germany)

PureFection transfection reagent (System Biosciences, Mountain View, Calif., USA)

Cells, Growth Conditions, Plasmids and Oligomers

The bacterial hosts used were E. coli K12 strain TAP114 (lacZ) deltaM15 (Dorgai, L., et al. (1995) J. Mol. Biol., 252, 178-188) and E. coli 517-1 Lambda pir (Steyert, 2007). The bacterial host used was E. coli DH5alfa phi80lacZdeltaM15 delta(lacZYA-argF)U169 deoR recA1 endA1 hsdR17(rk− mk+) phoA supE44 lambda-thi-1 gyrA96 relA1.

Plasmid transformations were performed by electroporation (Sambrook, J., et al (1989) Cold Spring Harbor, N.Y.). Plasmids and oligomers are listed in Tables 1,3,5 and 2,4,6 respectively. Human embryonic kidney cells HEK293, 293T, and 293 Flp-In were cultured in Dulbecco's modified Eagle's medium (DMEM). For transient transfection 293T cells (˜6×10⁵) were plated in a 6 well plate and 24 h later treated with 3 μg of the proper plasmid DNA using PureFection Transfection Reagent (System Biosciences, Mountain View, Calif., USA). For the model chromosomal assay transfection, 293 Flp-In cells (˜6×10⁵) were plated in a 6 well plate and 24 h later treated with 5.5 μg of the proper plasmid DNA using Mirus Transfection Reagent (Mirus, Wis., USA). For the CTNS and DMD chromosomal assay transfection, HEK293 cells (˜6×10⁵) were plated in a 6 well plate and 24 h later treated with 3 μg of the proper plasmid DNA using PureFection Transfection Reagent.

TABLE 2
List of oligomers that were
used as primers for the PCR reactions

| Oligo- | SEQ | | |
|---|---|---|---|
| mer | ID NO: | SEQUENCE | Location |
| 204 | NO: 1 | ATTGACGTCAATGGGAGTTTGTTT | pCMV |
| | | TGGC | |
| 469 | NO: 2 | GCATTTAGGTGACACTATAGAATA | pSP6 |
| | | GGG | |
| 894 | NO: 3 | GATCAGGGTGAGGAACAGCACACT | attB |
| | | TTACCAATGAAAGTCGTGACCAGG | HEXA5 |
| | | CCACGTT | |
| 895 | NO: 4 | AGCTAACGTGGCCTGGTCACGACT | attB |
| | | TTCATTGGTAAAGTGTGCTGTTCC | HEXA5 |
| | | TCACCCT | |
| 944 | NO: 5 | CCTTTTTAACCCATCACATATACC | P-part |
| | | TGCCGTTCTCAGGTCACTAATACT | |
| | | ATCTAAGTAGTTG | |
| 945 | NO: 6 | CGTTTGGATTGCAACTGGTCTATT | P′-part |
| | | TTCCTCTCGACAAATGATTTTATT | |
| | | TTGACTAATAATGACC | |
| 1021 | NO: 7 | GCAGCAGTGCAGAGGCGCCAGCAG | E264G |
| | | CAGCGAG | gag > ggc |
| 1022 | NO: 8 | CTCGCTGCTGCTGGCGCCTCTGCA | E264G |
| | | CTGCTGC | gag > ggc |
| 1023 | NO: 9 | CTGCCAGGCTGTACGGCAACCAGA | RR319G |
| | | TCGGCGACE | cgg > ggc |
| 1024 | NO: 10 | GTCGCCGATCTGGTTGCCGTACAG | R319G |
| | | CCTGGCAG | cgg > ggc |
| 1025 | NO: 11 | CTGGGCCACAAGAGCGTGAGCATG | D336V |
| | | GCCGCCAGD | gac > gtg |
| 1026 | NO: 12 | CTGGCGGCCATGCTCACGCTCTTG | D336V |
| | | TGGCCCAG | gac > gtg |
| 1030 | NO: 34 | GGACCGCCAAGAGCAAAGTGCGGC | E174K |
| | | GGAGCAGG | gaa > aaa |
| 1031 | NO: 35 | CCTGCTCCGCCGCACTTTGCTCTT | E174K |
| | | GGCGGTCC | gaa > aaa |
| 1032 | NO: 36 | CTGGGCCGGGACAGGCGGTTCGCC | I43F |
| | | ATCACCGAGGCCATCC | atc > ttc |
| 1033 | NO: 37 | GGATGGCCTCGGTGATGGCGAACC | I43F |
| | | GCCTGTCCCGGCCCAG | atc > ttc |
| 1051 | NO: 38 | ATGTATTTAGAAAAATAAACAAAT | pEF1alfa |
| | | AGGGGTCGTGAGGCTCCGGTGCCC | |
| | | GTC | |
| 1052 | NO: 39 | ATCTCCCGATCCGTCGACGTCAGG | pEF1alfa |
| | | TGGCACACCTAGCCAGCTTGGGTC | |
| | | TCCC | |
| 1064 | NO: 40 | TCGAGTCTAGAGGGCCCGTTTAAA | mCherry |
| | | CCCGCTATGGTGAGCAAGGGCGAG | |
| | | GAGG | |
| 1065 | NO: 41 | GTCAAGGAAGGCACGGGGGAGGGG | mCherry |
| | | CAAACAGGACAAACCACAACTAGA | |
| | | ATGCAGTG | |
| 1069 | NO: 74 | GAAAGCAGGTAGCTTGCAGTGGGC | KmR |
| 1070 | NO: 75 | GGCGACACGGAAATGTTGAATACT | KmR |
| | | CATAC | |
| 1143 | NO: 76 | TCAGGTTACTCATATATACTTTAG | P′ part |
| | | ATTGATGAATTCCAGGATATCCGA | |
| | | CAAAT GATTTTATTTTGACTAAT | |
| | | AATGACC | |
| 1144 | NO: 77 | ACGGGGTCTGACGCTCAGTGGAAC | P part |
| | | GAAAACCCGCGGCAGCCCGGGCTC | |
| | | AGGT CACTAATACTATCTAAGTA | |
| | | GTTG | |
| 1167 | NO: 78 | CAGGTTACTCATATATACTTTAGA | pCMV |
| | | TTGATGAATTCCGCGATGTACGGG | |
| | | CCAGATATAC | |
| 1169 | NO: 79 | CATTATTAGTCAAAATAAAATCAT | pCMV |
| | | TTGTCGGATATCGCAGTGGGTTCT | |
| | | CTAGTTAGCC | |

Mutation positioins are underlined, p-promoter

Off-Target Integration Assays in E. coli

Cells of E. coli strain TAP114 that carried w.t. Int or E174K mutant expressing plasmid (pMK22 or pMK174, respectively) were transformed with the relevant attP plasmid constructed on the base of pSSK10 and plated on LB rich medium supplemented with Km and Ap. The Km, Ap resistant colonies were checked for pSSK10 plasmid presence by KmR gene PCR analysis using primers oEY1069+1070, as denoted by SEQ ID NO: 74 and 75 respectively. Site-specific integration of the wild type Km^{R} attP plasmid into the native attB was confirmed by colony PCR analysis using primers oEY958+1080, as denoted by SEQ ID NO. 90 and 91 respectively (for attL) and oEY788+1069, as denoted by SEQ ID NO: 124 and 74 respectively (for attR) followed by sequencing.

Plasmid Construction

All plasmids (see List in Table 1) were verified by DNA sequencing. The relevant attP plasmids used in the off-target experiments were constructed by RF cloning (Unger, T., et al (2010) J. Struct. Biol., 172, 34-44) using the appropriate primers and plasmids as template (Tables 1 and 2) and the pSSK10 vector. These plasmid were propagated in S17-1 lambda pir as host.

The w.t. Int-expressing plasmid pMK22 was constructed by cloning of the Int fragment into the NcoI-HindIII sites of pKK322-2.

Construction of Int Mutants

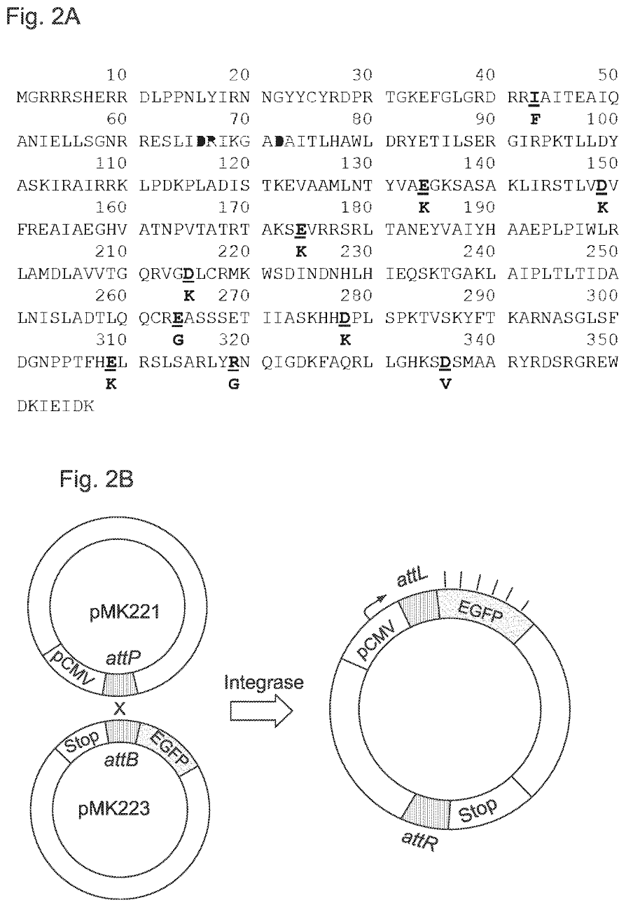

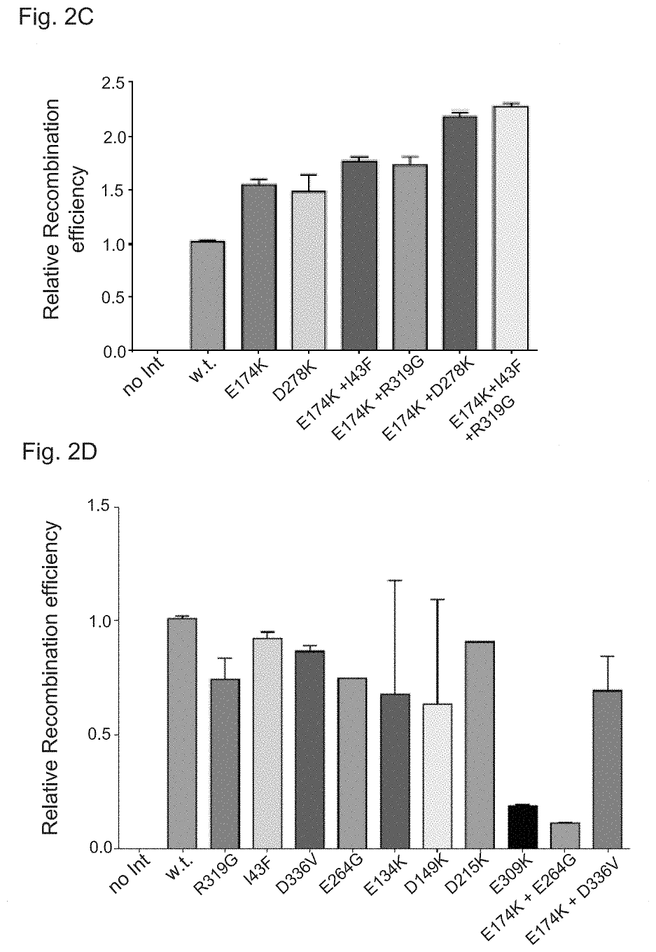

All Int mutants as presented by FIG. 2A were built by the same two steps procedure. First, by two PCR reactions with the relevant oligomers that contain the desired point mutation or double mutations using Int w.t. expression plasmid pNA979 as template (as oEY204 and 1033 as denoted by SEQ ID NO: 1 and SEQ ID NO: 37 respectively and primers oEY1032 and 469 as denoted by SEQ ID NO: 36 and SEQ ID NO: 2 respectively for I43F mutation. Then, these two PCR reactions were assembled also by PCR using oligomers 204 and 469 as denoted by SEQ ID NO: 1 and SEQ ID NO: 2 respectively and after restriction with EcoRI+HindIII enzymes, ligated to the pcDNA3 vector. All Int double mutants were constructed in the same way using the plasmid pNG1862 as a template. The triple mutant was constructed in the same way using the plasmid pAE2029 as a template. To construct E174K Int mutant-expressing plasmid for E. coli two PCR reaction with primers 513+144 as denoted by SEQ ID NO: 173 and SEQ ID NO: 171 respectively and 143+203 as denoted by SEQ ID NO: 170 and SEQ ID NO: 172 respectively using pKH70 plasmid [15] were assembled by PCR with primers 513+203 as denoted by SEQ ID NO: 173 and SEQ ID NO: 172 respectively, cut with NdeI and HindIII and cloned between the same enzymes in pKH70.

Plasmid Construction for DMD and CTNS Experiments

All plasmid constructs were verified by DNA sequencing. (Table 3, List of plasmids).

The plasmids used as substrates in the E. coli in cis integration assays were constructed by a triple ligation of the SalI-HindIII fragment of plasmid pXLPB with a SalI-DraI BOB′-t1t2-PO fragment obtained by PCR using plasmid pOK1205 as template with the relevant primers (Table 4) and a DraI-HindIII fragment that carried the P′ sequence obtained by PCR using plasmid pMK218 as template and primers oEY736 and oEY204 as denoted by SEQ ID NO: 138 and SEQ ID NO: 1 respectively.

The two plasmids that were used as substrates in the transient human HEK293T cells recombination assays were constructed as follows. To construct the plasmid that carried the relevant Stop-“attP”-GFP sequence a PstI-AgeI PCR fragment carried the appropriate “attP” was cloned into the same sites of plasmid pMK223. In these PCR reactions, the relevant E. coli substrate plasmids (Table 3) were used as template with primers oEY674 and oEY675 as denoted by SEQ ID NO: 136 and SEQ ID NO: 137 respectively. The plasmid that carried an appropriate “attB” downstream to the CMV promoter was constructed by ligation of the HindIII-EcoRI “attB” fragment obtained by annealing of the appropriate oligomers (Table 4) into the same sites of plasmid pCDNA3.

To construct the “docking” plasmid pAE1901 coding EF1alfa-attBHEXA3-PuroR-attBATM4-mCherry cassette. HEXA3 attB fragment obtained by annealing of oligomers 894+895 as denoted by SEQ ID NO: 3 and SEQ ID NO: 4 respectively, was cloned between HindIII and BglII of pcDNA5/frt (pAE1627). Next, EF1alfa promoter fragment obtained by PCR with primers 1051+1052 as denoted by SEQ ID NO: 38 and SEQ ID NO: 39 respectively, and pEF6_v5-His-Topo plasmid as template was inserted by RF cloning in pAE1627 (pAE1874) followed by PuroR fragment (from pMK1347, lab collection) cloning between EcoRV and BamHI (pAE1881). Next, mCherry fragment obtained by PCR with primers 1064+1065 as denoted by SEQ ID NO: 40 and SEQ ID NO: 41 respectively, from CMV-mCherry plasmid (lab collection) was inserted by RF cloning (pAE1883) followed by STOP-attB ATM4 fragment (from pNG1755, lab collection) cloning between EcoRV and NotI (pAE1901).

DMD RMCE incoming plasmid carried attPDMD2-SA+P2A+EGFP(ORF)+Poly A-attPDMD3 cassette construction was performed as follows. First, plasmid pCDNA3.1 carried CD:: UPRT gene (gift of Dr. Dr J Hiscott, Vaccine and Gene Therapy Institute of Florida, Port St Lucie, Fla., USA) was cut by EcoRI and HindIII, blunted by Klenow and self-ligated resulting to EcoRI-HindIII fragment deletion (pAE1999). Next, attPDMD2 fragment obtained by PCR with primers 1202+1203 as denoted by SEQ ID NO: 142 and SEQ ID NO: 143 respectively, using pNG1826 plasmid as template cut with XbaI was ligated with fragment of pAE1999 obtained by PCR with primers 1192+1201 as denoted by SEQ ID NO: 140 and SEQ ID NO: 141 respectively, cut with the same restriction enzyme (pAE2008). Next, attPDMD3 fragment obtained by PCR with primers 1215+931 as denoted by SEQ ID NO: 144 and SEQ ID NO: 139 respectively, using pAE2074 plasmid as a template cut with SacII and EcoRI was ligated with SacII+EcoRI pAE2008 fragment obtained by PCR with primers 1216+1217 as denoted by SEQ ID NO: 145 and SEQ ID NO: 146 respectively (pAE2032). Next, the pAE2032 cut with BglII and XbaI was blunted and self-ligated (pAE2086). Finally, the full cassette fragment carried SA made by PCR with primers 1240+1241 as denoted by SEQ ID NO: 149 and SEQ ID NO: 150 respectively on human genome DNA, P2A obtained by PCR with primers 1242+1243 as denoted by SEQ ID NO: 151 and SEQ ID NO: 152 respectively, on pAE2139 (lab collection) and EGFP made by PCR with primers oEY1244+1245 as denoted by SEQ ID NO: 153 and SEQ ID NO: 154 respectively, on pEGFPN1 was assembled by PCR with primers 1240+1245 as denoted by SEQ ID NO: 149 and SEQ ID NO: 154 respectively. BamHI+HindIII full cassette fragment was cloned between the same sites of pAE2086 (pAE2091).

CTNS RMCE incoming plasmid carried attPCTNS4-pCMV-GFP(ORF)-P2A-SD-attPCTNS1 cassette construction was performed as follows: First, attPCTNS4 fragment obtained by PCR with primers 1237+1238 as denoted by SEQ ID NO: 147 and SEQ ID NO: 148 respectively, using pAE2077 as template cut with XbaI and BamHI was cloned between the same sites of pAE2032 (pAE2045). Next, attPCTNS1 fragment obtained by PCR with primers oEY931+1215 as denoted by SEQ ID NO: 139 and SEQ ID NO: 144 respectively, using pAE2076 as template cut with SacII and EcoRI was cloned between the same sites of pAE2045 (pAE2047). Next, Stop (transcription terminator) fragment obtained by PCR with primers 606+1246 as denoted by SEQ ID NO: 135 and SEQ ID NO: 155 respectively, using pMK189 as a template cut with BglII and XbaI was cloned between the same sites of pAE2047 (pAE2049). Next, pAE2049 cut with EcoRI and BamHI was assembled with a GFP PCR fragment obtained with 1254+1255 primers as denoted by SEQ ID NO: 156 and SEQ ID NO: 157 respectively, using pEGFP-N1 as template and P2A-SD of exon 3 CTNS PCR fragment obtained with oEY1256+1257 as denoted by SEQ ID NO: 158 and SEQ ID NO: 159 respectively, on pADN171 (lab collection) by Gibson reaction (pAE2053). Finally, the BamHI CMV promoter fragment obtained by PCR with primers 400+416 as denoted by SEQ ID NO: 133 and SEQ ID NO: 134 respectively, using pCDNA3 cut with BamHI was inserted into the same site of pAE2053 in the right orientation (pAE258).

The relevant attP plasmids pNG1924 (HEXA3) and pNG1926 (ATM2) used in the off-target experiments were constructed by RF cloning (Unger, T., et al (2010) J. Struct. Biol., 172, 34-44) using the primers 944 and 945 as denoted by SEQ ID NO: 5 and SEQ ID NO: 6 respectively and plasmids as a template (Tables 1 and 2) and the pSSKre vector. These plasmids were propagated in S17-1 lambda pir as host.

TABLE 3
List of plasmids

| | | |
|---|---|---|
| Plasmid | Relevant genotype | Source |

a. Plasmids for E. coli assays:

| | | |
|---|---|---|
| pMK155 | Int-expressing plasmid, Km^{R} | [12] |
| pXLPB | pBAD24-t₁t₂-lacZ, Ap^{R} | [13] |
| pOK1205 | attB-t₁t₂-attP in pXLPB | [14] |
| pNG1770 | “attB”-t₁t₂-“attP”(CTNS1) | present application |
| | in pXLPB | |
| pNA1780 | “attB”-t₁t₂-“attP”(CTNS4) | present application |
| | in pXLPB | |
| pNG1819 | “attB”-t₁t₂-“attP”(DMD2) | present application |
| | in pXLPB | |
| pAE1843 | “attB”-t₁t₂-“attP”(DMD3) | present application |
| | in pXLPB | |
| pAE2010 | “attB”-t₁t₂-“attP”(DMD4) | present application |
| | in pXLPB | |
| pAE2014 | “attB”-t₁t₂-“attP”(DMD5) | present application |
| | in pXLPB | |
| pAE2012 | “attB”-t₁t₂-“attP”(DMD6) | present application |
| | in pXLPB | |
| pAE2013 | “attB”-t₁t₂-“attP”(DMD7) | present application |
| | in pXLPB | |

b. Plasmids for transient tests in human cells:

| | | |
|---|---|---|
| pCDNA3 | Neo^{R} Ap^{R} | Invitrogen |
| pEGFP-N1 | Neo^{R} Ap^{R} | Clonetech |
| pMK218 | pCMV-attP-STOP-attB- | [11] |
| | GFP, Km^{R} | |
| pMK223 | STOP-attB-GFP, Km^{R} | [11] |
| pNA979 | Int-expressing plasmid, Ap^{R} | [9] |
| pNG1825 | “attP”(DMD2)-GFP | present application |
| pNG1832 | pCMV-“attB”(DMD2) | present application |
| pAE1992 | “attP”(DMD3)-GFP | present application |
| pAE1994 | pCMV-“attB”(DMD3) | present application |
| pAE2016 | “attP”(DMD4)-GFP | present application |
| pAE2018 | “attP”(DMD5)-GFP | present application |
| pAE2020 | “attP”(DMD6)-GFP | present application |
| pAE2022 | “attP”(DMD7)-GFP | present application |
| pAE2024 | “attP”(CTNS1)-GFP | present application |
| pAE2025 | pCMV-“attB”(DMD4) | present application |
| pAE2026 | pCMV-“attB”(DMD5) | present application |
| pAE2027 | pCMV-“attB”(DMD6) | present application |
| pAE2036 | “attP”(CTNS4)-GFP | present application |
| pAE2038 | pCMV-“attB”(DMD7) | present application |
| pAE2042 | pCMV-“attB”(CTNS4) | present application |
| pAE2043 | pCMV-“attB”(CTNS1) | present application |

c. Incoming plasmids for chromosomal Int-catalyzed DMD and CTNS1
“attB”s activity detection in RMCE reactions

| | | |
|---|---|---|
| pCDNA3.1 | NeoR, ApR | Invitrogen |
| pAE1999 | ApR | present application |
| pAE2008 | “attP”DMD2 | present application |
| pAE2032 | “attP”DMD2-“attP”DMD3 | present application |
| pAE2045 | “attP”CTNS4 | present application |
| pAE2047 | “attP”CTNS4-“attP”CTNS1 | present application |
| pAE2049 | STOP-“attP”CTNS4-“attP” | present application |
| | CTNS1 | |
| pAE2053 | EGFP-P2A-SD in pAE2049 | present application |
| pAE2058 | “attP”(CTNS4)-CMV-GFP | present application |
| | (ORF)-P2A-exon3 | |
| | SD-“attP” (CTNS1) | |
| pAE2086 | “attP”DMD2-“attP”DMD, | present application |
| | BglII-XbaI deletion in #2032 | present application |
| pAE2091 | “attP”(DMD2)-exon44 SA- | present application |
| | P2A-GFP-polyA-“attP” | present application |
| | (DMD3) | |
| pAE2151 | SA+T2A+turboGFP+P2A+SD | present application |

*t₁t₂ is the rrnB terminator

TABLE 4
List of oligomers that were
used as primers for the PCR reactions

| | Sequence ID | | |
|---|---|---|---|
| Primer | NO: | Sequence | Location |
| oEY204 | SEQ ID NO: 1 | ATTGACGTCAATGGG | CMV |
| | | AGTTTGTTTTGGC | |
| oEY400 | SEQ ID NO: 133 | CGGGATCCGATGTAC | CMV |
| | | GGGCCAGATATAC | |
| oEY416 | SEQ ID NO: 134 | GCGGATCCGGGTCTC | CMV |
| | | CCTATAGTGAGTCG | |
| oEY606 | SEQ ID NO: 135 | GGGAGATCTACTTAC | STOP |
| | | CATGTCAGATCCAG | |
| oEY674 | SEQ ID NO: 136 | GGACCGGTCAAATGA | P′-part |
| | | TTTTATTTTGACTAA | |
| | | TAATGACC | |
| oEY675 | SEQ ID NO: 137 | GGGGCTGCAGAGGTC | P-part |
| | | ACTAATACTATCTAA | |
| | | GTAGTTG | |
| oEY736 | SEQ ID NO: 138 | AGGTCACTAATACTA | P-part |
| | | TCTAAGTAGTTGATT | |
| | | CATAGTGACTGG | |
| oEY931 | SEQ ID NO: 139 | CGTGCCAGCTGCATT | P′-part |
| | | AATGAATCGGCCAAC | |
| | | GAATTCCAGAAGCTT | |
| | | CGACAAATGATTTTA | |
| | | TTTTGACTAATAATG | |
| | | ACC | |
| oEY1192 | SEQ ID NO: 140 | GTAGCGGTCACGCTG | pCDNA3.1 |
| | | CGCGTAACCACCACA | |
| oEY1201 | SEQ ID NO: 141 | CCCGGATCCTTAGGG | pCDNA3.1 |
| | | TTCCGATTTAGTGCT | |
| | | TTACGGC | |
| oEY1202 | SEQ ID NO: 142 | GGGTCTAGACAAATG | P′-part |
| | | ATTTTATTTTGACTA | |
| | | ATAATGACC | |
| oEY1203 | SEQ ID NO: 143 | CCCGGATCCAGGTCA | P-part |
| | | CTAATACTATCTAAG | |
| | | TAGTTGATTCATAGT | |
| | | GACTGG | |
| oEY1215 | SEQ ID NO: 144 | GGGCCGCGGCTCAGG | P-part |
| | | TCACTAATACTATCT | |
| | | AAGTAGTTG | |
| oEY1216 | SEQ ID NO: 145 | GGGCCGCGGCTCAAA | pCDNA3.1 |
| | | GGCGGTAATACGGTT | |
| | | ATCCACA | |
| oEY1217 | SEQ ID NO: 146 | CCCGAATTCGTTGGC | pCDNA3.1 |
| | | CGATTCATTAATGCA | |
| | | GCTGG | |
| oEY1237 | SEQ ID NO: 147 | CCCGGATCCCAAATG | P′-part |
| | | ATTTTATTTTGACTA | |
| | | ATAATGACCTAC | |
| oEY1238 | SEQ ID NO: 148 | CCCTCTAGAAGGTCA | P-part |
| | | CTAATACTATCTAAG | |
| | | TAGTTGATTCATAGT | |
| | | GACTGG | |
| oEY1240 | SEQ ID NO: 149 | CTACTTAGATAGTAT | SADMD |
| | | TAGTGACCTGGATCC | exon44 |
| | | CTCTGCAAATGCAGG | |
| | | AAACTATCAGAG | |
| oEY1241 | SEQ ID NO: 150 | TTCGCGCGCTCAACA | DMD |
| | | GATCTGTCAAATCGC | exon44 |
| | | CTSA | |
| oEY1242 | SEQ ID NO: 151 | TGTTGAGCGCGCGAA | P2A |
| | | ACGCGG | |
| oEY1243 | SEQ ID NO: 152 | GCTCACCATAGGTCC | P2A |
| | | AGGGTTCTCCTCC | |
| oEY1244 | SEQ ID NO: 153 | CTGGACCTATGGTGA | EGFP |
| | | GCAAGGGCGAG | |
| oEY1245 | SEQ ID NO: 154 | AAATCATTTGTCGAA | EGFP |
| | | GCTTCTGGAATTCGG | |
| | | ACAAACCACAACTGA | |
| | | ATGCAGT | |
| oEY1246 | SEQ ID NO: 155 | GGGTCTAGAGCTGCC | STOP |
| | | ACCGTTGTTTCCACC | |
| | | GAG | |
| oEY1254 | SEQ ID NO: 156 | TATTAGTCAAAATAA | EGFP |
| | | AATCATTTGGGATCC | |
| | | ATGGTGAGCAAGGGC | |
| | | G | |
| oEY1255 | SEQ ID NO: 157 | TTCGCGCGCTTGTAC | EGFP |
| | | AGCTCGTCCATGC | |
| oEY1256 | SEQ ID NO: 158 | GTACAAGCGCGCGAA | P2A |
| | | ACGCGG | |
| oEY1257 | SEQ ID NO: 159 | ATTTGTCGAAGCTTC | P2A |
| | | TGGAATTCAACTTAC | |
| | | CACATTTAGGTCCAG | |
| | | GGTTCTCCTCC | |
| oEY206 | SEQ ID NO: 160 | | |

Plasmid Construction for Ctns1 Experiments

All plasmid constructs were verified by DNA sequencing. (Table 5, List of plasmids). The two plasmids that were used as substrates in the transient human HEK293T cells recombination assays were constructed as follows. To construct the plasmid that carried the relevant Stop-“attP”-GFP sequence a PstI-AgeI PCR fragment carried the appropriate “attP” was cloned into the same sites of plasmid pMK223. In these PCR reactions, the relevant E. coli substrate plasmids (Table 5) were used as template with primers oEY674 and oEY675 as denoted by SEQ ID NO: 136 and SEQ ID NO: 137 respectively. The plasmid that carried an appropriate “attB” downstream to the CMV promoter was constructed by ligation of the HindIII-EcoRI “attB” fragment obtained by annealing of the appropriate oligomers (Table 6) into the same sites of plasmid pCDNA3.

TABLE 5
List of plasmids

| | | |
|---|---|---|
| Plasmid | Relevant genotype Source | SOURCE |

a. Plasmids for transient tests in human cells:

| | | |
|---|---|---|
| pCDNA3 | Neo^{R} Ap^{R} | Invitrogen |
| pEGFP-N1 | Neo^{R} Ap^{R} | Clonetech |
| pMK218 | pCMV-attP-STOP-attB-GFP, Km^{R} | [11] |
| pMK223 | STOP-attB-GFP, Km^{R} | [11] |
| pAE2087 | pCMV-“attB”(CFTR10) | present application |
| pAE2089 | pCMV-“attB”(CFTR12) | present application |
| pAS2093 | “attP”(CFTR10)-GFP | present application |
| pAS2095 | “attP”(CFTR12)-GFP | present application |

c. Plasmids for Int expression

| | | |
|---|---|---|
| pNA979 | oInt w.t.-expressing plasmid, Ap^{R} | [9] |
| pNG1862 | E174K oInt | present application |
| pNG1870 | I43F E174K oInt | present application |
| pAE2029 | E174K E319G oInt | present application |
| pAE2055 | I43F E174K R319G oInt | present application |
| pAE2071 | E174K D278KoInt | present application |

TABLE 6
List of oligomers that were
used as primers for the PCR reactions

| Oligo- | SEQ | | |
|---|---|---|---|
| mer | ID NO: | SEQUENCE | Location |
| 143 | 170 | GCAAAATCAAAAGTAAGGC | E174K gaa > aaa |
| | | GTTC | |
| 144 | 171 | GAACGCCTTACTTTTGATT | E174K gaa > aaa |
| | | TTGC | |
| 203 | 172 | GCTAGTTATTGCTCAGCGG | T7 terminator |
| 204 | 1 | ATTGACGTCAATGGGAGTT | pCMV |
| | | TGTTTTGGC | |
| 469 | 2 | GCATTTAGGTGACACTATA | pSP6 |
| | | GAATAGGG | |
| 513 | 173 | AAGAGGATCACATATGGG | Int N-terminus |
| 1023 | 9 | CTGCCAGGCTGTACGGCAA | RR319G cgg > ggc |
| | | CCAGATCGGCGACE | |
| 1024 | 10 | GTCGCCGATCTGGTTGCCG | R319G cgg > ggc |
| | | TACAGCCTGGCAG | |
| 1030 | 34 | GGACCGCCAAGAGCAAAGT | E174K gaa > aaa |
| | | GCGGCGGAGCAGG | |
| 1031 | 35 | CCTGCTCCGCCGCACTTTG | E174K gaa > aaa |
| | | CTCTTGGCGGTCC | |
| 1032 | 36 | CTGGGCCGGGACAGGCGGT | I43F atc > ttc |
| | | TCGCCATCACCGAGGCCAT | |
| | | CC | |
| 1033 | 37 | GGATGGCCTCGGTGATGGC | I43F atc > ttc |
| | | GAACCGCCTGTCCCGGCCC | |
| | | AG | |
| 1265 | 164 | CCAGCAAGCACCACAAACC | D278K gac > aaa |
| | | CCTGAGCCCC | |
| 1266 | 165 | GGGGCTCAGGGGTTTGTGG | D278K gac > aaa |
| | | TGCTTGCTGG | |
| 1280 | 166 | AGCTTTGATAGTTTATGCC | attB CFTR10 |
| | | TCTACTTTTAAAAACAAAG | |
| | | TCTAACAGATTTTTCTCAG | |
| 1281 | 167 | AATTCTGAGAAAAATCTGT | attB CFTR10 |
| | | TAGACTTTGTTTTTAAAAG | |
| | | TAGAGGCATAAACTATCAA | |
| 1282 | 168 | AGCTTTGAGATGATGGAAA | attB CFTR12 |
| | | CACGCTTTCCCCTTCAAAG | |
| | | GTGCTGCTAGTTCCAAAGG | |
| 1283 | 169 | AATTCCTTTGGAACTAGCA | attB CFTR12 |
| | | GCACCTTTGAAGGGGAAAG | |
| | | CGTGTTTCCATCATCTCAA | |
| 1351 | 174 | TTTGACAGATCTGTTGAGG | DMD exon 44 SA- |
| | | AGAGCCAAGAGAGGCTCTG | T2A |
| | | G | |
| 1352 | 175 | GAGCCTCTCTTGGCTCTCC | DMD exon 44 SA |
| | | TCAACAGATCTGTCAAATC | |
| | | GCC | |
| 1353 | 176 | CTTAAGCTTGGACTCACCT | P2A-DMD exon 44 |
| | | GACGAGGTCCAGGGTTCTC | SD |
| | | CTC | |

Mutation positioins are underlined

Fluorescent-Activated Cell Sorting (FACS) Analysis

About 2×10⁶ cells from one well of a 6-well plate were collected following trypsin treatment of which 10⁴ cells were selected by the FACS sorter (Becton Dickinson Instrument) for fluorescent measurements. Data analysis was performed using the Flowing Software (University of Turku and Åbo Akademi University). Forward and side-scatter profiles were obtained from the same samples.

DNA Manipulations

Plasmid DNA from E. coli was prepared using a DNA Spin Plasmid DNA purification Kit (Intron Biotechnology, Korea) or a NucleoBond™ Xtra Maxi Plus EF kit (Macherey-Nagel, Germany). Gibson reaction was performed using the NEBBuilder HiFi DNA assembly master mix (NEB, MA, USA). General genetic engineering experiments were performed as described by Sambrook and Russell (Sambrook, J., et al (1989) Cold Spring Harbor, N.Y.).

Statistical Analysis

Data were presented as the mean±SD.

### Example 1

Int Activity Optimization in Human Cells

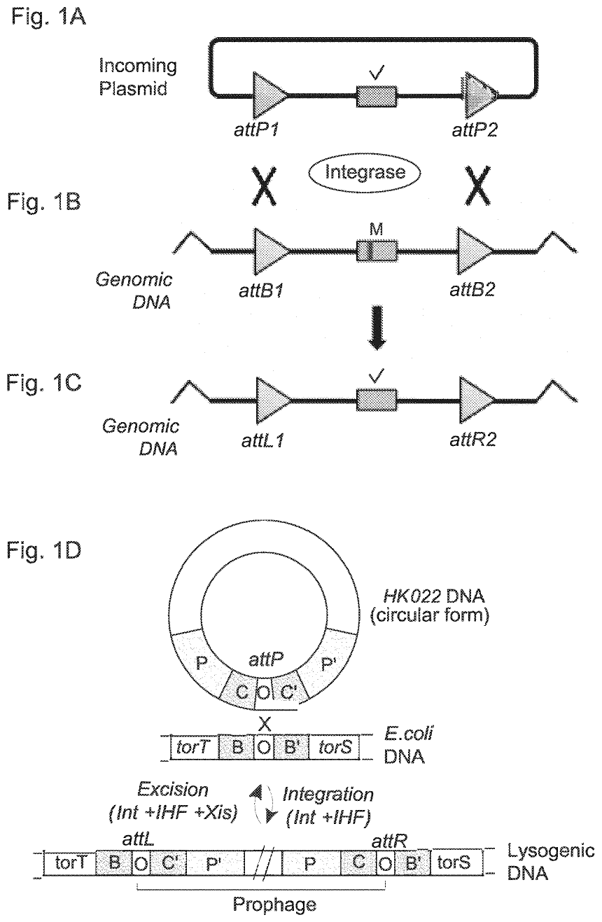

The unique benefits of SSRs for genome manipulation repose on their efficiency and specificity for recombining only their respective RSs. SSRs are non-viral and do not rely on host cell machinery to achieve transgenesis, hence providing attractive alternatives for the use in human cells. RMCE is based on using one or two different recombinases and allows replacing a genomic sequence containing a harmful mutation, deletion or insertion that is flanked by two incompatible RSs with a plasmid-borne sequence of interest flanked by matching RSs resulting a “clean” correction as no selection markers or undesired sequences is inserted [3] (FIG. 1A, 1B, 1C). E. coli HK022 bacteriophage SSR Integrase (Int) belongs to the tyrosine family of SSRs and naturally catalyzes phage integration between HK022 bacterial recombination site attB (BOB′, 21 bp long) and phage recombination site attP (POP, 230 bp long with COC′ core 21 bp) into the E. coli chromosome. B, B′ and C, C′ are palindrome 7 bp sites served for Int binding that flank a 7 bp overlap sequence (O) identical for both recombination sites (FIG. 1D). The inventors have previously shown that w.t. Int is active in human cells without the need to supply any of the prokaryotic accessory proteins [7,11]. Furthermore, the w.t. HK022 Int gene was adopted for the human codon usage (oInt) [9]. To harness the Int-based RMCE technology for therapy of human genetic diseases, several native active secondary attB sites (“attB”) were identified that flank variety of human deleterious mutations associated with genetic disorders, raising the prospect of using such sites to cure the “attB”-flanked mutations by Int catalyzed RMCE [8]. However, the oInt exhibits low RMCE efficiency in human cells.

The structure of Lambda's Int and its closely related Int of HK022 include three different domains (FIG. 2A) which coordinate actions both in cis and in trans reaction and facilitate assembly and function of a higher order tetrameric complex with the DNA attP substrate known as the intasome [5-6]. The N-terminal DNA binding domain (ND) (residues 1-63) as denoted by SEQ ID NO: 177 recognizes ‘arm-type’ DNA sequences adjacent to the attP core-site. Binding results in allosteric permitting of core-binding (CB) domain (residues 75-175) as denoted by SEQ ID NO: 178 and C-terminal catalytic domain (CD) (residues 176-356) as denoted by SEQ ID NO: 179 function. The CB domain recognizes the C and C′ core binding sites of attP and those of attB (B and B′) core DNA sequences and in association with the CD domain which is responsible for DNA cleavage and rejoining in the site-specific recombination reaction [5]. In aspiration to further optimize Int activity in human cells 10 different single mutated Ints were constructed (FIG. 2A): I43F (in the ND), E174K (CB) and E264G, R319G, D336V (CD), mutations. The inventors were also interested some other replacements of acidic residue. Thus, the mutants E134K, D149K (CB) and D215K, D278K, E309K (CD) were constructed (as denoted by SEQ ID NOs: 180, 188, 190, 182 and 192.

To examine the activity of these Int variants, an analytic assay was performed of a transient trans integrative recombination reaction using the wild type attB and attP sites in human HEK293T cells in which each att site is located on a different substrate plasmid (FIG. 2B). The first substrate (pMK221) carries the attP site downstream to the cytomegalovirus promoter (CMV). The second plasmid (pMK223) carries the attB downstream to the open reading frame (ORF) of the green fluorescent protein (GFP) and upstream to a transcription terminator (Stop).

A productive attB×attP reaction forms a dimer plasmid encoding CMV-promoted GFP expression (FIG. 2B). HEK293T cells were co-transfected with these two substrate plasmids, with or without an Int-expressing plasmid (the oInt pNA979, or one of its Int mutant derivatives). 48 hours post-transfection GFP expressing cells were analyzed by fluorescence-activated cell sorting (FACS). The quantified FACS data showed that only two single Int mutants E174K and D278K demonstrated a substantially increased integration activity (1.54 and 1.48 folds, respectively) compared to the oInt (FIG. 2C). On the other hand, all other 8 single mutants possessed lower activities (between 0.18 and 0.98 folds) compared to the oInt (FIG. 2D).

Since the E174K and D278K each showed about 1.5 folds elevated activity and the single mutation of I43F, R319G, E264G and D336V showed moderate activity, double mutants were constructed based on E174K variant. The double mutants E174K+I43F, E174K+R319G, and E174k+D278K showed an elevated activity between 1.7 to 2.3 folds over the oInt (FIG. 2C). However, E174K+E264G and E174K+D336V showed significantly lower activity (FIG. 2D). Lastly, based on the double mutants data, an E174K+I43F+R319G triple mutant (SEQ ID NO. 185) was constructed showing increased activity by 2.3 folds compare to the oInt.

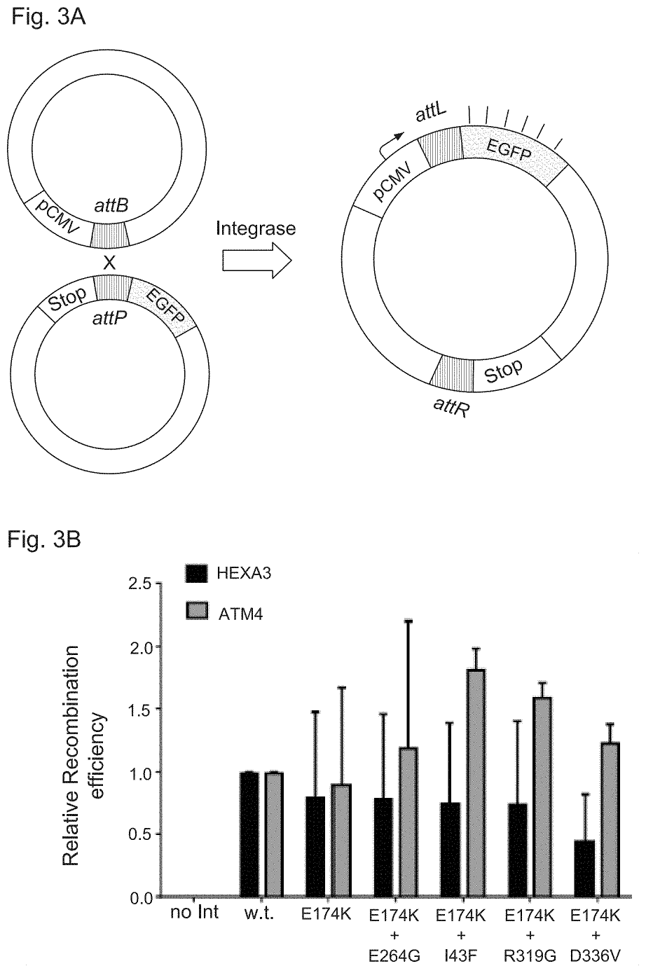

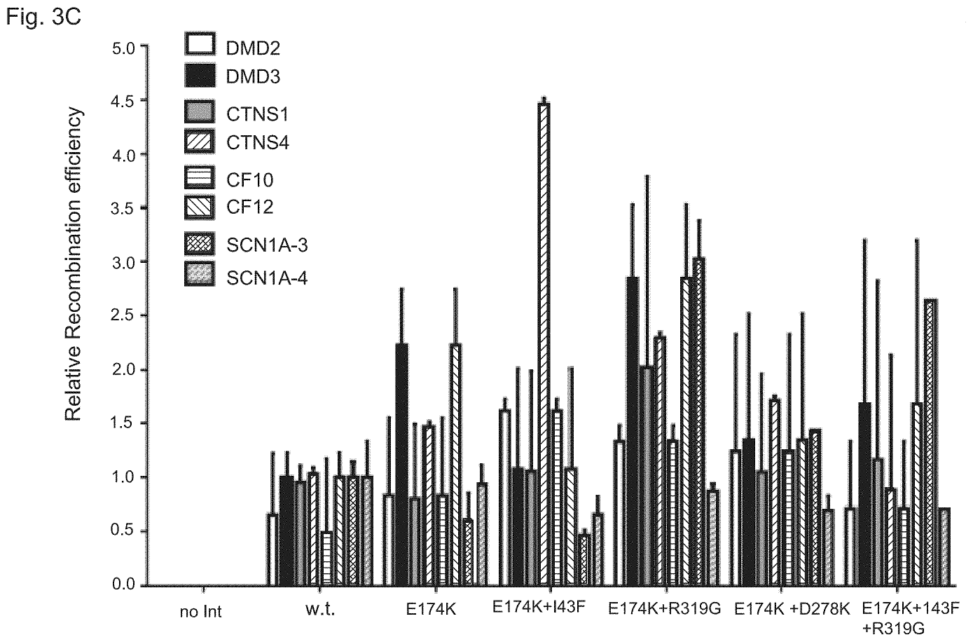

Next, using the same assay, the recombination activity of the various Int variants was examined on 10 different active “attB” sites (FIG. 3A, 3B, 3C) of which two (HEXA3 and ATM4, FIG. 3B) were previously reported [8]. The other three “attB” pairs flank common mutational regions in the genes of CTNS (chromosome 17), DMD (chromosome X), CFTR (chromosome 7) and SCN1A (chromosome 2), that cause the Cystinosis, Duchene muscular dystrophy, Cystic fibrosis and Dravet syndrome diseases, respectively (Shotelersuk, V., et al (1998). Am. J. Hum. Genet., 63, 1352-1362; Koenig, M., et al (1987) Cell, 50, 509-517; Kerem, B., et al (1989) Science, 245, 1073-1080).

Notably, Int-mutants showed variable efficiencies with the different “att” sites. For instance, the triple mutant Int was the most efficient Int with the wild type att sites (FIG. 2C). Although, with the HEXA3 and ATM4 “att” sites, the oInt and E174K+I43F were the most efficient ones, respectively (FIG. 3B). However, with CTNS1, DMD3, CF12 and SCN1A-3, the E174K+R319G Int mutant was the most efficient (FIG. 3C) and with SCN1A-4 the oInt was the most efficient one. Though, with CTNS4, DMD2, and CF10 the E174K+I43F Int was the most efficient (FIG. 3C). This data indicates that Int mutants have variable efficiency contribution toward the different “att” sites. This combination may give the prospect to achieve more efficient site-specific recombination toward the targeted “attB”s.

### Example 2

RMCE Reaction Catalyzed by Int Using Human Native attB Sites in Human Cells

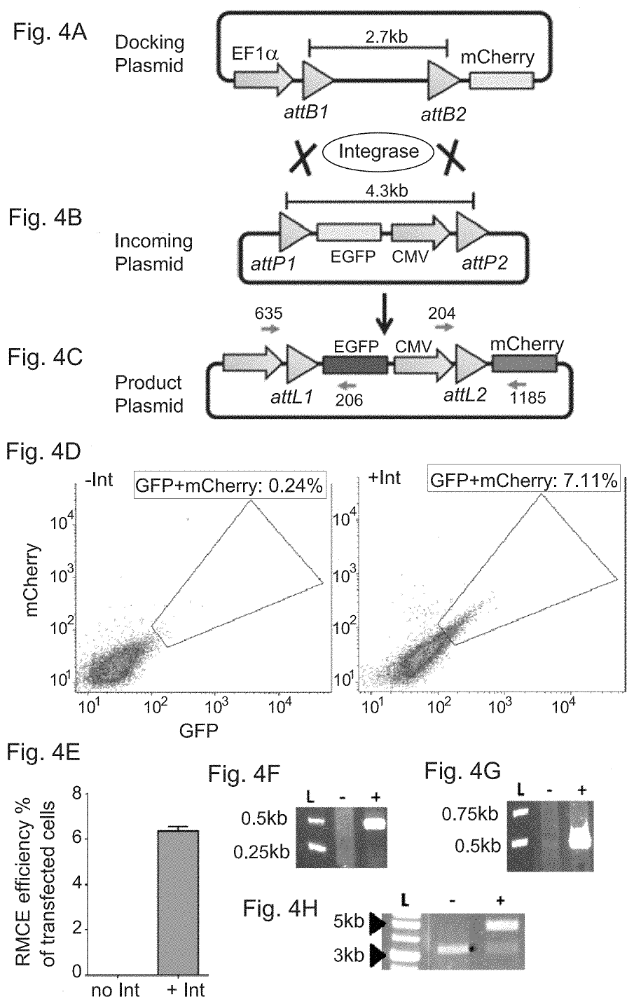

To examine if genomic “attB” sites that flank human deleterious mutations can serve as productive Int-catalyzed RMCE reaction substrates, a chromosomal RMCE reaction model was first designed. A “docking” RMCE substrate plasmid (FIG. 4A) was constructed to be inserted into the human chromosomal locus containing the SV40 promoter-frt site of the 293 Flp-In cells. This docking plasmid encodes two different “attB”s that are 2.7 Kb apart. attB1 presents the HEXA3 “attB” that is located downstream to the EF1alpha promoter, and attB2 presents the ATM4 “attB” located upstream to promoter-less mCherry ORF (FIG. 4A). An “incoming” plasmid (FIG. 4B) encodes the relevant compatible “attP” sites (attP1 and attP2 for HEXA3 and ATM4, respectively) which are 4.3 Kb apart. attP1 is located upstream to promoter-less ORF of EGFP and attP2 is located downstream to CMV promoter (FIG. 4B). A dual promoter trap Int-catalyzed RMCE reactions between these two plasmids are expected to form a recombinant product that co-expresses both green GFP and red mCherry fluorescent products (FIG. 4C). This was firstly tested by co-transfecting HEK293T cells with the docking and the incoming plasmids with or without Int, followed by 48 hours post-transfection FACS analyses. The quantified FACS data showed 6% of mCherry and GFP co-expression as a result of Int RMCE activity compare to the no Int treated cells (FIG. 4D-4E). The best Int variant for this reaction was the E174K mutant. To further verify that the elevated increase in dual fluorescence has indeed indicated the occurrence of the expected RMCE reaction, extrachromosomal DNA extracted from the transfected cells was tested by PCR. The PCR analysis with the appropriate primers confirmed by sequencing and demonstrated the formation of the expected recombination junctions: EF1α-attL-EGFP (500 bp) (FIG. 4C and FIG. 4F), CMV-attL-mCherry (486 bp) (FIG. 4C and FIG. 4G) and complete RMCE product (4.6 Kb) (FIG. 4H).

These results have demonstrated the validity of the two plasmids as proper substrates in proceeding towards a chromosomal RMCE reaction (FIG. 5). Hence, the HEK293 Flp-in cell line was used (FIG. 5B); these cells model carries a chromosomal locus of frt recombination site downstream to the SV40 promoter, known to be a model for high chromosomal expression (Invitrogen). HEK293 Flp-in cells were co-transformed with the docking plasmid (FIG. 5A) that also carried an frt site upstream to the hygromycin-resistance (HygR) ORF along with a plasmid pOG-Flp that expresses the Flp site-specific recombinase (Anderson, R. P., et al (2012) Nucleic Acids Res., 40, e62). The transformed Flp-in cells were plated on hygromycin contained medium that selected for Flp-catalyzed SV40 promoter-trap HygR recombinants carrying the integrated docking plasmid (FIG. 5C). The correct insertion of the docking plasmid was confirmed by the sequence of a 415 bp PCR product (FIG. 5C and FIG. 5H) using a chromosomal DNA template extracted from a HygR recombinant colony. Next, these cells docked with the chromosomal RMCE dual “attB” substrate (FIG. 5C), were co-transfected with the dual “attP” incoming plasmid (FIG. 5D) and the E174K Int-expressing plasmid followed by FACS analysis 48 hours post-transfection. Similarly to the extrachromosomal assay described above, the cells containing Int-catalyzed chromosomal RMCE products are expected to co-express EGFP and mCherry genes promoted by EF1α and CMV, respectively (FIG. 5E). The FACS analysis has shown that the efficiency of the Int-catalyzed chromosomal RMCE reaction achieved more than 1%, without any selection enrichment (FIG. 5F-5G). PCR and sequencing analyses by the appropriate primers using the chromosomal DNA of the transfected cells as a template, confirmed the expected recombination junction products EF1α-attL-EGFP (500 bp) (FIG. 5I) and EF1α-mCherry (273 bp) (FIG. 5J). Moreover, PCR analysis of expected full 4.6 kb RMCE product (FIG. 5E) has revealed the weak expected product dominated by the shorter 3.2 Kb PCR product of the non-recombined “docking” chromosomal cassette (FIG. 5C). Therefore, the 4.6 Kb product was gel-purified (FIG. 5K, gel on the left side) and used as a template for the nested PCR reaction that has confirmed the presence of the expected recombination junctions (FIG. 5K, the gel on the right side). The correct sequence of all PCR products was confirmed by sequencing. These results have confirmed that in this model experiment an Int-catalyzed chromosomal RMCE reaction product could be identified without any selection force.

### Example 3

Off-Target Int Activity Analysis in E. coli

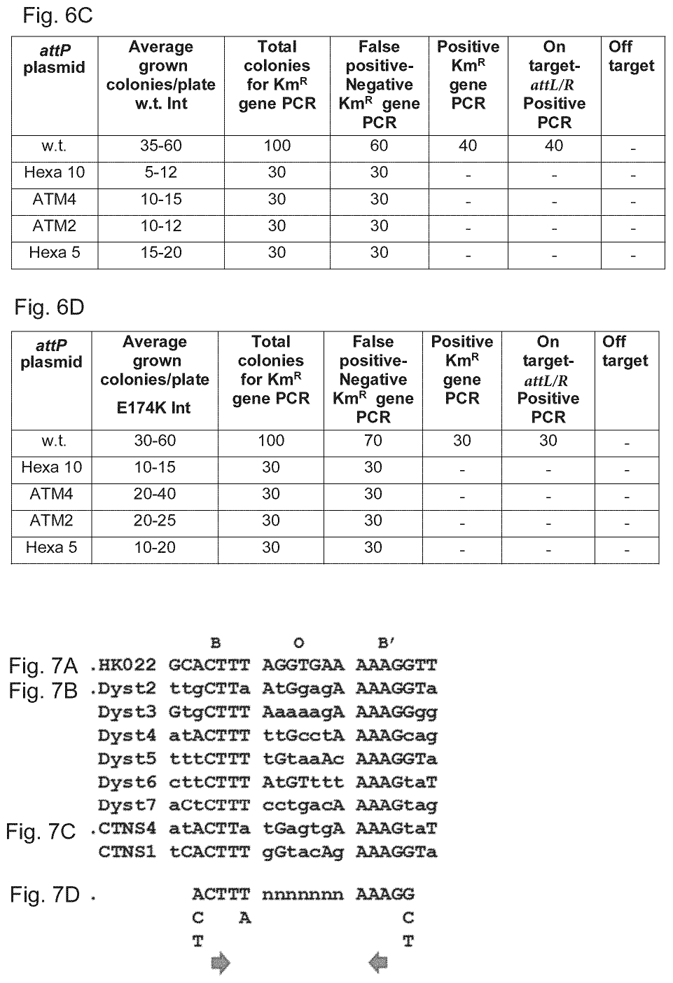

To re-examine the substantial level of Int-catalyzed human native “attP” sites off target integration activity (about 8.5%) in the E. coli described in the previous paper (8) the inventors applied more restrictive two steps assay (FIG. 6). Km^{R} pSSK10 plasmid that carries the wild type attP site (FIG. 6A) or the human “attP”s (HEXA 3 and HEXA 7, SEQ ID NO: 26 and 27 or ATM 2 and ATM 4, SEQ ID NO: 50 and 28) (FIG. 6B) was transformed into TAP114 strain that carries Ap^{R} w.t. or E174K Int-expressing plasmid. To avoid the interference of possible fouls-positive colonies, obtained Ap+Km resistant colonies were tested for the pSSK10 plasmid KmR gene presence by PCR analysis (FIG. 6A, Step 1). The positive PCR colonies obtained on the first step were used for the Int-catalyzed integration activity analysis by a second PCR for the presence of attR and attL recombination sites (FIG. 6A, Step 2). In three independent experiments, the plasmid that carried the w.t. attP yielded 30-60 positive colonies and 5-20 in the absence of Int. 30-70 Ap+Km resistance colonies in a repeated independent experiments obtained regardless of the Int plasmid presence were PCR negative thus are considered as fouls-positive colonies. Of 30 with E174K Int and 40 with w.t. Int Km^{R} positive PCR colonies, all proved to have resulted from the expected integration of the plasmid into E. coli's native attB site by an Int-catalyzed site-specific recombination reaction. Plasmids that carried human HEXA (5 and 10) or ATM (2 and 4) “attP” sites yielded 5-40 Ap+Km resistance colonies in the repeated independent experiments regardless of the Int plasmid presence. Km^{R} gene PCR (with the same primers used for w.t. attP plasmid) of 30 such colonies (FIG. 6C and FIG. 6D) were all negative indicating fouls-positive phenotype of these colonies.

These data confirm the absence of w.t. and E174K Ints catalyzed human native “attP” sites off target integration activity in the E. coli.

### Example 4

Active Human DMD and CTNS “attB” Sites

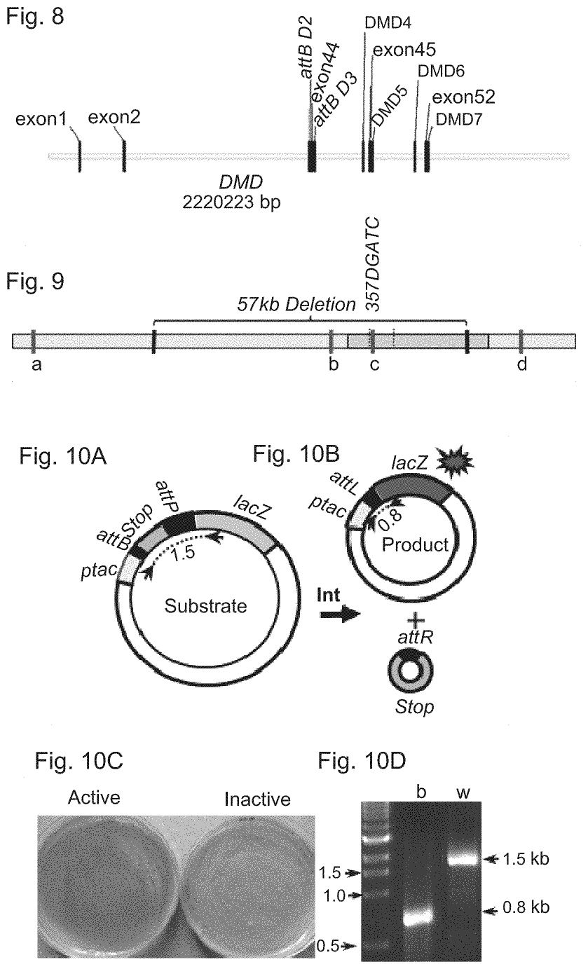

Using a computer assisted search for active human “attB” sites described in a previous work of the inventors [8], six potential “attB” sites were located in DMD gene flanked the exon 44 [DMD2 and DMD3 (23 kb apart), also denoted by SEQ ID NO. 92, 93, respectively], the exon 45 [DMD4 and DMD5 (41 kb apart) also denoted by SEQ ID NO. 108, 110, respectively] and the exon 52 [DMD6 and 7 flank exon 52 (58 kb apart), also denoted by SEQ ID NO. 112, 114, respectively](see FIGS. 7A, 7B, 7C, 7D and FIG. 8). Two potential “attB” sites were localized in CTNS gene flanked the mutation in exon 3 [CTNS4 and CTNS1 (7.6 kb apart), also denoted by SEQ ID NO. 72, 116, respectively] (see FIGS. 7A-7D and FIG. 9).

These sites were used by the inventors to assess the feasibility of natural sites for gene therapy of congenital disorders.

### Example 5

Cis Integration Reaction in E. coli

The activity of these “attB”s in the Int-catalyzed site-specific recombination was first tested in cis integration reaction in E. coli (FIG. 10). In this reaction, the recombining partner of each “attB” was the wild type attP except that its overlap was identical with the overlap of the appropriate “attB” (henceforth “attP”). This recombination reporter plasmid (FIG. 10A) carries the lacZ open reading frame that encodes beta-galactosidase separated from its pBAD promoter by a transcription terminator t₁t₂ from the rrnB gene (Glaser G. et al. 1983; Nature, 302: 74-76) flanked in tandem by an “attB” and the relevant “attP”. E. coli cells carried a compatible plasmid that express Int (pMK155) were transformed with this reporter plasmid and plated on LB rich medium supplemented with the X-gal (5-bromo-4-chloro-3-indolyl-β-D-galactopyranoside) indicator to detect blue colonies of cells in which Int-mediated recombination occurred and allowed beta-galactosidase expression (FIG. 10B). Recombination competent “attB” s were considered only those that yielded entirely blue colonies in which recombination was nearly or fully completed (FIG. 10C). PCR analysis of the blue colonies confirmed the presence of the product only in all tested substrates (FIG. 10D, line b). Accordingly, all tested DMD and CTNS “attB” s demonstrated high recombination activities.

### Example 6

RMCE Reactions Using “attB” Sites in the Native Location of Human Genes CTNS and DMD

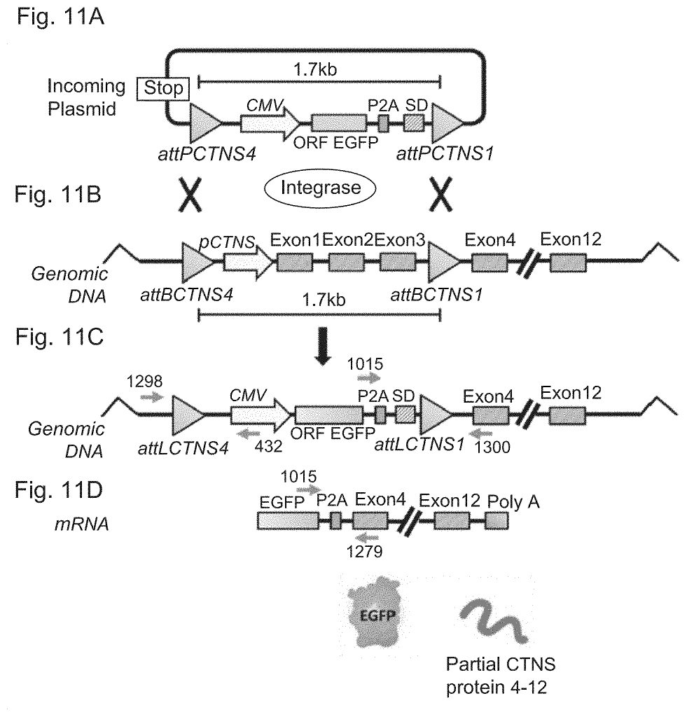

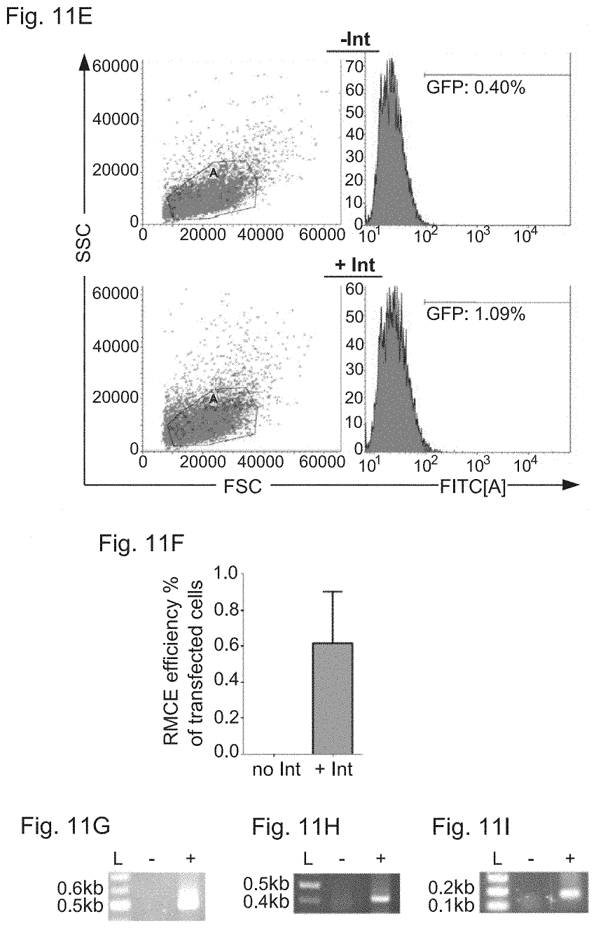

Next, it was aimed to demonstrate that the Int-based RMCE reactions may be potentially applicable for human gene therapy. Hence, Int-RMCE reactions was examined in the CTNS and DMD human genes using the appropriate “attB” sites described above in HEK293 cells by GFP trap assay. In The CTNS model, CTNS1 and CTNS4 “attB” sites (SEQ ID NO: 116, and 72) were chosen which are 7.6 Kb apart and flank a region containing the CTNS promoter and exons 1 to 3 (FIG. 11B). The relevant deletion mutation located in exon 3 is described (GM17886, Coriell institute). The appropriate incoming plasmid (FIG. 11A) carried a CMV-promoted EGFP ORF followed by a P2A sequence (for ribosomal skipping) and the splice donor of CTNS exon 3 (for RNA splicing), all flanked by the relevant “attP”s (CTNS4 and CTNS1) 1.7 Kb apart. HEK293 cells were co-transfected with the described incoming plasmid along with a plasmid expressing one of the Tnt variants. Positive Int-catalyzed RMCE is expected to replace the genomic sequence between the two “attB”s (CTNS4 and CTNS1) with the incoming sequence between its two “attP”s (FIG. 11C). Thus, the RMCE genomic recombinant is expected to transcribe an mRNA of the EGFP-P2A-exons 4-12 sequence (FIG. 11D) that owing to the P2A ribosomal skipping site will lead a translation of two peptides, GFP and a proximal portion of CTNS. FACS analyses of transformed cells has shown that the E174K+I43F Int variant has revealed the highest RMCE efficiency of 0.6% GFP fluorescence (FIG. 11E-11F). In addition, chromosomal DNA and mRNA were extracted from the transfected cells and served as template for PCR reactions with the proper primers (FIG. 11C-11D) that have demonstrated the formation of the expected recombinant junctions attL-CMV of 500 bp (FIG. 11C and FIG. 11G) and EGFP-2A-SD-attL-Intron of 400 bp (FIG. 11C and FIG. 11H). The mRNA PCR has revealed the expected EGFP-exon 4 junction of 177 bp (FIG. 11D and FIG. 11I). The correct sequence of all PCR products was confirmed by next-generation sequencing (NGS).

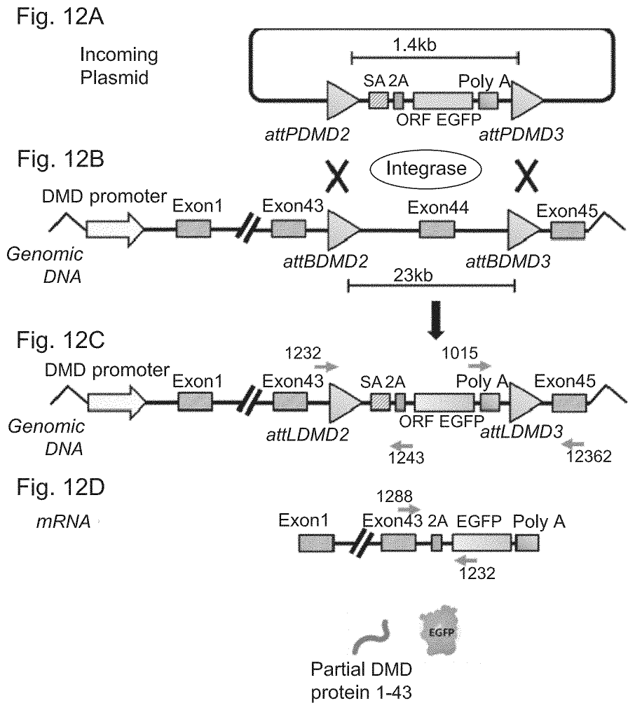

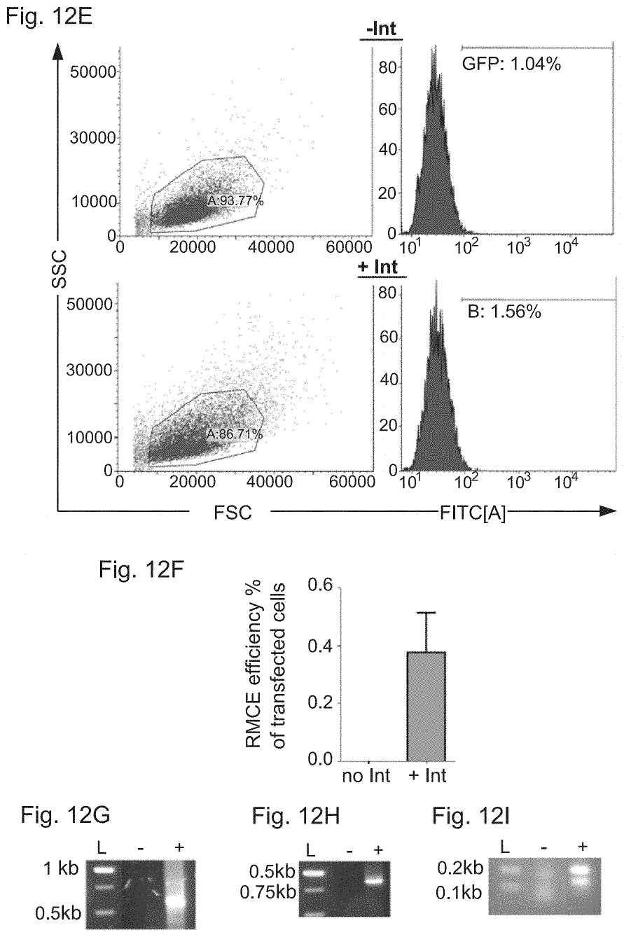

In the DMD model, DMD2 and DMD3 “attB” sites were chosen which are 23 Kb apart located in introns 43 and 44 respectively that flank exon 44 (FIG. 12B). The relevant deletion mutation located in exon 44 is described (GM23715, Coriell institute). A GFP promoter trap whose incoming plasmid carried a splicing acceptor (SA), a ribosomal skipping site (2A) and the ORF of EGFP with a polyA sequence (FIG. 12A) was used. All are flanked with the two relevant “attP”s 1.4 Kb apart. FACS analyses of transformed HEK293 cells as above showed that the highest 0.4% RMCE efficiency reached with the Int mutants E174K+D278K and E174K+I43F+R319G (FIG. 12E-12F). Chromosomal DNA and mRNA extracted from the transfected cells and served as template for PCR reactions with the proper primers have demonstrated the expected recombinant attL-SA junctions (700 bp) (FIG. 12C and FIG. 12G), EGFP-attR-exon 45 (800 bp) (FIG. 12C and FIG. 12H) and the mRNA exon 43-EGFP junction (229 bp) (FIG. 12D and FIG. 12I). The correct sequence of all PCR products was confirmed by NGS.

In conclusion, this data demonstrates the HK022 Int-RMCE system prospects to exchange a native genomic sequence with another sequence of interest in a stable manner without adding any selection marker or other undesired sequences. Furthermore, it can swap large transgene cassettes (over 20 kb).

### Example 7

Active Human CFTR “attB” Sites and Cis Integration Reaction in E. coli

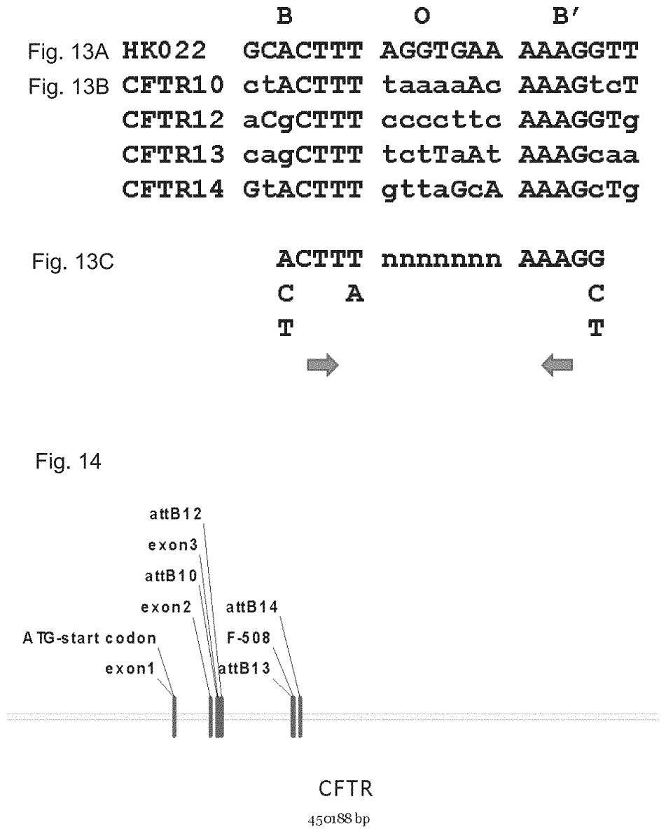

Using a computer search for active human “attB” sites as described previously [8], four potential “attB” sites were located in CFTR gene: CFTR10 and CFTR12 flanked the exon 3 (3 kb apart) and CFTR13 and CFTR14 flanked most common F-508 mutation (FIG. 13A, 13B, 13C and FIG. 14). The activity of CFTR10,12 and 13 “attB”s in the Int-catalyzed site-specific recombination was first tested in cis integration reaction in E. coli similarly to the experiment presented in FIG. 10. In this reaction, the recombining partner of each “attB” was the wild type attP except that its overlap was identical with the overlap of the appropriate “attB” (henceforth “attP”). This recombination reporter plasmid (as shown in the scheme of FIG. 10A) carries the lacZ open reading frame that encodes beta-galactosidase separated from its pBAD promoter by a transcription terminator t₁t₂ from the rrnB gene (Glaser, G., et al. (1983) Nature, 302, 74-76) flanked in tandem by an “attB” and the relevant “attP”. E. coli cells carried a compatible plasmid that express Int (pMK155) were transformed with this reporter plasmid and plated on LB rich medium supplemented with the X-gal (5-bromo-4-chloro-3-indolyl-β-D-galactopyranoside) indicator to detect blue colonies of cells in which Int-mediated recombination occurred and allowed beta-galactosidase expression (as shown in the scheme of FIG. 10B). Recombination competent “attB”s were considered only those that yielded entirely blue colonies in which recombination was nearly or fully completed. PCR analysis of the blue colonies confirmed the presence of the product only in all tested substrates. Accordingly, all tested CFTR “attB”s demonstrated high recombination activities.

### Example 8

Mapping HK022 Mutations Based in the Crystal Structure of Lambda Integrase

It appears that E174K mutant can potentially enhance the in trans Int mediated RMCE reaction. The data described in the present study shows that E174K Int enhanced RMCE efficiency (147%) compared to the oInt. The E174K mutation in HK022 Int is located in the inter-domain linker (I160-R176). It is assumed that the linker flexibility generates partial constraints on the relative orientations of the Int's central and catalytic domains. Moreover, this flexibility probably increases the entropic rate of DNA binding and thereby decreases DNA binding affinity. Without wishing to be bound by theory, it was estimated that lysine residue substitution might enhance the DNA binding affinity by stabilizing interaction with the DNA and/or by constraining the movement of the inter-domain linker [5]. It seems that E174K and D278K, which are substitutions of positively charged lysine for negatively charged Glu/Asp near DNA, enhance Int activity most likely by introducing new ionic interactions with the DNA backbone.

The same could have been expected for E309K as it is also near the DNA backbone. However, E309 is close to the active site and is hydrogen-bonded to R179, an important residue for positioning Tyr342 and it might explain why E309K is must less active than oInt. 143 is away from the arm-site DNA but it's facing the adjacent N-terminal domain within the Int tetramer. The R319G mutation located in CD is proximal to D336 and Y342 nucleophile. This region plays a key role in catalytic activity and regulation of site-specific recombination.

Thus, in the present study, an Integrase variants were constructed based on the E174K Int (E174K+I43F, E174K+E264G, E174K+R319G, E174K+D278K, E174K+I43F+D336V, as denoted by SEQ ID NO. 83, 87, 85, 184, 185, respectively) showed higher recombination active with the different “attB” sites (HEXA3, ATM4, DMD2, DMD3, CTNS1, CTNS4, CF10, CF12, SCN1A-3 and SCN1A-4) compared to the oInt (FIG. 3B-3C).

1. A HK022 bacteriophage site specific recombinase Integrase (HK-Int) variant and/or mutated molecule or any functional fragments or peptides thereof, wherein said variant comprise at least one substituted amino acid residue in at least one of the core-binding domain (CB), the N-terminal DNA binding domain (ND) and the C-terminal catalytic domain (CD) of the Wild type HK-Int molecule.

2. The HK-Int variant and/or mutated molecule according to claim 1, wherein said HK-Int variant comprises at least one substitution in at least one of residues 174, 278, 43, 319, 134, 149, 215, 264, 303, 309, 336, of the amino acid sequence of the Wild type HK-Int molecule as denoted by SEQ ID NO. 13 and any combinations thereof.

3. The HK-Int variant and/or mutated molecule according to claim 1, wherein said HK-Int variant comprises at least one substitution at the CB domain of the amino acid sequence of the Wild type HK-Int molecule as denoted by SEQ ID NO. 13, said HK-Int variant comprises at least one substitution in at least one of residues 174, 134, 149 and any combinations thereof, optionally wherein said HK-Int variant comprises at least one substitution at position 174 of said Wild type HK-Int molecule, wherein said variant comprises at least one substitution replacing glutamic acid (E) with lysine (K) at position 174 of the Wild type HK-Int molecule as denoted by SEQ ID NO. 13, and any variants, homologs or derivatives thereof.

4-5. (canceled)

6. The HK-Int variant and/or mutated molecule according to claim 1, further comprising at least one of: a substitution replacing Aspartic acid (D) with Lysine (K) at position 278, a substitution replacing Isoleucine (I) with Phenyl alanine (F) at position 43, a substitution replacing glutamic acid (E) with Glycine (G) at position 319, a substitution replacing glutamic acid (E) with Glycine (G) at position 264 and a substitution replacing Aspartic acid (D) with Valine (V) at position 336 of the Wild type HK-Int molecule, as denoted by SEQ ID NO. 13 and any variants, homologs or derivatives thereof.

7. (canceled)

8. The HK-Int variant and/or mutated molecule according to claim 1, wherein said HK-Int variant comprises at least one substitution at the CD domain of the amino acid sequence of the Wild type HK-Int molecule as denoted by SEQ ID NO. 13, said HK-Int variant comprises at least one substitution in at least one of residues 278, 215, 264, 303, 309, 319, 336, and any combinations thereof, optionally, said HK-Int variant comprises at least one substitution at position 278 of the Wild type HK-Int molecule as denoted by SEQ ID NO. 13 and any variants, homologs or derivatives thereof, said HK-Int variant comprises at least one substitution replacing Aspartic acid (D) with Lysine (K) at position 278 of the Wild type HK-Int molecule.

9-10. (canceled)

11. A nucleic acid molecule comprising a nucleic acid sequence encoding a HK-Int variant and/or mutated molecule according to claim 1, or any functional fragments or peptides thereof, or any vector or nucleic acid cassette thereof.

12. (canceled)

13. A host cell comprising at least one HK-Int variant and/or mutated molecule according to claim 1, or any functional fragments or peptides thereof, or any nucleic acid sequence encoding said at least one HK-Int variant, any combinations thereof, or with any vector, vehicle, matrix, nano- or micro-particle comprising the same, wherein said HK-Int variant comprises at least one substituted amino acid residue in at least one of the CB, ND and the CD of the Wild type HK-Int molecule.

14-15. (canceled)

16. The host cell according to claim 1, wherein said cell further comprise at least one nucleic acid molecule or any nucleic acid cassette or vector comprising a replacement-sequence flanked by a first and a second Int recognition sites, said first site attP1, comprises a first overlap sequence O1 and said second site attP2, comprises a second overlap sequence O2, wherein said first O1 and said second O2 overlap sequences are different, each consisting of seven nucleotides, said O1 is identical to an overlap sequence O1 comprised within a first Int recognition site attE1 in a eukaryotic cell and said O2 is identical to an overlap sequence O2 comprised within a second Int recognition site attE2 in said eukaryotic cell, said eukaryotic recognition sites attE1 and attE2 flank a target nucleic acid sequence of interest or any fragment thereof in said eukaryotic cell, wherein said O1 and O2 overlap sequences are each flanked by a first E and a second E′ Int binding sites, said first binding sites E comprise the sequence of C1-T2-T3-W4, as denoted by SEQ ID NO. 16, and said second binding sites E′ comprise the sequence of A12-A13-A14-G15, as denoted by SEQ ID NO. 17, optionally, wherein at least one of:
(a) wherein said first overlap sequence O₁ and said second overlap sequence O2 comprise a nucleic acid sequence as denoted by any one of SEQ ID NO. 98, SEQ ID NO. 99, SEQ ID NO. 127, SEQ ID NO. 128, SEQ ID NO. 117, SEQ ID NO. 70, SEQ ID NO. 71, SEQ ID NO. 73, SEQ ID NO. 131, SEQ ID NO. 132, SEQ ID NO. 104, SEQ ID NO. 105, SEQ ID NO. 94, SEQ ID NO. 95, SEQ ID NO. 109, SEQ ID NO. 111, SEQ ID NO. 113 and SEQ ID NO. 115, and wherein said O1 and said O2 are different; and
(b) wherein said replacement-sequence comprise a nucleic acid sequence that differs in at least one nucleotide from said target nucleic acid sequence of interest or any fragments thereof.

17-18. (canceled)

19. The host cell according to claim 16, wherein said target nucleic acid sequence of interest in said eukaryotic cell comprises or is is any one of:
(a) comprised within the human cystic fibrosis transmembrane conductance regulator (CFTR) gene or any fragment thereof, said nucleic acid sequence of interest is flanked by a first Int recognition site attE1 comprising the nucleic acid sequence as denoted by SEQ ID NO. 96 and a second Int recognition site attE2 comprising the nucleic acid sequence as denoted by SEQ ID NO. 97, and wherein said O1 comprises the nucleic acid sequence as denoted by SEQ ID NO. 98 and said O2 comprises the nucleic acid sequence as denoted by SEQ ID NO. 99;
(b) comprised within the human cystinosin (CTNS) gene or any fragment thereof, said nucleic acid sequence of interest is flanked by a first Int recognition site attE₁ comprising the nucleic acid sequence as denoted by SEQ ID NO. 116 and a second Int recognition site attE₂ comprising the nucleic acid sequence as denoted by of SEQ ID NO. 72, and wherein said O₁ comprises the nucleic acid sequence as denoted by SEQ ID NO. 117 and said O₂ comprises the nucleic acid sequence as denoted by SEQ ID NO. 73;
(c) comprised within the human sodium channel, voltage-gated, type I, alpha subunit (SCN1A) gene or any fragment thereof, said nucleic acid sequence of interest is flanked by a first Int recognition site attE₁ comprising the nucleic acid sequence as denoted by SEQ ID NO. 120 and a second Int recognition site attE₂ comprising the nucleic acid sequence as denoted by SEQ ID NO. 121, and wherein said O₁ comprises the nucleic acid sequence as denoted by SEQ ID NO. 104 and said O₂ comprises the nucleic acid sequence as denoted by SEQ ID NO. 105; or
(d) comprised within the human dystrophin (DMD) gene or any fragment thereof, said nucleic acid sequence of interest is flanked by a first Int recognition site attE₁ comprising the nucleic acid sequence as denoted by SEQ ID NO. 92 and a second Int recognition site attE₂ comprising the nucleic acid sequence as denoted by SEQ ID NO. 93, and wherein said O₁ comprises the nucleic acid sequence as denoted by SEQ ID NO. 94 and said O₂ comprises the nucleic acid sequence as denoted by SEQ ID NO. 95.

20-22. (canceled)

23. A system or kit comprising at least one of:
(a) at least one nucleic acid molecule or any nucleic acid cassette or vector thereof, comprising a replacement-sequence flanked by a first and a second Int recognition sites, said first site attP1, comprises a first overlap sequence O1 and said second site attP2, comprises a second overlap sequence O2, wherein said first O1 and said second O2 overlap sequences are different, each consisting of seven nucleotides, said O1 is identical to an overlap sequence O1 comprised within a first Int recognition site attE1 in a eukaryotic cell and said O2 is identical to an overlap sequence O2 comprised within a second Int recognition site attE2 in said eukaryotic cell, said eukaryotic recognition sites attE1 and attE2 flank a target nucleic acid sequence of interest or any fragment thereof in said eukaryotic cell; and
(b) at least one HK-Int variant and/or mutated molecule or any functional fragments or peptides thereof, any nucleic acid molecule comprising a sequence encoding said HK-Int variant and/or mutated molecule or any vector, vehicle, matrix, nano- or micro-particle comprising the same, wherein said variant comprise at least one substituted amino acid residue in at least one of the CB, ND and the CD of the Wild type HK-Int molecule.

24. (canceled)

25. The system or kit according claim 23, wherein wherein at least one of:
(a) said HK-Int variant and/or mutated molecule comprises the amino acid sequence as denoted by at least one of SEQ ID NO. 14, SEQ ID NO. 182, SEQ ID NO. 184, SEQ ID NO. 185, SEQ ID NO. 83, SEQ ID NO. 85, SEQ ID NO. 87 and SEQ ID NO. 89, or any combinations or any functional fragments, variants, fusion proteins or derivatives thereof;
(b) said nucleic acid sequence encoding said HK-Int variant comprises the nucleic acid sequence as denoted by any one of SEQ ID NO. 15, SEQ ID NO. 183, SEQ ID NO. 43, SEQ ID NO. 45, SEQ ID NO. 47, SEQ ID NO. 49, SEQ ID NO. 82, SEQ ID NO. 84, SEQ ID NO. 86, SEQ ID NO. 88, SEQ ID NO. 186, SEQ ID NO. 187, SEQ ID NO. 193 and SEQ ID NO. 224, or any derivatives, homologs, fusion proteins or variants thereof.

26. (canceled)

27. The system or kit according to claim 23, wherein said first overlap sequence O1 and said second overlap sequence O2 comprise a nucleic acid sequence as denoted by any one of SEQ ID NO. 98, SEQ ID NO. 99, SEQ ID NO. 127, SEQ ID NO. 128, SEQ ID NO. 117, SEQ ID NO. 70, SEQ ID NO. 71, SEQ ID NO. 73, SEQ ID NO. 131, SEQ ID NO. 132, SEQ ID NO. 104, SEQ ID NO. 105, SEQ ID NO. 94, SEQ ID NO. 95, SEQ ID NO. 109, SEQ ID NO. 111, SEQ ID NO. 113 and SEQ ID NO. 115, and wherein said O1 and said O2 are different, and
(b) said replacement sequence comprise a nucleic acid sequence that differs in at least one nucleotide from said at least one target nucleic acid sequence of interest or any fragments thereof.

28. (canceled)

29. The system or kit according claim 23, wherein said target nucleic acid sequence of interest in said eukaryotic cell comprises, or is comprised within, any one of:
(a) the human CFTR gene, said nucleic acid sequence of interest is flanked by a first Int recognition site attE1 comprising the nucleic acid sequence as denoted by SEQ ID NO. 96 and a second Int recognition site attE2 comprising the nucleic acid sequence as denoted by SEQ ID NO. 97, and wherein said O1 comprises the nucleic acid sequence as denoted by SEQ ID NO. 98 and said O2 comprises the nucleic acid sequence as denoted by SEQ ID NO. 99;
(b) the human CTNS gene or any fragment thereof, said nucleic acid sequence of interest is flanked by a first Int recognition site attE₁ comprising the nucleic acid sequence as denoted by SEQ ID NO. 116 and a second Int recognition site attE₂ comprising the nucleic acid sequence as denoted by SEQ ID NO. 72, and wherein said O₁ comprises the nucleic acid sequence as denoted by SEQ ID NO. 117 and said O₂ comprises the nucleic acid sequence as denoted by SEQ ID NO. 73;
(c) the human SCN1A gene or any fragment thereof, said nucleic acid sequence of interest is flanked by a first Int recognition site attE₁ comprising the nucleic acid sequence as denoted by SEQ ID NO. 120 and a second Int recognition site attE₂ comprising the nucleic acid sequence as denoted by SEQ ID NO. 121, and wherein said O₁ comprises the nucleic acid sequence as denoted by SEQ ID NO. 104 and said O₂ comprises the nucleic acid sequence as denoted by SEQ ID NO. 105; and
(d) the human DMD gene or any fragment thereof, said nucleic acid sequence of interest is flanked by a first Int recognition site attE1 comprising the nucleic acid sequence as denoted by SEQ ID NO. 92 and a second Int recognition site attE2 comprising the nucleic acid sequence as denoted by SEQ ID NO. 93, and wherein said O1 comprises the nucleic acid sequence as denoted by SEQ ID NO. 94 and said O2 comprises the nucleic acid sequence as denoted by SEQ ID NO. 95.

30-31. (canceled)

32. A composition comprising as an active ingredient an effective amount of:
(a) at least one HK-Int variant and/or mutated molecule or any functional fragments or peptides thereof, any nucleic acid molecule comprising a sequence encoding said HK-Int variant, or any vector, vehicle, matrix, nano- or micro-particle comprising the same, or any host cell comprising said HK-Int variant or nucleic acid sequence encoding said HK-Int variant, wherein said HK-Int variant comprises at least one substituted amino acid residue in at least one of the CB, ND and the CD of the Wild type HK-Int molecule; and
(b) at least one nucleic acid molecule or nucleic acid cassette comprising a replacement-sequence flanked by a first and a second Int recognition sites, said first site attP1, comprises a first overlap sequence O1 and said second site attP2, comprises a second overlap sequence O2, wherein said first O1 and said second O2 overlap sequences are different, each consisting of seven nucleotides, said O1 is identical to an overlap sequence O1 comprised within a first Int recognition site attE1 in a eukaryotic cell and said O2 is identical to an overlap sequence O2 comprised within a second Int recognition site attE2 in said eukaryotic cell, said eukaryotic recognition sites attE1 and attE2 flank a target nucleic acid sequence of interest or any fragment thereof in said eukaryotic cell; or a kit or system comprising (a) and (b).

33. (canceled)

34. A method for replacing at least one target nucleic acid sequence of interest with at least one a replacement-sequence, by site specific recombination of DNA in at least one eukaryotic cell, said method comprising the step of contacting said cell with:
(a) at least one nucleic acid molecule or nucleic acid cassette comprising said at least one replacement-sequence, wherein said replacement sequence is flanked by a first and a second Int recognition sites, said first site attP1, comprises a first overlap sequence O1 and said second site attP2, comprises a second overlap sequence O2, wherein said first O1 and said second O2 overlap sequences are different, each consisting of seven nucleotides, said O1 is identical to an overlap sequence O1 comprised within a first Int recognition site attE1 in said eukaryotic cell and said O2 is identical to an overlap sequence O2 comprised within a second Int recognition site attE2 in said eukaryotic cell, said eukaryotic recognition sites attE1 and attE2 flank said target nucleic acid sequence of interest or any fragment thereof in said eukaryotic cell; and
(b) at least one HK-Int variant and/or mutated molecule or any functional fragments or peptides thereof, any nucleic acid molecule comprising a sequence encoding said HK-Int variant or any vector, vehicle, matrix, nano- or micro-particle comprising the same, said variant comprise at least one substituted amino acid residue in at least one of the CB, ND and CD domains of said HK-Int; or any kit or system or composition comprising (a) and (b); thereby allowing replacement of said target nucleic acid sequence of interest flanked by said attE1 and attE2 recognition sites, with said replacement sequence in said eukaryotic cell.

35. (canceled)

36. The method according to claim 34, wherein at least one of:
(a) said HK-Int variant comprises the amino acid sequence as denoted by at least one of SEQ ID NO. 14, SEQ ID NO. 182, SEQ ID NO. 184, SEQ ID NO. 83, SEQ ID NO. 85, SEQ ID NO. 87, SEQ ID NO. 89, SEQ ID NO. 185, SEQ ID NO. 42, SEQ ID NO. 44, SEQ ID NO. 48, SEQ ID NO. 180, SEQ ID NO. 188, SEQ ID NO. 190, SEQ ID NO. 192, and SEQ ID NO. 193, or any functional fragments, variants, fusion proteins or derivatives thereof;
(b) said nucleic acid sequence encoding said HK-Int variant comprises the nucleic acid sequence as denoted by any one of SEQ ID NO. 15, SEQ ID NO. 183, SEQ ID NO. 43, SEQ ID NO. 45, SEQ ID NO. 47, SEQ ID NO. 49, SEQ ID NO. 82, SEQ ID NO. 84, SEQ ID NO. 86, SEQ ID NO. 88, SEQ ID NO. 186, SEQ ID NO. 187, SEQ ID NO. 193 and SEQ ID NO. 224, or any functional fragments, variants, or derivatives thereof;
(c) said first overlap sequence O1 and said second overlap sequence O2 comprise a nucleic acid sequence as denoted by any one of SEQ ID NO. 98, SEQ ID NO. 99, SEQ ID NO. 127, SEQ ID NO. 128, SEQ ID NO. 117, SEQ ID NO. 70, SEQ ID NO. 71, SEQ ID NO. 73, SEQ ID NO. 131, SEQ ID NO. 132, SEQ ID NO. 104, SEQ ID NO. 105, SEQ ID NO. 94, SEQ ID NO. 95, SEQ ID NO. 109, SEQ ID NO. 111, SEQ ID NO. 113 and SEQ ID NO. 115, and wherein said O₁ and said O₂ are different; and
(d) wherein said replacement-sequence comprises a nucleic acid sequence that differs in at least one nucleotide from said target nucleic acid sequence of interest or any fragments thereof.

37-39. (canceled)

40. The method according to claim 34, wherein said target nucleic acid sequence of interest in said eukaryotic cell comprises, or is comprised within, any one of:
(a) the human CFTR gene, said nucleic acid sequence of interest is flanked by a first Int recognition site attE1 comprising the nucleic acid sequence as denoted by SEQ ID NO. 96 and a second Int recognition site attE2 comprising the nucleic acid sequence as denoted by any one of SEQ ID NO. 97, and wherein said O1 comprises the nucleic acid sequence as denoted by SEQ ID NO. 98 and said O2 comprises the nucleic acid sequence as denoted by SEQ ID NO. 99;
(b) the human CTNS gene or any fragment thereof, said nucleic acid sequence of interest is flanked by a first Int recognition site attE₁ comprising the nucleic acid sequence as denoted by SEQ ID NO. 116 and a second Int recognition site attE₂ comprising the nucleic acid sequence as denoted by SEQ ID NO. 72, and wherein said O₁ comprises the nucleic acid sequence as denoted by SEQ ID NO. 117 and said O₂ comprises the nucleic acid sequence as denoted by SEQ ID NO. 73;
(c) the human SCN1A gene or any fragment thereof, said nucleic acid sequence of interest is flanked by a first Int recognition site attE₁ comprising the nucleic acid sequence as denoted by SEQ ID NO. 120 and a second Int recognition site attE₂ comprising the nucleic acid sequence as denoted by SEQ ID NO. 121, and wherein said O₁ comprises the nucleic acid sequence as denoted by SEQ ID NO. 104 and said O₂ comprises the nucleic acid sequence as denoted by SEQ ID NO. 105; and
(d) the human DMD gene or any fragment thereof, said nucleic acid sequence of interest is flanked by a first Int recognition site attE1 comprising the nucleic acid sequence as denoted by SEQ ID NO. 92 and a second Int recognition site attE2 comprising the nucleic acid sequence as denoted by SEQ ID NO. 93, and wherein said O1 comprises the nucleic acid sequence as denoted by SEQ ID NO. 94 and said O2 comprises the nucleic acid sequence as denoted by SEQ ID NO. 95.

41. The method according to claim 34, wherein the method is for curing or treating, preventing, inhibiting, reducing, eliminating, protecting or delaying the onset of a genetic disorder or condition in a subject in need thereof by replacing at least one target nucleic acid sequence of interest with at least one a replacement-sequence in at least one cell in said subject, wherein said step of contacting the cell is performed by, the steps of administering to said subject an effective amount of at least one of:
(i) (a) at least one nucleic acid molecule or nucleic acid cassette comprising a replacement-sequence for at least one target nucleic acid sequence of interest, said replacement sequence is flanked by a first and a second Int recognition sites, said first site attP₁, comprises a first overlap sequence O₁ and said second site attP₂, comprises a second overlap sequence O₂, wherein said first O₁ and said second O₂ overlap sequences are different, each consisting of seven nucleotides, said O₁ is identical to an overlap sequence O₁ comprised within a first Int recognition site attE₁ in at least one cell of said subject, and said O₂ is identical to an overlap sequence O₂ comprised within a second Int recognition site attE₂ in said cell, said recognition sites attE₁ and attE₂ flank said target nucleic acid sequence of interest or any fragment thereof in said cell; and (b) at least one HK-Int variant and/or mutated molecule or any functional fragments or peptides thereof, any nucleic acid molecule comprising a sequence encoding said HK-Int variant or any vector, vehicle, matrix, nano- or micro-particle comprising the same, wherein said HK-Int variant comprises at least one substituted amino acid residue in at least one of the CB, ND and the CD of the Wild type HK-Int molecule;
(ii) at least one kit and/or system or composition comprising (a) and (b); and
(iii) at least one cell comprising the nucleic acid molecule or nucleic acid cassette of (a), and at least one HK-Int variant or nucleic acid molecule encoding said Int variant of (b), or any system, kit or composition thereof;
thereby allowing replacement of said at least one target nucleic acid sequence of interest flanked by said attE₁ and attE₂ sites, with said replacement sequence, in said subject, or in at least one cell of said subject.

42. (canceled)

43. The method according to claim 41, wherein at least one of:
(a) said HK-Int variant and/or mutated molecule comprises the amino acid sequence as denoted by any one of SEQ ID NO. 14, SEQ ID NO. 182, SEQ ID NO. 184, SEQ ID NO. 83, SEQ ID NO. 85, SEQ ID NO. 87, SEQ ID NO. 89, SEQ ID NO. 185, SEQ ID NO. 42, SEQ ID NO. 44, SEQ ID NO. 48, SEQ ID NO. 180, SEQ ID NO. 188, SEQ ID NO. 190, SEQ ID NO. 192, and SEQ ID NO. 193, or any functional fragments, variants, fusion proteins or derivatives thereof;
(b) said first overlap sequence O₁ and said second overlap sequence O₂ comprise a nucleic acid sequence as denoted by any one of SEQ ID NO. 98, SEQ ID NO. 99, SEQ ID NO. 127, SEQ ID NO. 128, SEQ ID NO. 117, SEQ ID NO. 70, SEQ ID NO. 71, SEQ ID NO. 73, SEQ ID NO. 131, SEQ ID NO. 132, SEQ ID NO. 104, SEQ ID NO. 105, SEQ ID NO. 94, SEQ ID NO. 95, SEQ ID NO. 109, SEQ ID NO. 111, SEQ ID NO. 113 and SEQ ID NO. 115, and wherein said O₁ and said O₂ are different; and
(c) said replacement-sequence comprise a nucleic acid sequence that differs in at least one nucleotide from said target nucleic acid sequence of interest or any fragments thereof.

44-45. (canceled)

46. The method according claim 41, wherein said genetic disorder or condition is a hereditary disease or condition associated with a single gene disorder or with a polygenic disorder, wherein said hereditary disease or condition is any one of Cystic Fibrosis (CF), Cystinosis, SCN1A-related seizure disorders and Duchenne Muscular Dystrophy (DMD), and wherein at least one of:
(a) said genetic disorder or condition is CF, and wherein said target nucleic acid sequence of interest comprises or is comprised within the human CFTR gene or any fragment thereof, said target nucleic acid sequence of interest is flanked by a first Int recognition site attE1 comprising the nucleic acid sequence as denoted by SEQ ID NO. 96 and a second Int recognition site attE2 comprising the nucleic acid sequence as denoted by SEQ ID NO. 97, and wherein said O1 comprises the nucleic acid sequence as denoted by SEQ ID NO. 98 and said O2 comprises the nucleic acid sequence as denoted by SEQ ID NO. 99;
(b) said genetic disorder or condition is Cystinosis, and wherein said target nucleic acid sequence of interest comprises or is comprised within the human CTNS gene or any fragment thereof, said target nucleic acid sequence of interest is flanked by a first Int recognition site attE₁ comprising the nucleic acid sequence as denoted by SEQ ID NO. 116 and a second Int recognition site attE₂ comprising the nucleic acid sequence as denoted by SEQ ID NO. 72, and wherein said O₁ comprises the nucleic acid sequence as denoted by SEQ ID NO. 117 and said O₂ comprises the nucleic acid sequence as denoted by SEQ ID NO. 73;
(c) said genetic disorder or condition is at least one SCN1A-related seizure disorder, and wherein said target nucleic acid sequence of interest comprises or is comprised within the human SCN1A gene or any fragment thereof, said target nucleic acid sequence of interest is flanked by a first Int recognition site attE₁ comprising the nucleic acid sequence as denoted by SEQ ID NO. 120 and a second Int recognition site attE₂ comprising the nucleic acid sequence as denoted by any one of SEQ ID NO. 121, and wherein said O₁ comprises the nucleic acid sequence as denoted by SEQ ID NO. 104 and said O₂ comprises the nucleic acid sequence as denoted by SEQ ID NO. 105; and
(d) said genetic disorder or condition is DMD, and wherein said target nucleic acid sequence of interest comprises or is comprised within the human DMD gene or any fragment thereof, said target nucleic acid sequence of interest is flanked by a first Int recognition site attE1 comprising the nucleic acid sequence as denoted by SEQ ID NO. 92 and a second Int recognition site attE2 comprising the nucleic acid sequence as denoted by SEQ ID NO. 93, and wherein said O1 comprises the nucleic acid sequence as denoted by SEQ ID NO. 94 and said O2 comprises the nucleic acid sequence as denoted by SEQ ID NO. 95.

47-55. (canceled)
